(19) United States
(12) Patent Application Publication
(10) Pub. No.: US 2024/0199644 A1
(43) Pub. Date: Jun. 20, 2024

(54) Fused Ring Derivatives Useful as KRAS G12D Inhibitors

(71) Applicant: JACOBIO PHARMACEUTICALS CO., LTD., Beijing (CN)

(72) Inventors: Huifeng HAN, Beijing (CN); Panliang GAO, Beijing (CN); Cunbo MA, Beijing (CN); Peng WANG, Beijing (CN); Wei LONG, Beijing (CN); Hao ZHANG, Beijing (CN); Lei ZHANG, Beijing (CN); Dan LIU, Beijing (CN)

(21) Appl. No.: 18/280,487

(22) PCT Filed: Mar. 7, 2022
(86) PCT No.: PCT/CN2022/079476

§ 371 Date: Sep. 6, 2023

(30) Foreign Application Priority Data
Mar. 7, 2021 (WO) PCT/CN2021/079457
Jun. 18, 2021 (WO) PCT/CN2021/100923
Sep. 27, 2021 (WO) PCT/CN2021/120980
Jan. 11, 2022 (WO) PCT/CN2022/071298
Jan. 21, 2022 (WO) PCT/CN2022/073228

(51) Int. Cl. C07D 498/16; A61K 31/553; C07D 498/22; C07D 519/00

(52) U.S. Cl. CPC C07D 498/16; A61K 31/553; C07D 498/22; C07D 519/00

(57) ABSTRACT

The invention relates to a KRAS G12D inhibitors of formula (I) or formula (IV), a composition containing the inhibitor and the use thereof

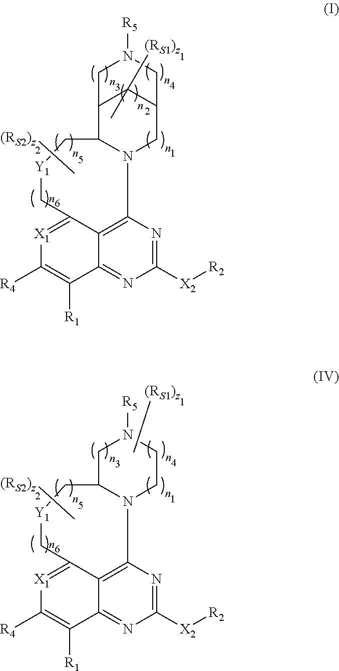

### CROSS-REFERENCES TO RELATED APPLICATIONS

This application claims priority to PCT/CN2021/079457, filed Mar. 7, 2021; PCT/CN2021/100923, filed Jun. 18, 2021; PCT/CN2021/120980, filed Sep. 27, 2021; PCT/CN2022/071298, filed Jan. 11, 2022; and PCT/CN2022073228, filed Jan. 21, 2022, each of which is incorporated herein in its entirety and for all purposes.

### TECHNICAL FIELD

The invention relates to fused ring derivatives useful as KRAS G12D inhibitors, a composition containing the inhibitor and the use thereof.

### BACKGROUND ART

Cancer related KRAS mutation is hard to cure. Some progresses have been taken recently after many years of efforts, for example some promising clinical data have been reported when using Amg-510 and MRT-849 as the therapeutic agent. However, these compounds are directed to KRAS G12C mutation, the development of KRAS G12D (glycine to aspartic acid) inhibitors is extraordinarily hard. Thus, there remains a need in the art for improved compounds and methods for treating KRAS G12D mutated cancer. The present invention fulfills this need and provides other related advantages.

### SUMMARY OF INVENTION

Provided herein is the following aspects:

[1]. A compound of formula (I) or formula (IV), a stereoisomer thereof, an atropisomer thereof, a pharmaceutically acceptable salt thereof, a pharmaceutically acceptable salt of the stereoisomer thereof, or a pharmaceutically acceptable salt of the atropisomer thereof:

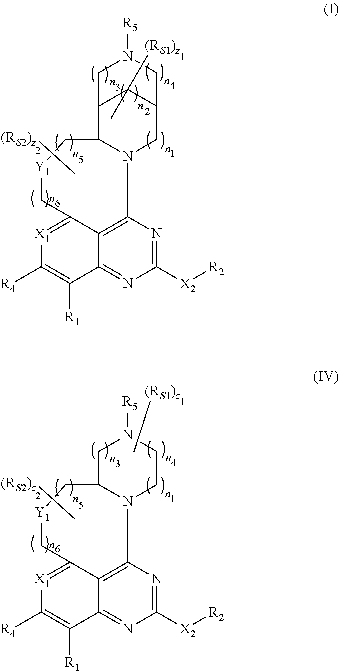

wherein,
R₁ is selected from hydrogen, halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl; wherein, said —C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
Y₁ is selected from NH, O, S, SO, SO₂, NHCO, CONH, NHSO₂ or SO₂NH; when Y₁ is selected from NH, NHCO, CONH, NHSO₂ or SO₂NH, each of which is optionally substituted with R_{S2};
X₁ is selected from CR₃ or N;
n₁, n₂, n₃, n₄, n₅ and n₆ are each independently selected from 0, 1, 2, 3, 4, 5 or 6;
R₃ is selected from hydrogen, halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl; wherein said —C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
X₂ is selected from CR₂₁R₂₂, NR₂₃, O, S, SO or SO₂;
R₂₁ and R₂₂ are each independently selected from hydrogen, halogen, —C₁₋₆alkyl, —OH, —OC₁₋₆alkyl, —SH, —SC₁₋₆alkyl, —NH₂, —NH(C₁₋₆alkyl) or —N(C₁₋₆alkyl)₂;
R₂₃ is selected from hydrogen or —C₁₋₆alkyl;
R₂ is selected from

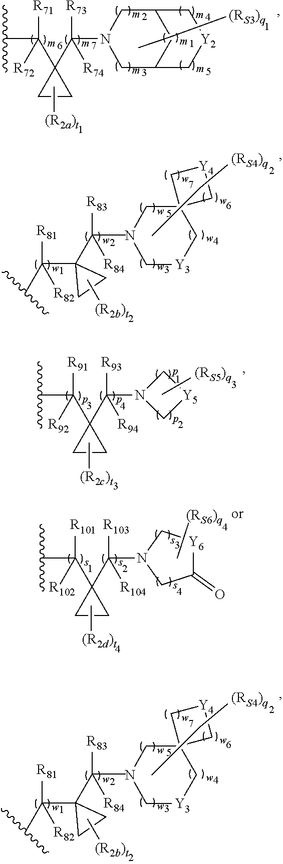

-L-(3-12 membered heterocyclyl), -L-(3-12 membered cycloalkyl), -L-(6-12 member aryl), -L-(5-12 membered heteroaryl) or -L-NR₂₄R₂₅;
Each L is independently selected from a bond or C₁₋₁₀ alkylene optionally substituted with one or more R_{S9};
R₂₄ and R₂₅ are each independently selected from hydrogen or —C₁₋₁₀alkyl optionally substituted with one or more R_{S10};
Said 3-12 membered heterocyclyl in -L-(3-12 membered heterocyclyl) is optionally substituted with one or more R_{S11};
Said 3-12 membered cycloalkyl in -L-(3-12 membered cycloalkyl) is optionally substituted with one or more R_{S12};
Said 6-12 member aryl in -L-(6-12 member aryl) is optionally substituted with one or more R_{S13};
Said 5-12 membered heteroaryl in -L-(5-12 membered heteroaryl) is optionally substituted with one or more R_{S14};
Y₂ is selected from O, S, SO, SO₂, C═O, NH or CH₂, when Y₂ is selected from NH or CH₂, the NH or CH₂ is optionally substituted with 1 or 2 R_{S3};
m₁, m₂, m₃, m₄ or m₅ is independently selected from 0, 1, 2, 3, 4, 5 or 6;
m₆ or m₇ is independently selected from 0, 1, 2, 3, 4, 5 or 6;
Y₃ and Y₄ are each independently selected from O, S, SO, SO₂, C═O, NH or CH₂, when Y₃ and Y₄ is selectd from NH or CH₂, the NH or CH₂ is optionally substituted with 1 or 2 R_{S4};
w₁ and w₂ are each independently selected from 0, 1, 2, 3, 4, 5 or 6;
w₃, w₄, w₅, w₆ and w₇ are each independently selected from 0, 1, 2, 3, 4, 5 or 6; provided that w₆ and w₇ are not 0 at the same time;
Y₅ is selected from O, S, SO, SO₂, C═O, NH or CH₂, when Y₅ is selected from NH or CH₂, the NH or CH₂ is optionally substituted with 1 or 2 R_{S5};
p₁ and p₂ are each independently selected from 0, 1, 2, 3, 4, 5, or 6, provided that p₁ and p₂ are not 0 at the same time;
p₃ and p₄ are each independently selected from 0, 1, 2, 3, 4, 5 or 6;
Y₆ is selected from O, S, NH or CH₂, when Y₆ is selected from NH or CH₂, the NH or CH₂ is optionally substituted with 1 or 2 R_{S6};
s₁ and s₂ are each independently selected from 0, 1, 2, 3, 4, 5 or 6;
s₃ and s₄ are each independently selected from 0, 1, 2, 3, 4, 5, or 6, provided that s₃ and s₄ are not 0 at the same time;
r₁ and r₂ are each independently selected from 0, 1, 2, 3, 4, 5 or 6;
r₃ and r₄ are each independently selected from 0, 1, 2, 3, 4, 5, or 6;
v is selected from 0, 1, 2, 3, 4, 5 or 6;
ring A is selected from a 3-10 membered carbocyclic ring, a 3-10 membered heterocyclic ring, a 6-10 membered aryl ring or a 5-10 membered heteroaryl ring, said heterocyclic or heteroaryl ring at each occurrence is independently contains 1, 2, 3 or 4 heteroatoms selected from N, O or S;
Ring B and ring C are each independently selected from a 3-10 membered heterocyclic ring which is optionally further contains 1, 2, or 3 heteroatoms selected from N, O or S except the fused N atom;
R_{S1}, R_{S2}, R_{S3}, R_{S4}, R_{S5}, R_{S6}, R_{S7}, R_{S8}, R_{S9}, R_{S10}, R_{S11}, R_{S12}, R_{S13} and R_{S14} are each independently selected from halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), 13 S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein, said —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally substituted with one or more R_{Sa};
Each of R_{Sa} is independently selected from halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —NH₂, —NH(C₁₋₆alkyl), —N(C₁₋₆alkyl)₂, —OH, —(C₁₋₆alkyl), —SH, —S(C₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), (═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, phenyl or 5-6 membered heteroaryl; wherein said 3-6 membered cycloalkyl, 3-6 membered heterocyclyl, phenyl or 5-6 membered heteroaryl is independently optionally substituted with 1, 2, or 3 R_{Sb};
Each of R_{Sb} is independently selected from halogen; —C₁₋₆alkyl; haloC₁₋₆alkyl; —CN; —OH; —NH₂; —NH(C₁₋₆alkyl); —NH(C₁₋₆alkyl)₂; —OC₁₋₆alkyl; or —C₁₋₆alkyl substituted with 1, 2 or 3 substituents selected form halogen, haloC₁₋₆alkyl, —CN, —OH, —NH₂, —NH(C₁₋₆alkyl), —NH(C₁₋₆alkyl)₂ or —OC₁₋₆alkyl;
q₁, q₂, q₃, q₄, q₅ and q₆ are each independently selected from 0, 1, 2, 3, 4, 5 or 6;
R₂ₐ, R_{2b}, R_{2c}, R_{2d} and R₂ₑ are each independently selected from is selected from halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3 -10 membered cycloalkyl, 3-10 membered heterocyclyl, 6-10 membered aryl or 5-10 membered heteroaryl; wherein, said —C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), (═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
t₁, t₂, t₃, t₄ and t₅ are each independently selected from 0, 1, 2, 3, 4, 5 or 6;
R₆₁, R₆₂, R₇₁, R₇₂, R₇₃, R₇₄, R₈₁, R₈₂, R₈₃, R₈₄, R₉₁, R₉₂, R₉₃, R₉₄, R₁₀₁, R₁₀₂, R₁₀₃, R₁₀₄, R₁₁₁, R₁₁₂, R₁₁₃ and R₁₁₄ are each independently selected from are independently selected from hydrogen, halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3 -10 membered cycloalkyl, 3-10 membered heterocyclyl, 6-10 membered aryl or 5-10 membered heteroaryl; wherein, said —C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C (═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
R₄ is selected from phenyl, naphthyl, 5 membered heteroaryl, 6 membered heteroaryl, 9 membered heteroaryl or 10 membered heteroaryl, wherein said R₄ is independently optionally substituted with 1, 2, 3, 4, 5, or 6 R₄ₐ;
Each of R₄ₐ is independently selected from halogen, —C₁₋₆alkyl, haloC₁₋₆alkyl, haloC₁₋₆alkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl, 5-10 membered heteroaryl or R₄₁, wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered heterocyclyl, 6-10 membered aryl or 5-10 membered heteroaryl is independently optionally substituted with 1, 2 or 3 R_{4b};
Each of R_{4b} is independently selected from halogen; —C₁₋₆alkyl; haloC₁₋₆alkyl; —CN; oxo; —OH; —NH₂; —NH(C₁₋₆alkyl); —NH(C₁₋₆alkyl)₂; —OC₁₋₆alkyl; or —C₁₋₆alkyl substituted with 1, 2 or 3 substituents selected form halogen, haloC₁₋₆alkyl, —CN, —OH, —NH₂, —NH(C₁₋₆alkyl), —NH(C₁₋₆alkyl)₂ or —OC₁₋₆alkyl;
R₄₁ is selected from

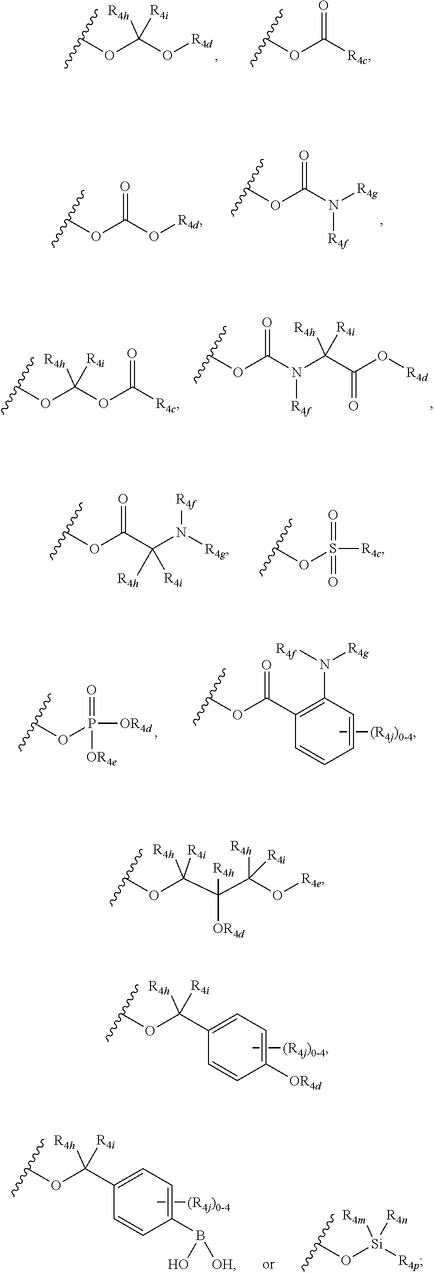

R_{4c} is selected from hydrogen, —C₁₋₃₀alkyl, —C₂₋₃₀alkenyl, —C₂₋₃₀alkynyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₄ⱼ;
R_{4d} and R₄ₑ are each selected from hydrogen, —C₁₋₃₀alkyl, —C₂₋₃₀alkenyl, —C₂₋₃₀alkynyl, —C(═O)C₁₋₆alkyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₄ⱼ;
R_{4f} and R_{4g} are each selected from hydrogen, —C₁₋₃₀alkyl, —C₂₋₃₀alkenyl, —C₂₋₃₀alkynyl, —C(═O)C₁₋₆alkyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₄ⱼ;
R₄ₕ, R₄ᵢ, R₄ₘ, R₄ₙ and R₄ₚ are each selected from hydrogen, halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3 -10 membered cycloalkyl, 3-10 membered heterocyclyl, 6-10 membered aryl or 5-10 membered heteroaryl; wherein, said -C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C (═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
Optionally, R_{4f} and R_{4g} together with the atoms to which they are respectively attached form a 4-10 membered heterocyclyl ring, said 4-10 membered heterocyclyl ring optionally further contains 1 or 2 heteratoms selected from N, O, S, S(═O) or S(═O)₂ and optionally substituted with one or more R₄ⱼ;
Optionally, R_{4f} and R₄ₕ together with the atoms to which they are respectively attached form a 4-10 membered heterocyclyl ring, said 4-10 membered heterocyclyl ring optionally further contains 1 or 2 heteratoms selected from N, O, S, S(═O) or S(═O)₂ and optionally substituted with one or more R₄ⱼ;
R₄ⱼ at each occurrence is independently selected from halogen, —C₁₋₆alkyl, haloC₁₋₆alkyl, haloC₁₋₆alkoxy, —C₂₋₆alkenyl, —C₂₋₆alkynyl, —CN, oxo, —NO₂, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3 -10 membered cycloalkyl, 3-10 membered heterocyclyl, 6-10 membered aryl, or 5-10 membered heteroaryl, wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered heterocyclyl, 6-10 membered aryl or 5-10 membered heteroaryl is independently optionally substituted with 1, 2 or 3 substituents selected from halogen; —C₁₋₆alkyl; haloC₁₋₆alkyl; —CN; oxo; —OH; —NH₂; —NH(C₁₋₆alkyl); —NH(C₁₋₆alkyl)₂; —OC₁₋₆alkyl; or —C₁₋₆alkyl substituted with 1, 2 or 3 substituents selected form halogen, haloC₁₋₆alkyl, —CN, —OH, —NH₂, —NH(C₁₋₆alkyl), —NH(C₁₋₆alkyl)₂ or —OC₁₋₆alkyl;
R₅ is selected from hydrogen or

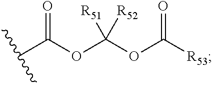

R₅₁ and R₅₂ are each independently selected from are independently selected from hydrogen, halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C (═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3 -10 membered cycloalkyl, 3-10 membered heterocyclyl, 6-10 membered aryl or 5-10 membered heteroaryl; wherein, said —C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
R₅₃ is selected from hydrogen, —C₁₋₃₀alkyl, —C₂₋₃₀alkenyl, —C₂₋₃₀alkynyl, —C(═O)C₁₋₆alkyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₅ₐ;
R₅ₐ at each occurrence is independently selected from halogen, —C₁₋₆alkyl, haloC₁₋₆alkyl, haloC₁₋₆alkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
z₁ and z₂ are each independently selected from 0, 1, 2, 3, 4, 5 or 6;
Each of (heterocyclyl and heteroaryl) at each occurrence is independently contain 1, 2, 3 or 4 heteroatoms selected from N, O, S, S(═O) or S(═O)₂.

[2]. The compound of aspect [1], wherein, the compound is any one of the formulas in the following Table 1:

TABLE 1

| | |
|---|---|
| 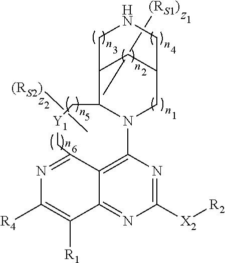 | (I-1) |
| 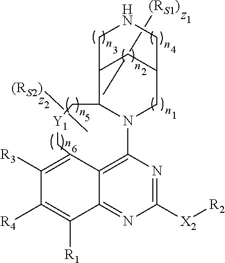 | (I-2) |
| 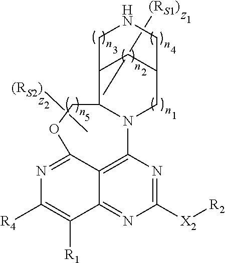 | (I-1a) |
| 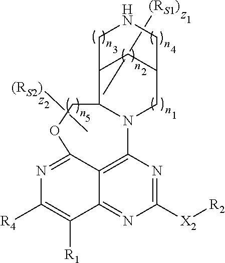 | (I-2a) |
| 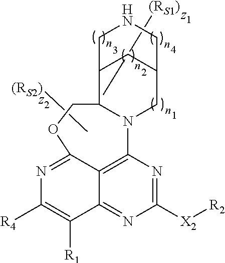 | (II-1a) |
| 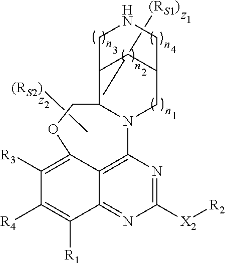 | (II-2a) |
| 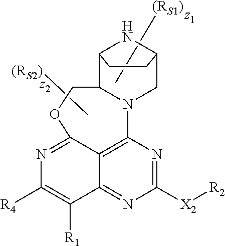 | (III-1a) |
| 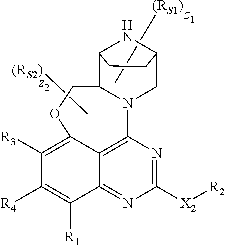 | (III-2a) |
| 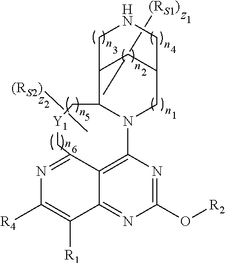 | (A) |
| 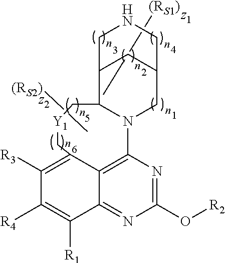 | (B) |
| 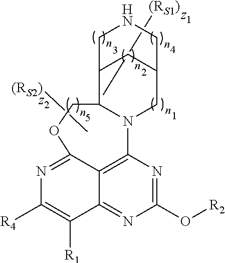 | (C) |
| 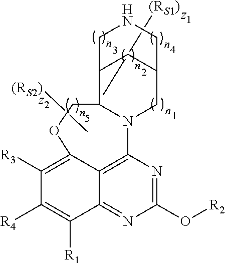 | (D) |
| 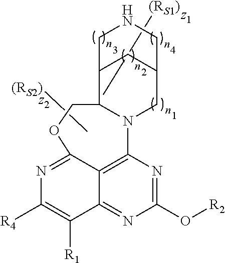 | (E) |
| 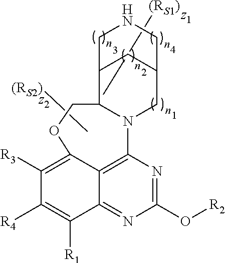 | (F) |
| 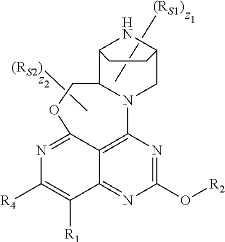 | (G) |
| 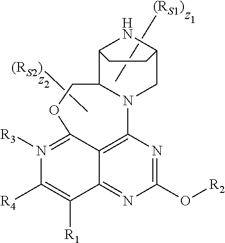 | (H) |
| 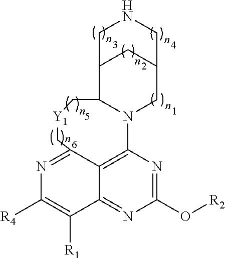 | (A-1) |
| 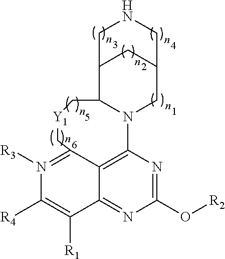 | (B-1) |
| 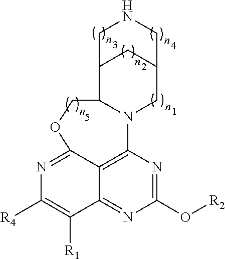 | (C-1) |
| 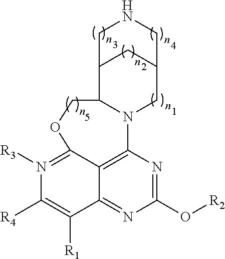 | (D-1) |
| 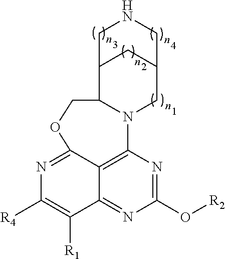 | (E-1) |
| 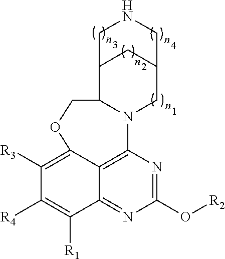 | (F-1) |
| 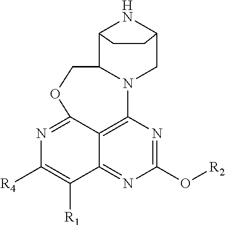 | (G-1) |
| 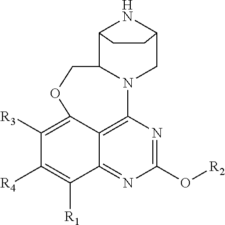 | (H-1) |
| 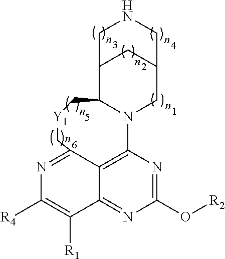 | (a-1) |
| 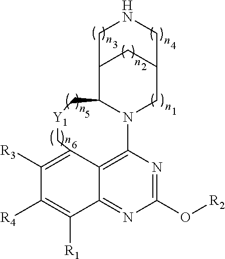 | (b-1) |
| 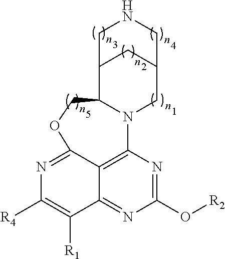 | (c-1) |
| 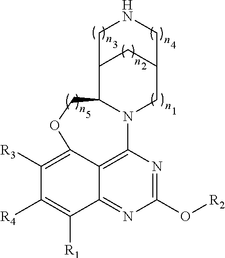 | (d-1) |
| 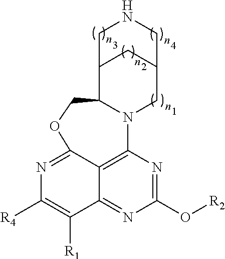 | (e-1) |
| 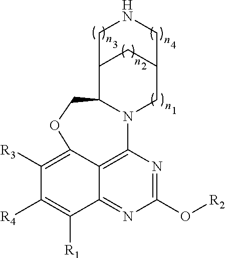 | (f-1) |
| 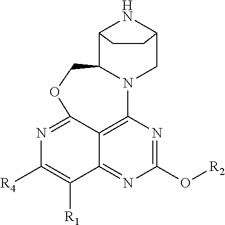 | (g-1) |
| 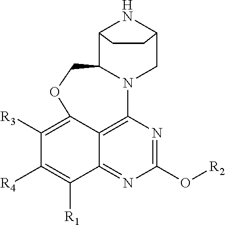 | (h-1) |
| 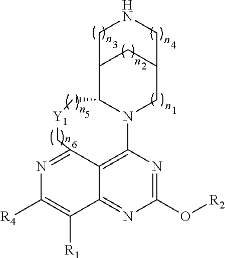 | (a-2) |
| 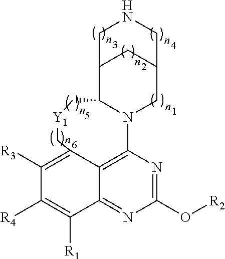 | (b-2) |
| 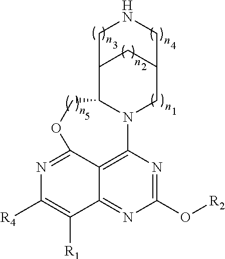 | (c-2) |
| 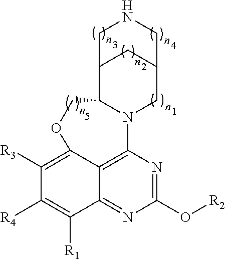 | (d-2) |
| 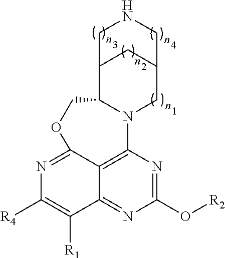 | (e-2) |
| 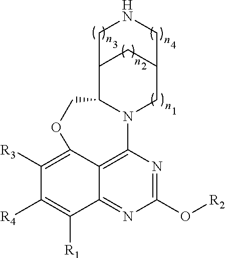 | (f-2) |
| 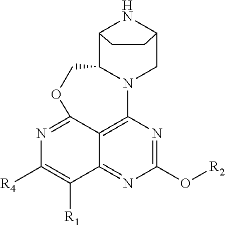 | (g-2) |
| 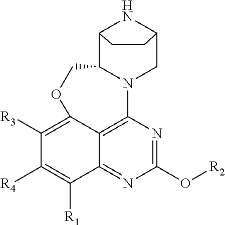 | (h-2) |
| 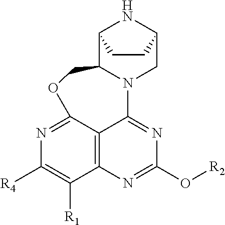 | (i-1) |
| 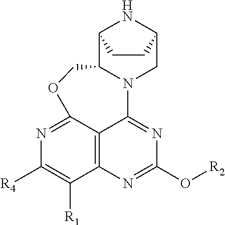 | (i-2) |
| 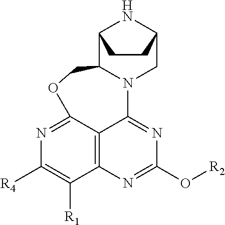 | (i-3) |
| 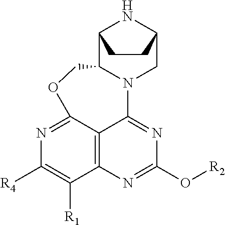 | (i-4) |
| 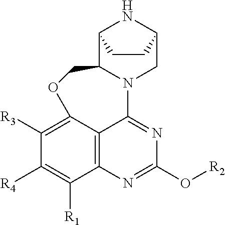 | (i-5) |
| 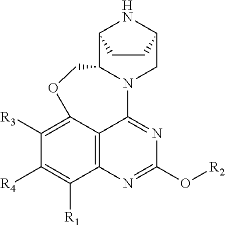 | (i-6) |
| 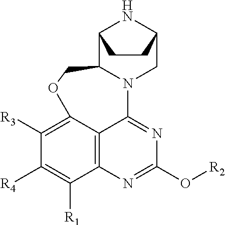 | (i-7) |
| 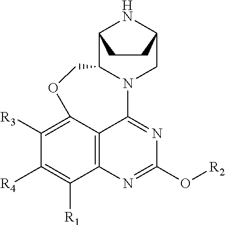 | (i-8) |
| 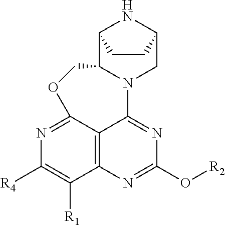 | (ii-1) |
| 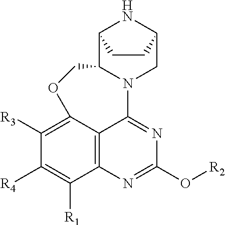 | (ii-2) |
| 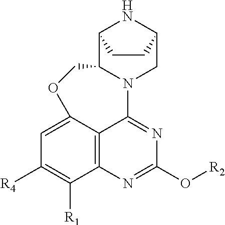 | (iii-1) |
| 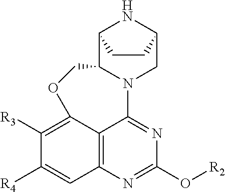 | (iii-2) |
| 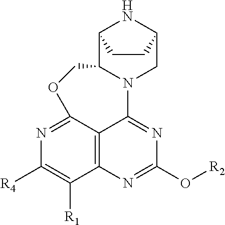 | (iii-3) |
| 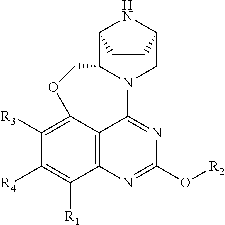 | (iii-4) |
| 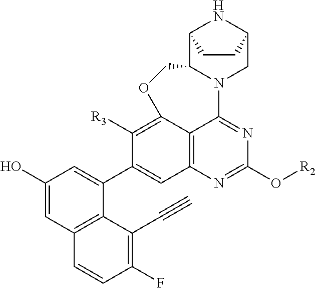 | (iii-5) |
| 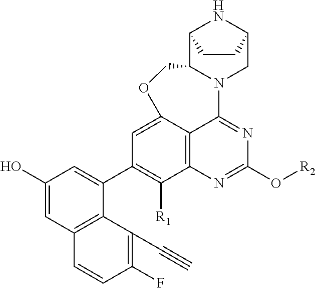 | (iii-6) |
| 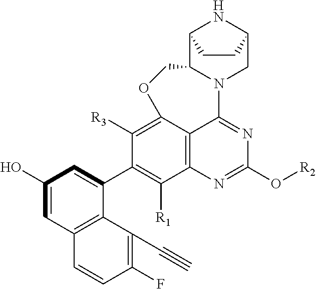 | (iii-7) |
| 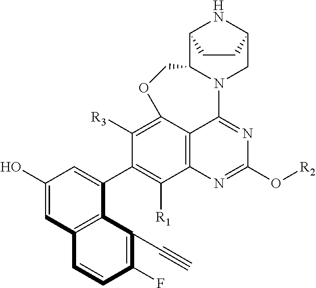 | (iii-8) |
| 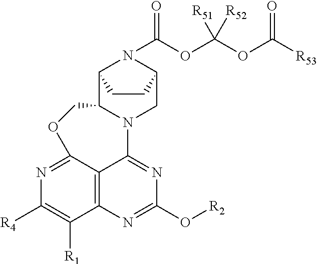 | (i-2a) |
| 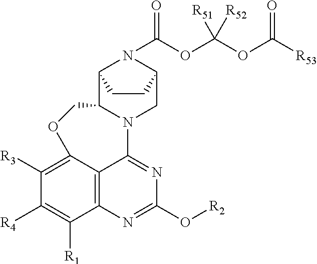 | (i-6a) |

[3]. The compound of aspect [1] or [2], wherein, R₁ is selected from hydrogen, —F, —Cl, —Br, —C₁₋₃alkyl, haloC₁₋₃alkyl, haloC₁₋₃alkoxy, —C₂₋₃alkenyl, —C₂₋₃alkynyl, —CN, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C (═O)(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), 6-10 membered cycloalkyl, 6-10 membered heterocyclyl, 6-8 membered aryl or 5-8 membered heteroaryl.

[4]. The compound of any one of aspects [1] to [3], wherein, R₁ is selected from —H, —F, —Cl, —CH₃, —CH₂CH₃, —CN, —COOH, —CH₂OH, —OH, —OCH₃, —OCH₂CH₃, —CF₃, —CHF₂, —NH₂, —NHCH₃, —N(CH₃)₂, —CH₂NH₂, —CH₂CH₂NH₂, —CH₂OH, —CH₂CH₂OH, —SH, —S—CH₃, —CH₂SH, —CH₂CH₂SH, —CH═CH₂, —C≡CH, —CHCH═CH₂, —OCF₃, —OCHF₂, —C(═O)NH₂, —C(═O)OCH₃,

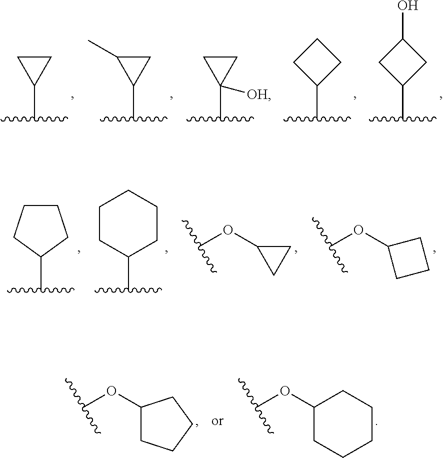

[5]. The compound of any one of aspects [1] to [4], wherein, R₁ is —H or —F.

[6]. The compound of any one of aspects [1] to [5], wherein, R₃ is selected from hydrogen, —F, —Cl, —Br, —C₁₋₃alkyl, haloC₁₋₃alkyl, haloC₁₋₃alkoxy, —C₂₋₃alkenyl, —C₂₋₃alkynyl, —CN, oxo, —NH₂, —NH(C₁₋₃alkyl), —N(C₁₋₃alkyl)₂, —OH, —O(C₁₋₃alkyl), —SH, —S(C₁₋₃alkyl), —S(haloC₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), 3-10 membered cycloalkyl, 3-10 membered heterocyclyl, 6-10 membered aryl or 5-8 membered heteroaryl.

[7]. The compound of any one of aspects [1] to [6], wherein, R₃ is selected from —H, —F, —Cl, —CH₃, —CH₂CH₃, —CH(CH₃)₂, —CN, —COOH, —CH₂OH, —OH, —OCH₃, —OCH₂CH₃, —CF₃, —CHF₂, —NH₂, —NHCH₃, —N(CH₃)₂, —CH₂NH₂, —CH₂CH₂NH₂, —CH₂OH, —CH₂CH₂OH, —SH, —S—CH₃, —S—CF₃, —CH₂SH, —CH₂CH₂SH, —CH═CH₂, —CH≡CH═CH₂, —OCF₃, —OCHF₂, —C(═O)NH₂, —C(═O)OCH₃,

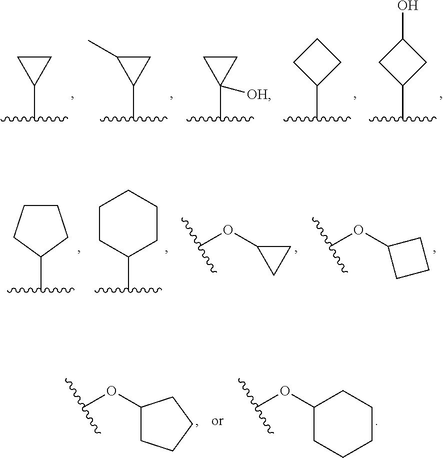

[8]. The compound of any one of aspects [1] to [7], wherein, R₃ is selected from —H, —F, —Cl, —CH₃, —CH(CH₃)₂, —CF₃, —S—CF₃ or

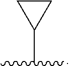

[9]. The compound of any one of aspects [1] to [8], wherein, R₃ is selected from —H.

[10]. The compound of any one of aspects [1] to [9], wherein, the moiety of —X₂—R₂ or —O—R₂ is selected from

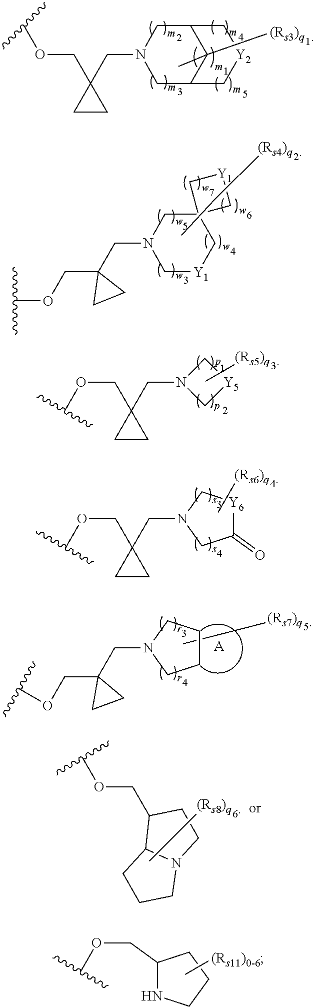

Y₂ is selected from O, S, SO, SO₂, C═O, NH or CH₂, when Y₂ is selected from NH or CH₂, the NH or CH₂ is optionally substituted with 1 or 2 R_{S3};
m₁, m₂, m₃, m₄or m₅ is independently selected from 0, 1, 2, 3, 4, 5 or 6;
Y₃ and Y₄ are each independently selected from O, S, SO, SO₂, C═O, NH or CH₂, when Y₃ and Y₄ is selectd from NH or CH₂, the NH or CH₂ is optionally substituted with 1 or 2 R_{S4};
w₃, w₄, w₅, w₆ and w₇ are each independently selected from 0, 1, 2, 3, 4, 5 or 6; provided that w₆ and w₇ are not 0 at the same time;
Y₅ is selected from O, S, SO, SO₂, C═O, NH or CH₂, when Y₅ is selected from NH or CH₂, the NH or CH₂ is optionally substituted with 1 or 2 R_{S5};
p₁ and p₂ are each independently selected from 0, 1, 2, 3, 4, 5, or 6, provided that p₁ and p₂ are not 0 at the same time;
Y₆ is selected from O, S, NH or CH₂, when Y₆ is selected from NH or CH₂, the NH or CH₂ is optionally substituted with 1 or 2 R_{S6};
s₃ and s₄ are each independently selected from 0, 1, 2, 3, 4, 5, or 6, provided that s₃ and s₄ are not 0 at the same time;
r₃ and r₄ are each independently selected from 0, 1, 2, 3, 4, 5, or 6;
ring A at each occurrence is independently selected from a 4 membered carbocyclic ring, 5 membered carbocyclic ring, a, 6 membered carbocyclic ring, a 4 membered heterocyclic ring including 1 ring member selected from N, a 5 membered heterocyclic ring including 1 to 2 ring members selected from N, or O, a 6 membered heterocyclic ring including 1 to 2 ring members selected from N, O or S, a phenyl ring, a 5 membered heteroaryl ring including 1 to 2 ring members selected from N, O, or S, or a 6 membered heteroaryl ring including 1 ring member selected from N.

[11]. The compound of any one of aspects [1] to [10], wherein:
Y₂ is selected from O, C═O, NH or CH₂, when Y₂ is selected from NH or CH₂, the NH or CH₂ is optionally substituted with 1 or 2 R_{S3};
m₁ is selected from 0, 1, 2 or 3; m₂ is selected from 0, 1, 2 or 3; m₃ is selected from 0, 1, 2 or 3; m₄ is selected from 0, 1, 2 or 3; m₅ is selected from 0, 1, 2 or 3;
Y₃ and Y₄ are each independently selected from O, S, SO₂, NH or CH₂, when Y₃ and Y₄ is selectd from NH or CH₂, the NH or CH₂ is optionally substituted with 1 or 2 R_{S4};
w₃ is selected from 0, 1, 2 or 3; w₄ is selected from 0, 1, 2 or 3; w₅ is selected from 0, 1, 2 or 3; w₆ is selected from 0, 1, 2 or 3; w₇ is selected from 0, 1, 2 or 3; provided that w₆ and w₇ is not 0 at the same time;
Y₅ is selected from O, S, SO₂, NH, CH₂ or C═O, when Y₅ is selected from NH or CH₂, the NH or CH₂ is optionally substituted with 1 or 2 R_{S5};
p₁ is selected from 1, 2, 3 or 4; p₂ is selected from 1, 2, 3 or 4;
Y₆ is selected from O, S or NH, when Y₆ is selected from NH or CH₂, the NH or CH₂ is optionally substituted with R_{S6};
s₁ is selected from 1, 2, 3 or 4; p₂ is selected from 1, 2, 3 or 4;
r₃ is selected from 1, 2 or 3; r₄ is selected from 1, 2 or 3;
ring A at each occurrence is independently selected from a 4 membered carbocyclic ring, 5 membered carbocyclic ring, a, 6 membered carbocyclic ring, a 4 membered heterocyclic ring including 1 ring member selected from N, a 5 membered heterocyclic ring including 1 to 2 ring members selected from N, or O, a 6 membered heterocyclic ring including 1 to 2 ring members selected from N, O or S, a phenyl ring, a 5 membered heteroaryl ring including 1 to 2 ring members selected from N, O, or S, or a 6 membered heteroaryl ring including 1 ring member selected from N.

[12]. The compound of any one of aspects [1] to [11], wherein:
Each of R_{S3} is independently selected from —F, —Cl, —CH₃, —CH₂CH₃, —CH₂CH₂CH₃, —CH(CH₃)₂, —CH₂Cl, —CHCl₂, —CCl₃, —CH₂F, —CHF₂, —CF₃, —CN, oxo, —NH₂, —NH(CH₃), —N(CH₃)₂, —OH, —O—CH₃, —O—CH₂CH₃, —SH, —S—CH₃, —S—CH₂CH₃, —COOH, —COO(CH₃), —COO(CH₂CH₃), —CH₂OH, —CH₂CH₂OH, —CH(CH₃)OH, —CH₂NH₂, —CH₂CH₂NH₂, or —CH(CH₃)NH₂; preferably, each of R_{S3} is independently selected from —F, —CH₃, —CH₂CH₃, —CF₃, —CN, oxo, —NH₂, —OH, —O—CH₃, —COOH, —COO(CH₃), —CH₂OH, or —CH₂NH₂;
q₁ is selected from 0, 1, or 2;
Each of R_{S4} is independently selected from —F, —Cl, —CH₃, —CH₂CH₃, —CH₂CH₂CH₃, —CH(CH₃)₂, —CH₂Cl, —CHCl₂, —CCl₃, —CH₂F, —CHF₂, —CF₃, —CN, oxo, —NH₂, —NH(CH₃), —N(CH₃)₂, —OH, —O—CH₃, —O—CH₂CH₃, —SH, —S—CH₃, —S—CH₂CH₃, —COOH, —COO(CH₃), —COO(CH₂CH₃), —CH₂OH, —CH₂CH₂OH, —CH(CH₃)OH, —CH₂NH₂, —CH₂CH₂NH₂, or —CH(CH₃)NH₂; preferably, each of R_{S4} is independently selected from —F, —CH₃, —CH₂CH₃, —CF₃, —CN, oxo, —NH₂, —OH, —O—CH₃, —COOH, —COO(CH₃), —CH₂OH, or —CH₂NH₂;
q₂ is selected from 0, 1, or 2;
Each of R_{S5} is independently selected from —F, —Cl, —CH₃, —CH₂CH₃, —CH₂CH₂CH₃, —CH(CH₃)₂, —CH₂Cl, —CHCl₂, —CCl₃, —CH₂F, —CHF₂, —CF₃, —CN, oxo, —NH₂, —NH(CH₃), —N(CH₃)₂, —OH, —O—CH₃, —O—CH₂CH₃, —SH, —S—CH₃, —S—CH₂CH₃, —COOH, —COO(CH₃), —COO(CH₂CH₃), —CH₂OH, —CH₂CH₂OH, —CH(CH₃)OH, —CH₂NH₂, —CH₂CH₂NH₂, or —CH(CH₃)NH₂, preferably, each of R_{S5} is independently selected from —F, —CH₃, —CH₂CH₃, —CF₃, —CN, oxo, —NH₂, —OH, —O—CH₃, —OCH₂—COO(CH₂CH₃), —COOH, —COO(CH₃), —CH₂OH, or —CH₂NH₂;
q₃ is selected from 0, 1, or 2;
Each of R_{S6} is independently selected from —F, —Cl, —CH₃, —CH₂CH₃, —CH₂CH₂CH₃, —CH(CH₃)₂, —CH₂Cl, —CHCl₂, —CCl₃, —CH₂F, —CHF₂, —CF₃, —CN, oxo, —NH₂, —NH(CH₃), —N(CH₃)₂, —OH, —O—CH₃, —O—CH₂CH₃, —SH, —S—CH₃, —S—CH₂CH₃, —COOH, —COO(CH₃), —COO(CH₂CH₃), —CH₂OH, —CH₂CH₂OH, —CH(CH₃)OH, —CH₂NH₂, —CH₂CH₂NH₂, or —CH(CH₃)NH₂, preferably, each of R_{S6} is independently selected from —F, —CH₃, —CH₂CH₃, —CF₃, —CN, oxo, —NH₂, —OH, —O—CH₃, —OCH₂—COO(CH₂CH₃), —COOH, —COO(CH₃), —CH₂OH, or —CH₂NH₂;
q₄ is selected from 0, 1, or 2; Each of R_{S7} is independently selected from —F, —Cl, —CH₃, —CH₂CH₃, —CH₂CH₂CH₃, —CH(CH₃)₂, —CH₂Cl, —CHCl₂, —CCl₃, —CH₂F, —CHF₂, —CF₃, —CN, oxo, —NH₂, —NH(CH₃), —N(CH₃)₂, —OH, —O—CH₃, —O—CH₂CH₃, —SH, —S—CH₃, —S—CH₂CH₃, —COOH, —COO(CH₃), —COO(CH₂CH₃), —CH₂OH, —CH₂CH₂OH, —CH(CH₃)OH, —CH₂NH₂, —CH₂CH₂NH₂, or —CH(CH₃)NH₂, preferably, each of R_{S7} is independently selected from —F, —CH₃, —CH₂CH₃, —CF₃, —CN, oxo, —NH₂, —OH, —O—CH₃, —COOH, —COO(CH₃), —CH₂OH, or —CH₂NH₂;
q₅ is selected from 0, 1, or 2;
Each of R_{S8} at each occurrence is independently selected from halogen, —C₁₋₆alkyl, haloC₁₋₆alkyl, haloC₁₋₆alkoxy, —CN, oxo, —NR_{N1}R_{N2}, —OR_{N1}, —C(═O)R_{N1}, —C(═O)OR_{N1}, —OC(═O)R_{N1}, —C(═O)NR_{N1}R_{N2}, —NR_{N1}C(═O)R_{N2}, —OC(═O)OR_{N1}, —NR_{N1}C(═O)OR_{N2}, —OC(═O)NR_{N1}R_{N2}, —NR_{N1}C(═O)NR_{N1}R_{N2}, 3-8 membered cycloalkyl, 4-8 membered heterocyclyl containing 1, 2 or 3 heteroatoms selected from N, O or S, phenyl or 5-6 membered heteroaryl containing 1, 2 or 3 heteroatoms selected from N, O or S, wherein said —C₁₋₆alkyl, haloC₁₋₆alkyl, haloC₁₋₆alkoxy, 3-8 membered cycloalkyl, 4-8 membered heterocyclyl, phenyl or 5-6 membered heteroaryl is optionally independently substituted with 1, 2 or 3 substituents selected from halogen, —C₁₋₆alkyl, haloC₁₋₆alkyl, haloC₁₋₆alkoxy, —CN, oxo, —NR_{N1}R_{N2}, —OR_{N1}, —C(═O)R_{N1}, —C(═O)OR_{N1}, —OC(═O)R_{N1}, —C(═O)NR_{N1}R_{N2}, —NR_{N1}C(═O)R_{N2}, —OC(═O)OR_{N1}, —NR_{N1}C(═O)OR_{N2}, —OC(═O)NR_{N1}R_{N2}, —NR_{N1}C(═O)NR_{N1}R_{N2}, 3-6 membered cycloalkyl, 4-6 membered heterocyclyl, phenyl or 5-6 membered heteroaryl;
Each of (R_{N1} or R_{N2}) in R_{S8} is independently selected from hydrogen or —C₁₋₆alkyl;
Optionally, (R_{N1} and R_{N2}) in R_{S8} together with the nitrogen atom to which they are both attached form a 3-6 membered heterocyclic ring which is optionally substituted with one or more substituents selected from halogen, —C₁₋₆alkyl, —OH, —OC₁₋₆alkyl, —SH, —SC₁₋₆alkyl, —NH₂, —NH(C₁₋₆alkyl) or —N(C₁₋₆alkyl)₂;
q₆ is selected from 0, 1, 2 or 3;
Each of R_{S11} is selected from —C₁₋₃alkyl.

[13]. The compound of any one of aspects [1] to [12], wherein, the moiety of —X₂—R₂ or —O—R₂ is selected from any one of the structures in the following Table 2:

TABLE 2

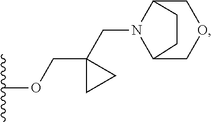 [SMILES: *OCC1(CN2C3CCC2COC3)CC1]

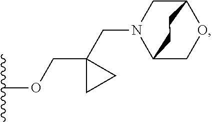 [SMILES: *OCC1(CN2C[C@H]3CC[C@@H]2CO3)CC1]

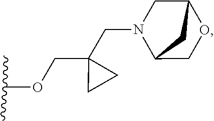 [SMILES: *OCC1(CN2C[C@H]3C[C@@H]2CO3)CC1]

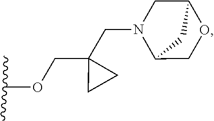 [SMILES: *OCC1(CN2C[C@@H]3C[C@H]2CO3)CC1]

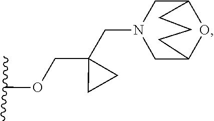 [SMILES: *OCC1(CN2CC3CCCC(C2)O3)CC1]

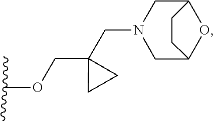 [SMILES: *OCC1(CN2CC3CCC(C2)O3)CC1]

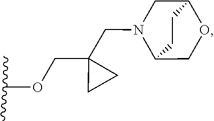 [SMILES: *OCC1(CN2C[C@@H]3CC[C@H]2CO3)CC1]

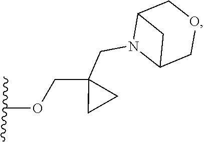 [SMILES: *OCC1(CN2C3COCC2C3)CC1]

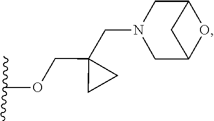 [SMILES: *OCC1(CN2CC3CC(C2)O3)CC1]

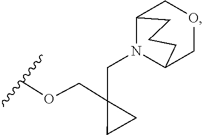 [SMILES: *OCC1(CN2C3CCCC2COC3)CC1]

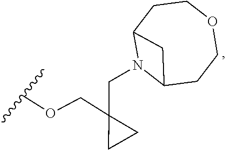 [SMILES: *OCC1(CN2C3CCOCCC2C3)CC1]

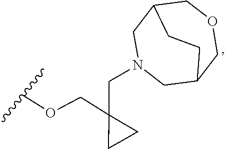 [SMILES: *OCC1(CN2CC3CCC(COC3)C2)CC1]

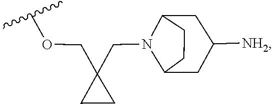 [SMILES: *OCC1(CN2C3CCC2CC(N)C3)CC1]

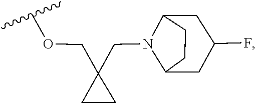 [SMILES: *OCC1(CN2C3CCC2CC(F)C3)CC1]

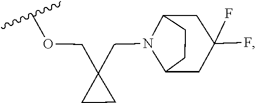 [SMILES: *OCC1(CN2C3CCC2CC(F)(F)C3)CC1]

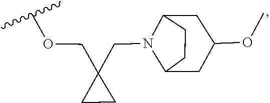 [SMILES: *OCC1(CN2C3CCC2CC(OC)C3)CC1]

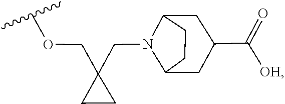 [SMILES: *OCC1(CN2C3CCC2CC(C(=O)O)C3)CC1]

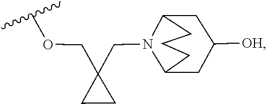 [SMILES: *OCC1(CN2C3CCCC2CC(O)C3)CC1]

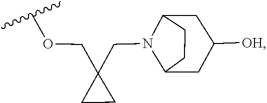 [SMILES: *OCC1(CN2C3CCC2CC(O)C3)CC1]

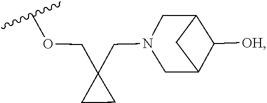 [SMILES: *OCC1(CN2CC3CC(C2)C3O)CC1]

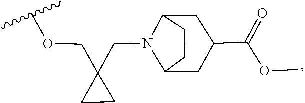 [SMILES: *OCC1(CN2C3CCC2CC(C(=O)OC)C3)CC1]

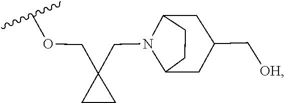 [SMILES: *OCC1(CN2C3CCC2CC(CO)C3)CC1]

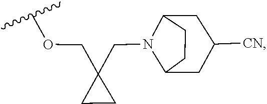 [SMILES: *OCC1(CN2C3CCC2CC(C#N)C3)CC1]

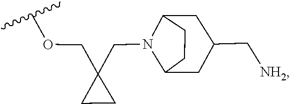 [SMILES: *OCC1(CN2C3CCC2CC(CN)C3)CC1]

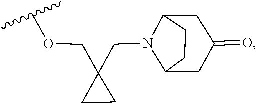 [SMILES: *OCC1(CN2C3CCC2CC(=O)C3)CC1]

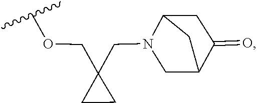 [SMILES: *OCC1(CN2CC3CC2CC3=O)CC1]

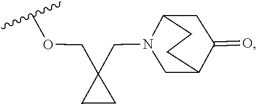 [SMILES: *OCC1(CN2CC3CCC2CC3=O)CC1]

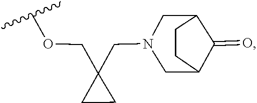 [SMILES: *OCC1(CN2CC3CCC(C2)C3=O)CC1]

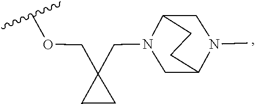 [SMILES: *OCC1(CN2CC3CCC2CN3C)CC1]

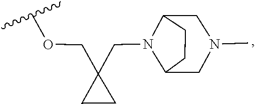 [SMILES: *OCC1(CN2C3CCC2CN(C)C3)CC1]

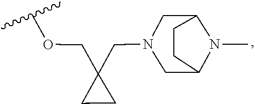 [SMILES: *OCC1(CN2CC3CCC(C2)N3C)CC1]

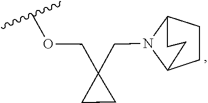 [SMILES: *OCC1(CN2C3CCC2CC3)CC1]

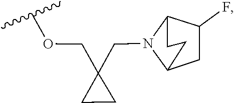 [SMILES: *OCC1(CN2C3CCC2C(F)C3)CC1]

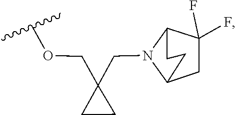 [SMILES: *OCC1(CN2C3CCC2C(F)(F)C3)CC1]

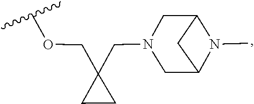 [SMILES: *OCC1(CN2CC3CC(C2)N3C)CC1]

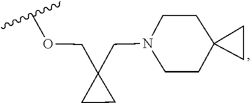 [SMILES: *OCC1(CN2CCC3(CC2)CC3)CC1]

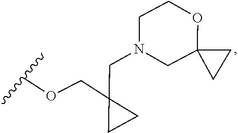 [SMILES: *OCC1(CN2CCOC3(CC3)C2)CC1]

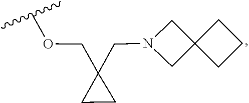 [SMILES: *OCC1(CN2CC3(CCC3)C2)CC1]

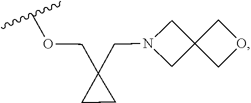 [SMILES: *OCC1(CN2CC3(COC3)C2)CC1]

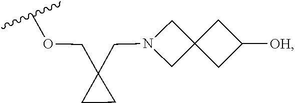 [SMILES: *OCC1(CN2CC3(CC(O)C3)C2)CC1]

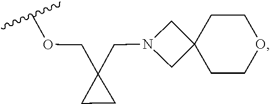 [SMILES: *OCC1(CN2CC3(CCOCC3)C2)CC1]

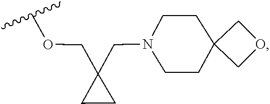 [SMILES: *OCC1(CN2CCC3(CC2)COC3)CC1]

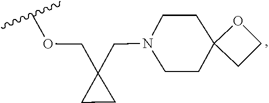 [SMILES: *OCC1(CN2CCC3(CCO3)CC2)CC1]

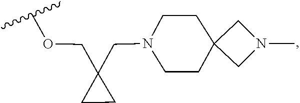 [SMILES: *OCC1(CN2CCC3(CC2)CN(C)C3)CC1]

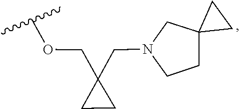 [SMILES: *OCC1(CN2CCC3(CC3)C2)CC1]

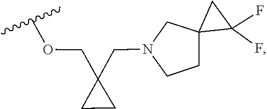 [SMILES: *OCC1(CN2CCC3(C2)CC3(F)F)CC1]

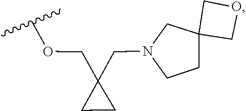 [SMILES: *OCC1(CN2CCC3(COC3)C2)CC1]

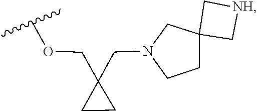 [SMILES: *OCC1(CN2CCC3(CNC3)C2)CC1]

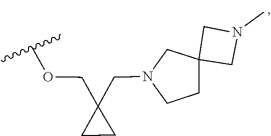 [SMILES: *OCC1(CN2CCC3(CN(C)C3)C2)CC1]

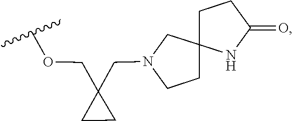 [SMILES: *OCC1(CN2CCC3(CCC(=O)N3)C2)CC1]

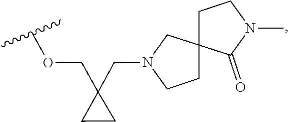 [SMILES: *OCC1(CN2CCC3(CCN(C)C3=O)C2)CC1]

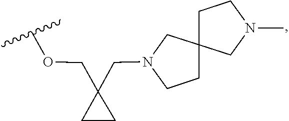 [SMILES: *OCC1(CN2CCC3(CCN(C)C3)C2)CC1]

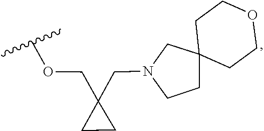 [SMILES: *OCC1(CN2CCC3(CCOCC3)C2)CC1]

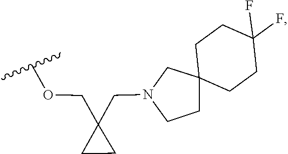 [SMILES: *OCC1(CN2CCC3(CCC(F)(F)CC3)C2)CC1]

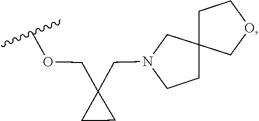 [SMILES: *OCC1(CN2CCC3(CCOC3)C2)CC1]

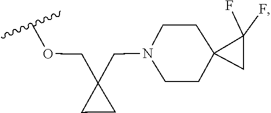 [SMILES: *OCC1(CN2CCC3(CC2)CC3(F)F)CC1]

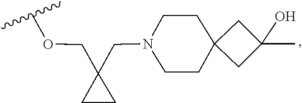 [SMILES: *OCC1(CN2CCC3(CC2)CC(C)(O)C3)CC1]

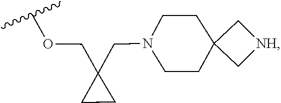 [SMILES: *OCC1(CN2CCC3(CC2)CNC3)CC1]

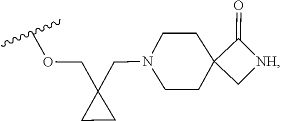 [SMILES: *OCC1(CN2CCC3(CC2)CNC3=O)CC1]

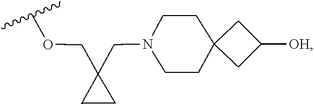 [SMILES: *OCC1(CN2CCC3(CC2)CC(O)C3)CC1]

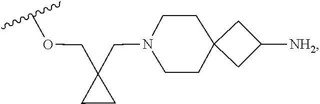 [SMILES: *OCC1(CN2CCC3(CC2)CC(N)C3)CC1]

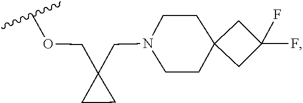 [SMILES: *OCC1(CN2CCC3(CC2)CC(F)(F)C3)CC1]

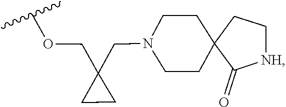 [SMILES: *OCC1(CN2CCC3(CCNC3=O)CC2)CC1]

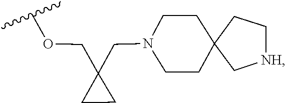 [SMILES: *OCC1(CN2CCC3(CCNC3)CC2)CC1]

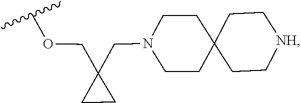 [SMILES: *OCC1(CN2CCC3(CCNCC3)CC2)CC1]

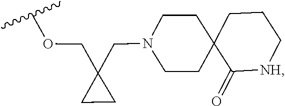 [SMILES: *OCC1(CN2CCC3(CCCNC3=O)CC2)CC1]

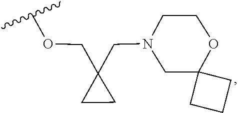 [SMILES: *OCC1(CN2CCOC3(CCC3)C2)CC1]

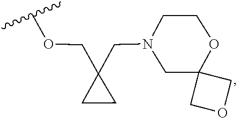 [SMILES: *OCC1(CN2CCOC3(COC3)C2)CC1]

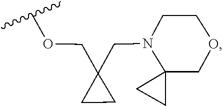 [SMILES: *OCC1(CN2CCOCC23CC3)CC1]

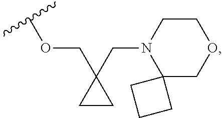 [SMILES: *OCC1(CN2CCOCC23CCC3)CC1]

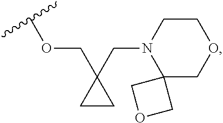 [SMILES: *OCC1(CN2CCOCC23COC3)CC1]

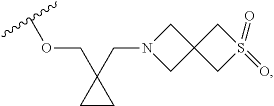 [SMILES: *OCC1(CN2CC3(C2)CS(=O)(=O)C3)CC1]

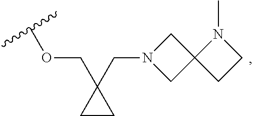 [SMILES: *OCC1(CN2CC3(CCN3C)C2)CC1]

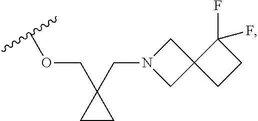 [SMILES: *OCC1(CN2CC3(CCC3(F)F)C2)CC1]

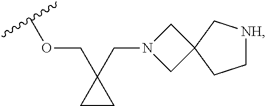 [SMILES: *OCC1(CN2CC3(CCNC3)C2)CC1]

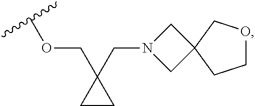 [SMILES: *OCC1(CN2CC3(CCOC3)C2)CC1]

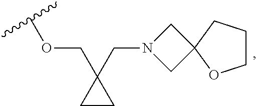 [SMILES: *OCC1(CN2CC3(CCCO3)C2)CC1]

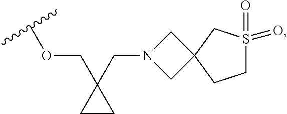 [SMILES: *OCC1(CN2CC3(CCS(=O)(=O)C3)C2)CC1]

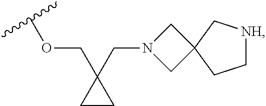 [SMILES: *OCC1(CN2CC3(CCNC3)C2)CC1]

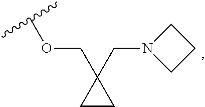 [SMILES: *OCC1(CN2CCC2)CC1]

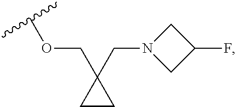 [SMILES: *OCC1(CN2CC(F)C2)CC1]

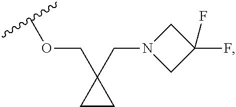 [SMILES: *OCC1(CN2CC(F)(F)C2)CC1]

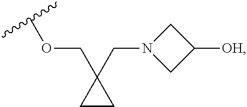 [SMILES: *OCC1(CN2CC(O)C2)CC1]

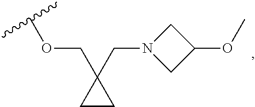 [SMILES: *OCC1(CN2CC(OC)C2)CC1]

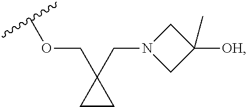 [SMILES: *OCC1(CN2CC(C)(O)C2)CC1]

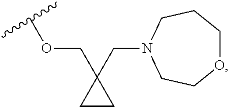 [SMILES: *OCC1(CN2CCCOCC2)CC1]

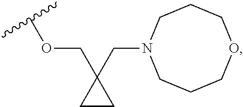 [SMILES: *OCC1(CN2CCCOCCC2)CC1]

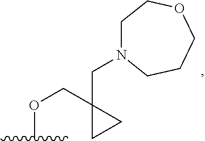 [SMILES: *OCC1(CN2CCCOCC2)CC1]

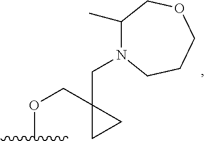 [SMILES: *OCC1(CN2CCCOCC2C)CC1]

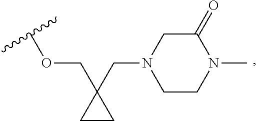 [SMILES: *OCC1(CN2CCN(C)C(=O)C2)CC1]

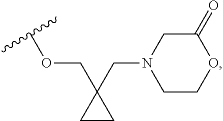 [SMILES: *OCC1(CN2CCOC(=O)C2)CC1]

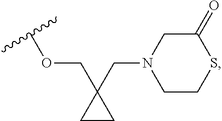 [SMILES: *OCC1(CN2CCSC(=O)C2)CC1]

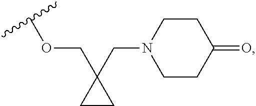 [SMILES: *OCC1(CN2CCC(=O)CC2)CC1]

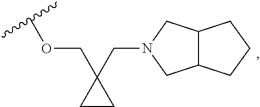 [SMILES: *OCC1(CN2CC3CCCC3C2)CC1]

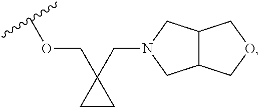 [SMILES: *OCC1(CN2CC3COCC3C2)CC1]

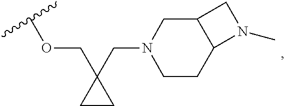 [SMILES: *OCC1(CN2CCC3C(C2)CN3C)CC1]

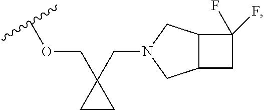 [SMILES: *OCC1(CN2CC3CC(F)(F)C3C2)CC1]

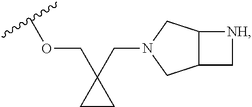 [SMILES: *OCC1(CN2CC3CNC3C2)CC1]

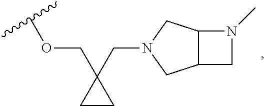 [SMILES: *OCC1(CN2CC3CN(C)C3C2)CC1]

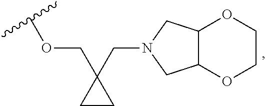 [SMILES: *OCC1(CN2CC3OCCOC3C2)CC1]

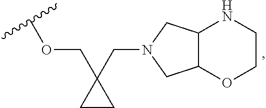 [SMILES: *OCC1(CN2CC3NCCOC3C2)CC1]

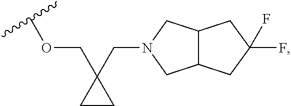 [SMILES: *OCC1(CN2CC3CC(F)(F)CC3C2)CC1]

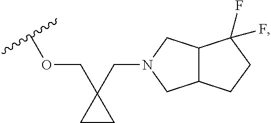 [SMILES: *OCC1(CN2CC3CCC(F)(F)C3C2)CC1]

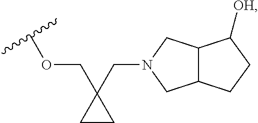 [SMILES: *OCC1(CN2CC3CCC(O)C3C2)CC1]

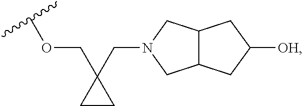 [SMILES: *OCC1(CN2CC3CC(O)CC3C2)CC1]

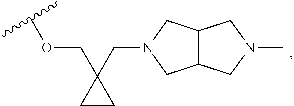 [SMILES: *OCC1(CN2CC3CN(C)CC3C2)CC1]

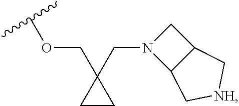 [SMILES: *OCC1(CN2CC3CNCC32)CC1]

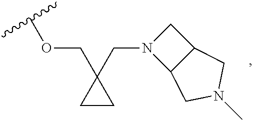 [SMILES: *OCC1(CN2CC3CN(C)CC32)CC1]

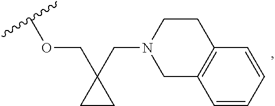 [SMILES: *OCC1(CN2CCc3ccccc3C2)CC1]

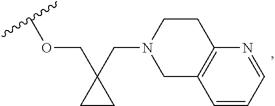 [SMILES: *OCC1(CN2CCc3ncccc3C2)CC1]

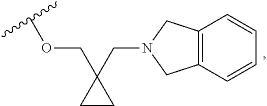 [SMILES: *OCC1(CN2Cc3ccccc3C2)CC1]

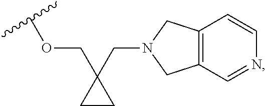 [SMILES: *OCC1(CN2Cc3ccncc3C2)CC1]

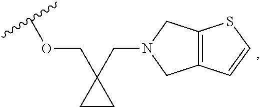 [SMILES: *OCC1(CN2Cc3ccsc3C2)CC1]

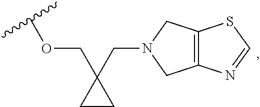 [SMILES: *OCC1(CN2Cc3ncsc3C2)CC1]

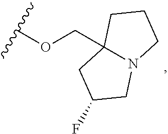 [SMILES: *OCC12CCCN1C[C@H](F)C2]

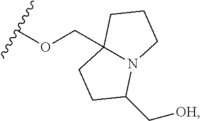 [SMILES: *OCC12CCCN1C(CO)CC2]

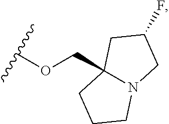 [SMILES: *OC[C@]12CCCN1C[C@@H](F)C2]

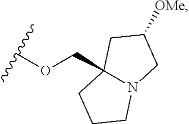 [SMILES: *OC[C@]12CCCN1C[C@@H](OC)C2]

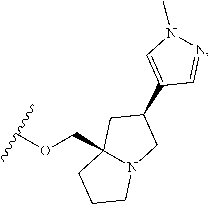 [SMILES: *OC[C@]12CCCN1C[C@H](c1cnn(C)c1)C2]

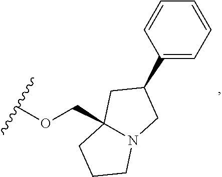 [SMILES: *OC[C@]12CCCN1C[C@H](c1ccccc1)C2]

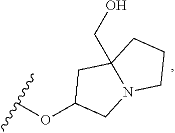 [SMILES: *OC1CN2CCCC2(CO)C1]

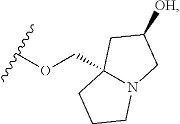 [SMILES: *OC[C@@]12CCCN1C[C@H](O)C2]

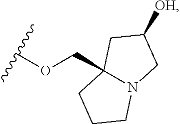 [SMILES: *OC[C@]12CCCN1C[C@H](O)C2]

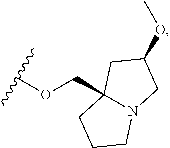 [SMILES: *OC[C@]12CCCN1C[C@H](OC)C2]

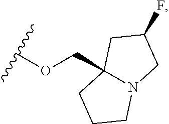 [SMILES: *OC[C@]12CCCN1C[C@H](F)C2]

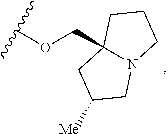 [SMILES: *OC[C@@]12CCCN1C[C@H](C)C2]

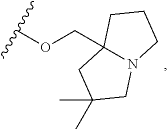 [SMILES: *OCC12CCCN1CC(C)(C)C2]

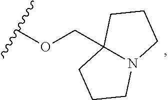 [SMILES: *OCC12CCCN1CCC2]

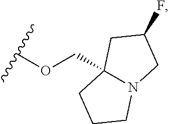 [SMILES: *OC[C@@]12CCCN1C[C@H](F)C2]

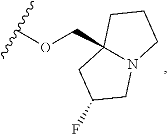 [SMILES: *OC[C@@]12CCCN1C[C@H](F)C2]

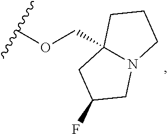 [SMILES: *OC[C@]12CCCN1C[C@@H](F)C2]

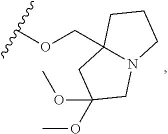 [SMILES: *OCC12CCCN1CC(OC)(OC)C2]

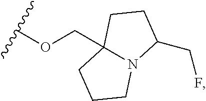 [SMILES: *OCC12CCCN1C(CF)CC2]

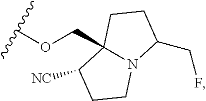 [SMILES: *OC[C@@]12CCC(CF)N1CC[C@@H]2C#N]

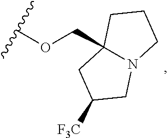 [SMILES: *OC[C@@]12CCCN1C[C@@H](C(F)(F)F)C2]

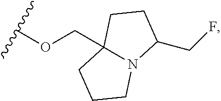 [SMILES: *OCC12CCCN1C(CF)CC2]

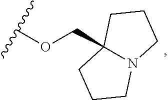

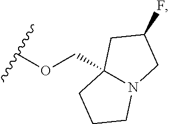 [SMILES: *OC[C@@]12CCCN1C[C@H](F)C2]

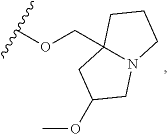 [SMILES: *OCC12CCCN1CC(OC)C2]

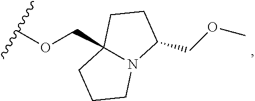 [SMILES: *OC[C@]12CCCN1[C@@H](COC)CC2]

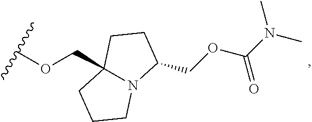 [SMILES: *OC[C@]12CCCN1[C@@H](COC(=O)N(C)C)CC2]

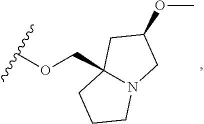 [SMILES: *OC[C@]12CCCN1C[C@H](OC)C2]

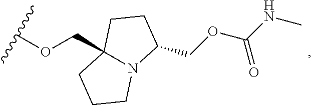 [SMILES: *OC[C@]12CCCN1[C@@H](COC(=O)NC)CC2]

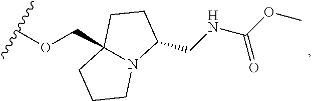 [SMILES: *OC[C@]12CCCN1[C@@H](CNC(=O)OC)CC2]

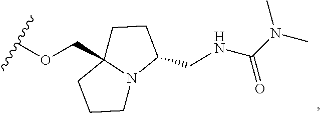 [SMILES: *OC[C@]12CCCN1[C@@H](CNC(=O)N(C)C)CC2]

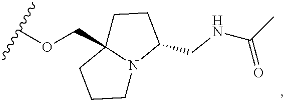 [SMILES: *OC[C@]12CCCN1[C@@H](CNC(C)=O)CC2]

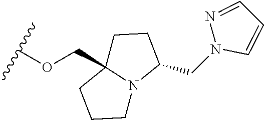 [SMILES: *OC[C@]12CCCN1[C@@H](Cn1cccn1)CC2]

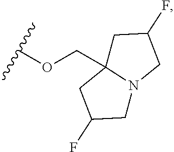 [SMILES: *OCC12CC(F)CN1CC(F)C2]

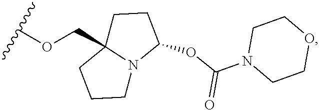 [SMILES: *OC[C@]12CCCN1[C@@H](OC(=O)N1CCOCC1)CC2]

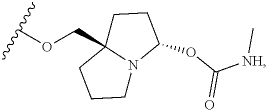 [SMILES: *OC[C@]12CCCN1[C@@H](OC(=O)NC)CC2]

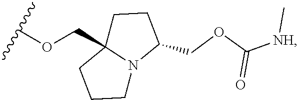 [SMILES: *OC[C@]12CCCN1[C@@H](COC(=O)NC)CC2]

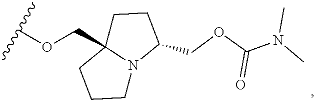 [SMILES: *OC[C@]12CCCN1[C@@H](COC(=O)N(C)C)CC2]

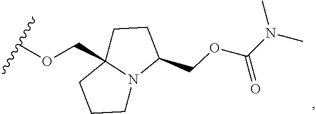 [SMILES: *OC[C@]12CCCN1[C@H](COC(=O)N(C)C)CC2]

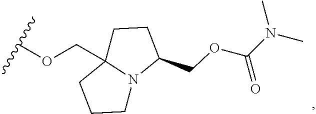 [SMILES: *OCC12CCCN1[C@H](COC(=O)N(C)C)CC2]

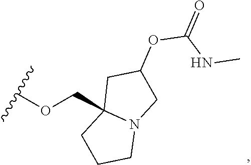 [SMILES: *OC[C@]12CCCN1CC(OC(=O)NC)C2]

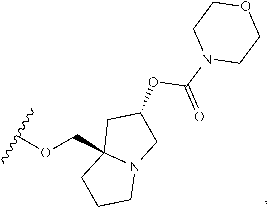 [SMILES: *OC[C@]12CCCN1C[C@@H](OC(=O)N1CCOCC1)C2]

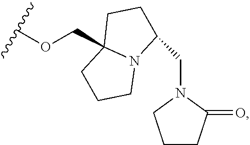 [SMILES: *OC[C@]12CCCN1[C@@H](CN1CCCC1=O)CC2]

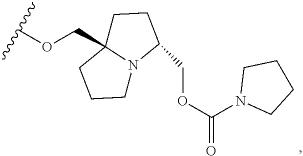 [SMILES: *OC[C@]12CCCN1[C@@H](COC(=O)N1CCCC1)CC2]

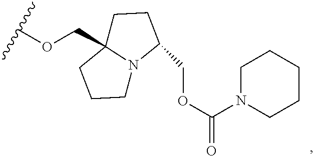 [SMILES: *OC[C@]12CCCN1[C@@H](COC(=O)N1CCCCC1)CC2]

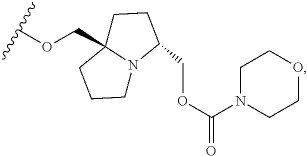 [SMILES: *OC[C@]12CCCN1[C@@H](COC(=O)N1CCOCC1)CC2]

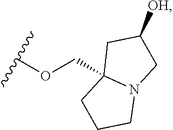 [SMILES: *OC[C@@]12CCCN1C[C@H](O)C2]

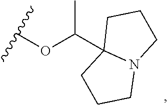 [SMILES: *OC(C)C12CCCN1CCC2]

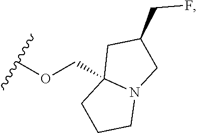 [SMILES: *OC[C@@]12CCCN1C[C@H](CF)C2]

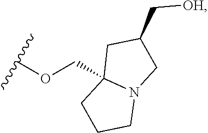 [SMILES: *OC[C@@]12CCCN1C[C@H](CO)C2]

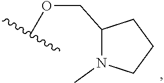 [SMILES: *OCC1CCCN1C]

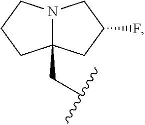 [SMILES: *C[C@@]12CCCN1C[C@H](F)C2]

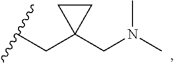 [SMILES: *CC1(CN(C)C)CC1]

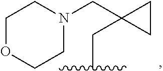 [SMILES: *CC1(CN2CCOCC2)CC1]

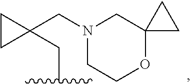 [SMILES: *CC1(CN2CCOC3(CC3)C2)CC1]

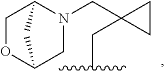 [SMILES: *CC1(CN2C[C@@H]3C[C@H]2CO3)CC1]

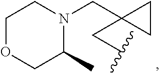 [SMILES: *CC1(CN2CCOC[C@@H]2C)CC1]

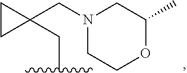 [SMILES: *CC1(CN2CCO[C@@H](C)C2)CC1]

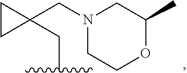 [SMILES: *CC1(CN2CCO[C@H](C)C2)CC1]

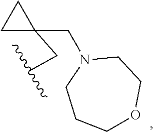 [SMILES: *CC1(CN2CCCOCC2)CC1]

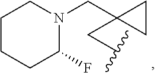 [SMILES: *CC1(CN2CCCC[C@H]2F)CC1]

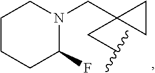 [SMILES: *CC1(CN2CCCC[C@@H]2F)CC1]

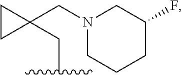 [SMILES: *CC1(CN2CCC[C@@H](F)C2)CC1]

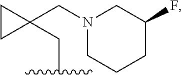 [SMILES: *CC1(CN2CCC[C@H](F)C2)CC1]

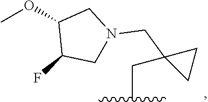 [SMILES: *CC1(CN2C[C@@H](F)[C@H](OC)C2)CC1]

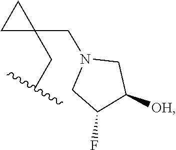 [SMILES: *CC1(CN2C[C@@H](O)[C@H](F)C2)CC1]

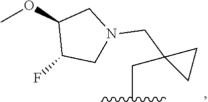 [SMILES: *CC1(CN2C[C@H](OC)[C@@H](F)C2)CC1]

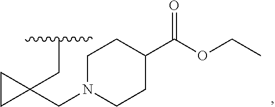 [SMILES: *CC1(CN2CCC(C(=O)OCC)CC2)CC1]

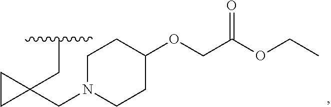 [SMILES: *CC1(CN2CCC(OCC(=O)OCC)CC2)CC1]

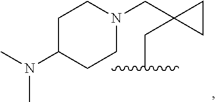 [SMILES: *CC1(CN2CCC(N(C)C)CC2)CC1]

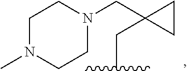 [SMILES: *CC1(CN2CCN(C)CC2)CC1]

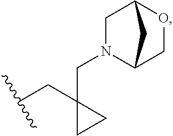 [SMILES: *CC1(CN2C[C@H]3C[C@@H]2CO3)CC1]

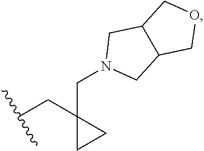 [SMILES: *CC1(CN2CC3COCC3C2)CC1]

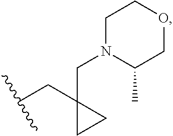 [SMILES: *CC1(CN2CCOC[C@@H]2C)CC1]

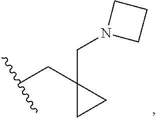 [SMILES: *CC1(CN2CCC2)CC1]

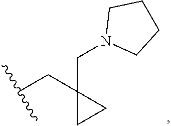 [SMILES: *CC1(CN2CCCC2)CC1]

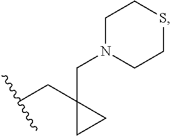 [SMILES: *CC1(CN2CCSCC2)CC1]

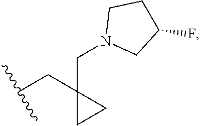 [SMILES: *CC1(CN2CC[C@H](F)C2)CC1]

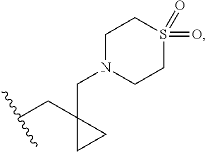 [SMILES: *CC1(CN2CCS(=O)(=O)CC2)CC1]

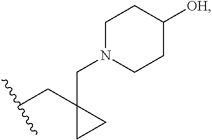 [SMILES: *CC1(CN2CCC(O)CC2)CC1]

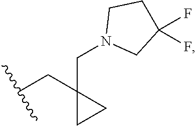 [SMILES: *CC1(CN2CCC(F)(F)C2)CC1]

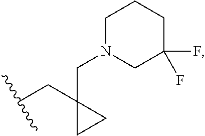 [SMILES: *CC1(CN2CCCC(F)(F)C2)CC1]

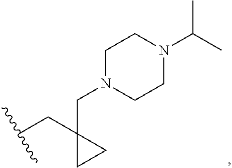 [SMILES: *CC1(CN2CCN(C(C)C)CC2)CC1]

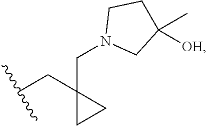 [SMILES: *CC1(CN2CCC(C)(O)C2)CC1]

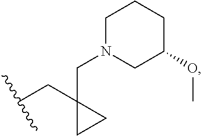 [SMILES: *CC1(CN2CCC[C@H](OC)C2)CC1]

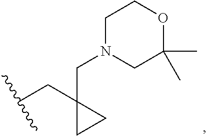 [SMILES: *CC1(CN2CCOC(C)(C)C2)CC1]

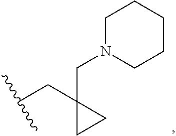 [SMILES: *CC1(CN2CCCCC2)CC1]

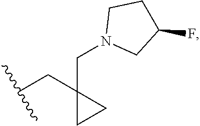 [SMILES: *CC1(CN2CC[C@@H](F)C2)CC1]

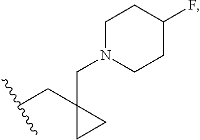 [SMILES: *CC1(CN2CCC(F)CC2)CC1]

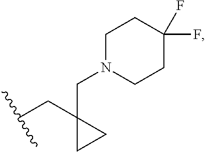 [SMILES: *CC1(CN2CCC(F)(F)CC2)CC1]

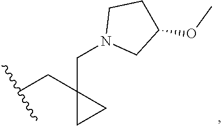 [SMILES: *CC1(CN2CC[C@H](OC)C2)CC1]

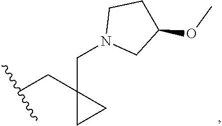 [SMILES: *CC1(CN2CC[C@@H](OC)C2)CC1]

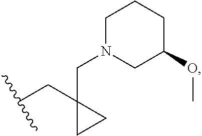 [SMILES: *CC1(CN2CCC[C@@H](OC)C2)CC1]

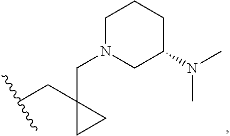 [SMILES: *CC1(CN2CCC[C@H](N(C)C)C2)CC1]

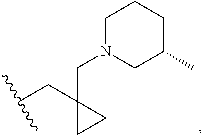 [SMILES: *CC1(CN2CCC[C@H](C)C2)CC1]

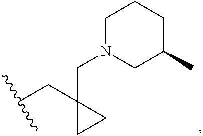 [SMILES: *CC1(CN2CCC[C@@H](C)C2)CC1]

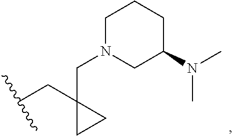 [SMILES: *CC1(CN2CCC[C@@H](N(C)C)C2)CC1]

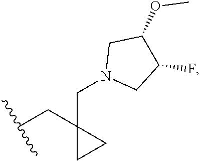 [SMILES: *CC1(CN2C[C@@H](F)[C@@H](OC)C2)CC1]

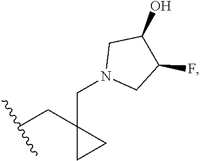 [SMILES: *CC1(CN2C[C@@H](O)[C@@H](F)C2)CC1]

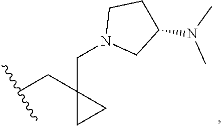 [SMILES: *CC1(CN2CC[C@H](N(C)C)C2)CC1]

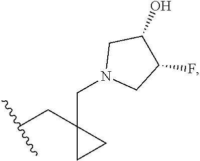 [SMILES: *CC1(CN2C[C@@H](F)[C@@H](O)C2)CC1]

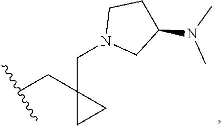 [SMILES: *CC1(CN2CC[C@@H](N(C)C)C2)CC1]

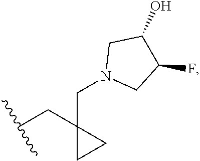 [SMILES: *CC1(CN2C[C@H](O)[C@@H](F)C2)CC1]

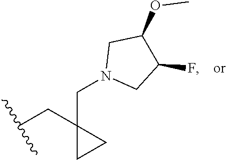 [SMILES: *CC1(CN2C[C@H](F)[C@H](OC)C2)CC1]

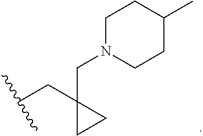 [SMILES: *CC1(CN2CCC(C)CC2)CC1]

[14]. The compound of any one of aspects [1] to [12], wherein, the moiety of —X₂—R₂ or —O—R2 is selected from

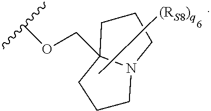

[15]. The compound of any one of aspects [1] to [14], wherein, each of R_{S8} at each occurrence is independently selected from halogen, —C₁₋₆alkyl, haloC₁₋₆alkyl, haloC₁₋₆alkoxy, —CN, oxo, —NR_{N1}R_{N2}, —OR_{N1}, —C(═O)R_{N1}, —C(═O)OR_{N1}, —OC(═O)R_{N1}, —C(═O)NR_{N1}R_{N2}, —NR_{N1}C(═O)R_{N2}, —OC(═O)OR_{N1}, —NR_{N1}C(═O)OR_{N2}, —OC(═O)NR_{N1}R_{N2}, —NR_{N1}C(═O)NR_{N1}R_{N2}, 3-8 membered cycloalkyl, 4-8 membered heterocyclyl containing 1, 2 or 3 heteroatoms selected from N, O or S, phenyl or 5-6 membered heteroaryl containing 1, 2 or 3 heteroatoms selected from N, O or S, wherein said —C₁₋₆alkyl, haloC₁₋₆alkyl, haloC₁₋₆alkoxy, 3-8 membered cycloalkyl, 4-8 membered heterocyclyl, phenyl or 5-6 membered heteroaryl is optionally independently substituted with 1, 2 or 3 substituents selected from halogen, —C₁₋₆alkyl, haloC₁₋₆alkyl, haloC₁₋₆alkoxy, —CN, oxo, —NR_{N1}R_{N2}, —OR_{N1}, —C(═O)R_{N1}, —C(═O)OR_{N1}, —OC(═O)R_{N1}, —C(═O)NR_{N1}R_{N2}, —NR_{N1}C(═O)R_{N2}, —OC(═O)OR_{N1}, —NR_{N1}C(═O)OR_{N2}, —OC(═O)NR_{N1}R₂, —NR_{N1}C(═O)NR_{N1}R_{N2}, 3-6 membered cycloalkyl, 4-6 membered heterocyclyl, phenyl or 5-6 membered heteroaryl;
Each of (R_{N1} or R_{N2}) in R_{S8} is independently selected from hydrogen or —C₁₋₆alkyl;
Optionally, (R_{N1} and R_{N2}) in R_{S8} together with the nitrogen atom to which they are both attached form a 3-6 membered heterocyclic ring which is optionally substituted with one or more substituents selected from halogen, —C₁₋₆alkyl, —OH, —OC₁₋₆alkyl, —SH, —SC₁₋₆alkyl, —NH₂, —NH(C₁₋₆alkyl) or —N(C₁₋₆alkyl)₂;
q₆ is selected from 0, 1, 2 or 3.

[16]. The compound of any one of aspects [1] to [15], wherein, each of R_{S8} at each occurrence is independently selected from —F; methyl; —CF₃; —CN; oxo; —OH; —NH₂; —OCH₃; —NHC(═O)CH₃; —NHC(═O)OCH₃; —OC(═O)N(CH₃)₂; —NHC(═O)N(CH₃)₂;

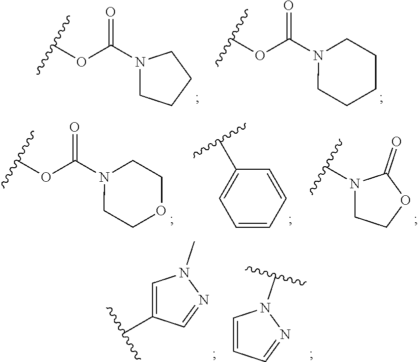

or methyl substituted with —F, —Cl, methyl, —CF₃, —CN, oxo, —OH, —NH₂, —OCH₃, —NHC(═O)CH₃, —NHC(═O)OCH₃, —OC(═O)N(CH₃)₂, —NHC(═O)N(CH₃)₂,

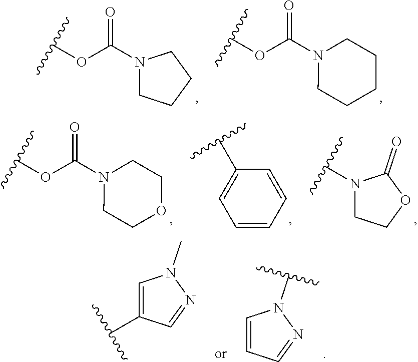

[17]. The compound of any one of aspects [1] to [16], wherein, each of R_{S8} at each occurrence is independently selected from —F, methyl, —CF₃, —CN, oxo, —OH, —NH₂, —OCH₃, —NHC(═O)CH₃, —NHC(═O)OCH₃, —OC(═O)N(CH₃)₂, —NHC(═O)N(CH₃)₂,

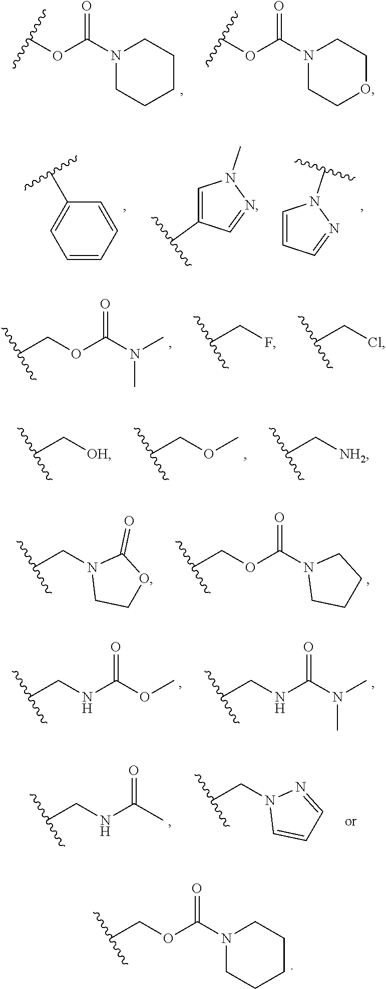

[18]. The compound of any one of aspects [1] to [17], wherein, the moiety of —X₂—R₂ or —O—R₂ is selected from any one of structures in the following Table 3:

TABLE 3

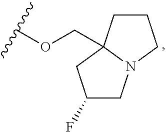 [SMILES: *OCC12CCCN1C[C@H](F)C2]

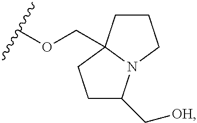 [SMILES: *OCC12CCCN1C(CO)CC2]

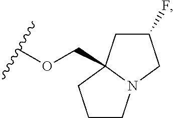 [SMILES: *OC[C@]12CCCN1C[C@@H](F)C2]

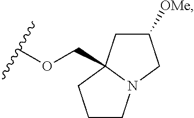 [SMILES: *OC[C@]12CCCN1C[C@@H](OC)C2]

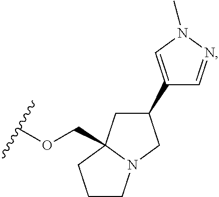 [SMILES: *OC[C@]12CCCN1C[C@H](c1cnn(C)c1)C2]

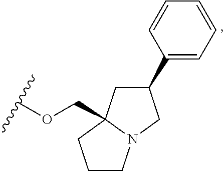 [SMILES: *OC[C@]12CCCN1C[C@H](c1ccccc1)C2]

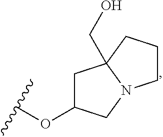 [SMILES: *OC1CN2CCCC2(CO)C1]

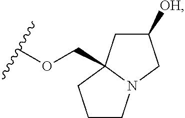 [SMILES: *OC[C@]12CCCN1C[C@H](O)C2]

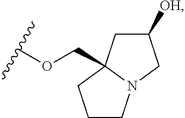 [SMILES: *OC[C@]12CCCN1C[C@H](O)C2]

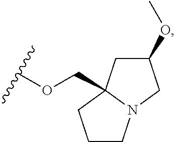 [SMILES: *OC[C@]12CCCN1C[C@H](OC)C2]

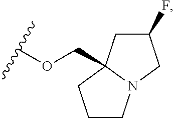 [SMILES: *OC[C@]12CCCN1C[C@H](F)C2]

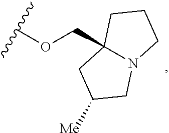 [SMILES: *OC[C@@]12CCCN1C[C@H](C)C2]

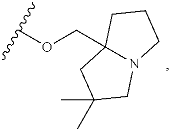 [SMILES: *OCC12CCCN1CC(C)(C)C2]

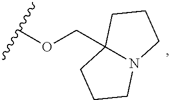 [SMILES: *OCC12CCCN1CCC2]

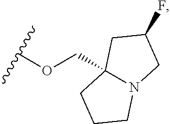 [SMILES: *OC[C@@]12CCCN1C[C@H](F)C2]

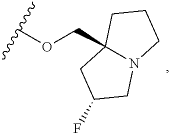 [SMILES: *OC[C@@]12CCCN1C[C@H](F)C2]

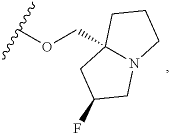 [SMILES: *OC[C@]12CCCN1C[C@@H](F)C2]

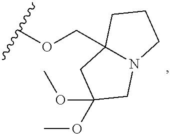 [SMILES: *OCC12CCCN1CC(OC)(OC)C2]

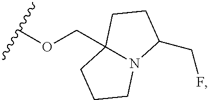 [SMILES: *OCC12CCCN1C(CF)CC2]

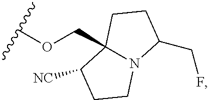 [SMILES: *OC[C@@]12CCC(CF)N1CC[C@@H]2C#N]

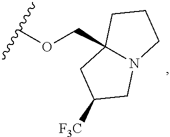 [SMILES: *OC[C@@]12CCCN1C[C@@H](C(F)(F)F)C2]

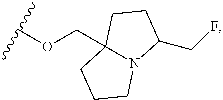 [SMILES: *OCC12CCCN1C(CF)CC2]

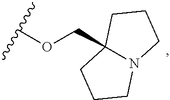

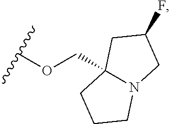 [SMILES: *OC[C@@]12CCCN1C[C@H](F)C2]

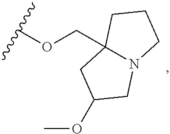 [SMILES: *OCC12CCCN1CC(OC)C2]

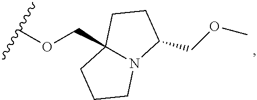 [SMILES: *OC[C@]12CCCN1[C@@H](COC)CC2]

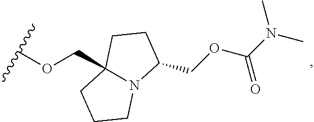 [SMILES: *OC[C@]12CCCN1[C@@H](COC(=O)N(C)C)CC2]

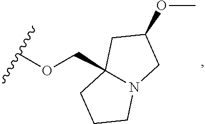 [SMILES: *OC[C@]12CCCN1C[C@H](OC)C2]

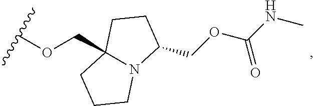 [SMILES: *OC[C@]12CCCN1[C@@H](COC(=O)NC)CC2]

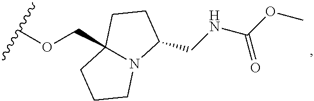 [SMILES: *OC[C@]12CCCN1[C@@H](CNC(=O)OC)CC2]

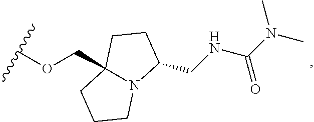 [SMILES: *OC[C@]12CCCN1[C@@H](CNC(=O)N(C)C)CC2]

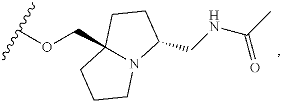 [SMILES: *OC[C@]12CCCN1[C@@H](CNC(C)=O)CC2]

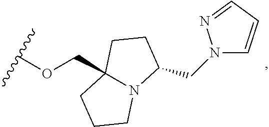 [SMILES: *OC[C@]12CCCN1[C@@H](Cn1cccn1)CC2]

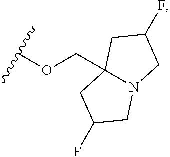 [SMILES: *OCC12CC(F)CN1CC(F)C2]

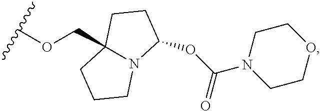 [SMILES: *OC[C@]12CCCN1[C@@H](OC(=O)N1CCOCC1)CC2]

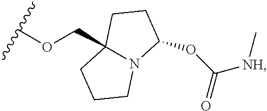 [SMILES: *OC[C@]12CCCN1[C@@H](OC(=O)NC)CC2]

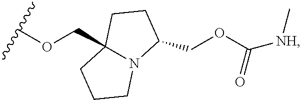 [SMILES: *OC[C@]12CCCN1[C@@H](COC(=O)NC)CC2]

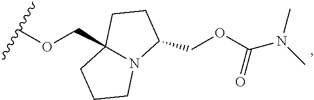 [SMILES: *OC[C@]12CCCN1[C@@H](COC(=O)N(C)C)CC2]

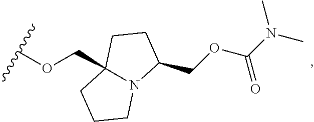 [SMILES: *OC[C@]12CCCN1[C@H](COC(=O)N(C)C)CC2]

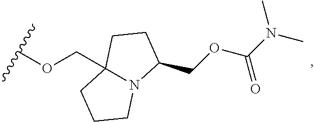 [SMILES: *OCC12CCCN1[C@H](COC(=O)N(C)C)CC2]

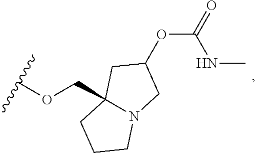 [SMILES: *OC[C@]12CCCN1CC(OC(=O)NC)C2]

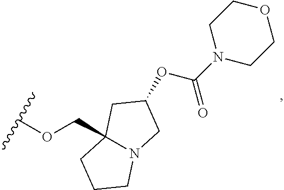 [SMILES: *OC[C@]12CCCN1C[C@@H](OC(=O)N1CCOCC1)C2]

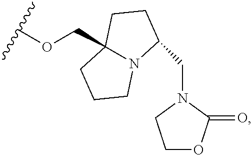 [SMILES: *OC[C@]12CCCN1[C@@H](CN1CCOC1=O)CC2]

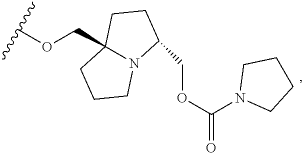 [SMILES: *OC[C@]12CCCN1[C@@H](COC(=O)N1CCCC1)CC2]

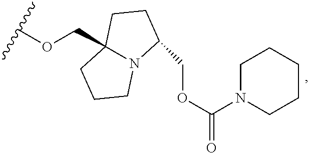 [SMILES: *OC[C@]12CCCN1[C@@H](COC(=O)N1CCCCC1)CC2]

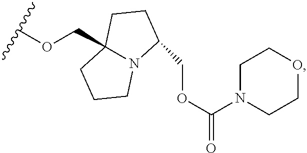 [SMILES: *OC[C@]12CCCN1[C@@H](COC(=O)N1CCOCC1)CC2]

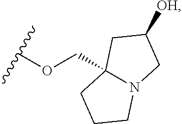 [SMILES: *OC[C@@]12CCCN1C[C@H](O)C2]

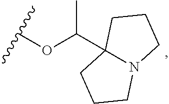 [SMILES: *OC(C)C12CCCN1CCC2]

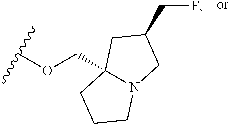 [SMILES: *OC[C@@]12CCCN1C[C@H](CF)C2 ; "or"]

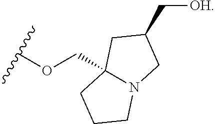 [SMILES: *OC[C@@]12CCCN1C[C@H](CO)C2]

[19]. The compound of any one of aspects [1] to [18], wherein, the moiety of —X₂—R₂ or —O—R₂ is selected from

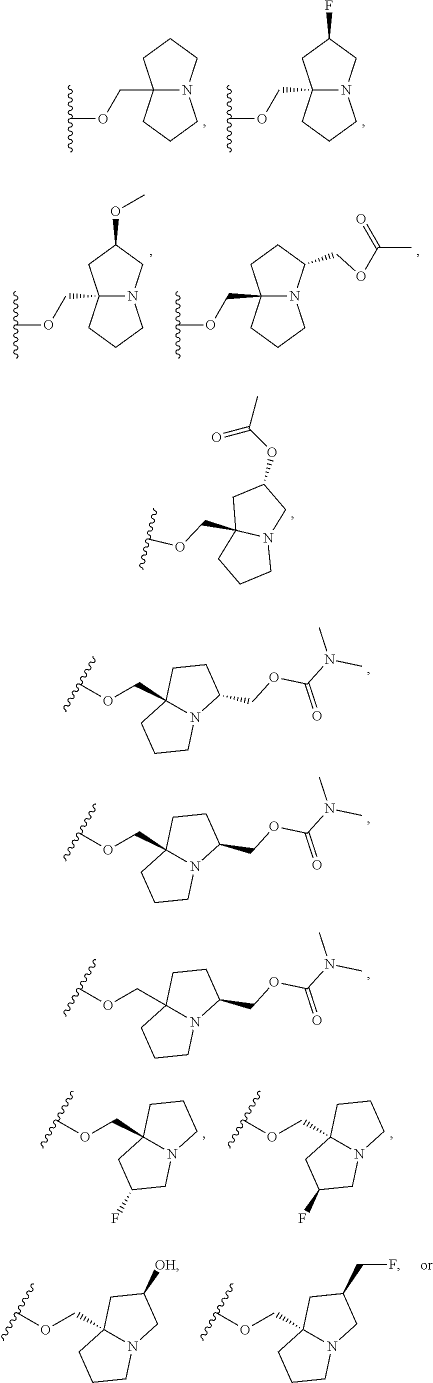

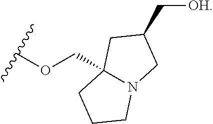 [SMILES: *OC[C@@]12CCCN1C[C@H](CO)C2]

[20]. The compound of any one of aspects [1] to [19], wherein, the moiety of —X₂—R₂ or —O—R₂ is selected from

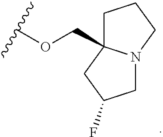 [SMILES: *OC[C@@]12CCCN1C[C@H](F)C2]

[21]. The compound of any one of aspects [1] to [20], wherein, R₄ is selected from

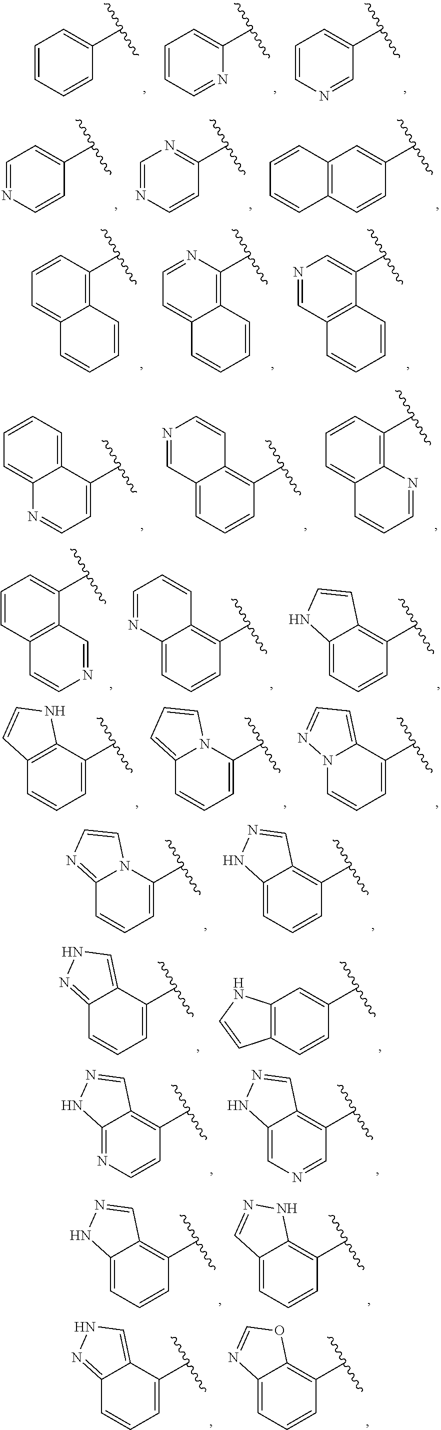

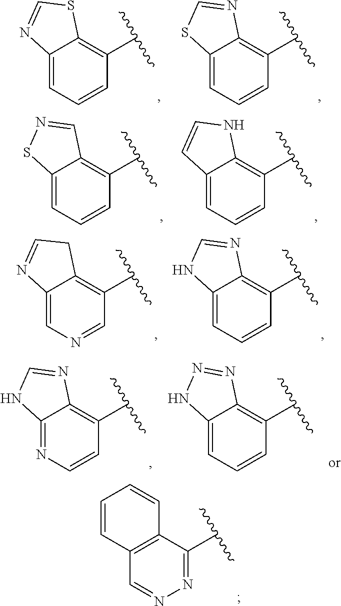

wherein said R₄ is independently optionally substituted with 1, 2, 3, 4, 5 or 6 R₄ₐ;
Each of R₄ₐ is independently selected from —F, —Cl, —C₁₋₃alkyl, haloC₁₋₃alkyl, haloC₁₋₃alkoxy, —C₂₋₃alkenyl, —C₂₋₃alkynyl, —CN, —NH₂, —NH(C₁₋₃alkyl), —N(C₁₋₃alkyl)₂, —OH, —O(C₁₋₃alkyl), —SH, —S(C₁₋₃alkyl), 3-6 membered cycloalkyl or 3-6 membered heterocyclyl, wherein said —C₁₋₃alkyl, haloC₁₋₃alkyl, haloC₁₋₃alkoxy, —C₂₋₃alkenyl, —C₂₋₆alkynyl, 3-6 membered cycloalkyl or 3-6 membered heterocyclyl is independently optionally substituted with 1, 2 or 3 R_{4b};
Each of R_{4b} is independently selected from —F; —C₁₋₃alkyl; haloC₁₋₃alkyl; —CN; —OH; —NH₂; —NH(C₁₋₃alkyl); —NH(C₁₋₃alkyl)₂; —OC₁₋₃alkyl; or —C₁₋₃alkyl substituted with 1, 2 or 3 substituents selected form —F, haloC₁₋₃alkyl, —CN, —OH, —NH₂, —NH(C₁₋₃alkyl), —NH(C₁₋₃alkyl)₂ or —OC₁₋₃alkyl.

[22]. The compound of any one of aspects [1] to [21], wherein, R₄ is selected from

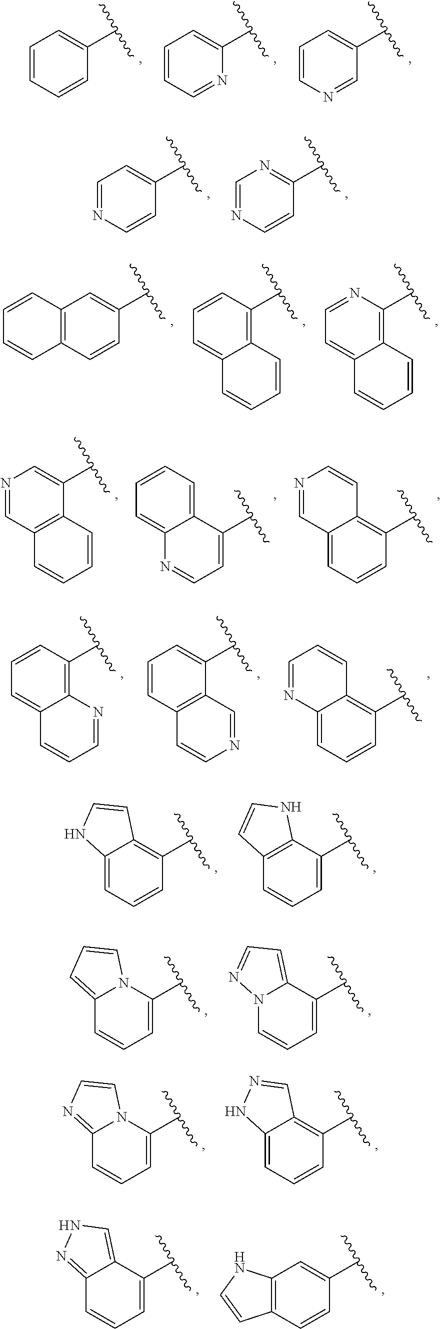

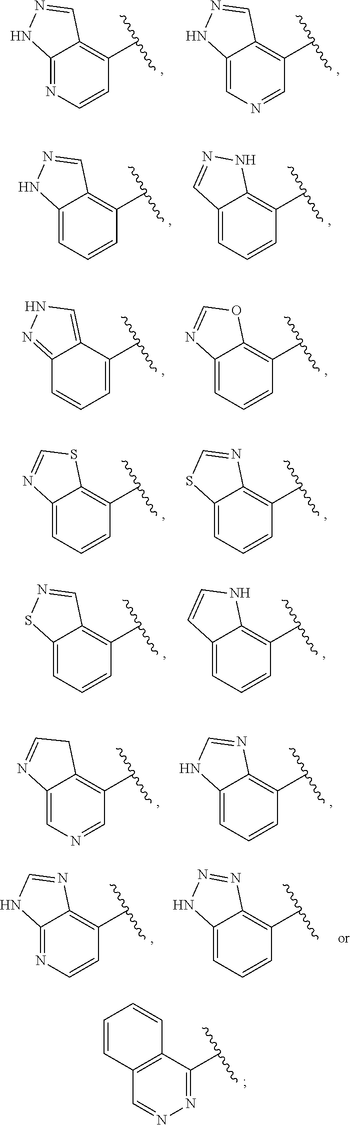

wherein said R₄ is independently optionally substituted with 1, 2 or 3 R₄ₐ;
Each of R₄ₐ is independently selected from —F, —Cl, methyl, ethyl, isopropyl, —CH═CH₂, —C≡CH, —C≡CCH₃, —C≡CD, —CH₂C≡CH, —CHF₂, —CF₃, —CH₂CF₃, —CH₂CHF₂, —CH₂CH₂F, —CH₂CH₂CH₂F, —OCF₃, —CN, —CH₂CH₂CN, —NH₂, —N(CH₃)₂, —NHCH₂CH₃, —CH₂—N(CH₃)₂, —OH, —CH₂OH, —CH₂CH₂OH, —OCH₃, —OC(CH₃)₂, —CH₂CH(CH₃)₂, —CH(CH₃)CH₂CH₃, —CH₂OCH₃, —SH, —SCH₃, —SCF₃, —OCHF₂, —CH(CF₃)OCH₃, —C(CH₃)₂OH, —CF(CH₃)₂, —OCH(CH₃)₂, cyclopropyl,

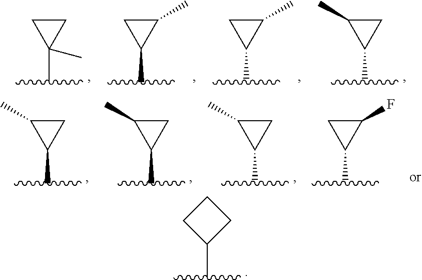

[23]. The compound of any one of aspects [1] to [22], wherein, R₄ is selected from any one of the structures in the following Table 4:

TABLE 4

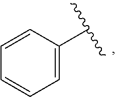 [SMILES: *c1ccccc1]

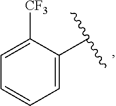 [SMILES: *c1ccccc1C(F)(F)F]

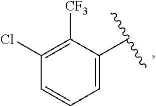 [SMILES: *c1cccc(Cl)c1C(F)(F)F]

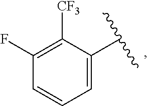 [SMILES: *c1cccc(F)c1C(F)(F)F]

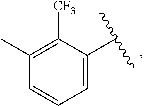 [SMILES: *c1cccc(C)c1C(F)(F)F]

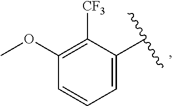 [SMILES: *c1cccc(OC)c1C(F)(F)F]

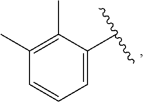 [SMILES: *c1cccc(C)c1C]

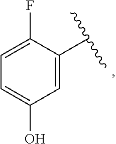 [SMILES: *c1cc(O)ccc1F]

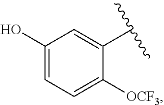 [SMILES: *c1cc(O)ccc1OC(F)(F)F]

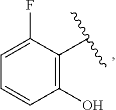 [SMILES: *c1c(O)cccc1F]

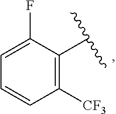 [SMILES: *c1c(F)cccc1C(F)(F)F]

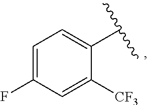 [SMILES: *c1ccc(F)cc1C(F)(F)F]

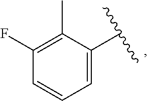 [SMILES: *c1cccc(F)c1C]

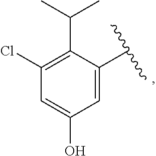 [SMILES: *c1cc(O)cc(Cl)c1C(C)C]

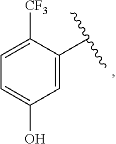 [SMILES: *c1cc(O)ccc1C(F)(F)F]

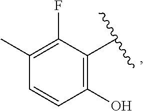 [SMILES: *c1c(O)ccc(C)c1F]

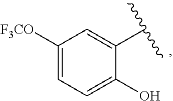 [SMILES: *c1cc(OC(F)(F)F)ccc1O]

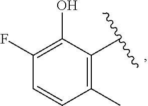 [SMILES: *c1c(C)ccc(F)c1O]

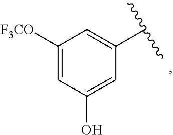 [SMILES: *c1cc(O)cc(OC(F)(F)F)c1]

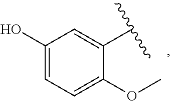 [SMILES: *c1cc(O)ccc1OC]

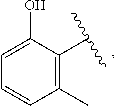 [SMILES: *c1c(C)cccc1O]

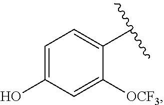 [SMILES: *c1ccc(O)cc1OC(F)(F)F]

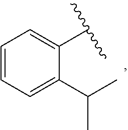 [SMILES: *c1ccccc1C(C)C]

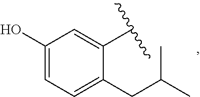 [SMILES: *c1cc(O)ccc1CC(C)C]

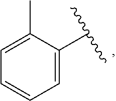 [SMILES: *c1ccccc1C]

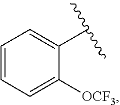 [SMILES: *c1ccccc1OC(F)(F)F]

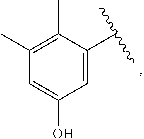 [SMILES: *c1cc(O)cc(C)c1C]

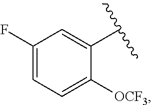 [SMILES: *c1cc(F)ccc1OC(F)(F)F]

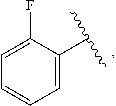 [SMILES: *c1ccccc1F]

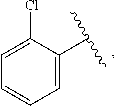 [SMILES: *c1ccccc1Cl]

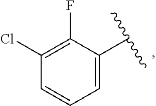 [SMILES: *c1cccc(Cl)c1F]

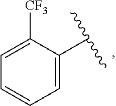 [SMILES: *c1ccccc1C(F)(F)F]

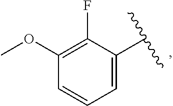 [SMILES: *c1cccc(OC)c1F]

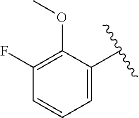 [SMILES: *c1cccc(F)c1OC]

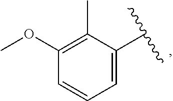 [SMILES: *c1cccc(OC)c1C]

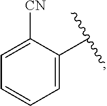

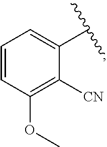

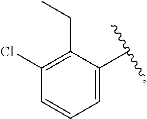

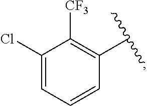

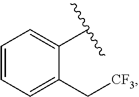 [SMILES: *c1ccccc1CC(F)(F)F]

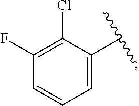

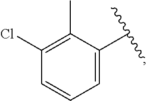

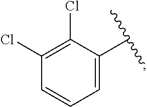

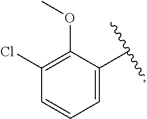

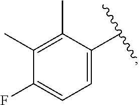

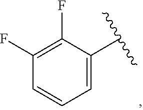 [SMILES: *c1cccc(F)c1F]

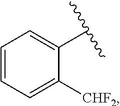 [SMILES: *c1ccccc1C(F)F]

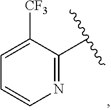 [SMILES: *c1ncccc1C(F)(F)F]

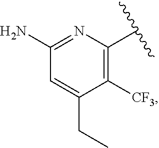 [SMILES: *c1nc(N)cc(CC)c1C(F)(F)F]

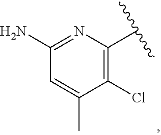 [SMILES: *c1nc(N)cc(C)c1Cl]

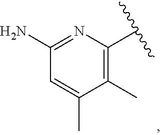 [SMILES: *c1nc(N)cc(C)c1C]

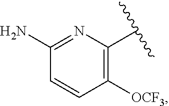 [SMILES: *c1nc(N)ccc1OC(F)(F)F]

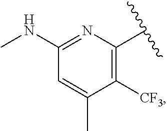 [SMILES: *c1nc(NC)cc(C)c1C(F)(F)F]

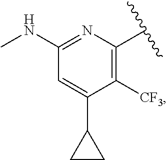 [SMILES: *c1nc(NC)cc(C2CC2)c1C(F)(F)F]

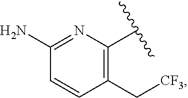 [SMILES: *c1nc(N)ccc1CC(F)(F)F]

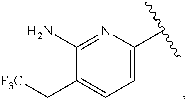 [SMILES: *c1ccc(CC(F)(F)F)c(N)n1]

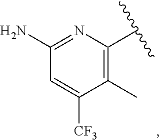 [SMILES: *c1nc(N)cc(C(F)(F)F)c1C]

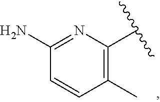 [SMILES: *c1nc(N)ccc1C]

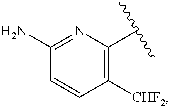 [SMILES: *c1nc(N)ccc1C(F)F]

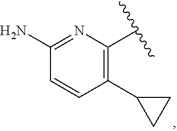 [SMILES: *c1nc(N)ccc1C1CC1]

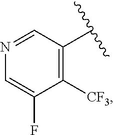 [SMILES: *c1cncc(F)c1C(F)(F)F]

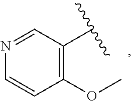 [SMILES: *c1cnccc1OC]

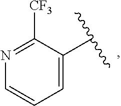 [SMILES: *c1cccnc1C(F)(F)F]

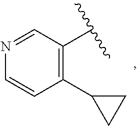 [SMILES: *c1cnccc1C1CC1]

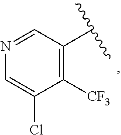 [SMILES: *c1cncc(Cl)c1C(F)(F)F]

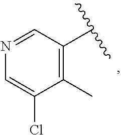 [SMILES: *c1cncc(Cl)c1C]

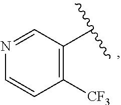 [SMILES: *c1cnccc1C(F)(F)F]

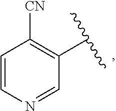 [SMILES: *c1cnccc1C#N]

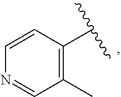 [SMILES: *c1ccncc1C]

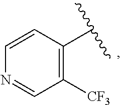 [SMILES: *c1ccncc1C(F)(F)F]

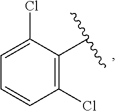 [SMILES: *c1c(Cl)cccc1Cl]

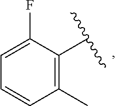 [SMILES: *c1c(C)cccc1F]

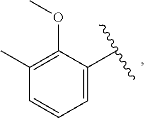 [SMILES: *c1cccc(C)c1OC]

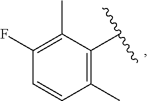 [SMILES: *c1c(C)ccc(F)c1C]

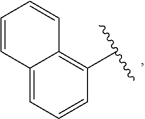 [SMILES: *c1cccc2ccccc12]

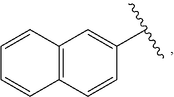 [SMILES: *c1ccc2ccccc2c1]

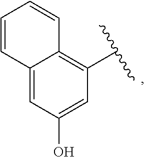 [SMILES: *c1cc(O)cc2ccccc12]

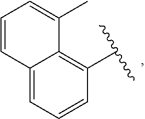 [SMILES: *c1cccc2cccc(C)c12]

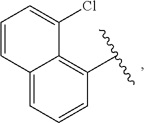 [SMILES: *c1cccc2cccc(Cl)c12]

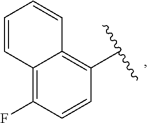 [SMILES: *c1ccc(F)c2ccccc12]

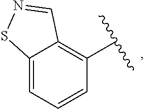 [SMILES: *c1cccc2sncc12]

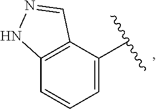 [SMILES: *c1cccc2[nH]ncc12]

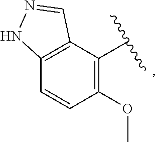 [SMILES: *c1c(OC)ccc2[nH]ncc12]

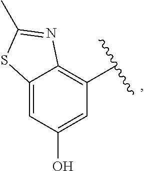 [SMILES: *c1cc(O)cc2sc(C)nc12]

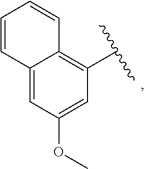 [SMILES: *c1cc(OC)cc2ccccc12]

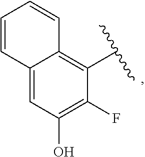 [SMILES: *c1c(F)c(O)cc2ccccc12]

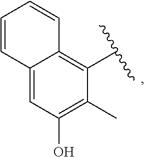 [SMILES: *c1c(C)c(O)cc2ccccc12]

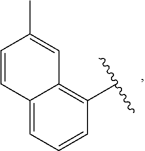 [SMILES: *c1cccc2ccc(C)cc12]

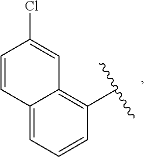 [SMILES: *c1cccc2ccc(Cl)cc12]

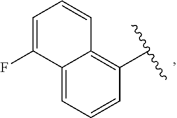 [SMILES: *c1cccc2c(F)cccc12]

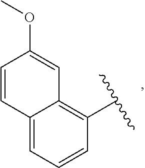 [SMILES: *c1cccc2ccc(OC)cc12]

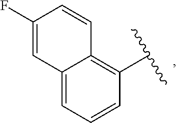 [SMILES: *c1cccc2cc(F)ccc12]

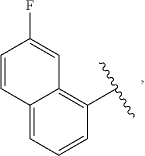 [SMILES: *c1cccc2ccc(F)cc12]

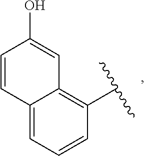 [SMILES: *c1cccc2ccc(O)cc12]

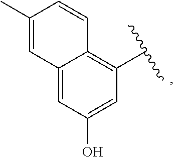 [SMILES: *c1cc(O)cc2cc(C)ccc12]

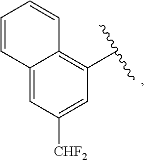 [SMILES: *c1cc(C(F)F)cc2ccccc12]

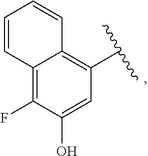 [SMILES: *c1cc(O)c(F)c2ccccc12]

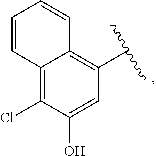 [SMILES: *c1cc(O)c(Cl)c2ccccc12]

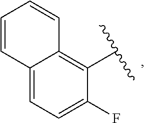 [SMILES: *c1c(F)ccc2ccccc12]

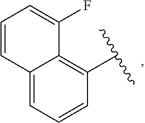 [SMILES: *c1cccc2cccc(F)c12]

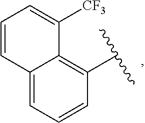 [SMILES: *c1cccc2cccc(C(F)(F)F)c12]

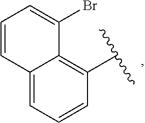 [SMILES: *c1cccc2cccc(Br)c12]

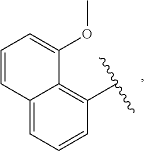 [SMILES: *c1cccc2cccc(OC)c12]

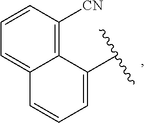 [SMILES: *c1cccc2cccc(C#N)c12]

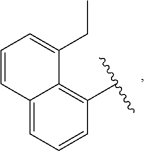 [SMILES: *c1cccc2cccc(CC)c12]

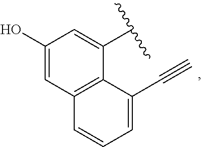 [SMILES: *c1cc(O)cc2cccc(C#C)c12]

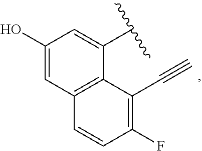 [SMILES: *c1cc(O)cc2ccc(F)c(C#C)c12]

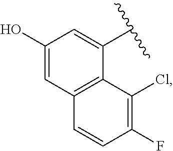 [SMILES: *c1cc(O)cc2ccc(F)c(Cl)c12]

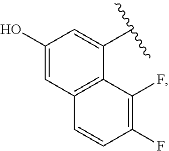 [SMILES: *c1cc(O)cc2ccc(F)c(F)c12]

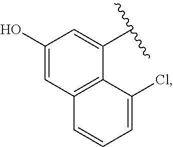 [SMILES: *c1cc(O)cc2cccc(Cl)c12]

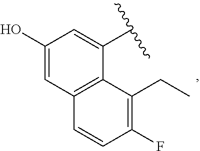 [SMILES: *c1cc(O)cc2ccc(F)c(CC)c12]

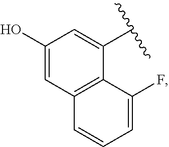 [SMILES: *c1cc(O)cc2cccc(F)c12]

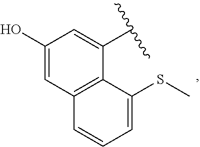 [SMILES: *c1cc(O)cc2cccc(SC)c12]

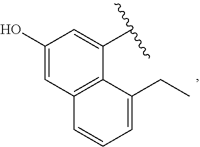 [SMILES: *c1cc(O)cc2cccc(CC)c12]

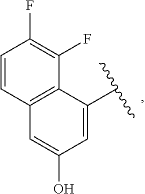 [SMILES: *c1cc(O)cc2ccc(F)c(F)c12]

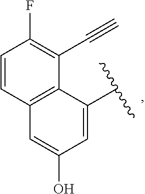 [SMILES: *c1cc(O)cc2ccc(F)c(C#C)c12]

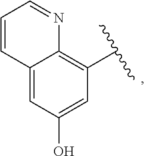 [SMILES: *c1cc(O)cc2cccnc12]

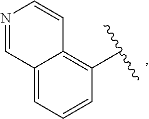 [SMILES: *c1cccc2cnccc12]

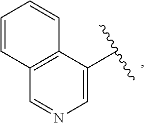 [SMILES: *c1cncc2ccccc12]

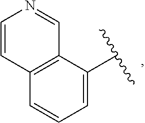 [SMILES: *c1cccc2ccncc12]

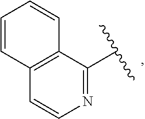 [SMILES: *c1nccc2ccccc12]

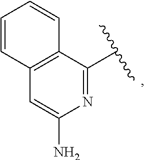 [SMILES: *c1nc(N)cc2ccccc12]

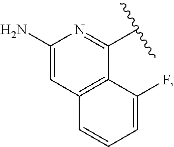 [SMILES: *c1nc(N)cc2cccc(F)c12]

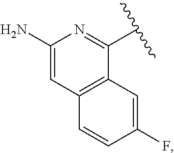 [SMILES: *c1nc(N)cc2ccc(F)cc12]

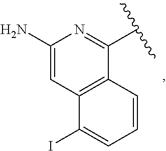 [SMILES: *c1nc(N)cc2c(I)cccc12]

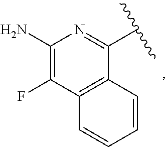 [SMILES: *c1nc(N)c(F)c2ccccc12]

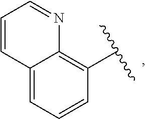 [SMILES: *c1cccc2cccnc12]

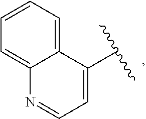 [SMILES: *c1ccnc2ccccc12]

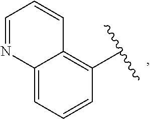 [SMILES: *c1cccc2ncccc12]

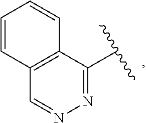 [SMILES: *c1nncc2ccccc12]

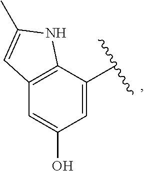 [SMILES: *c1cc(O)cc2cc(C)[nH]c12]

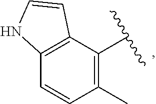 [SMILES: *c1c(C)ccc2[nH]ccc12]

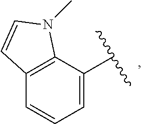 [SMILES: *c1cccc2ccn(C)c12]

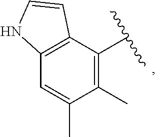 [SMILES: *c1c(C)c(C)cc2[nH]ccc12]

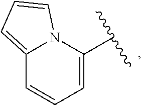 [SMILES: *c1cccc2cccn12]

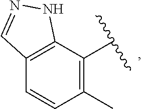 [SMILES: *c1c(C)ccc2cn[nH]c12]

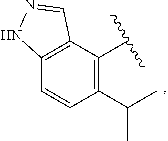 [SMILES: *c1c(C(C)C)ccc2[nH]ncc12]

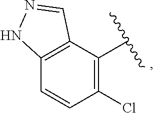 [SMILES: *c1c(Cl)ccc2[nH]ncc12]

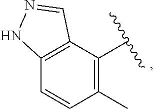 [SMILES: *c1c(C)ccc2[nH]ncc12]

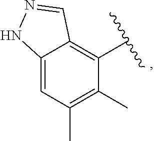 [SMILES: *c1c(C)c(C)cc2[nH]ncc12]

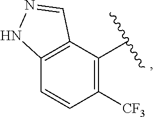 [SMILES: *c1c(C(F)(F)F)ccc2[nH]ncc12]

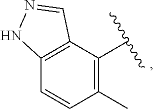 [SMILES: *c1c(C)ccc2[nH]ncc12]

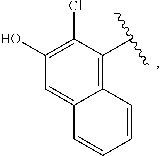 [SMILES: *c1c(Cl)c(O)cc2ccccc12]

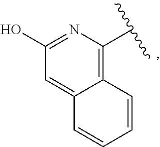 [SMILES: *c1nc(O)cc2ccccc12]

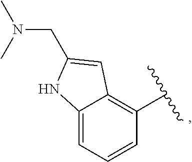 [SMILES: *c1cccc2[nH]c(CN(C)C)cc12]

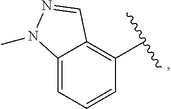 [SMILES: *c1cccc2c1cnn2C]

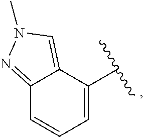 [SMILES: *c1cccc2nn(C)cc12]

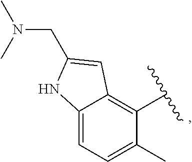

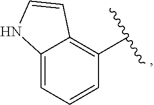 [SMILES: *c1cccc2[nH]ccc12]

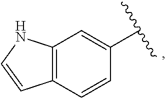 [SMILES: *c1ccc2cc[nH]c2c1]

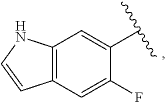 [SMILES: *c1cc2[nH]ccc2cc1F]

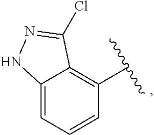 [SMILES: *c1cccc2[nH]nc(Cl)c12]

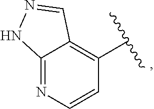 [SMILES: *c1ccnc2[nH]ncc12]

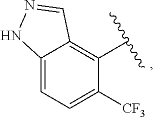 [SMILES: *c1c(C(F)(F)F)ccc2[nH]ncc12]

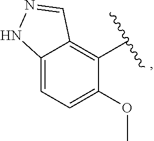 [SMILES: *c1c(OC)ccc2[nH]ncc12]

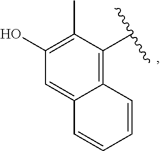 [SMILES: *c1c(C)c(O)cc2ccccc12]

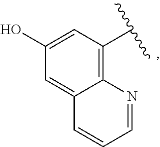 [SMILES: *c1cc(O)cc2cccnc12]

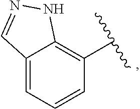 [SMILES: *c1cccc2cn[nH]c12]

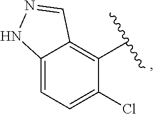 [SMILES: *c1c(Cl)ccc2[nH]ncc12]

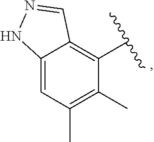 [SMILES: *c1c(C)c(C)cc2[nH]ncc12]

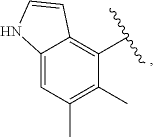 [SMILES: *c1c(C)c(C)cc2[nH]ccc12]

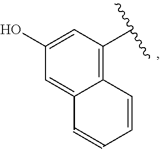 [SMILES: *c1cc(O)cc2ccccc12]

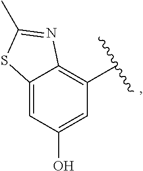 [SMILES: *c1cc(O)cc2sc(C)nc12]

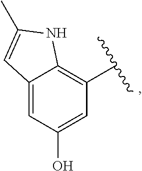 [SMILES: *c1cc(O)cc2cc(C)[nH]c12]

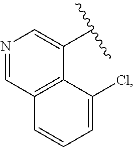 [SMILES: *c1cncc2cccc(Cl)c12]

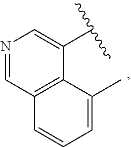 [SMILES: *c1cncc2cccc(C)c12]

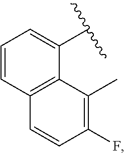 [SMILES: *c1cccc2ccc(F)c(C)c12]

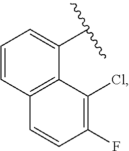 [SMILES: *c1cccc2ccc(F)c(Cl)c12]

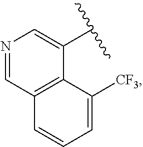 [SMILES: *c1cncc2cccc(C(F)(F)F)c12]

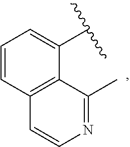 [SMILES: *c1cccc2ccnc(C)c12]

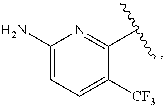 [SMILES: *c1nc(N)ccc1C(F)(F)F]

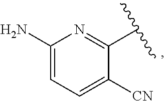 [SMILES: *c1nc(N)ccc1C#N]

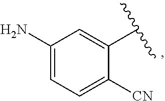 [SMILES: *c1cc(N)ccc1C#N]

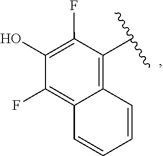 [SMILES: *c1c(F)c(O)c(F)c2ccccc12]

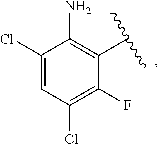 [SMILES: *c1c(N)c(Cl)cc(Cl)c1F]

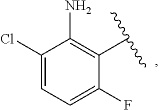 [SMILES: *c1c(F)ccc(Cl)c1N]

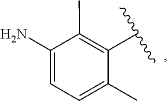 [SMILES: *c1c(C)ccc(N)c1C]

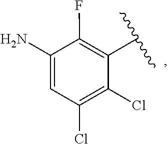 [SMILES: *c1c(F)c(N)cc(Cl)c1Cl]

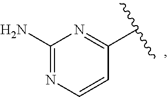 [SMILES: *c1ccnc(N)n1]

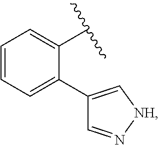 [SMILES: *c1ccccc1-c1cn[nH]c1]

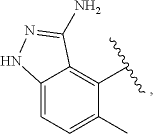 [SMILES: *c1c(C)ccc2[nH]nc(N)c12]

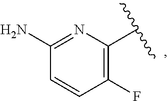 [SMILES: *c1nc(N)ccc1F]

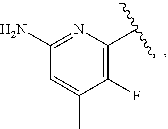 [SMILES: *c1nc(N)cc(C)c1F]

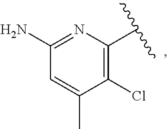 [SMILES: *c1nc(N)cc(C)c1Cl]

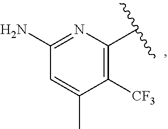 [SMILES: *c1nc(N)cc(C)c1C(F)(F)F]

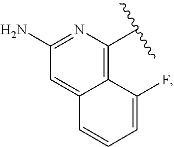 [SMILES: *c1nc(N)cc2cccc(F)c12]

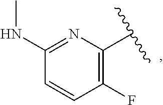 [SMILES: *c1nc(NC)ccc1F]

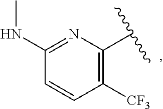 [SMILES: *c1nc(NC)ccc1C(F)(F)F]

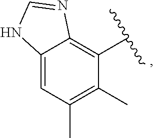 [SMILES: *c1c(C)c(C)cc2[nH]cnc12]

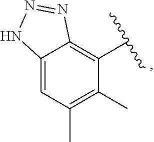 [SMILES: *c1c(C)c(C)cc2[nH]nnc12]

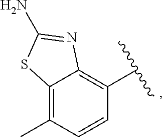 [SMILES: *c1ccc(C)c2sc(N)nc12]

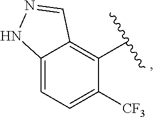 [SMILES: *c1c(C(F)(F)F)ccc2[nH]ncc12]

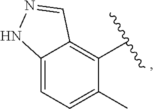 [SMILES: *c1c(C)ccc2[nH]ncc12]

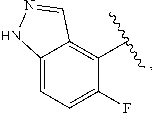 [SMILES: *c1c(F)ccc2[nH]ncc12]

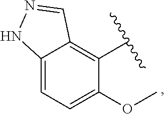 [SMILES: *c1c(OC)ccc2[nH]ncc12]

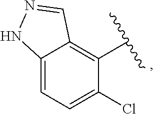 [SMILES: *c1c(Cl)ccc2[nH]ncc12]

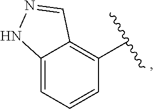 [SMILES: *c1cccc2[nH]ncc12]

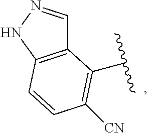 [SMILES: *c1c(C#N)ccc2[nH]ncc12]

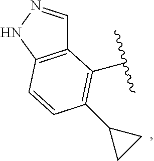 [SMILES: *c1c(C2CC2)ccc2[nH]ncc12]

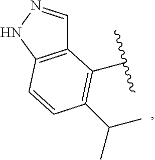 [SMILES: *c1c(C(C)C)ccc2[nH]ncc12]

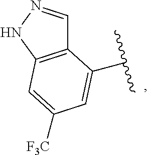 [SMILES: *c1cc(C(F)(F)F)cc2[nH]ncc12]

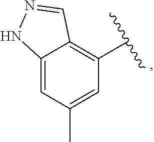 [SMILES: *c1cc(C)cc2[nH]ncc12]

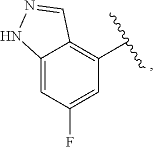 [SMILES: *c1cc(F)cc2[nH]ncc12]

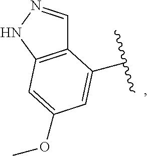 [SMILES: *c1cc(OC)cc2[nH]ncc12]

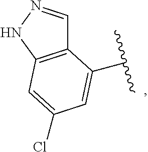 [SMILES: *c1cc(Cl)cc2[nH]ncc12]

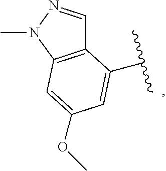 [SMILES: *c1cc(OC)cc2c1cnn2C]

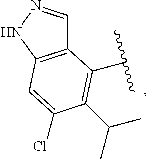 [SMILES: *c1c(C(C)C)c(Cl)cc2[nH]ncc12]

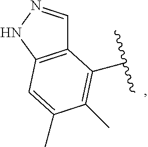 [SMILES: *c1c(C)c(C)cc2[nH]ncc12]

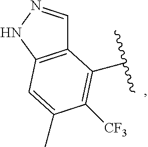 [SMILES: *c1c(C(F)(F)F)c(C)cc2[nH]ncc12]

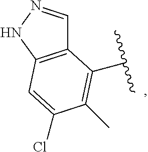 [SMILES: *c1c(C)c(Cl)cc2[nH]ncc12]

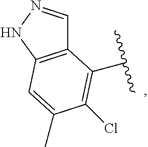 [SMILES: *c1c(Cl)c(C)cc2[nH]ncc12]

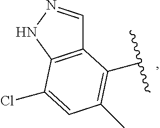 [SMILES: *c1c(C)cc(Cl)c2[nH]ncc12]

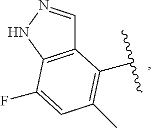 [SMILES: *c1c(C)cc(F)c2[nH]ncc12]

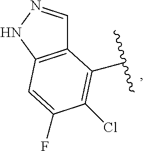 [SMILES: *c1c(Cl)c(F)cc2[nH]ncc12]

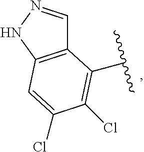 [SMILES: *c1c(Cl)c(Cl)cc2[nH]ncc12]

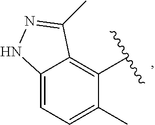 [SMILES: *c1c(C)ccc2[nH]nc(C)c12]

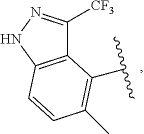 [SMILES: *c1c(C)ccc2[nH]nc(C(F)(F)F)c12]

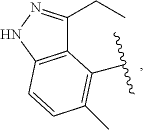 [SMILES: *c1c(C)ccc2[nH]nc(CC)c12]

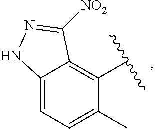 [SMILES: *c1c(C)ccc2[nH]nc([N+](=O)[O-])c12]

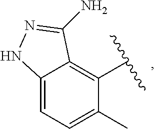 [SMILES: *c1c(C)ccc2[nH]nc(N)c12]

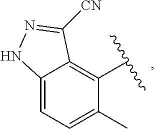 [SMILES: *c1c(C)ccc2[nH]nc(C#N)c12]

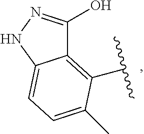 [SMILES: *c1c(C)ccc2[nH]nc(O)c12]

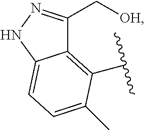 [SMILES: *c1c(C)ccc2[nH]nc(CO)c12]

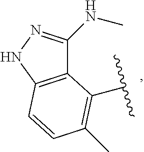 [SMILES: *c1c(C)ccc2[nH]nc(NC)c12]

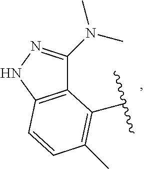 [SMILES: *c1c(C)ccc2[nH]nc(N(C)C)c12]

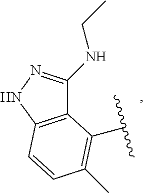 [SMILES: *c1c(C)ccc2[nH]nc(NCC)c12]

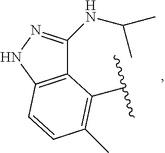 [SMILES: *c1c(C)ccc2[nH]nc(NC(C)C)c12]

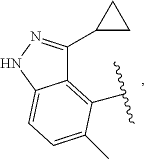 [SMILES: *c1c(C)ccc2[nH]nc(C3CC3)c12]

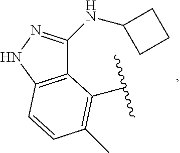 [SMILES: *c1c(C)ccc2[nH]nc(NC3CCC3)c12]

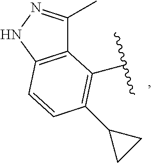 [SMILES: *c1c(C2CC2)ccc2[nH]nc(C)c12]

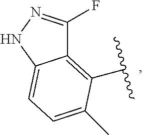 [SMILES: *c1c(C)ccc2[nH]nc(F)c12]

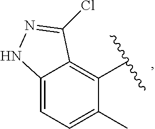 [SMILES: *c1c(C)ccc2[nH]nc(Cl)c12]

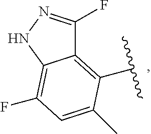 [SMILES: *c1c(C)cc(F)c2[nH]nc(F)c12]

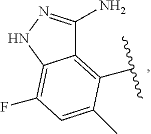 [SMILES: *c1c(C)cc(F)c2[nH]nc(N)c12]

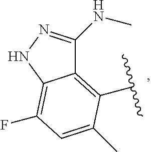 [SMILES: *c1c(C)cc(F)c2[nH]nc(NC)c12]

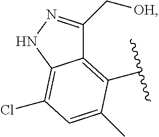 [SMILES: *c1c(C)cc(Cl)c2[nH]nc(CO)c12]

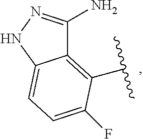 [SMILES: *c1c(F)ccc2[nH]nc(N)c12]

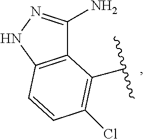 [SMILES: *c1c(Cl)ccc2[nH]nc(N)c12]

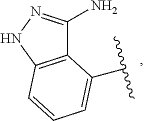 [SMILES: *c1cccc2[nH]nc(N)c12]

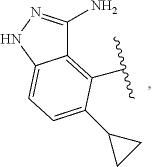 [SMILES: *c1c(C2CC2)ccc2[nH]nc(N)c12]

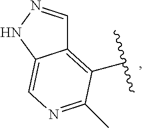 [SMILES: *c1c(C)ncc2[nH]ncc12]

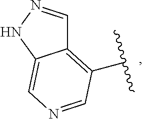 [SMILES: *c1cncc2[nH]ncc12]

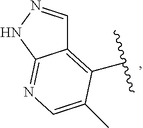 [SMILES: *c1c(C)cnc2[nH]ncc12]

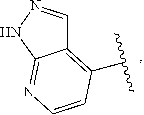 [SMILES: *c1ccnc2[nH]ncc12]

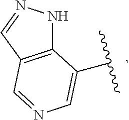 [SMILES: *c1cncc2cn[nH]c12]

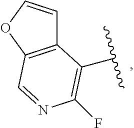 [SMILES: *c1c(F)ncc2occc12]

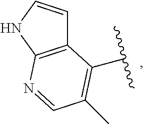 [SMILES: *c1c(C)cnc2[nH]ccc12]

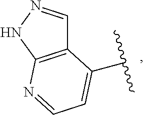 [SMILES: *c1ccnc2[nH]ncc12]

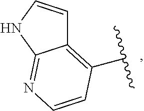 [SMILES: *c1ccnc2[nH]ccc12]

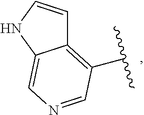 [SMILES: *c1cncc2[nH]ccc12]

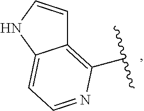 [SMILES: *c1nccc2[nH]ccc12]

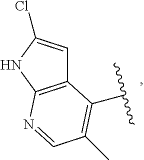 [SMILES: *c1c(C)cnc2[nH]c(Cl)cc12]

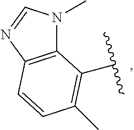 [SMILES: *c1c(C)ccc2ncn(C)c12]

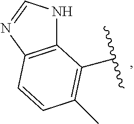 [SMILES: *c1c(C)ccc2nc[nH]c12]

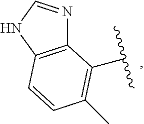 [SMILES: *c1c(C)ccc2[nH]cnc12]

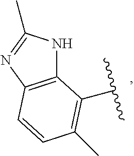 [SMILES: *c1c(C)ccc2nc(C)[nH]c12]

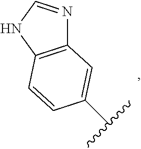 [SMILES: *c1ccc2[nH]cnc2c1]

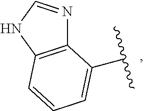 [SMILES: *c1cccc2[nH]cnc12]

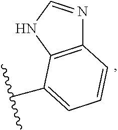 [SMILES: *c1cccc2nc[nH]c12]

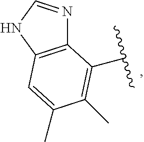 [SMILES: *c1c(C)c(C)cc2[nH]cnc12]

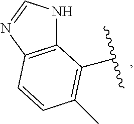 [SMILES: *c1c(C)ccc2nc[nH]c12]

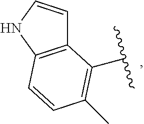 [SMILES: *c1c(C)ccc2[nH]ccc12]

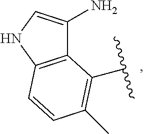 [SMILES: *c1c(C)ccc2[nH]cc(N)c12]

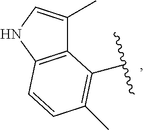 [SMILES: *c1c(C)ccc2[nH]cc(C)c12]

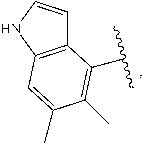 [SMILES: *c1c(C)c(C)cc2[nH]ccc12]

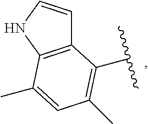 [SMILES: *c1c(C)cc(C)c2[nH]ccc12]

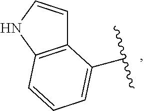 [SMILES: *c1cccc2[nH]ccc12]

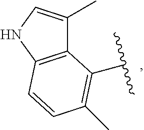 [SMILES: *c1c(C)ccc2[nH]cc(C)c12]

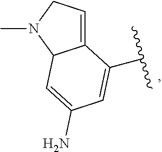 [SMILES: *C1=CC(N)=CC2C1=CCN2C]

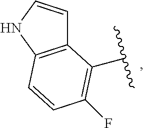 [SMILES: *c1c(F)ccc2[nH]ccc12]

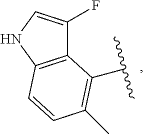 [SMILES: *c1c(C)ccc2[nH]cc(F)c12]

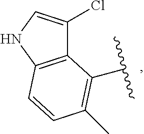 [SMILES: *c1c(C)ccc2[nH]cc(Cl)c12]

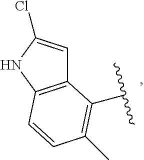 [SMILES: *c1c(C)ccc2[nH]c(Cl)cc12]

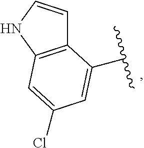 [SMILES: *c1cc(Cl)cc2[nH]ccc12]

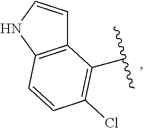 [SMILES: *c1c(Cl)ccc2[nH]ccc12]

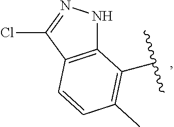 [SMILES: *c1c(C)ccc2c(Cl)n[nH]c12]

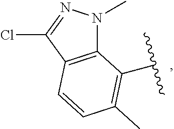 [SMILES: *c1c(C)ccc2c(Cl)nn(C)c12]

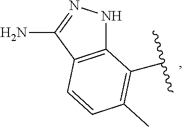 [SMILES: *c1c(C)ccc2c(N)n[nH]c12]

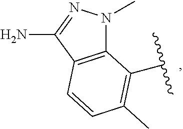 [SMILES: *c1c(C)ccc2c(N)nn(C)c12]

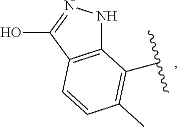 [SMILES: *c1c(C)ccc2c(O)n[nH]c12]

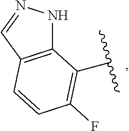 [SMILES: *c1c(F)ccc2cn[nH]c12]

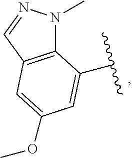 [SMILES: *c1cc(OC)cc2cnn(C)c12]

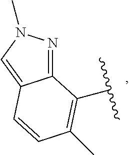 [SMILES: *c1c(C)ccc2cn(C)nc12]

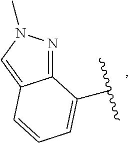 [SMILES: *c1cccc2cn(C)nc12]

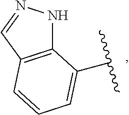 [SMILES: *c1cccc2cn[nH]c12]

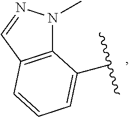 [SMILES: *c1cccc2cnn(C)c12]

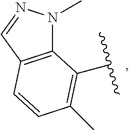 [SMILES: *c1c(C)ccc2cnn(C)c12]

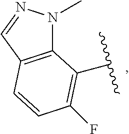 [SMILES: *c1c(F)ccc2cnn(C)c12]

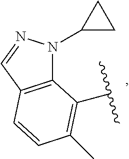 [SMILES: *c1c(C)ccc2cnn(C3CC3)c12]

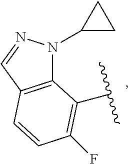 [SMILES: *c1c(F)ccc2cnn(C3CC3)c12]

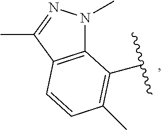 [SMILES: *c1c(C)ccc2c(C)nn(C)c12]

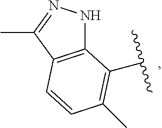 [SMILES: *c1c(C)ccc2c(C)n[nH]c12]

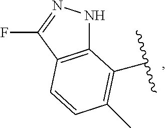 [SMILES: *c1c(C)ccc2c(F)n[nH]c12]

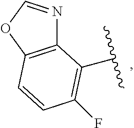 [SMILES: *c1c(F)ccc2ocnc12]

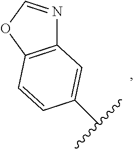 [SMILES: *c1ccc2ocnc2c1]

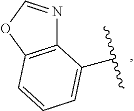 [SMILES: *c1cccc2ocnc12]

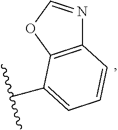 [SMILES: *c1cccc2ncoc12]

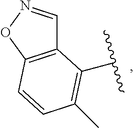 [SMILES: *c1c(C)ccc2oncc12]

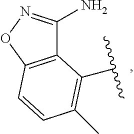 [SMILES: *c1c(C)ccc2onc(N)c12]

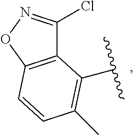 [SMILES: *c1c(C)ccc2onc(Cl)c12]

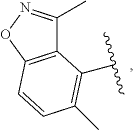 [SMILES: *c1c(C)ccc2onc(C)c12]

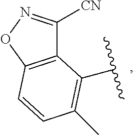 [SMILES: *c1c(C)ccc2onc(C#N)c12]

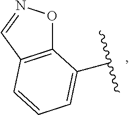 [SMILES: *c1cccc2cnoc12]

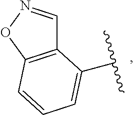 [SMILES: *c1cccc2oncc12]

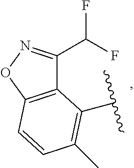 [SMILES: *c1c(C)ccc2onc(C(F)F)c12]

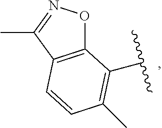 [SMILES: *c1c(C)ccc2c(C)noc12]

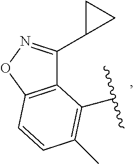 [SMILES: *c1c(C)ccc2onc(C3CC3)c12]

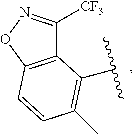 [SMILES: *c1c(C)ccc2onc(C(F)(F)F)c12]

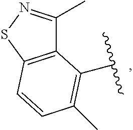 [SMILES: *c1c(C)ccc2snc(C)c12]

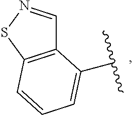 [SMILES: *c1cccc2sncc12]

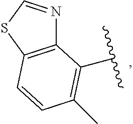 [SMILES: *c1c(C)ccc2scnc12]

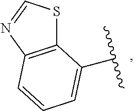 [SMILES: *c1cccc2ncsc12]

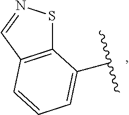 [SMILES: *c1cccc2cnsc12]

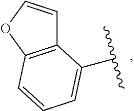 [SMILES: *c1cccc2occc12]

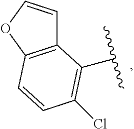 [SMILES: *c1c(Cl)ccc2occc12]

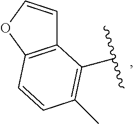 [SMILES: *c1c(C)ccc2occc12]

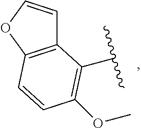 [SMILES: *c1c(OC)ccc2occc12]

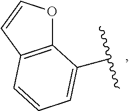 [SMILES: *c1cccc2ccoc12]

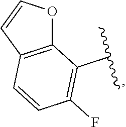 [SMILES: *c1c(F)ccc2ccoc12]

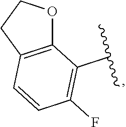 [SMILES: *c1c(F)ccc2c1OCC2]

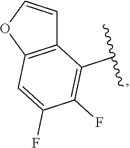 [SMILES: *c1c(F)c(F)cc2occc12]

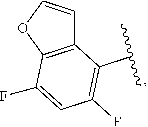 [SMILES: *c1c(F)cc(F)c2occc12]

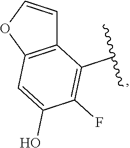 [SMILES: *c1c(F)c(O)cc2occc12]

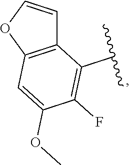 [SMILES: *c1c(F)c(OC)cc2occc12]

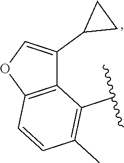 [SMILES: *c1c(C)ccc2occ(C3CC3)c12]

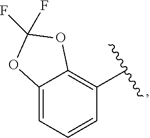 [SMILES: *c1cccc2c1OC(F)(F)O2]

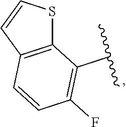 [SMILES: *c1c(F)ccc2ccsc12]

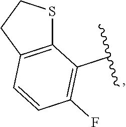 [SMILES: *c1c(F)ccc2c1SCC2]

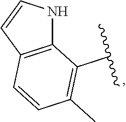 [SMILES: *c1c(C)ccc2cc[nH]c12]

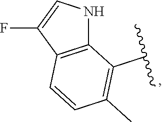 [SMILES: *c1c(C)ccc2c(F)c[nH]c12]

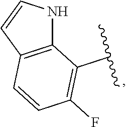 [SMILES: *c1c(F)ccc2cc[nH]c12]

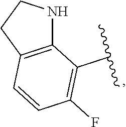 [SMILES: *c1c(F)ccc2c1NCC2]

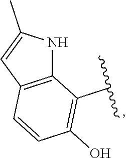 [SMILES: *c1c(O)ccc2cc(C)[nH]c12]

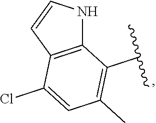 [SMILES: *c1c(C)cc(Cl)c2cc[nH]c12]

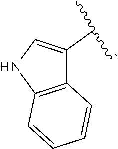 [SMILES: *c1c[nH]c2ccccc12]

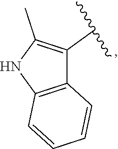 [SMILES: *c1c(C)[nH]c2ccccc12]

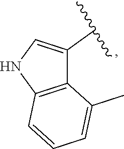 [SMILES: *c1c[nH]c2cccc(C)c12]

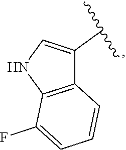 [SMILES: *c1c[nH]c2c(F)cccc12]

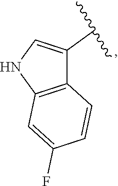 [SMILES: *c1c[nH]c2cc(F)ccc12]

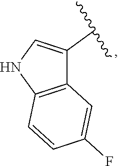 [SMILES: *c1c[nH]c2ccc(F)cc12]

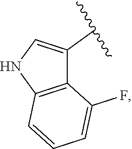 [SMILES: *c1c[nH]c2cccc(F)c12]

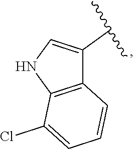 [SMILES: *c1c[nH]c2c(Cl)cccc12]

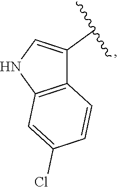 [SMILES: *c1c[nH]c2cc(Cl)ccc12]

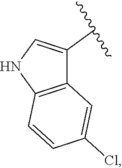 [SMILES: *c1c[nH]c2ccc(Cl)cc12]

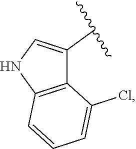 [SMILES: *c1c[nH]c2cccc(Cl)c12]

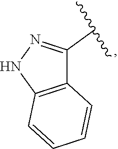 [SMILES: *c1n[nH]c2ccccc12]

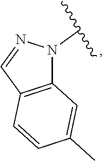 [SMILES: *n1ncc2ccc(C)cc21]

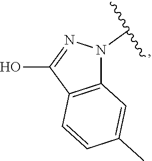 [SMILES: *n1nc(O)c2ccc(C)cc21]

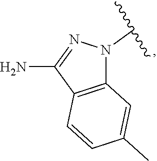 [SMILES: *n1nc(N)c2ccc(C)cc21]

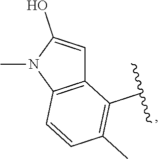 [SMILES: *c1c(C)ccc2c1cc(O)n2C]

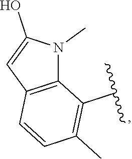 [SMILES: *c1c(C)ccc2cc(O)n(C)c12]

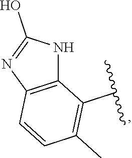 [SMILES: *c1c(C)ccc2nc(O)[nH]c12]

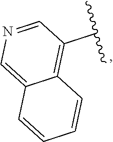 [SMILES: *c1cncc2ccccc12]

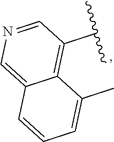 [SMILES: *c1cncc2cccc(C)c12]

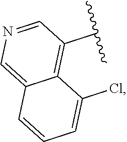 [SMILES: *c1cncc2cccc(Cl)c12]

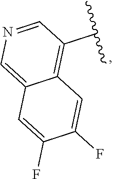 [SMILES: *c1cncc2cc(F)c(F)cc12]

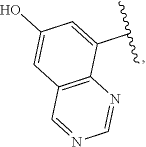 [SMILES: *c1cc(O)cc2cncnc12]

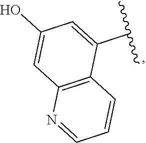 [SMILES: *c1cc(O)cc2ncccc12]

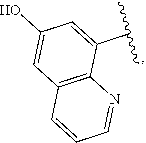 [SMILES: *c1cc(O)cc2cccnc12]

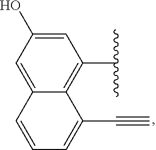 [SMILES: *c1cc(O)cc2cccc(C#C)c12]

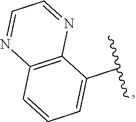 [SMILES: *c1cccc2nccnc12]

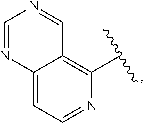 [SMILES: *c1nccc2ncncc12]

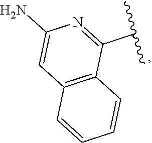 [SMILES: *c1nc(N)cc2ccccc12]

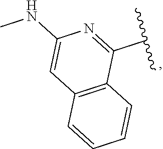 [SMILES: *c1nc(NC)cc2ccccc12]

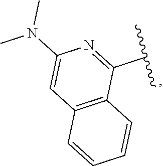 [SMILES: *c1nc(N(C)C)cc2ccccc12]

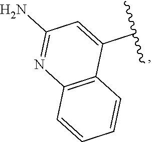 [SMILES: *c1cc(N)nc2ccccc12]

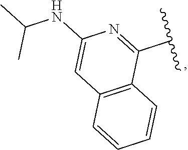 [SMILES: *c1nc(NC(C)C)cc2ccccc12]

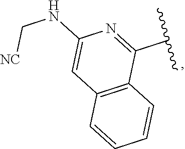 [SMILES: *c1nc(NCC#N)cc2ccccc12]

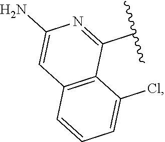 [SMILES: *c1nc(N)cc2cccc(Cl)c12]

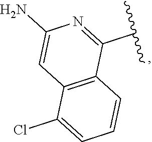 [SMILES: *c1nc(N)cc2c(Cl)cccc12]

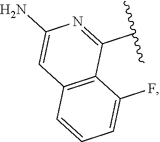 [SMILES: *c1nc(N)cc2cccc(F)c12]

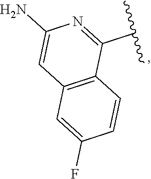 [SMILES: *c1nc(N)cc2cc(F)ccc12]

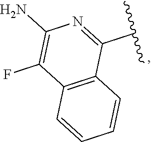 [SMILES: *c1nc(N)c(F)c2ccccc12]

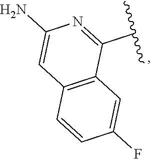 [SMILES: *c1nc(N)cc2ccc(F)cc12]

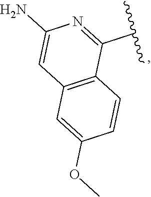 [SMILES: *c1nc(N)cc2cc(OC)ccc12]

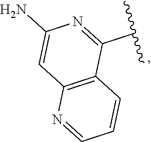 [SMILES: *c1nc(N)cc2ncccc12]

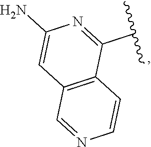 [SMILES: *c1nc(N)cc2cnccc12]

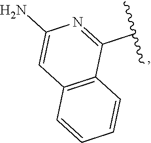 [SMILES: *c1nc(N)cc2ccccc12]

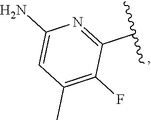 [SMILES: *c1nc(N)cc(C)c1F]

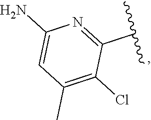 [SMILES: *c1nc(N)cc(C)c1Cl]

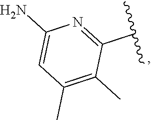 [SMILES: *c1nc(N)cc(C)c1C]

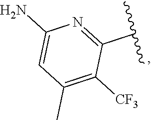 [SMILES: *c1nc(N)cc(C)c1C(F)(F)F]

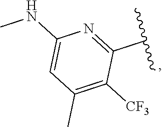 [SMILES: *c1nc(NC)cc(C)c1C(F)(F)F]

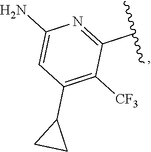 [SMILES: *c1nc(N)cc(C2CC2)c1C(F)(F)F]

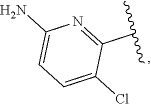 [SMILES: *c1nc(N)ccc1Cl]

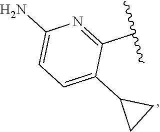 [SMILES: *c1nc(N)ccc1C1CC1]

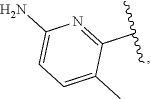 [SMILES: *c1nc(N)ccc1C]

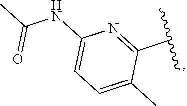 [SMILES: *c1nc(NC(C)=O)ccc1C]

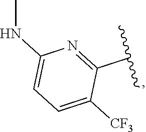 [SMILES: *c1nc(NC)ccc1C(F)(F)F]

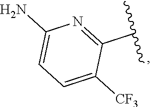 [SMILES: *c1nc(N)ccc1C(F)(F)F]

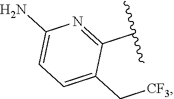 [SMILES: *c1nc(N)ccc1CC(F)(F)F]

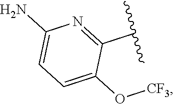 [SMILES: *c1nc(N)ccc1OC(F)(F)F]

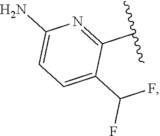 [SMILES: *c1nc(N)ccc1C(F)F]

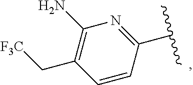 [SMILES: *c1ccc(CC(F)(F)F)c(N)n1]

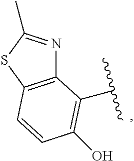 [SMILES: *c1c(O)ccc2sc(C)nc12]

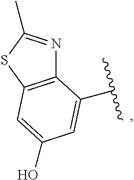 [SMILES: *c1cc(O)cc2sc(C)nc12]

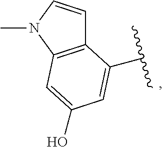 [SMILES: *c1cc(O)cc2c1ccn2C]

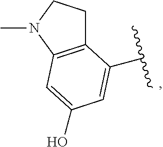 [SMILES: *c1cc(O)cc2c1CCN2C]

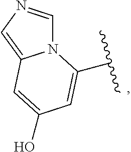 [SMILES: *c1cc(O)cc2cncn12]

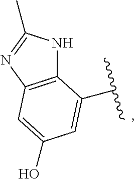 [SMILES: *c1cc(O)cc2nc(C)[nH]c12]

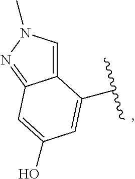 [SMILES: *c1cc(O)cc2nn(C)cc12]

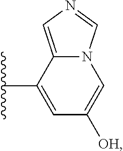 [SMILES: *c1cc(O)cn2cncc12]

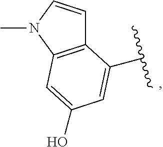 [SMILES: *c1cc(O)cc2c1ccn2C]

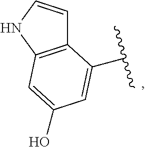 [SMILES: *c1cc(O)cc2[nH]ccc12]

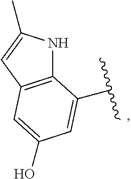 [SMILES: *c1cc(O)cc2cc(C)[nH]c12]

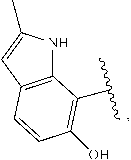 [SMILES: *c1c(O)ccc2cc(C)[nH]c12]

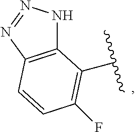 [SMILES: *c1c(F)ccc2nn[nH]c12]

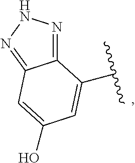 [SMILES: *c1cc(O)cc2n[nH]nc12]

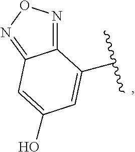 [SMILES: *c1cc(O)cc2nonc12]

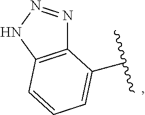 [SMILES: *c1cccc2[nH]nnc12]

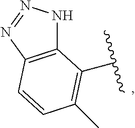 [SMILES: *c1c(C)ccc2nn[nH]c12]

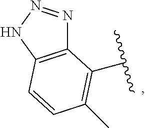 [SMILES: *c1c(C)ccc2[nH]nnc12]

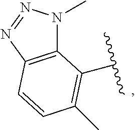 [SMILES: *c1c(C)ccc2nnn(C)c12]

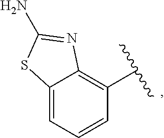 [SMILES: *c1cccc2sc(N)nc12]

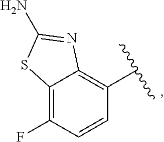 [SMILES: *c1ccc(F)c2sc(N)nc12]

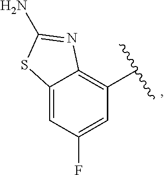 [SMILES: *c1cc(F)cc2sc(N)nc12]

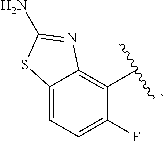 [SMILES: *c1c(F)ccc2sc(N)nc12]

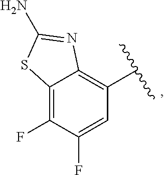 [SMILES: *c1cc(F)c(F)c2sc(N)nc12]

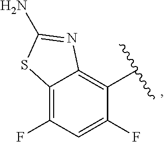 [SMILES: *c1c(F)cc(F)c2sc(N)nc12]

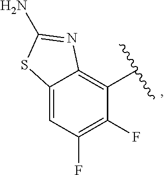 [SMILES: *c1c(F)c(F)cc2sc(N)nc12]

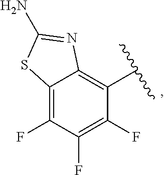 [SMILES: *c1c(F)c(F)c(F)c2sc(N)nc12]

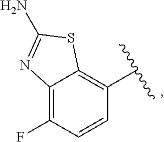 [SMILES: *c1ccc(F)c2nc(N)sc12]

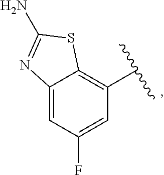 [SMILES: *c1cc(F)cc2nc(N)sc12]

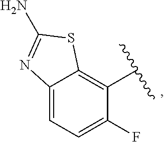 [SMILES: *c1c(F)ccc2nc(N)sc12]

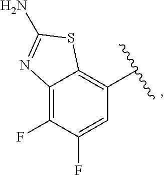 [SMILES: *c1cc(F)c(F)c2nc(N)sc12]

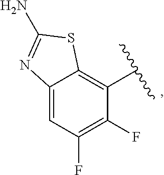 [SMILES: *c1c(F)c(F)cc2nc(N)sc12]

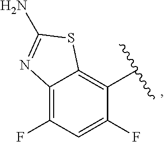 [SMILES: *c1c(F)cc(F)c2nc(N)sc12]

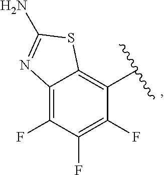 [SMILES: *c1c(F)c(F)c(F)c2nc(N)sc12]

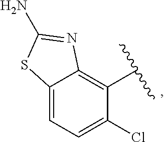 [SMILES: *c1c(Cl)ccc2sc(N)nc12]

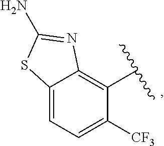 [SMILES: *c1c(C(F)(F)F)ccc2sc(N)nc12]

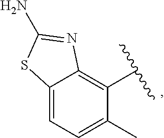 [SMILES: *c1c(C)ccc2sc(N)nc12]

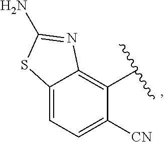 [SMILES: *c1c(C#N)ccc2sc(N)nc12]

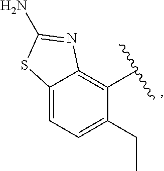 [SMILES: *c1c(CC)ccc2sc(N)nc12]

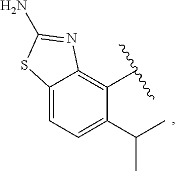 [SMILES: *c1c(C(C)C)ccc2sc(N)nc12]

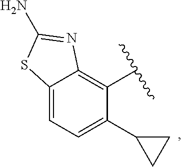 [SMILES: *c1c(C2CC2)ccc2sc(N)nc12]

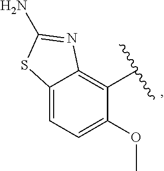 [SMILES: *c1c(OC)ccc2sc(N)nc12]

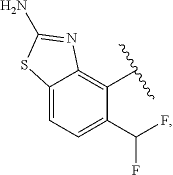 [SMILES: *c1c(C(F)F)ccc2sc(N)nc12]

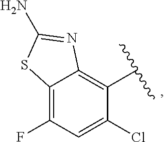 [SMILES: *c1c(Cl)cc(F)c2sc(N)nc12]

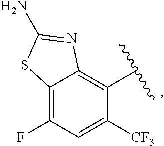 [SMILES: *c1c(C(F)(F)F)cc(F)c2sc(N)nc12]

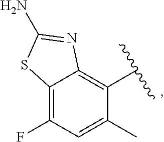 [SMILES: *c1c(C)cc(F)c2sc(N)nc12]

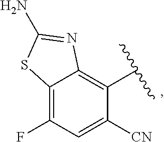 [SMILES: *c1c(C#N)cc(F)c2sc(N)nc12]

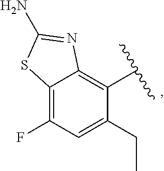 [SMILES: *c1c(CC)cc(F)c2sc(N)nc12]

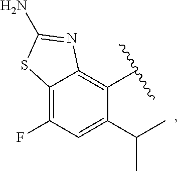 [SMILES: *c1c(C(C)C)cc(F)c2sc(N)nc12]

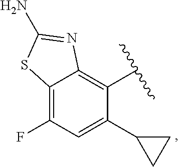 [SMILES: *c1c(C2CC2)cc(F)c2sc(N)nc12]

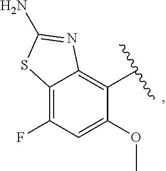 [SMILES: *c1c(OC)cc(F)c2sc(N)nc12]

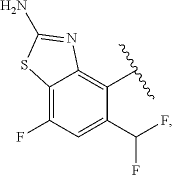 [SMILES: *c1c(C(F)F)cc(F)c2sc(N)nc12]

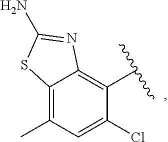 [SMILES: *c1c(Cl)cc(C)c2sc(N)nc12]

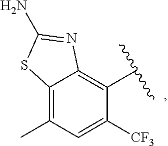 [SMILES: *c1c(C(F)(F)F)cc(C)c2sc(N)nc12]

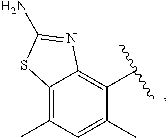

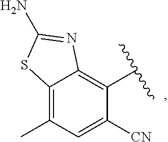 [SMILES: *c1c(C#N)cc(C)c2sc(N)nc12]

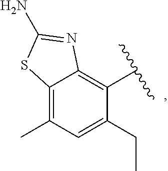 [SMILES: *c1c(CC)cc(C)c2sc(N)nc12]

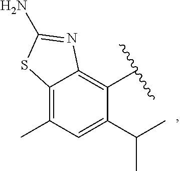 [SMILES: *c1c(C(C)C)cc(C)c2sc(N)nc12]

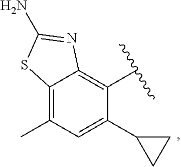 [SMILES: *c1c(C2CC2)cc(C)c2sc(N)nc12]

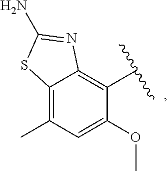

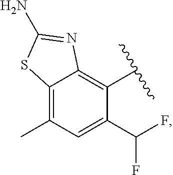 [SMILES: *c1c(C(F)F)cc(C)c2sc(N)nc12]

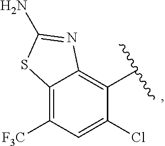 [SMILES: *c1c(Cl)cc(C(F)(F)F)c2sc(N)nc12]

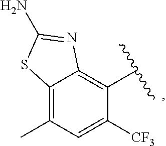 [SMILES: *c1c(C(F)(F)F)cc(C)c2sc(N)nc12]

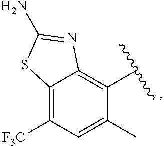 [SMILES: *c1c(C)cc(C(F)(F)F)c2sc(N)nc12]

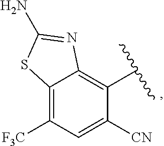 [SMILES: *c1c(C#N)cc(C(F)(F)F)c2sc(N)nc12]

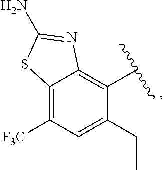 [SMILES: *c1c(CC)cc(C(F)(F)F)c2sc(N)nc12]

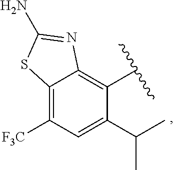 [SMILES: *c1c(C(C)C)cc(C(F)(F)F)c2sc(N)nc12]

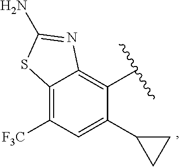 [SMILES: *c1c(C2CC2)cc(C(F)(F)F)c2sc(N)nc12]

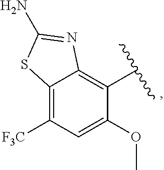 [SMILES: *c1c(OC)cc(C(F)(F)F)c2sc(N)nc12]

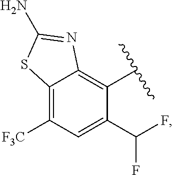 [SMILES: *c1c(C(F)F)cc(C(F)(F)F)c2sc(N)nc12]

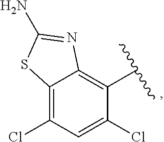 [SMILES: *c1c(Cl)cc(Cl)c2sc(N)nc12]

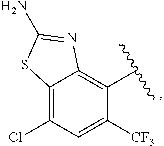 [SMILES: *c1c(C(F)(F)F)cc(Cl)c2sc(N)nc12]

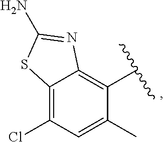 [SMILES: *c1c(C)cc(Cl)c2sc(N)nc12]

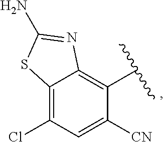 [SMILES: *c1c(C#N)cc(Cl)c2sc(N)nc12]

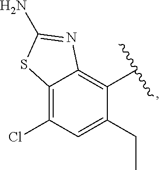 [SMILES: *c1c(CC)cc(Cl)c2sc(N)nc12]

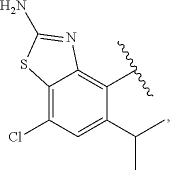 [SMILES: *c1c(C(C)C)cc(Cl)c2sc(N)nc12]

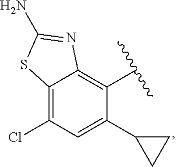 [SMILES: *c1c(C2CC2)cc(Cl)c2sc(N)nc12]

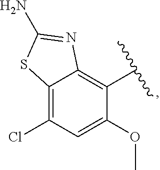 [SMILES: *c1c(OC)cc(Cl)c2sc(N)nc12]

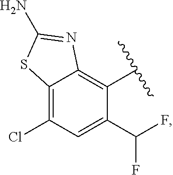 [SMILES: *c1c(C(F)F)cc(Cl)c2sc(N)nc12]

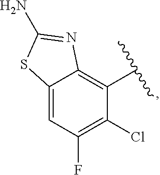 [SMILES: *c1c(Cl)c(F)cc2sc(N)nc12]

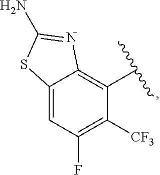 [SMILES: *c1c(C(F)(F)F)c(F)cc2sc(N)nc12]

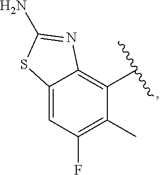 [SMILES: *c1c(C)c(F)cc2sc(N)nc12]

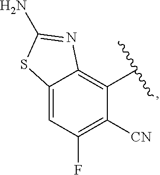 [SMILES: *c1c(C#N)c(F)cc2sc(N)nc12]

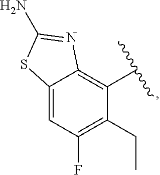 [SMILES: *c1c(CC)c(F)cc2sc(N)nc12]

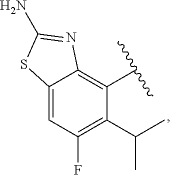 [SMILES: *c1c(C(C)C)c(F)cc2sc(N)nc12]

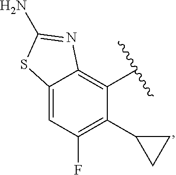 [SMILES: *c1c(C2CC2)c(F)cc2sc(N)nc12]

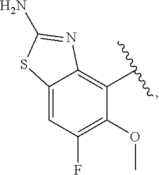 [SMILES: *c1c(OC)c(F)cc2sc(N)nc12]

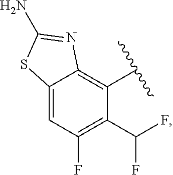 [SMILES: *c1c(C(F)F)c(F)cc2sc(N)nc12]

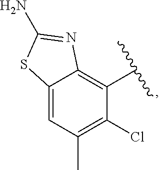 [SMILES: *c1c(Cl)c(C)cc2sc(N)nc12]

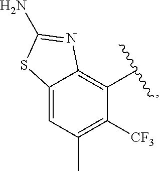 [SMILES: *c1c(C(F)(F)F)c(C)cc2sc(N)nc12]

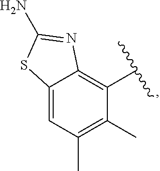 [SMILES: *c1c(C)c(C)cc2sc(N)nc12]

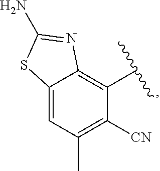 [SMILES: *c1c(C#N)c(C)cc2sc(N)nc12]

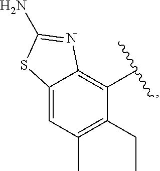 [SMILES: *c1c(CC)c(C)cc2sc(N)nc12]

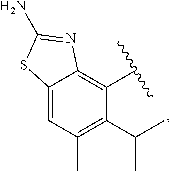 [SMILES: *c1c(C(C)C)c(C)cc2sc(N)nc12]

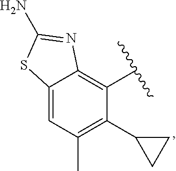 [SMILES: *c1c(C2CC2)c(C)cc2sc(N)nc12]

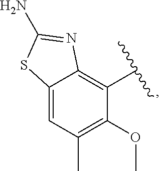 [SMILES: *c1c(OC)c(C)cc2sc(N)nc12]

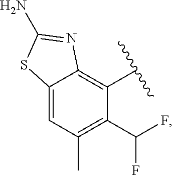 [SMILES: *c1c(C(F)F)c(C)cc2sc(N)nc12]

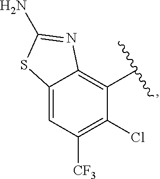 [SMILES: *c1c(Cl)c(C(F)(F)F)cc2sc(N)nc12]

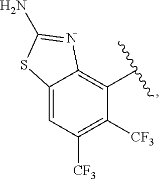 [SMILES: *c1c(C(F)(F)F)c(C(F)(F)F)cc2sc(N)nc12]

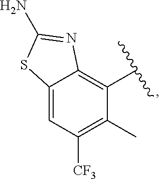 [SMILES: *c1c(C)c(C(F)(F)F)cc2sc(N)nc12]

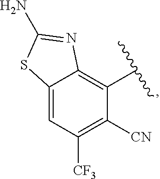 [SMILES: *c1c(C#N)c(C(F)(F)F)cc2sc(N)nc12]

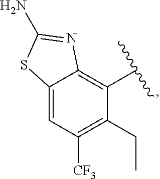 [SMILES: *c1c(CC)c(C(F)(F)F)cc2sc(N)nc12]

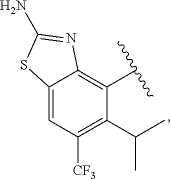 [SMILES: *c1c(C(C)C)c(C(F)(F)F)cc2sc(N)nc12]

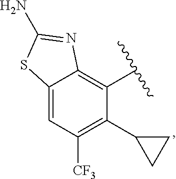 [SMILES: *c1c(C2CC2)c(C(F)(F)F)cc2sc(N)nc12]

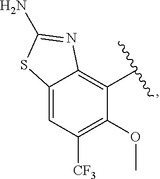 [SMILES: *c1c(OC)c(C(F)(F)F)cc2sc(N)nc12]

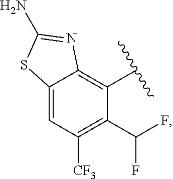 [SMILES: *c1c(C(F)F)c(C(F)(F)F)cc2sc(N)nc12]

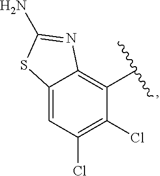 [SMILES: *c1c(Cl)c(Cl)cc2sc(N)nc12]

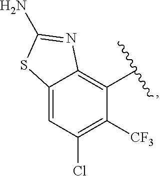 [SMILES: *c1c(C(F)(F)F)c(Cl)cc2sc(N)nc12]

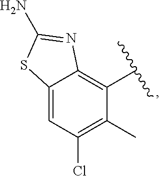 [SMILES: *c1c(C)c(Cl)cc2sc(N)nc12]

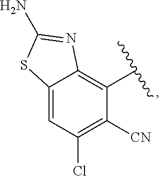 [SMILES: *c1c(C#N)c(Cl)cc2sc(N)nc12]

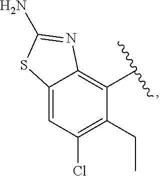 [SMILES: *c1c(CC)c(Cl)cc2sc(N)nc12]

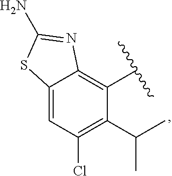 [SMILES: *c1c(C(C)C)c(Cl)cc2sc(N)nc12]

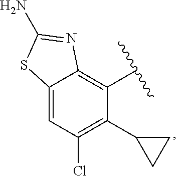 [SMILES: *c1c(C2CC2)c(Cl)cc2sc(N)nc12]

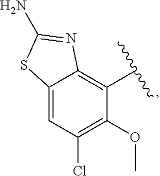 [SMILES: *c1c(OC)c(Cl)cc2sc(N)nc12]

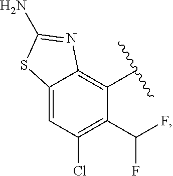 [SMILES: *c1c(C(F)F)c(Cl)cc2sc(N)nc12]

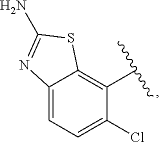 [SMILES: *c1c(Cl)ccc2nc(N)sc12]

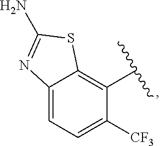 [SMILES: *c1c(C(F)(F)F)ccc2nc(N)sc12]

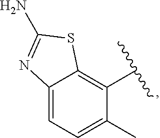 [SMILES: *c1c(C)ccc2nc(N)sc12]

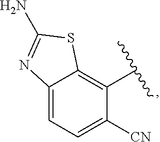 [SMILES: *c1c(C#N)ccc2nc(N)sc12]

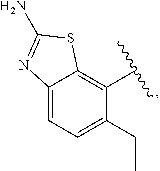 [SMILES: *c1c(CC)ccc2nc(N)sc12]

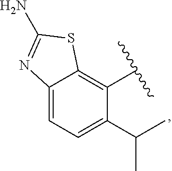 [SMILES: *c1c(C(C)C)ccc2nc(N)sc12]

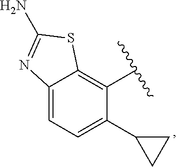 [SMILES: *c1c(C2CC2)ccc2nc(N)sc12]

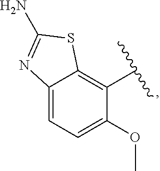 [SMILES: *c1c(OC)ccc2nc(N)sc12]

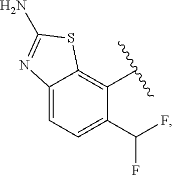 [SMILES: *c1c(C(F)F)ccc2nc(N)sc12]

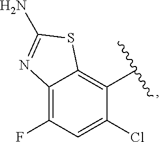 [SMILES: *c1c(Cl)cc(F)c2nc(N)sc12]

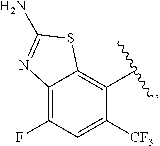 [SMILES: *c1c(C(F)(F)F)cc(F)c2nc(N)sc12]

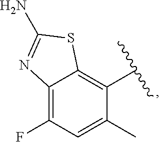 [SMILES: *c1c(C)cc(F)c2nc(N)sc12]

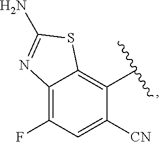 [SMILES: *c1c(C#N)cc(F)c2nc(N)sc12]

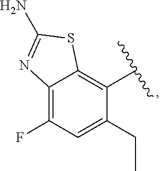 [SMILES: *c1c(CC)cc(F)c2nc(N)sc12]

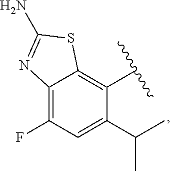 [SMILES: *c1c(C(C)C)cc(F)c2nc(N)sc12]

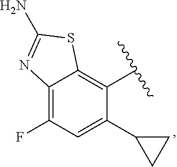 [SMILES: *c1c(C2CC2)cc(F)c2nc(N)sc12]

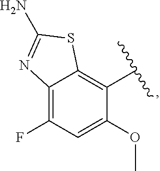 [SMILES: *c1c(OC)cc(F)c2nc(N)sc12]

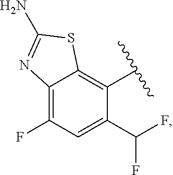 [SMILES: *c1c(C(F)F)cc(F)c2nc(N)sc12]

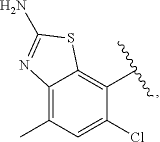 [SMILES: *c1c(Cl)cc(C)c2nc(N)sc12]

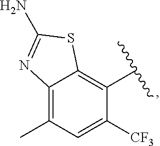 [SMILES: *c1c(C(F)(F)F)cc(C)c2nc(N)sc12]

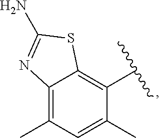

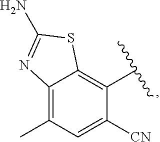 [SMILES: *c1c(C#N)cc(C)c2nc(N)sc12]

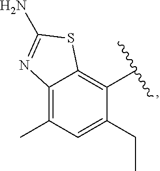 [SMILES: *c1c(CC)cc(C)c2nc(N)sc12]

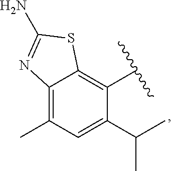 [SMILES: *c1c(C(C)C)cc(C)c2nc(N)sc12]

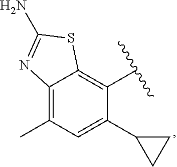 [SMILES: *c1c(C2CC2)cc(C)c2nc(N)sc12]

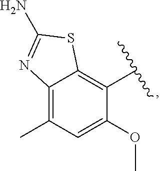

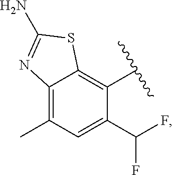 [SMILES: *c1c(C(F)F)cc(C)c2nc(N)sc12]

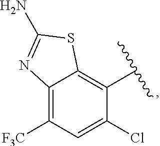 [SMILES: *c1c(Cl)cc(C(F)(F)F)c2nc(N)sc12]

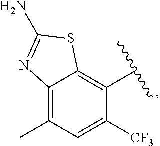 [SMILES: *c1c(C(F)(F)F)cc(C)c2nc(N)sc12]

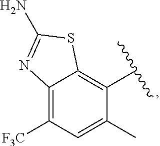 [SMILES: *c1c(C)cc(C(F)(F)F)c2nc(N)sc12]

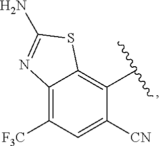 [SMILES: *c1c(C#N)cc(C(F)(F)F)c2nc(N)sc12]

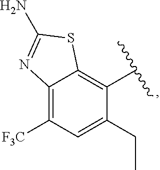 [SMILES: *c1c(CC)cc(C(F)(F)F)c2nc(N)sc12]

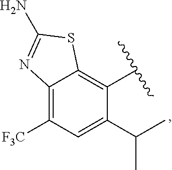 [SMILES: *c1c(C(C)C)cc(C(F)(F)F)c2nc(N)sc12]

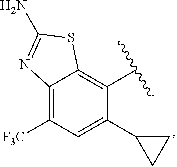 [SMILES: *c1c(C2CC2)cc(C(F)(F)F)c2nc(N)sc12]

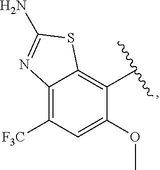 [SMILES: *c1c(OC)cc(C(F)(F)F)c2nc(N)sc12]

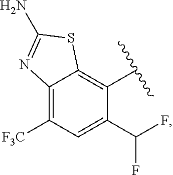 [SMILES: *c1c(C(F)F)cc(C(F)(F)F)c2nc(N)sc12]

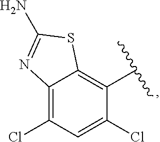 [SMILES: *c1c(Cl)cc(Cl)c2nc(N)sc12]

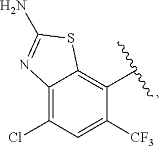 [SMILES: *c1c(C(F)(F)F)cc(Cl)c2nc(N)sc12]

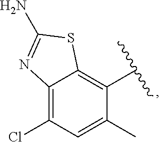 [SMILES: *c1c(C)cc(Cl)c2nc(N)sc12]

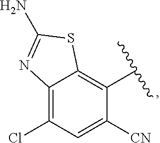 [SMILES: *c1c(C#N)cc(Cl)c2nc(N)sc12]

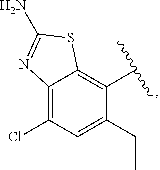 [SMILES: *c1c(CC)cc(Cl)c2nc(N)sc12]

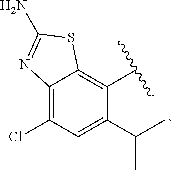 [SMILES: *c1c(C(C)C)cc(Cl)c2nc(N)sc12]

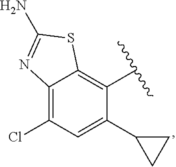 [SMILES: *c1c(C2CC2)cc(Cl)c2nc(N)sc12]

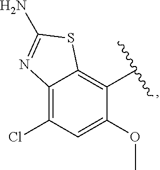 [SMILES: *c1c(OC)cc(Cl)c2nc(N)sc12]

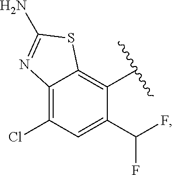 [SMILES: *c1c(C(F)F)cc(Cl)c2nc(N)sc12]

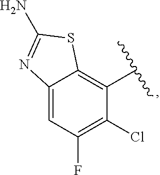 [SMILES: *c1c(Cl)c(F)cc2nc(N)sc12]

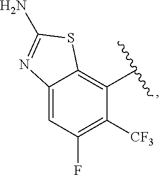 [SMILES: *c1c(C(F)(F)F)c(F)cc2nc(N)sc12]

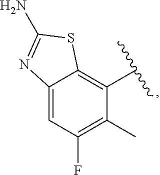 [SMILES: *c1c(C)c(F)cc2nc(N)sc12]

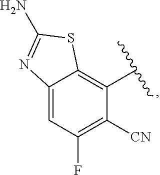 [SMILES: *c1c(C#N)c(F)cc2nc(N)sc12]

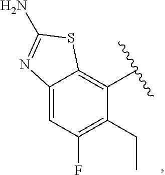 [SMILES: *c1c(CC)c(F)cc2nc(N)sc12]

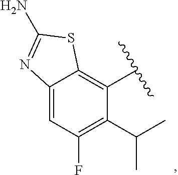 [SMILES: *c1c(C(C)C)c(F)cc2nc(N)sc12]

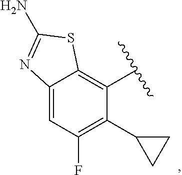 [SMILES: *c1c(C2CC2)c(F)cc2nc(N)sc12]

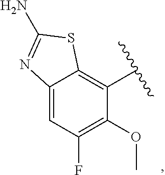 [SMILES: *c1c(OC)c(F)cc2nc(N)sc12]

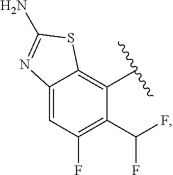 [SMILES: *c1c(C(F)F)c(F)cc2nc(N)sc12]

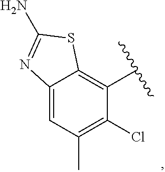 [SMILES: *c1c(Cl)c(C)cc2nc(N)sc12]

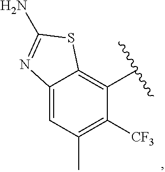 [SMILES: *c1c(C(F)(F)F)c(C)cc2nc(N)sc12]

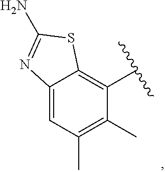 [SMILES: *c1c(C)c(C)cc2nc(N)sc12]

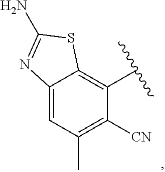 [SMILES: *c1c(C#N)c(C)cc2nc(N)sc12]

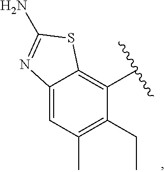 [SMILES: *c1c(CC)c(C)cc2nc(N)sc12]

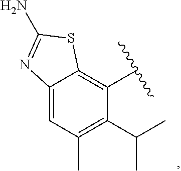 [SMILES: *c1c(C(C)C)c(C)cc2nc(N)sc12]

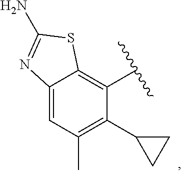 [SMILES: *c1c(C2CC2)c(C)cc2nc(N)sc12]

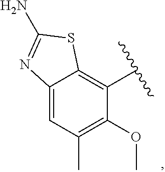 [SMILES: *c1c(OC)c(C)cc2nc(N)sc12]

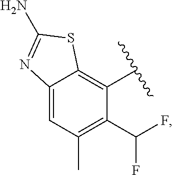 [SMILES: *c1c(C(F)F)c(C)cc2nc(N)sc12]

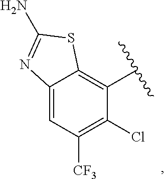 [SMILES: *c1c(Cl)c(C(F)(F)F)cc2nc(N)sc12]

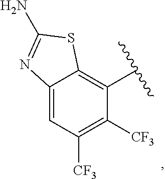 [SMILES: *c1c(C(F)(F)F)c(C(F)(F)F)cc2nc(N)sc12]

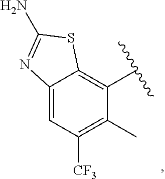 [SMILES: *c1c(C)c(C(F)(F)F)cc2nc(N)sc12]

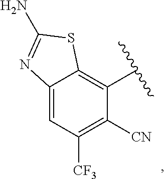 [SMILES: *c1c(C#N)c(C(F)(F)F)cc2nc(N)sc12]

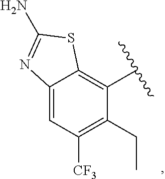 [SMILES: *c1c(CC)c(C(F)(F)F)cc2nc(N)sc12]

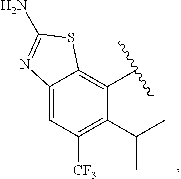 [SMILES: *c1c(C(C)C)c(C(F)(F)F)cc2nc(N)sc12]

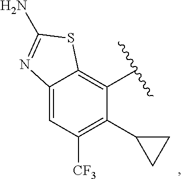 [SMILES: *c1c(C2CC2)c(C(F)(F)F)cc2nc(N)sc12]

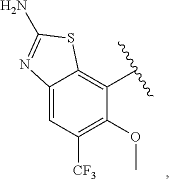 [SMILES: *c1c(OC)c(C(F)(F)F)cc2nc(N)sc12]

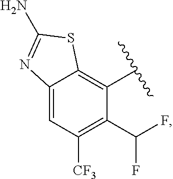 [SMILES: *c1c(C(F)F)c(C(F)(F)F)cc2nc(N)sc12]

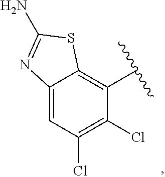 [SMILES: *c1c(Cl)c(Cl)cc2nc(N)sc12]

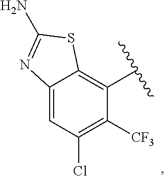 [SMILES: *c1c(C(F)(F)F)c(Cl)cc2nc(N)sc12]

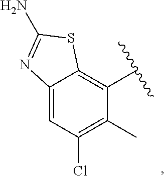 [SMILES: *c1c(C)c(Cl)cc2nc(N)sc12]

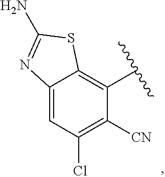 [SMILES: *c1c(C#N)c(Cl)cc2nc(N)sc12]

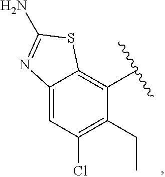 [SMILES: *c1c(CC)c(Cl)cc2nc(N)sc12]

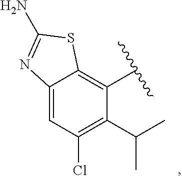 [SMILES: *c1c(C(C)C)c(Cl)cc2nc(N)sc12]

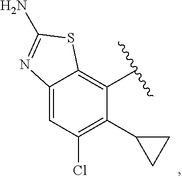 [SMILES: *c1c(C2CC2)c(Cl)cc2nc(N)sc12]

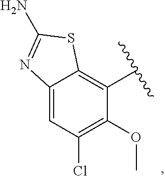 [SMILES: *c1c(OC)c(Cl)cc2nc(N)sc12]

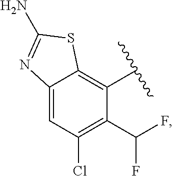 [SMILES: *c1c(C(F)F)c(Cl)cc2nc(N)sc12]

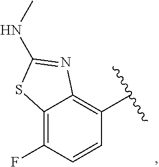 [SMILES: *c1ccc(F)c2sc(NC)nc12]

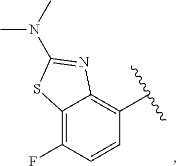 [SMILES: *c1ccc(F)c2sc(N(C)C)nc12]

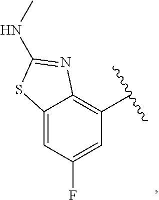 [SMILES: *c1cc(F)cc2sc(NC)nc12]

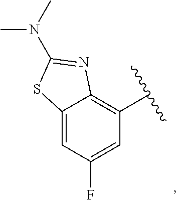 [SMILES: *c1cc(F)cc2sc(N(C)C)nc12]

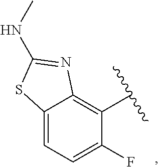 [SMILES: *c1c(F)ccc2sc(NC)nc12]

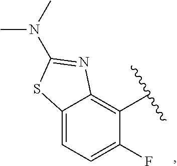 [SMILES: *c1c(F)ccc2sc(N(C)C)nc12]

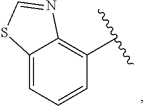 [SMILES: *c1cccc2scnc12]

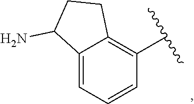 [SMILES: *c1cccc2c1CCC2N]

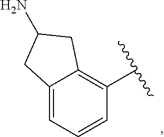 [SMILES: *c1cccc2c1CC(N)C2]

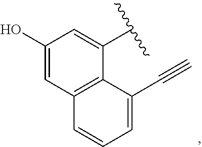 [SMILES: *c1cc(O)cc2cccc(C#C)c12]

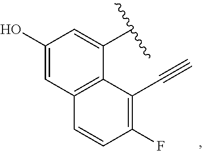 [SMILES: *c1cc(O)cc2ccc(F)c(C#C)c12]

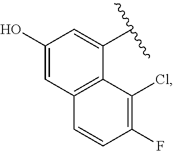 [SMILES: *c1cc(O)cc2ccc(F)c(Cl)c12]

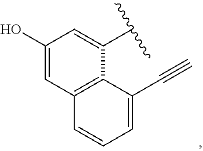

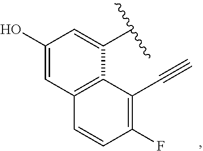

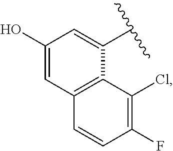

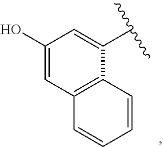

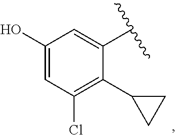 [SMILES: *c1cc(O)cc(Cl)c1C1CC1]

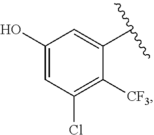 [SMILES: *c1cc(O)cc(Cl)c1C(F)(F)F]

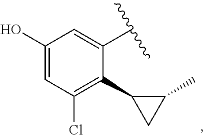 [SMILES: *c1cc(O)cc(Cl)c1[C@@H]1C[C@H]1C]

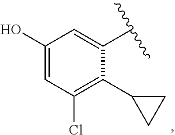

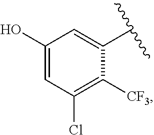

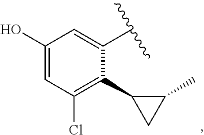

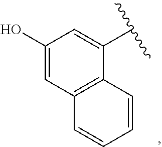 [SMILES: *c1cc(O)cc2ccccc12]

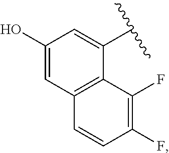 [SMILES: *c1cc(O)cc2ccc(F)c(F)c12]

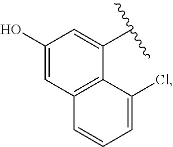 [SMILES: *c1cc(O)cc2cccc(Cl)c12]

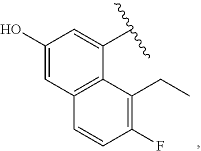 [SMILES: *c1cc(O)cc2ccc(F)c(CC)c12]

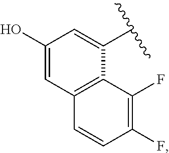

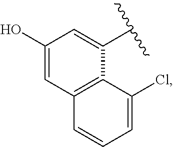

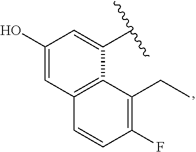

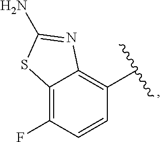 [SMILES: *c1ccc(F)c2sc(N)nc12]

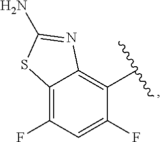 [SMILES: *c1c(F)cc(F)c2sc(N)nc12]

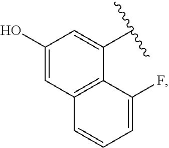 [SMILES: *c1cc(O)cc2cccc(F)c12]

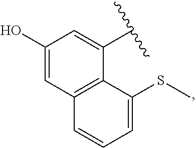 [SMILES: *c1cc(O)cc2cccc(SC)c12]

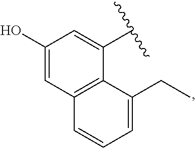 [SMILES: *c1cc(O)cc2cccc(CC)c12]

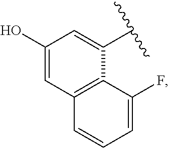

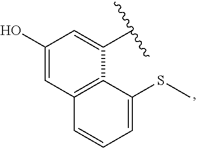

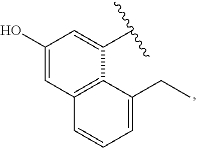

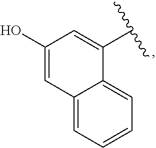 [SMILES: *c1cc(O)cc2ccccc12]

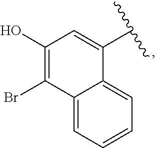 [SMILES: *c1cc(O)c(Br)c2ccccc12]

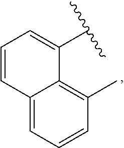 [SMILES: *c1cccc2cccc(C)c12]

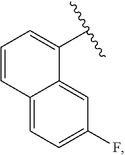 [SMILES: *c1cccc2ccc(F)cc12]

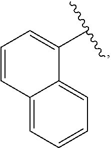 [SMILES: *c1cccc2ccccc12]

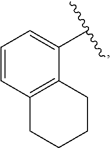 [SMILES: *c1cccc2c1CCCC2]

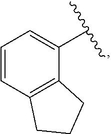 [SMILES: *c1cccc2c1CCC2]

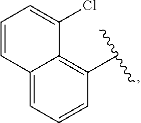 [SMILES: *c1cccc2cccc(Cl)c12]

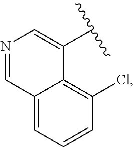 [SMILES: *c1cncc2cccc(Cl)c12]

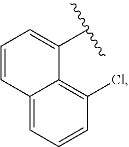 [SMILES: *c1cccc2cccc(Cl)c12]

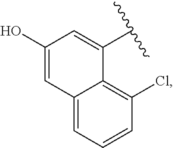 [SMILES: *c1cc(O)cc2cccc(Cl)c12]

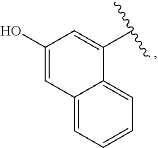 [SMILES: *c1cc(O)cc2ccccc12]

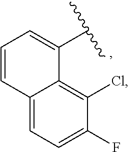 [SMILES: *c1cccc2ccc(F)c(Cl)c12]

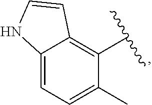 [SMILES: *c1c(C)ccc2[nH]ccc12]

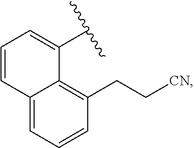 [SMILES: *c1cccc2cccc(CCC#N)c12]

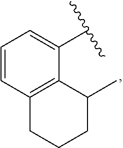 [SMILES: *c1cccc2c1C(C)CCC2]

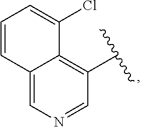 [SMILES: *c1cncc2cccc(Cl)c12]

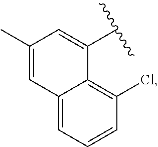 [SMILES: *c1cc(C)cc2cccc(Cl)c12]

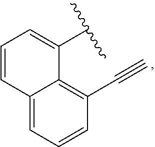 [SMILES: *c1cccc2cccc(C#C)c12]

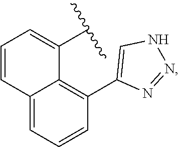 [SMILES: *c1cccc2cccc(-c3c[nH]nn3)c12]

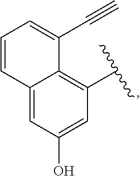 [SMILES: *c1cc(O)cc2cccc(C#C)c12]

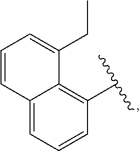 [SMILES: *c1cccc2cccc(CC)c12]

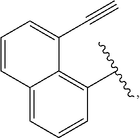 [SMILES: *c1cccc2cccc(C#C)c12]

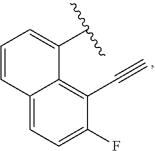 [SMILES: *c1cccc2ccc(F)c(C#C)c12]

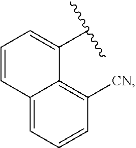 [SMILES: *c1cccc2cccc(C#N)c12]

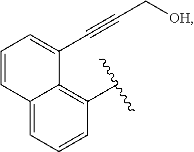 [SMILES: *c1cccc2cccc(C#CCO)c12]

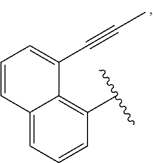 [SMILES: *c1cccc2cccc(C#CC)c12]

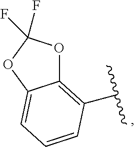 [SMILES: *c1cccc2c1OC(F)(F)O2]

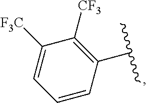 [SMILES: *c1cccc(C(F)(F)F)c1C(F)(F)F]

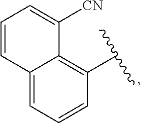 [SMILES: *c1cccc2cccc(C#N)c12]

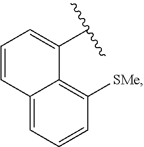 [SMILES: *c1cccc2cccc(SC)c12]

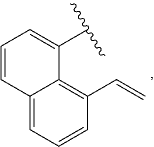 [SMILES: *c1cccc2cccc(C=C)c12]

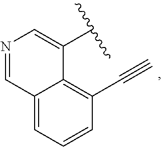 [SMILES: *c1cncc2cccc(C#C)c12]

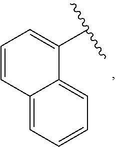 [SMILES: *c1cccc2ccccc12]

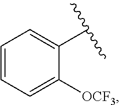 [SMILES: *c1ccccc1OC(F)(F)F]

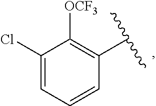 [SMILES: *c1cccc(Cl)c1OC(F)(F)F]

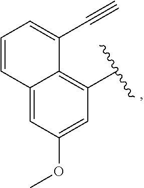 [SMILES: *c1cc(OC)cc2cccc(C#C)c12]

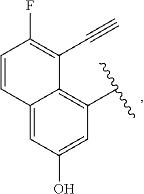 [SMILES: *c1cc(O)cc2ccc(F)c(C#C)c12]

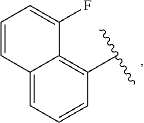 [SMILES: *c1cccc2cccc(F)c12]

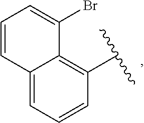 [SMILES: *c1cccc2cccc(Br)c12]

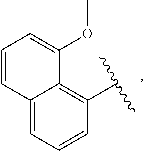 [SMILES: *c1cccc2cccc(OC)c12]

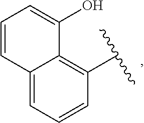 [SMILES: *c1cccc2cccc(O)c12]

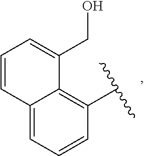 [SMILES: *c1cccc2cccc(CO)c12]

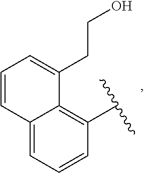 [SMILES: *c1cccc2cccc(CCO)c12]

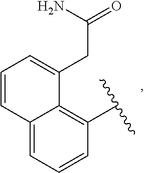 [SMILES: *c1cccc2cccc(CC(N)=O)c12]

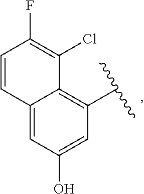 [SMILES: *c1cc(O)cc2ccc(F)c(Cl)c12]

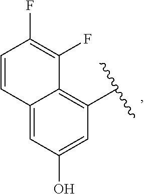 [SMILES: *c1cc(O)cc2ccc(F)c(F)c12]

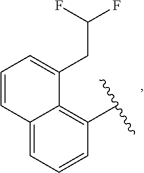 [SMILES: *c1cccc2cccc(CC(F)F)c12]

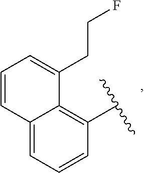 [SMILES: *c1cccc2cccc(CCF)c12]

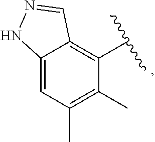 [SMILES: *c1c(C)c(C)cc2[nH]ncc12]

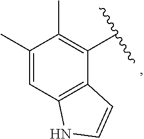 [SMILES: *c1c(C)c(C)cc2[nH]ccc12]

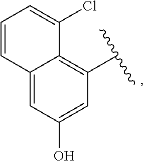 [SMILES: *c1cc(O)cc2cccc(Cl)c12]

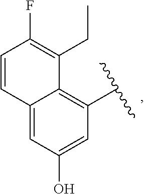 [SMILES: *c1cc(O)cc2ccc(F)c(CC)c12]

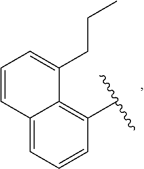 [SMILES: *c1cccc2cccc(CCC)c12]

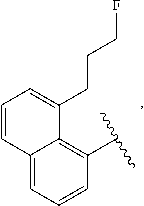 [SMILES: *c1cccc2cccc(CCCF)c12]

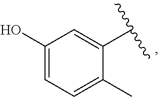 [SMILES: *c1cc(O)ccc1C]

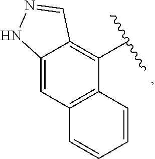 [SMILES: *c1c2ccccc2cc2[nH]ncc12]

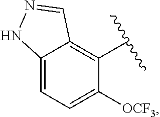 [SMILES: *c1c(OC(F)(F)F)ccc2[nH]ncc12]

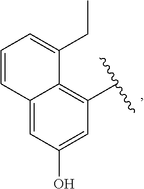 [SMILES: *c1cc(O)cc2cccc(CC)c12]

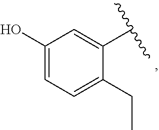 [SMILES: *c1cc(O)ccc1CC]

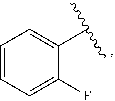 [SMILES: *c1ccccc1F]

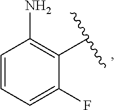 [SMILES: *c1c(N)cccc1F]

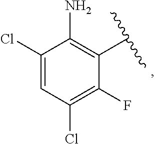 [SMILES: *c1c(N)c(Cl)cc(Cl)c1F]

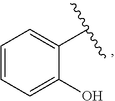 [SMILES: *c1ccccc1O]

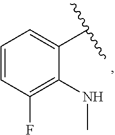 [SMILES: *c1cccc(F)c1NC]

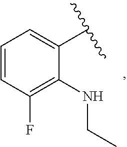 [SMILES: *c1cccc(F)c1NCC]

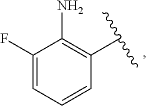 [SMILES: *c1cccc(F)c1N]

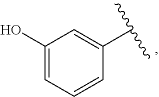 [SMILES: *c1cccc(O)c1]

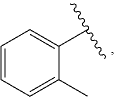 [SMILES: *c1ccccc1C]

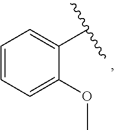 [SMILES: *c1ccccc1OC]

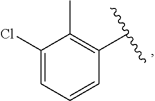 [SMILES: *c1cccc(Cl)c1C]

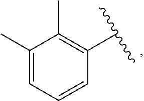 [SMILES: *c1cccc(C)c1C]

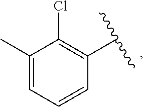 [SMILES: *c1cccc(C)c1Cl]

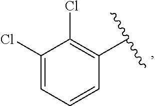 [SMILES: *c1cccc(Cl)c1Cl]

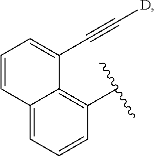

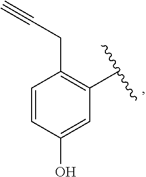 [SMILES: *c1cc(O)ccc1CC#C]

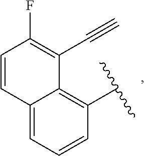 [SMILES: *c1cccc2ccc(F)c(C#C)c12]

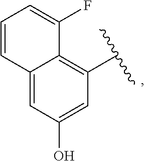 [SMILES: *c1cc(O)cc2cccc(F)c12]

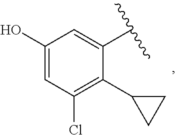 [SMILES: *c1cc(O)cc(Cl)c1C1CC1]

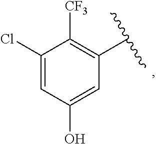 [SMILES: *c1cc(O)cc(Cl)c1C(F)(F)F]

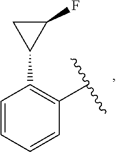 [SMILES: *c1ccccc1[C@@H]1C[C@H]1F]

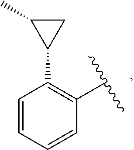 [SMILES: *c1ccccc1[C@H]1C[C@H]1C]

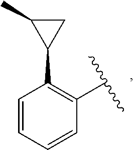 [SMILES: *c1ccccc1[C@@H]1C[C@@H]1C]

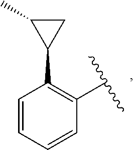 [SMILES: *c1ccccc1[C@@H]1C[C@H]1C]

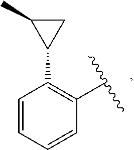 [SMILES: *c1ccccc1[C@H]1C[C@@H]1C]

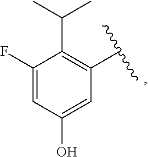 [SMILES: *c1cc(O)cc(F)c1C(C)C]

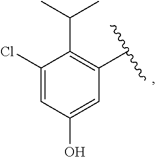 [SMILES: *c1cc(O)cc(Cl)c1C(C)C]

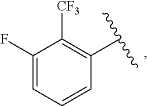 [SMILES: *c1cccc(F)c1C(F)(F)F]

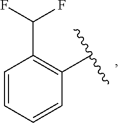 [SMILES: *c1ccccc1C(F)F]

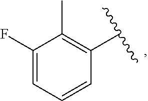 [SMILES: *c1cccc(F)c1C]

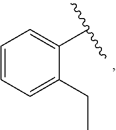 [SMILES: *c1ccccc1CC]

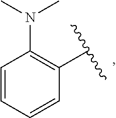 [SMILES: *c1ccccc1N(C)C]

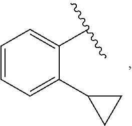 [SMILES: *c1ccccc1C1CC1]

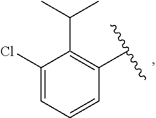 [SMILES: *c1cccc(Cl)c1C(C)C]

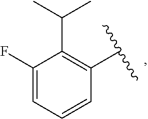 [SMILES: *c1cccc(F)c1C(C)C]

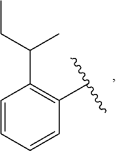 [SMILES: *c1ccccc1C(C)CC]

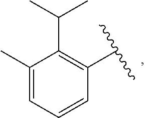 [SMILES: *c1cccc(C)c1C(C)C]

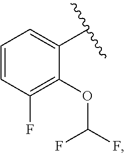 [SMILES: *c1cccc(F)c1OC(F)F]

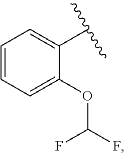 [SMILES: *c1ccccc1OC(F)F]

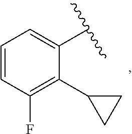 [SMILES: *c1cccc(F)c1C1CC1]

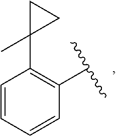 [SMILES: *c1ccccc1C1(C)CC1]

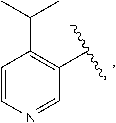 [SMILES: *c1cnccc1C(C)C]

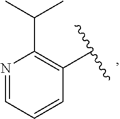 [SMILES: *c1cccnc1C(C)C]

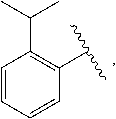 [SMILES: *c1ccccc1C(C)C]

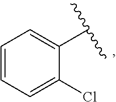 [SMILES: *c1ccccc1Cl]

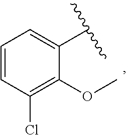 [SMILES: *c1cccc(Cl)c1OC]

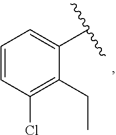 [SMILES: *c1cccc(Cl)c1CC]

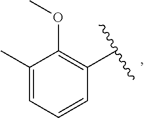 [SMILES: *c1cccc(C)c1OC]

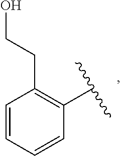 [SMILES: *c1ccccc1CCO]

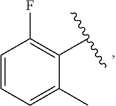 [SMILES: *c1c(C)cccc1F]

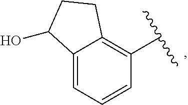 [SMILES: *c1cccc2c1CCC2O]

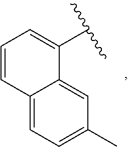 [SMILES: *c1cccc2ccc(C)cc12]

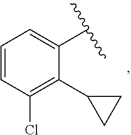 [SMILES: *c1cccc(Cl)c1C1CC1]

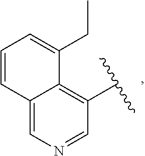 [SMILES: *c1cncc2cccc(CC)c12]

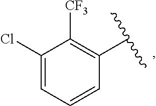 [SMILES: *c1cccc(Cl)c1C(F)(F)F]

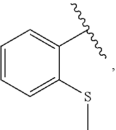 [SMILES: *c1ccccc1SC]

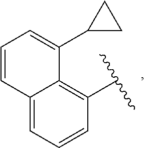 [SMILES: *c1cccc2cccc(C3CC3)c12]

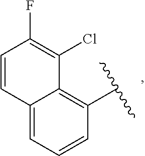 [SMILES: *c1cccc2ccc(F)c(Cl)c12]

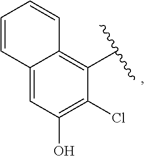 [SMILES: *c1c(Cl)c(O)cc2ccccc12]

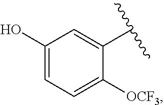 [SMILES: *c1cc(O)ccc1OC(F)(F)F]

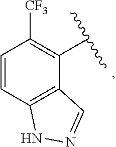 [SMILES: *c1c(C(F)(F)F)ccc2[nH]ncc12]

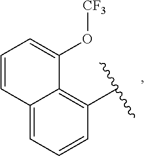 [SMILES: *c1cccc2cccc(OC(F)(F)F)c12]

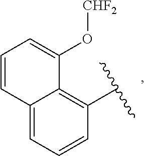 [SMILES: *c1cccc2cccc(OC(F)F)c12]

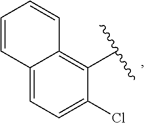 [SMILES: *c1c(Cl)ccc2ccccc12]

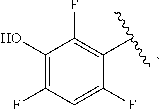 [SMILES: *c1c(F)cc(F)c(O)c1F]

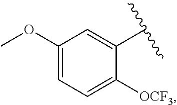 [SMILES: *c1cc(OC)ccc1OC(F)(F)F]

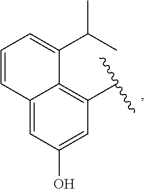 [SMILES: *c1cc(O)cc2cccc(C(C)C)c12]

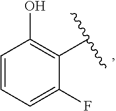 [SMILES: *c1c(O)cccc1F]

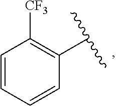 [SMILES: *c1ccccc1C(F)(F)F]

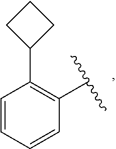 [SMILES: *c1ccccc1C1CCC1]

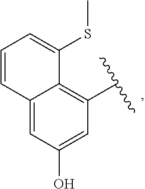 [SMILES: *c1cc(O)cc2cccc(SC)c12]

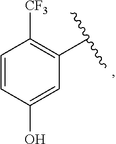 [SMILES: *c1cc(O)ccc1C(F)(F)F]

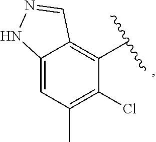 [SMILES: *c1c(Cl)c(C)cc2[nH]ncc12]

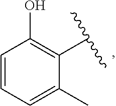 [SMILES: *c1c(C)cccc1O]

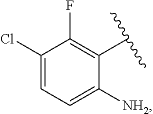 [SMILES: *c1c(N)ccc(Cl)c1F]

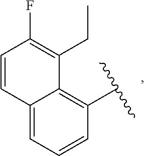 [SMILES: *c1cccc2ccc(F)c(CC)c12]

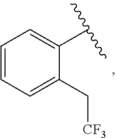 [SMILES: *c1ccccc1CC(F)(F)F]

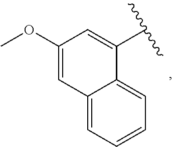 [SMILES: *c1cc(OC)cc2ccccc12]

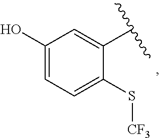 [SMILES: *c1cc(O)ccc1SC(F)(F)F]

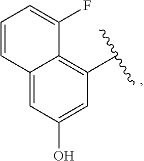 [SMILES: *c1cc(O)cc2cccc(F)c12]

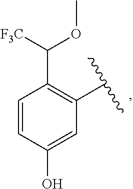 [SMILES: *c1cc(O)ccc1C(OC)C(F)(F)F]

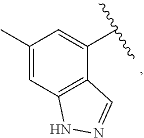 [SMILES: *c1cc(C)cc2[nH]ncc12]

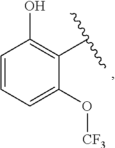 [SMILES: *c1c(O)cccc1OC(F)(F)F]

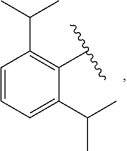 [SMILES: *c1c(C(C)C)cccc1C(C)C]

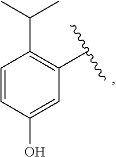 [SMILES: *c1cc(O)ccc1C(C)C]

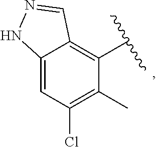 [SMILES: *c1c(C)c(Cl)cc2[nH]ncc12]

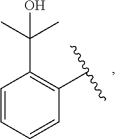 [SMILES: *c1ccccc1C(C)(C)O]

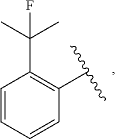 [SMILES: *c1ccccc1C(C)(C)F]

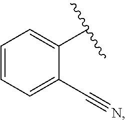 [SMILES: *c1ccccc1C#N]

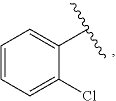 [SMILES: *c1ccccc1Cl]

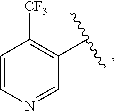 [SMILES: *c1cnccc1C(F)(F)F]

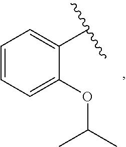 [SMILES: *c1ccccc1OC(C)C]

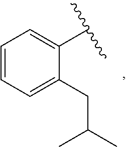 [SMILES: *c1ccccc1CC(C)C]

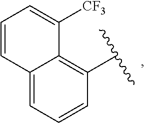 [SMILES: *c1cccc2cccc(C(F)(F)F)c12]

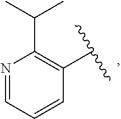 [SMILES: *c1cccnc1C(C)C]

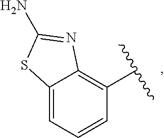 [SMILES: *c1cccc2sc(N)nc12]

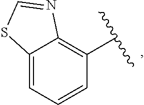 [SMILES: *c1cccc2scnc12]

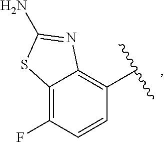 [SMILES: *c1ccc(F)c2sc(N)nc12]

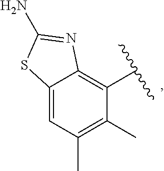 [SMILES: *c1c(C)c(C)cc2sc(N)nc12]

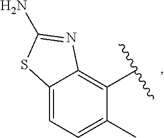 [SMILES: *c1c(C)ccc2sc(N)nc12]

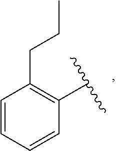 [SMILES: *c1ccccc1CCC]

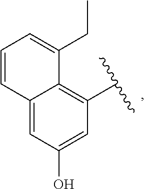 [SMILES: *c1cc(O)cc2cccc(CC)c12]

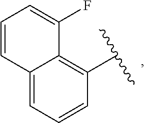 [SMILES: *c1cccc2cccc(F)c12]

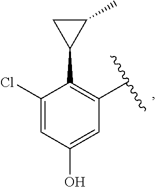 [SMILES: *c1cc(O)cc(Cl)c1[C@H]1C[C@@H]1C]

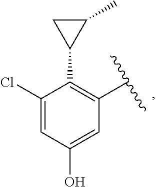 [SMILES: *c1cc(O)cc(Cl)c1[C@@H]1C[C@@H]1C]

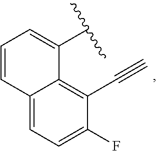 [SMILES: *c1cccc2ccc(F)c(C#C)c12]

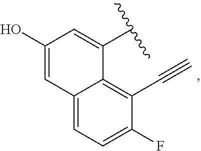 [SMILES: *c1cc(O)cc2ccc(F)c(C#C)c12]

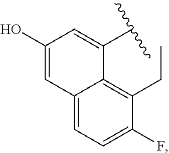 [SMILES: *c1cc(O)cc2ccc(F)c(CC)c12]

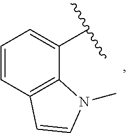 [SMILES: *c1cccc2ccn(C)c12]

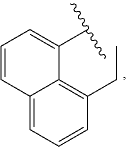 [SMILES: *c1cccc2cccc(CC)c12]

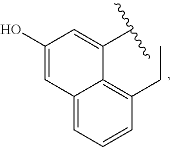 [SMILES: *c1cc(O)cc2cccc(CC)c12]

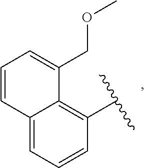 [SMILES: *c1cccc2cccc(COC)c12]

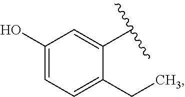 [SMILES: *c1cc(O)ccc1CC]

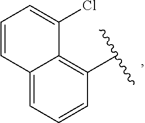 [SMILES: *c1cccc2cccc(Cl)c12]

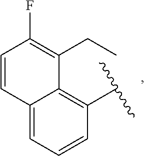 [SMILES: *c1cccc2ccc(F)c(CC)c12]

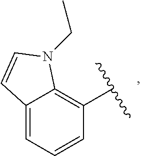 [SMILES: *c1cccc2ccn(CC)c12]

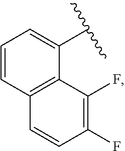 [SMILES: *c1cccc2ccc(F)c(F)c12]

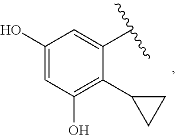 [SMILES: *c1cc(O)cc(O)c1C1CC1]

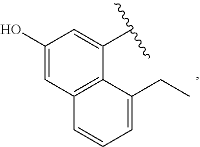 [SMILES: *c1cc(O)cc2cccc(CC)c12]

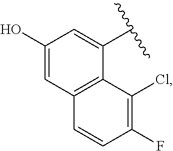 [SMILES: *c1cc(O)cc2ccc(F)c(Cl)c12]

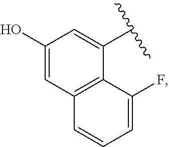 [SMILES: *c1cc(O)cc2cccc(F)c12]

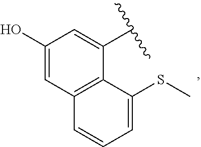 [SMILES: *c1cc(O)cc2cccc(SC)c12]

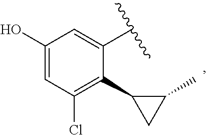 [SMILES: *c1cc(O)cc(Cl)c1[C@@H]1C[C@H]1C]

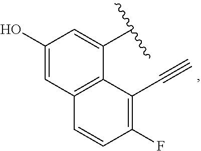 [SMILES: *c1cc(O)cc2ccc(F)c(C#C)c12]

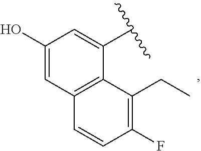 [SMILES: *c1cc(O)cc2ccc(F)c(CC)c12]

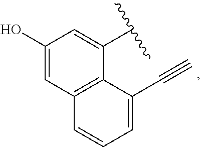 [SMILES: *c1cc(O)cc2cccc(C#C)c12]

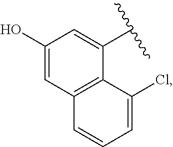 [SMILES: *c1cc(O)cc2cccc(Cl)c12]

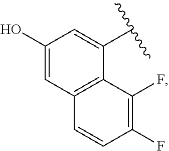 [SMILES: *c1cc(O)cc2ccc(F)c(F)c12]

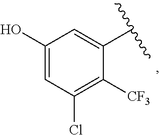 [SMILES: *c1cc(O)cc(Cl)c1C(F)(F)F]

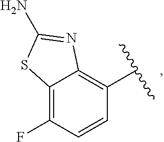 [SMILES: *c1ccc(F)c2sc(N)nc12]

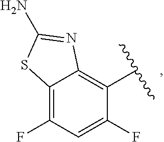 [SMILES: *c1c(F)cc(F)c2sc(N)nc12]

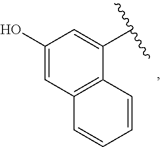 [SMILES: *c1cc(O)cc2ccccc12]

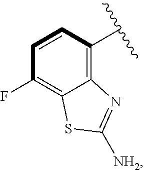

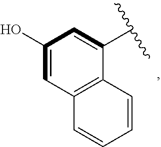

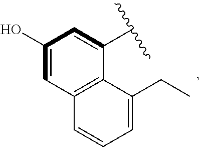

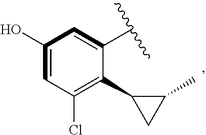

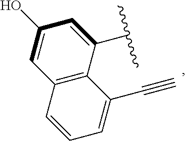

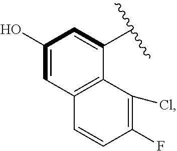

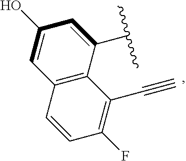

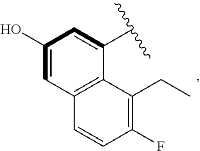

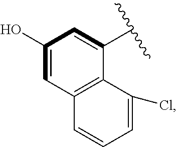

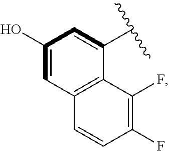

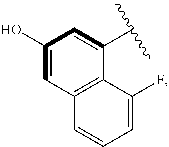

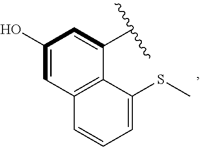

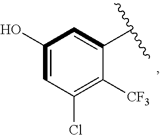

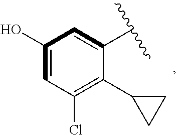

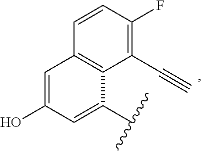

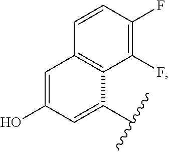

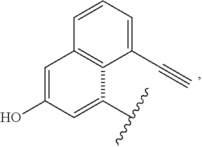

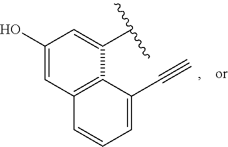

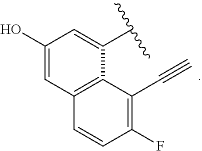

[24]. The compound of any one of aspects [1] to [23], wherein, R₄ is selected from any one of structrues in the following Table 5:

TABLE 5

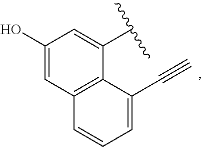 [SMILES: *c1cc(O)cc2cccc(C#C)c12]

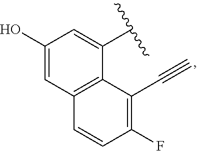 [SMILES: *c1cc(O)cc2ccc(F)c(C#C)c12]

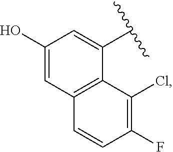 [SMILES: *c1cc(O)cc2ccc(F)c(Cl)c12]

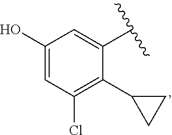 [SMILES: *c1cc(O)cc(Cl)c1C1CC1]

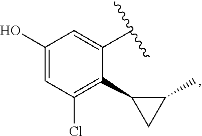 [SMILES: *c1cc(O)cc(Cl)c1[C@@H]1C[C@H]1C]

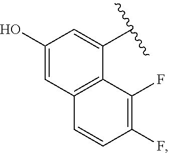 [SMILES: *c1cc(O)cc2ccc(F)c(F)c12]

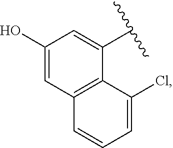 [SMILES: *c1cc(O)cc2cccc(Cl)c12]

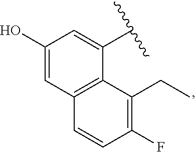 [SMILES: *c1cc(O)cc2ccc(F)c(CC)c12]

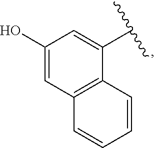 [SMILES: *c1cc(O)cc2ccccc12]

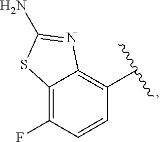 [SMILES: *c1ccc(F)c2sc(N)nc12]

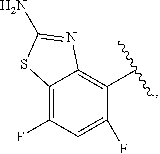 [SMILES: *c1c(F)cc(F)c2sc(N)nc12]

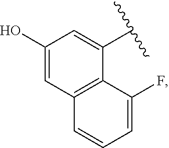 [SMILES: *c1cc(O)cc2cccc(F)c12]

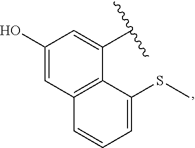 [SMILES: *c1cc(O)cc2cccc(SC)c12]

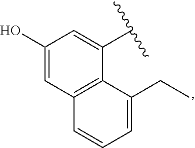 [SMILES: *c1cc(O)cc2cccc(CC)c12]

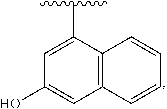 [SMILES: *c1cc(O)cc2ccccc12]

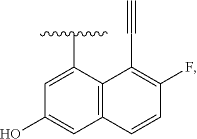 [SMILES: *c1cc(O)cc2ccc(F)c(C#C)c12]

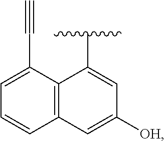 [SMILES: *c1cc(O)cc2cccc(C#C)c12]

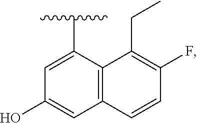 [SMILES: *c1cc(O)cc2ccc(F)c(CC)c12]

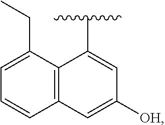 [SMILES: *c1cc(O)cc2cccc(CC)c12]

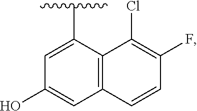 [SMILES: *c1cc(O)cc2ccc(F)c(Cl)c12]

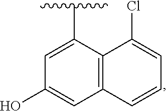 [SMILES: *c1cc(O)cc2cccc(Cl)c12]

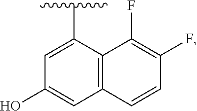 [SMILES: *c1cc(O)cc2ccc(F)c(F)c12]

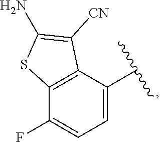 [SMILES: *c1ccc(F)c2sc(N)c(C#N)c12]

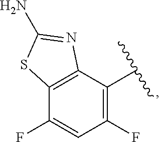 [SMILES: *c1c(F)cc(F)c2sc(N)nc12]

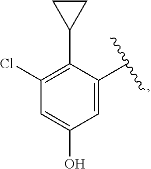 [SMILES: *c1cc(O)cc(Cl)c1C1CC1]

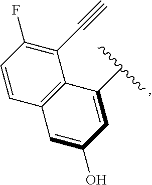

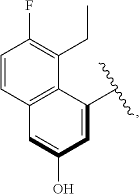

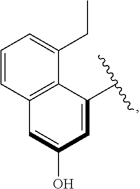

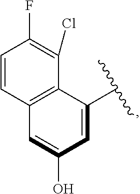

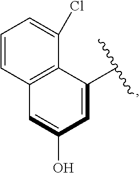

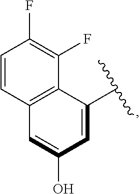

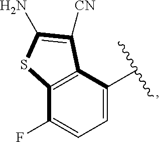

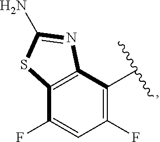

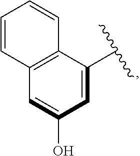

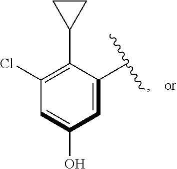

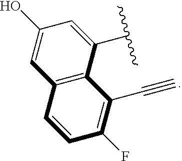

[25]. The compound of any one of aspects [1] to [24], wherein, R₄ is selected from any one of structures in the following Table 6:

TABLE 6

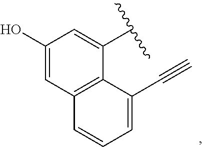 [SMILES: *c1cc(O)cc2cccc(C#C)c12]

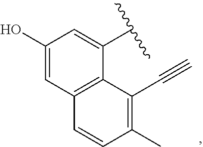 [SMILES: *c1cc(O)cc2ccc(C)c(C#C)c12]

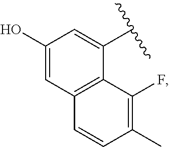 [SMILES: *c1cc(O)cc2ccc(C)c(F)c12]

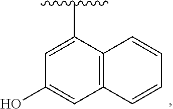 [SMILES: *c1cc(O)cc2ccccc12]

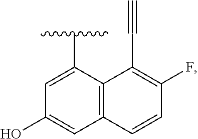 [SMILES: *c1cc(O)cc2ccc(F)c(C#C)c12]

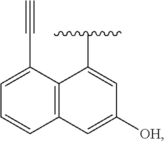 [SMILES: *c1cc(O)cc2cccc(C#C)c12]

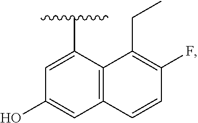 [SMILES: *c1cc(O)cc2ccc(F)c(CC)c12]

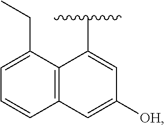 [SMILES: *c1cc(O)cc2cccc(CC)c12]

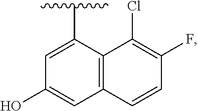 [SMILES: *c1cc(O)cc2ccc(F)c(Cl)c12]

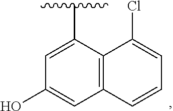 [SMILES: *c1cc(O)cc2cccc(Cl)c12]

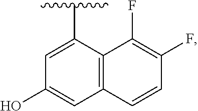 [SMILES: *c1cc(O)cc2ccc(F)c(F)c12]

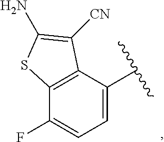 [SMILES: *c1ccc(F)c2sc(N)c(C#N)c12]

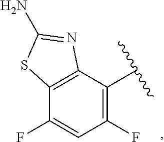 [SMILES: *c1c(F)cc(F)c2sc(N)nc12]

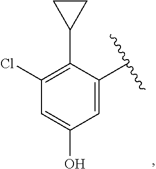 [SMILES: *c1cc(O)cc(Cl)c1C1CC1]

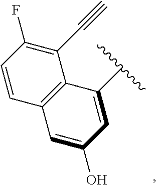

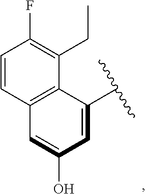

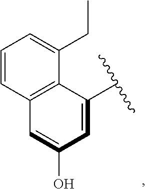

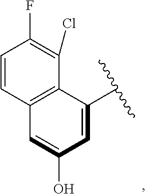

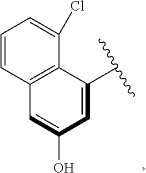

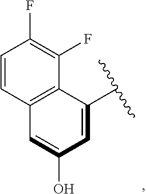

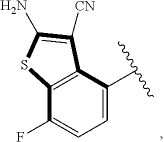

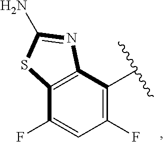

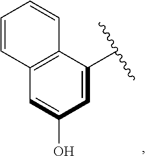

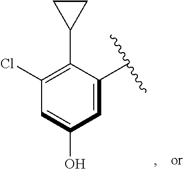

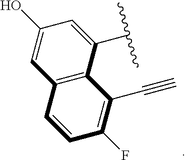

[26]. The compound of any one of aspects [1] to [25], wherein, R₄₁ is selected from any one of structures in the following Table 7:

TABLE 7

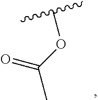 [SMILES: *OC(C)=O]

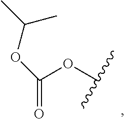 [SMILES: *OC(=O)OC(C)C]

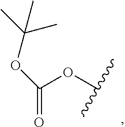 [SMILES: *OC(=O)OC(C)(C)C]

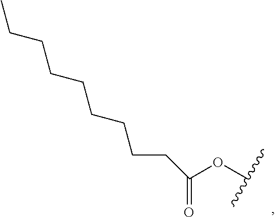 [SMILES: *OC(=O)CCCCCCCCC]

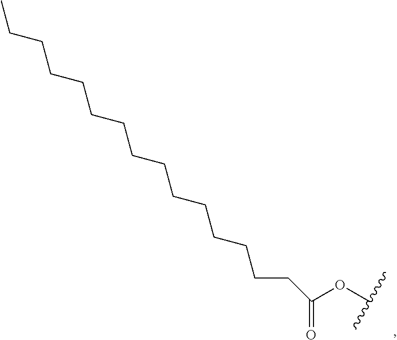 [SMILES: *OC(=O)CCCCCCCCCCCCCCC]

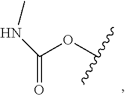 [SMILES: *OC(=O)NC]

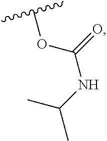 [SMILES: *OC(=O)NC(C)C]

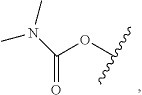 [SMILES: *OC(=O)N(C)C]

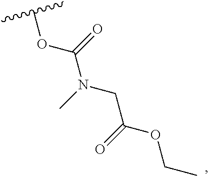 [SMILES: *OC(=O)N(C)CC(=O)OCC]

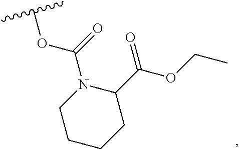 [SMILES: *OC(=O)N1CCCCC1C(=O)OCC]

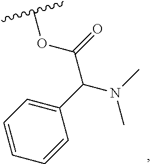 [SMILES: *OC(=O)C(c1ccccc1)N(C)C]

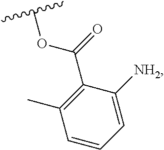 [SMILES: *OC(=O)c1c(C)cccc1N]

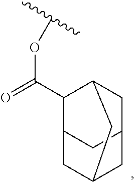 [SMILES: *OC(=O)C1C2CC3CC(C2)CC1C3]

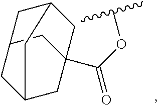 [SMILES: *OC(=O)C12CC3CC(CC(C3)C1)C2]

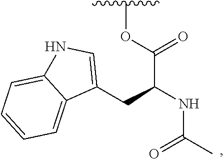 [SMILES: *OC(=O)[C@H](Cc1c[nH]c2ccccc12)NC(C)=O]

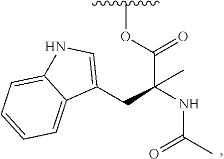 [SMILES: *OC(=O)[C@@](C)(Cc1c[nH]c2ccccc12)NC(C)=O]

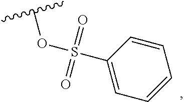 [SMILES: *OS(=O)(=O)c1ccccc1]

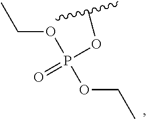 [SMILES: *OP(=O)(OCC)OCC]

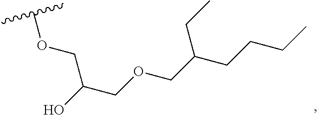 [SMILES: *OCC(O)COCC(CC)CCCC]

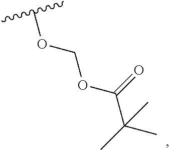 [SMILES: *OCOC(=O)C(C)(C)C]

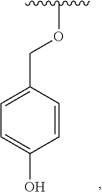 [SMILES: *OCc1ccc(O)cc1]

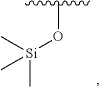 [SMILES: *O[Si](C)(C)C]

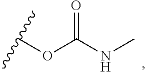 [SMILES: *OC(=O)NC]

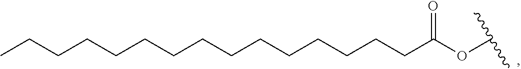 [SMILES: *OC(=O)CCCCCCCCCCCCCCC]

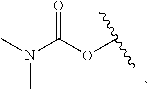 [SMILES: *OC(=O)N(C)C]

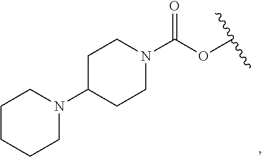 [SMILES: *OC(=O)N1CCC(N2CCCCC2)CC1]

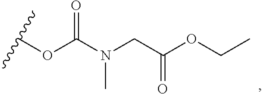 [SMILES: *OC(=O)N(C)CC(=O)OCC]

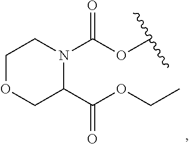 [SMILES: *OC(=O)N1CCOCC1C(=O)OCC]

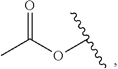 [SMILES: *OC(C)=O]

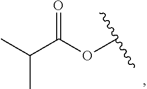 [SMILES: *OC(=O)C(C)C]

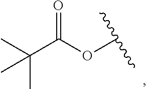 [SMILES: *OC(=O)C(C)(C)C]

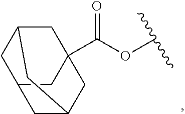 [SMILES: *OC(=O)C12CC3CC(CC(C3)C1)C2]

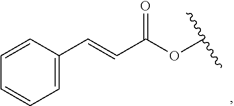 [SMILES: *OC(=O)/C=C/c1ccccc1]

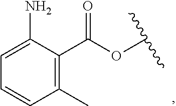 [SMILES: *OC(=O)c1c(C)cccc1N]

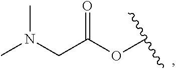 [SMILES: *OC(=O)CN(C)C]

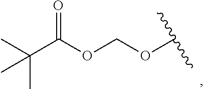 [SMILES: *OCOC(=O)C(C)(C)C]

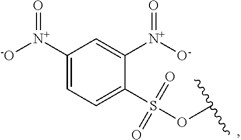

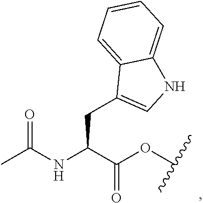 [SMILES: *OC(=O)[C@H](Cc1c[nH]c2ccccc12)NC(C)=O]

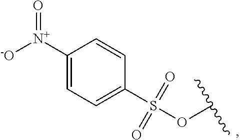

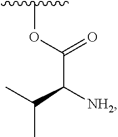 [SMILES: *OC(=O)[C@@H](N)C(C)C]

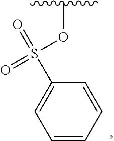 [SMILES: *OS(=O)(=O)c1ccccc1]

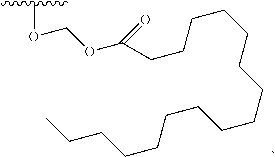 [SMILES: *OCOC(=O)CCCCCCCCCCCCCCCC]

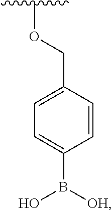 [SMILES: *OCc1ccc(B(O)O)cc1]

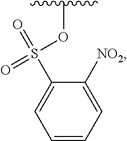 [SMILES: *OS(=O)(=O)c1ccccc1[N+](=O)[O-]]

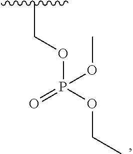 [SMILES: *COP(=O)(OC)OCC]

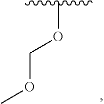 [SMILES: *OCOC]

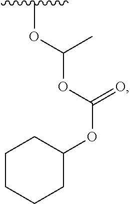 [SMILES: *OC(C)OC(=O)OC1CCCCC1]

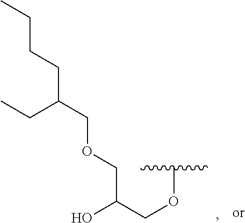 [SMILES: *OCC(O)COCC(CC)CCCC]

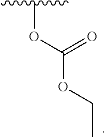 [SMILES: *OC(=O)OCC]

[27]. The compound of any one of aspects [1] to [26], wherein, R₄ is selected from any one of structures in the following Table 8:

TABLE 8

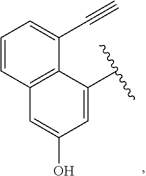 [SMILES: *c1cc(O)cc2cccc(C#C)c12]

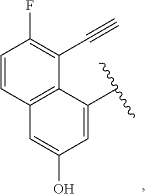 [SMILES: *c1cc(O)cc2ccc(F)c(C#C)c12]

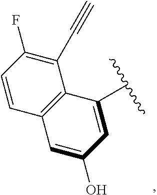

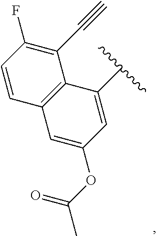 [SMILES: *c1cc(OC(C)=O)cc2ccc(F)c(C#C)c12]

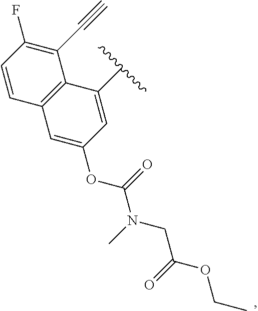 [SMILES: *c1cc(OC(=O)N(C)CC(=O)OCC)cc2ccc(F)c(C#C)c12]

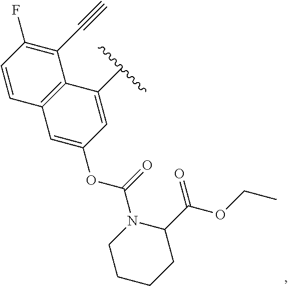 [SMILES: *c1cc(OC(=O)N2CCCCC2C(=O)OCC)cc2ccc(F)c(C#C)c12]

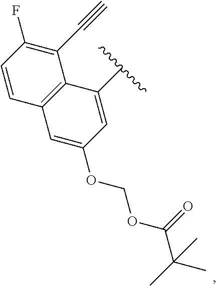 [SMILES: *c1cc(OCOC(=O)C(C)(C)C)cc2ccc(F)c(C#C)c12]

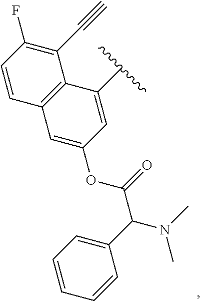 [SMILES: *c1cc(OC(=O)C(c2ccccc2)N(C)C)cc2ccc(F)c(C#C)c12]

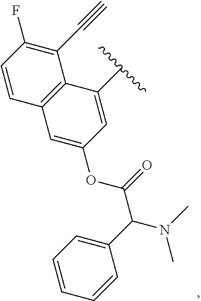 [SMILES: *c1cc(OC(=O)C(c2ccccc2)N(C)C)cc2ccc(F)c(C#C)c12]

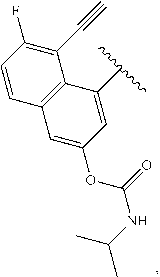 [SMILES: *c1cc(OC(=O)NC(C)C)cc2ccc(F)c(C#C)c12]

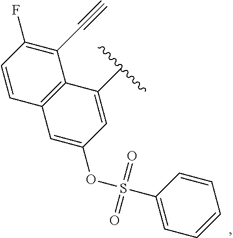 [SMILES: *c1cc(OS(=O)(=O)c2ccccc2)cc2ccc(F)c(C#C)c12]

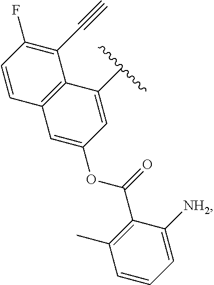 [SMILES: *c1cc(OC(=O)c2c(C)cccc2N)cc2ccc(F)c(C#C)c12]

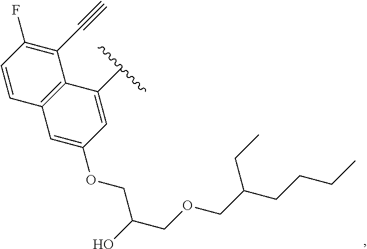 [SMILES: *c1cc(OCC(O)COCC(CC)CCCC)cc2ccc(F)c(C#C)c12]

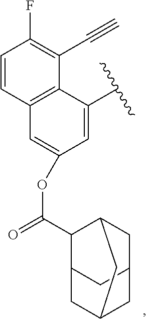 [SMILES: *c1cc(OC(=O)C2C3CC4CC(C3)CC2C4)cc2ccc(F)c(C#C)c12]

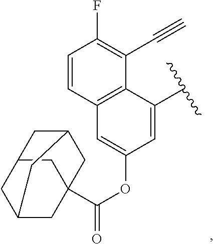 [SMILES: *c1cc(OC(=O)C23CC4CC(CC(C4)C2)C3)cc2ccc(F)c(C#C)c12]

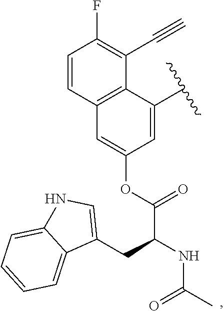 [SMILES: *c1cc(OC(=O)[C@H](Cc2c[nH]c3ccccc23)NC(C)=O)cc2ccc(F)c(C#C)c12]

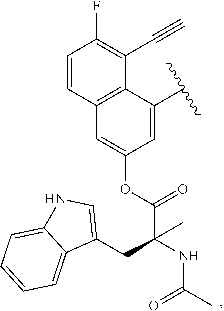 [SMILES: *c1cc(OC(=O)[C@@](C)(Cc2c[nH]c3ccccc23)NC(C)=O)cc2ccc(F)c(C#C)c12]

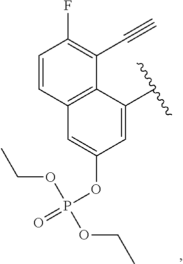 [SMILES: *c1cc(OP(=O)(OCC)OCC)cc2ccc(F)c(C#C)c12]

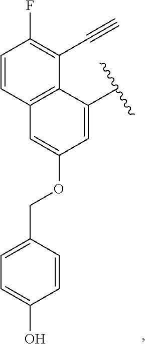 [SMILES: *c1cc(OCc2ccc(O)cc2)cc2ccc(F)c(C#C)c12]

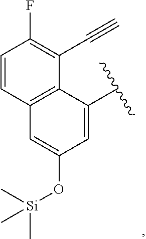 [SMILES: *c1cc(O[Si](C)(C)C)cc2ccc(F)c(C#C)c12]

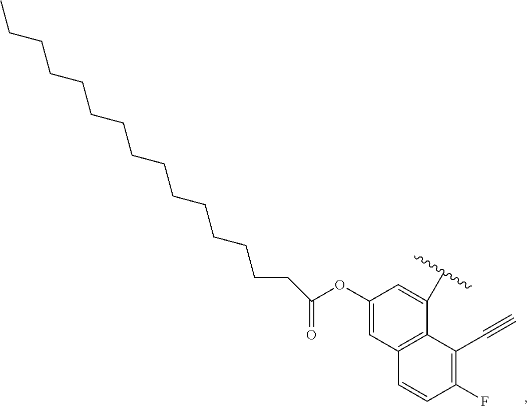 [SMILES: *c1cc(OC(=O)CCCCCCCCCCCCCCC)cc2ccc(F)c(C#C)c12]

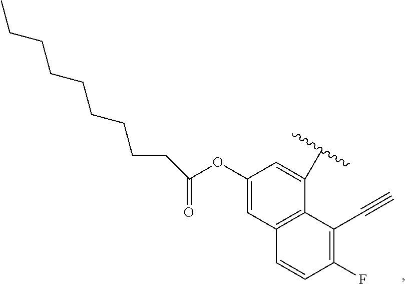 [SMILES: *c1cc(OC(=O)CCCCCCCCC)cc2ccc(F)c(C#C)c12]

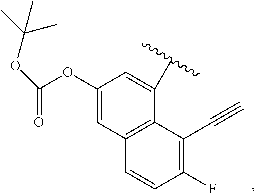 [SMILES: *c1cc(OC(=O)OC(C)(C)C)cc2ccc(F)c(C#C)c12]

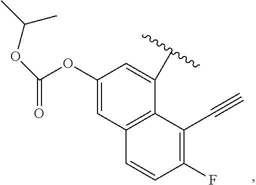 [SMILES: *c1cc(OC(=O)OC(C)C)cc2ccc(F)c(C#C)c12]

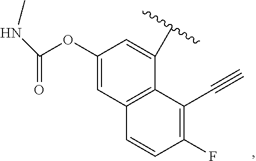 [SMILES: *c1cc(OC(=O)NC)cc2ccc(F)c(C#C)c12]

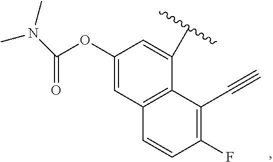 [SMILES: *c1cc(OC(=O)N(C)C)cc2ccc(F)c(C#C)c12]

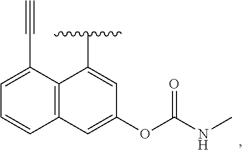 [SMILES: *c1cc(OC(=O)NC)cc2cccc(C#C)c12]

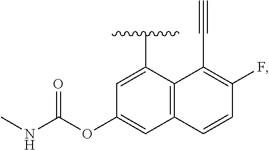 [SMILES: *c1cc(OC(=O)NC)cc2ccc(F)c(C#C)c12]

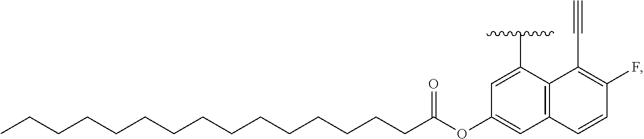 [SMILES: *c1cc(OC(=O)CCCCCCCCCCCCCCC)cc2ccc(F)c(C#C)c12]

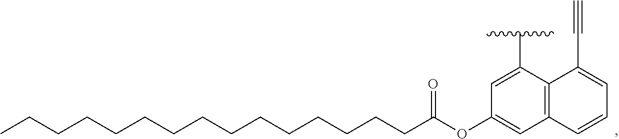 [SMILES: *c1cc(OC(=O)CCCCCCCCCCCCCCC)cc2cccc(C#C)c12]

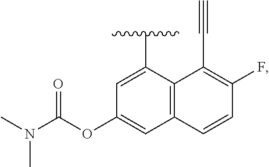 [SMILES: *c1cc(OC(=O)N(C)C)cc2ccc(F)c(C#C)c12]

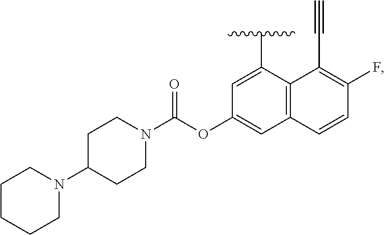 [SMILES: *c1cc(OC(=O)N2CCC(N3CCCCC3)CC2)cc2ccc(F)c(C#C)c12]

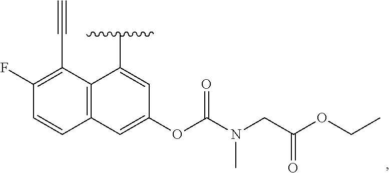 [SMILES: *c1cc(OC(=O)N(C)CC(=O)OCC)cc2ccc(F)c(C#C)c12]

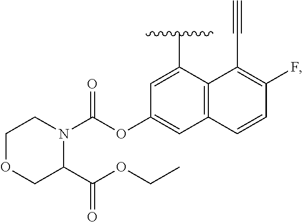 [SMILES: *c1cc(OC(=O)N2CCOCC2C(=O)OCC)cc2ccc(F)c(C#C)c12]

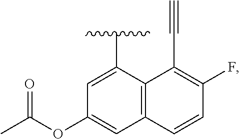 [SMILES: *c1cc(OC(C)=O)cc2ccc(F)c(C#C)c12]

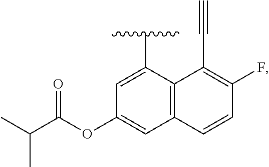 [SMILES: *c1cc(OC(=O)C(C)C)cc2ccc(F)c(C#C)c12]

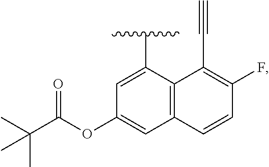 [SMILES: *c1cc(OC(=O)C(C)(C)C)cc2ccc(F)c(C#C)c12]

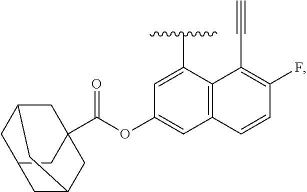 [SMILES: *c1cc(OC(=O)C23CC4CC(CC(C4)C2)C3)cc2ccc(F)c(C#C)c12]

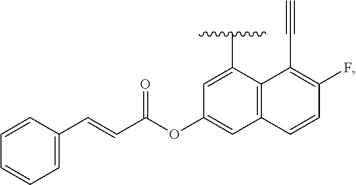 [SMILES: *c1cc(OC(=O)/C=C/c2ccccc2)cc2ccc(F)c(C#C)c12]

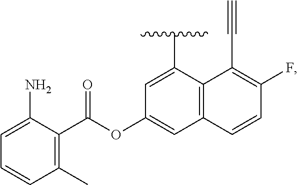 [SMILES: *c1cc(OC(=O)c2c(C)cccc2N)cc2ccc(F)c(C#C)c12]

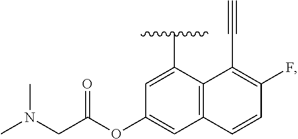 [SMILES: *c1cc(OC(=O)CN(C)C)cc2ccc(F)c(C#C)c12]

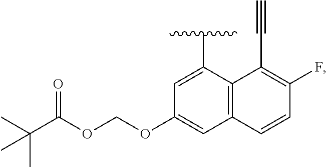 [SMILES: *c1cc(OCOC(=O)C(C)(C)C)cc2ccc(F)c(C#C)c12]

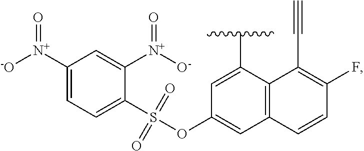

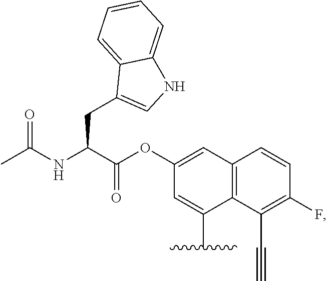 [SMILES: *c1cc(OC(=O)[C@H](Cc2c[nH]c3ccccc23)NC(C)=O)cc2ccc(F)c(C#C)c12]

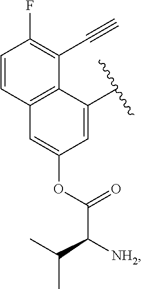 [SMILES: *c1cc(OC(=O)[C@@H](N)C(C)C)cc2ccc(F)c(C#C)c12]

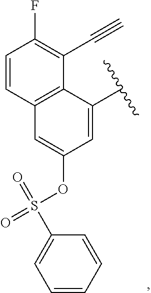 [SMILES: *c1cc(OS(=O)(=O)c2ccccc2)cc2ccc(F)c(C#C)c12]

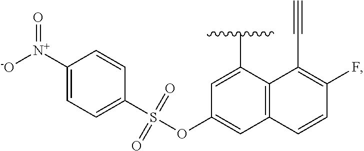

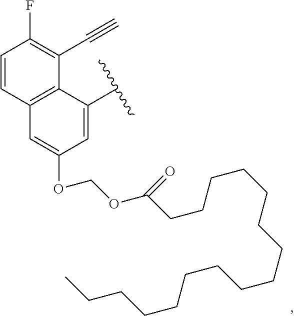 [SMILES: *c1cc(OCOC(=O)CCCCCCCCCCCCCCCC)cc2ccc(F)c(C#C)c12]

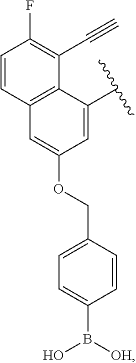 [SMILES: *c1cc(OCc2ccc(B(O)O)cc2)cc2ccc(F)c(C#C)c12]

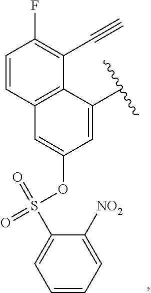 [SMILES: *c1cc(OS(=O)(=O)c2ccccc2[N+](=O)[O-])cc2ccc(F)c(C#C)c12]

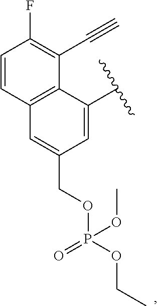 [SMILES: *c1cc(COP(=O)(OC)OCC)cc2ccc(F)c(C#C)c12]

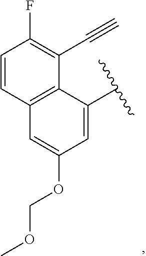 [SMILES: *c1cc(OCOC)cc2ccc(F)c(C#C)c12]

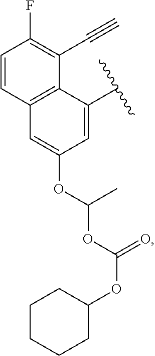 [SMILES: *c1cc(OC(C)OC(=O)OC2CCCCC2)cc2ccc(F)c(C#C)c12]

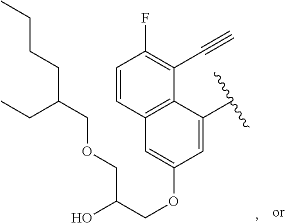 [SMILES: *c1cc(OCC(O)COCC(CC)CCCC)cc2ccc(F)c(C#C)c12]

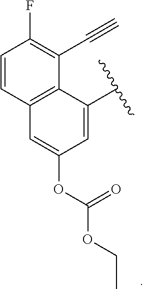 [SMILES: *c1cc(OC(=O)OCC)cc2ccc(F)c(C#C)c12]

[28]. The compound of any one of aspects [1] to [27], wherein, R₅ is selected from hydrogen or any one of structures in the following Table 9:

TABLE 9

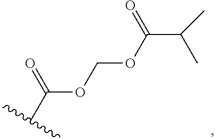 [SMILES: *C(=O)OCOC(=O)C(C)C]

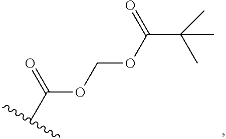 [SMILES: *C(=O)OCOC(=O)C(C)(C)C]

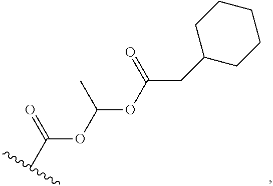 [SMILES: *C(=O)OC(C)OC(=O)CC1CCCCC1]

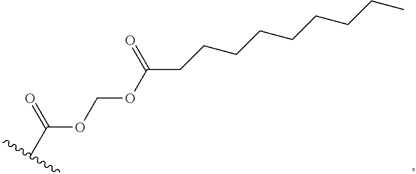 [SMILES: *C(=O)OCOC(=O)CCCCCCCCC]

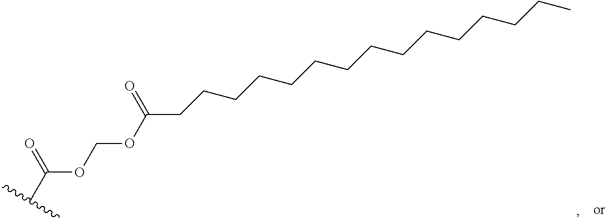 [SMILES: *C(=O)OCOC(=O)CCCCCCCCCCCCCCC]

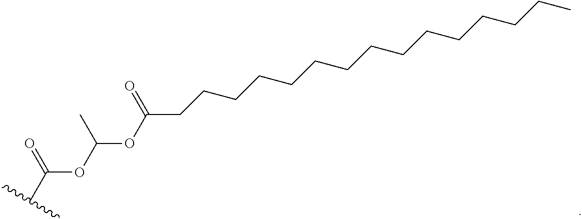 [SMILES: *C(=O)OC(C)OC(=O)CCCCCCCCCCCCCCC]

[29]. The compound of any one of aspects [1] to [28], wherein, the compound is selected from any one of compounds in the following Table 10:

TABLE 10

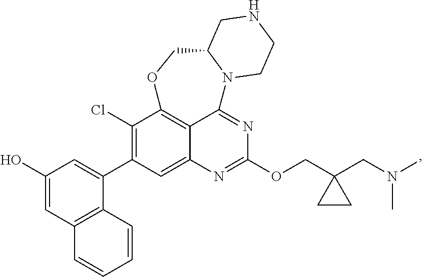

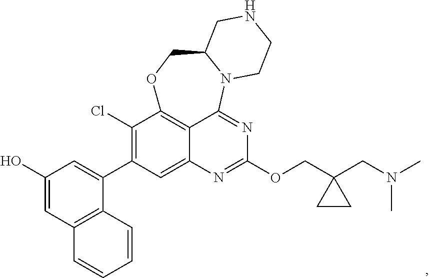 [SMILES: CN(C)CC1(COc2nc3c4c(c(Cl)c(-c5cc(O)cc6ccccc56)cc4n2)OC[C@H]2CNCCN32)CC1]

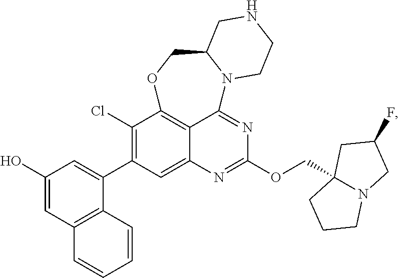 [SMILES: Oc1cc(-c2cc3nc(OC[C@@]45CCCN4C[C@H](F)C5)nc4c3c(c2Cl)OC[C@H]2CNCCN42)c2ccccc2c1]

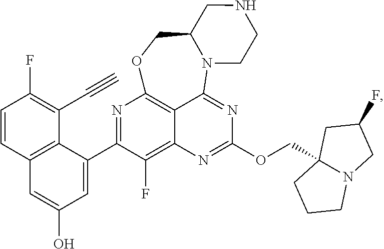 [SMILES: C#Cc1c(F)ccc2cc(O)cc(-c3nc4c5c(nc(OC[C@@]67CCCN6C[C@H](F)C7)nc5c3F)N3CCNC[C@@H]3CO4)c12]

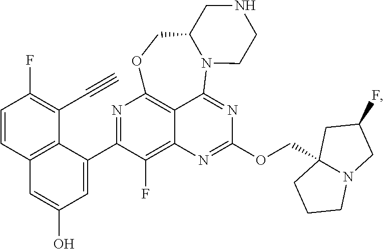 [SMILES: C#Cc1c(F)ccc2cc(O)cc(-c3nc4c5c(nc(OC[C@@]67CCCN6C[C@H](F)C7)nc5c3F)N3CCNC[C@H]3CO4)c12]

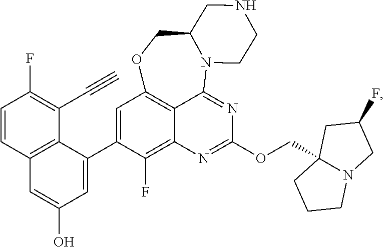 [SMILES: C#Cc1c(F)ccc2cc(O)cc(-c3cc4c5c(nc(OC[C@@]67CCCN6C[C@H](F)C7)nc5c3F)N3CCNC[C@@H]3CO4)c12]

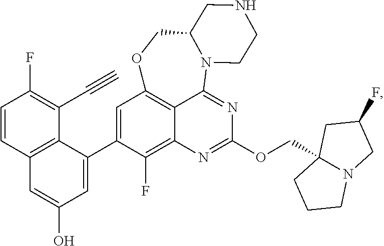 [SMILES: C#Cc1c(F)ccc2cc(O)cc(-c3cc4c5c(nc(OC[C@@]67CCCN6C[C@H](F)C7)nc5c3F)N3CCNC[C@H]3CO4)c12]

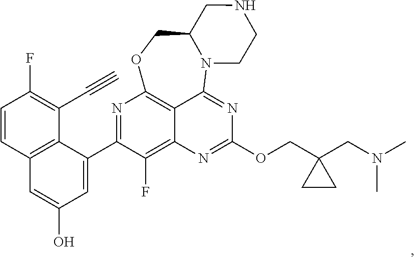 [SMILES: C#Cc1c(F)ccc2cc(O)cc(-c3nc4c5c(nc(OCC6(CN(C)C)CC6)nc5c3F)N3CCNC[C@@H]3CO4)c12]

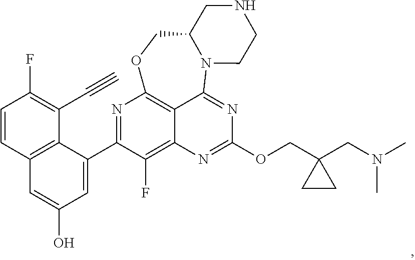 [SMILES: C#Cc1c(F)ccc2cc(O)cc(-c3nc4c5c(nc(OCC6(CN(C)C)CC6)nc5c3F)N3CCNC[C@H]3CO4)c12]

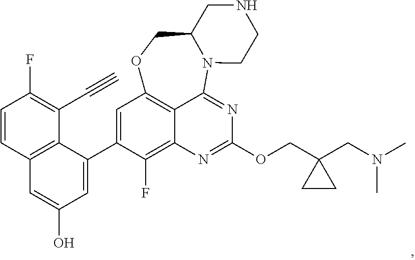 [SMILES: C#Cc1c(F)ccc2cc(O)cc(-c3cc4c5c(nc(OCC6(CN(C)C)CC6)nc5c3F)N3CCNC[C@@H]3CO4)c12]

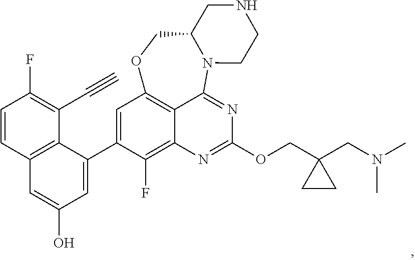 [SMILES: C#Cc1c(F)ccc2cc(O)cc(-c3cc4c5c(nc(OCC6(CN(C)C)CC6)nc5c3F)N3CCNC[C@H]3CO4)c12]

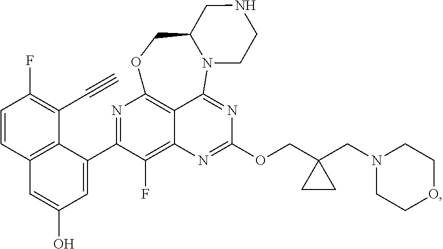 [SMILES: C#Cc1c(F)ccc2cc(O)cc(-c3nc4c5c(nc(OCC6(CN7CCOCC7)CC6)nc5c3F)N3CCNC[C@@H]3CO4)c12]

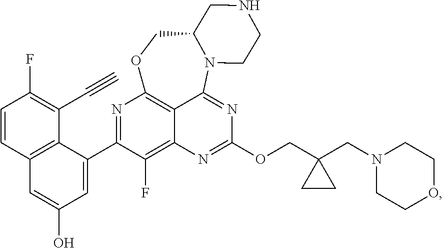 [SMILES: C#Cc1c(F)ccc2cc(O)cc(-c3nc4c5c(nc(OCC6(CN7CCOCC7)CC6)nc5c3F)N3CCNC[C@H]3CO4)c12]

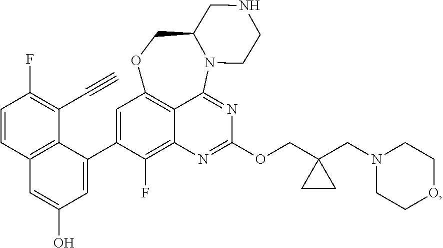 [SMILES: C#Cc1c(F)ccc2cc(O)cc(-c3cc4c5c(nc(OCC6(CN7CCOCC7)CC6)nc5c3F)N3CCNC[C@@H]3CO4)c12]

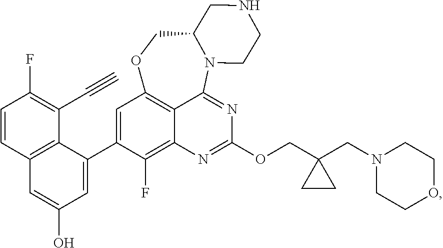 [SMILES: C#Cc1c(F)ccc2cc(O)cc(-c3cc4c5c(nc(OCC6(CN7CCOCC7)CC6)nc5c3F)N3CCNC[C@H]3CO4)c12]

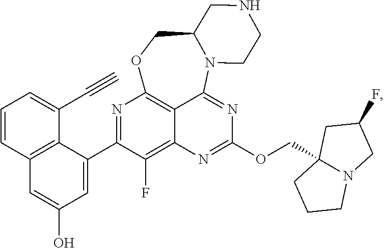 [SMILES: C#Cc1cccc2cc(O)cc(-c3nc4c5c(nc(OC[C@@]67CCCN6C[C@H](F)C7)nc5c3F)N3CCNC[C@@H]3CO4)c12]

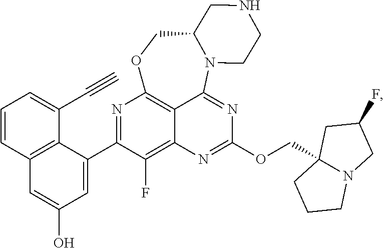 [SMILES: C#Cc1cccc2cc(O)cc(-c3nc4c5c(nc(OC[C@@]67CCCN6C[C@H](F)C7)nc5c3F)N3CCNC[C@H]3CO4)c12]

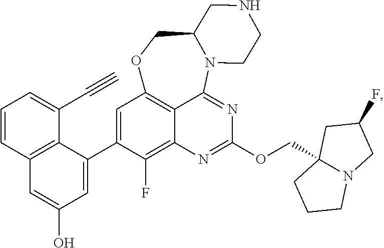 [SMILES: C#Cc1cccc2cc(O)cc(-c3cc4c5c(nc(OC[C@@]67CCCN6C[C@H](F)C7)nc5c3F)N3CCNC[C@@H]3CO4)c12]

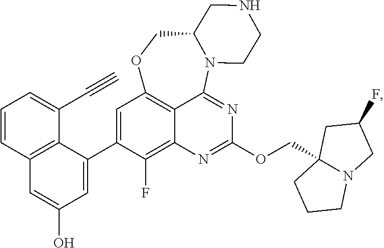 [SMILES: C#Cc1cccc2cc(O)cc(-c3cc4c5c(nc(OC[C@@]67CCCN6C[C@H](F)C7)nc5c3F)N3CCNC[C@H]3CO4)c12]

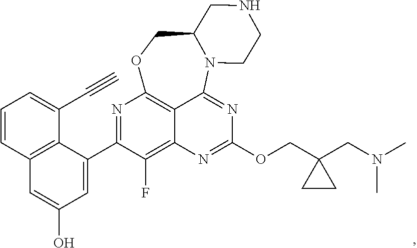 [SMILES: C#Cc1cccc2cc(O)cc(-c3nc4c5c(nc(OCC6(CN(C)C)CC6)nc5c3F)N3CCNC[C@@H]3CO4)c12]

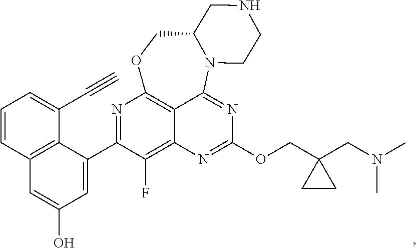 [SMILES: C#Cc1cccc2cc(O)cc(-c3nc4c5c(nc(OCC6(CN(C)C)CC6)nc5c3F)N3CCNC[C@H]3CO4)c12]

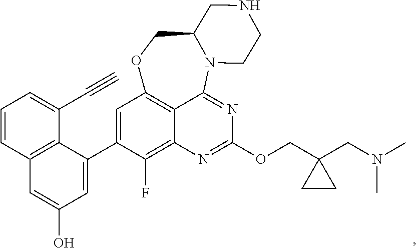 [SMILES: C#Cc1cccc2cc(O)cc(-c3cc4c5c(nc(OCC6(CN(C)C)CC6)nc5c3F)N3CCNC[C@@H]3CO4)c12]

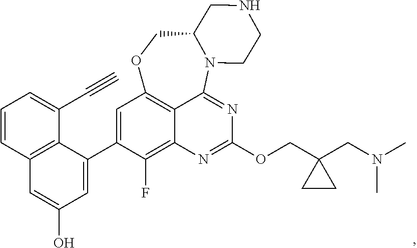 [SMILES: C#Cc1cccc2cc(O)cc(-c3cc4c5c(nc(OCC6(CN(C)C)CC6)nc5c3F)N3CCNC[C@H]3CO4)c12]

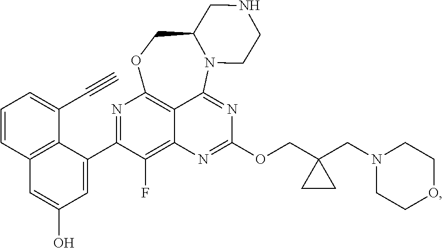 [SMILES: C#Cc1cccc2cc(O)cc(-c3nc4c5c(nc(OCC6(CN7CCOCC7)CC6)nc5c3F)N3CCNC[C@@H]3CO4)c12]

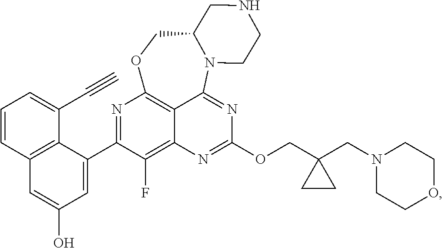 [SMILES: C#Cc1cccc2cc(O)cc(-c3nc4c5c(nc(OCC6(CN7CCOCC7)CC6)nc5c3F)N3CCNC[C@H]3CO4)c12]

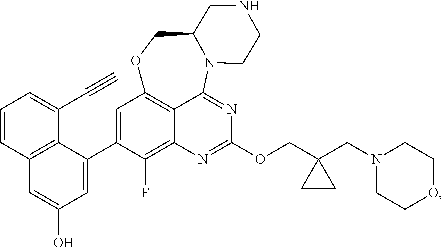 [SMILES: C#Cc1cccc2cc(O)cc(-c3cc4c5c(nc(OCC6(CN7CCOCC7)CC6)nc5c3F)N3CCNC[C@@H]3CO4)c12]

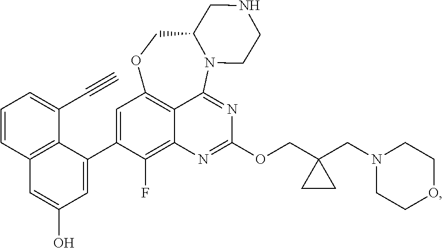 [SMILES: C#Cc1cccc2cc(O)cc(-c3cc4c5c(nc(OCC6(CN7CCOCC7)CC6)nc5c3F)N3CCNC[C@H]3CO4)c12]

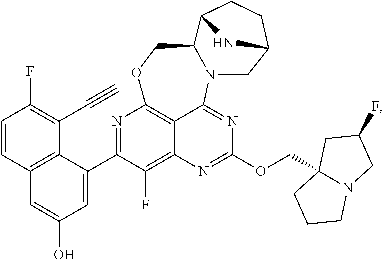 [SMILES: C#Cc1c(F)ccc2cc(O)cc(-c3nc4c5c(nc(OC[C@@]67CCCN6C[C@H](F)C7)nc5c3F)N3C[C@H]5CC[C@H](N5)[C@@H]3CO4)c12]

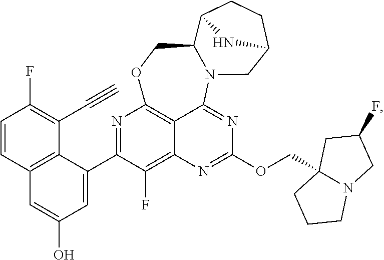 [SMILES: C#Cc1c(F)ccc2cc(O)cc(-c3nc4c5c(nc(OC[C@@]67CCCN6C[C@H](F)C7)nc5c3F)N3C[C@@H]5CC[C@@H](N5)[C@@H]3CO4)c12]

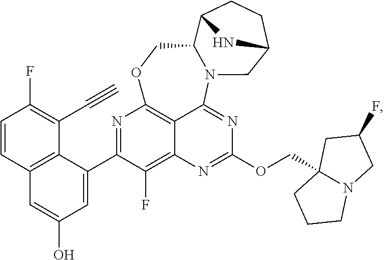 [SMILES: C#Cc1c(F)ccc2cc(O)cc(-c3nc4c5c(nc(OC[C@@]67CCCN6C[C@H](F)C7)nc5c3F)N3C[C@H]5CC[C@H](N5)[C@H]3CO4)c12]

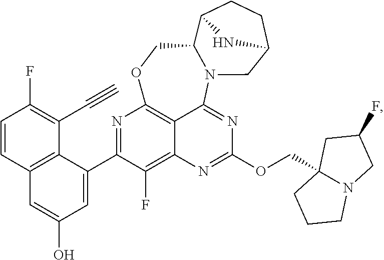 [SMILES: C#Cc1c(F)ccc2cc(O)cc(-c3nc4c5c(nc(OC[C@@]67CCCN6C[C@H](F)C7)nc5c3F)N3C[C@@H]5CC[C@@H](N5)[C@H]3CO4)c12]

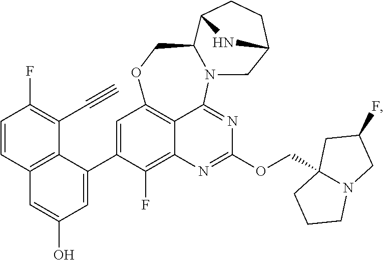 [SMILES: C#Cc1c(F)ccc2cc(O)cc(-c3cc4c5c(nc(OC[C@@]67CCCN6C[C@H](F)C7)nc5c3F)N3C[C@H]5CC[C@H](N5)[C@@H]3CO4)c12]

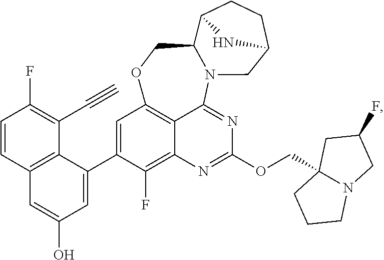 [SMILES: C#Cc1c(F)ccc2cc(O)cc(-c3cc4c5c(nc(OC[C@@]67CCCN6C[C@H](F)C7)nc5c3F)N3C[C@@H]5CC[C@@H](N5)[C@@H]3CO4)c12]

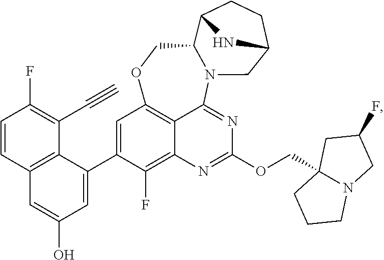 [SMILES: C#Cc1c(F)ccc2cc(O)cc(-c3cc4c5c(nc(OC[C@@]67CCCN6C[C@H](F)C7)nc5c3F)N3C[C@H]5CC[C@H](N5)[C@H]3CO4)c12]

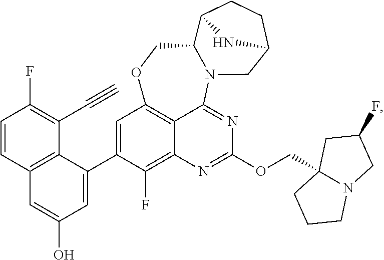 [SMILES: C#Cc1c(F)ccc2cc(O)cc(-c3cc4c5c(nc(OC[C@@]67CCCN6C[C@H](F)C7)nc5c3F)N3C[C@@H]5CC[C@@H](N5)[C@H]3CO4)c12]

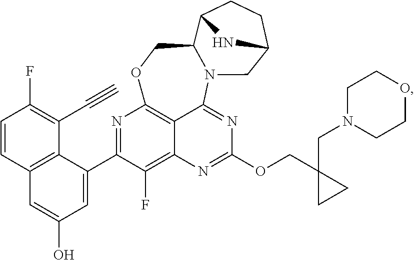 [SMILES: C#Cc1c(F)ccc2cc(O)cc(-c3nc4c5c(nc(OCC6(CN7CCOCC7)CC6)nc5c3F)N3C[C@H]5CC[C@H](N5)[C@@H]3CO4)c12]

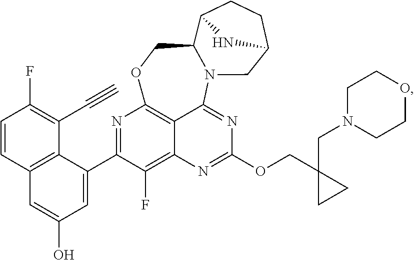 [SMILES: C#Cc1c(F)ccc2cc(O)cc(-c3nc4c5c(nc(OCC6(CN7CCOCC7)CC6)nc5c3F)N3C[C@@H]5CC[C@@H](N5)[C@@H]3CO4)c12]

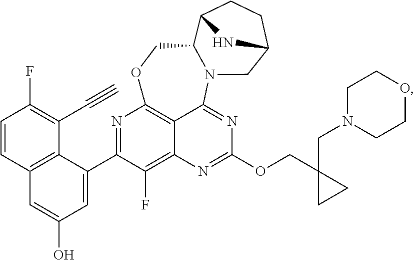 [SMILES: C#Cc1c(F)ccc2cc(O)cc(-c3nc4c5c(nc(OCC6(CN7CCOCC7)CC6)nc5c3F)N3C[C@H]5CC[C@H](N5)[C@H]3CO4)c12]

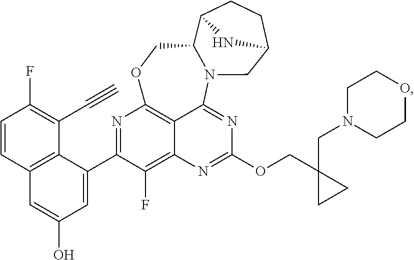 [SMILES: C#Cc1c(F)ccc2cc(O)cc(-c3nc4c5c(nc(OCC6(CN7CCOCC7)CC6)nc5c3F)N3C[C@@H]5CC[C@@H](N5)[C@H]3CO4)c12]

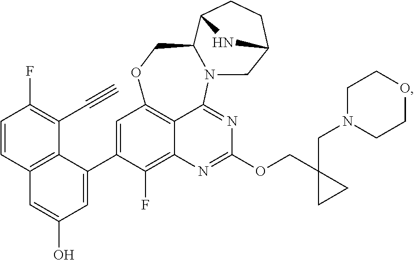 [SMILES: C#Cc1c(F)ccc2cc(O)cc(-c3cc4c5c(nc(OCC6(CN7CCOCC7)CC6)nc5c3F)N3C[C@H]5CC[C@H](N5)[C@@H]3CO4)c12]

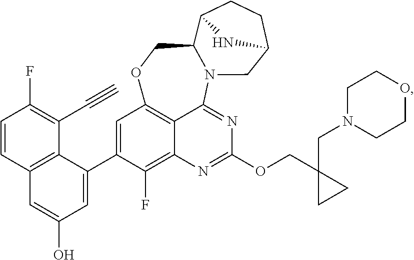 [SMILES: C#Cc1c(F)ccc2cc(O)cc(-c3cc4c5c(nc(OCC6(CN7CCOCC7)CC6)nc5c3F)N3C[C@@H]5CC[C@@H](N5)[C@@H]3CO4)c12]

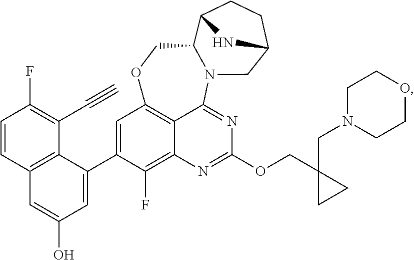 [SMILES: C#Cc1c(F)ccc2cc(O)cc(-c3cc4c5c(nc(OCC6(CN7CCOCC7)CC6)nc5c3F)N3C[C@H]5CC[C@H](N5)[C@H]3CO4)c12]

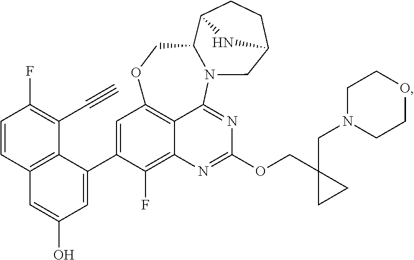 [SMILES: C#Cc1c(F)ccc2cc(O)cc(-c3cc4c5c(nc(OCC6(CN7CCOCC7)CC6)nc5c3F)N3C[C@@H]5CC[C@@H](N5)[C@H]3CO4)c12]

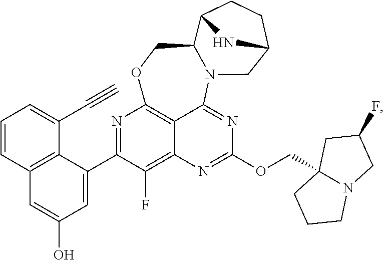 [SMILES: C#Cc1cccc2cc(O)cc(-c3nc4c5c(nc(OC[C@@]67CCCN6C[C@H](F)C7)nc5c3F)N3C[C@H]5CC[C@H](N5)[C@@H]3CO4)c12]

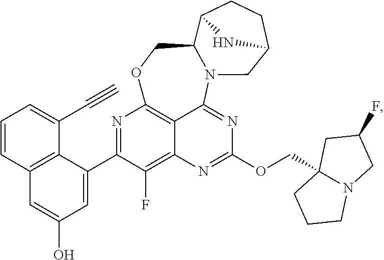 [SMILES: C#Cc1cccc2cc(O)cc(-c3nc4c5c(nc(OC[C@@]67CCCN6C[C@H](F)C7)nc5c3F)N3C[C@@H]5CC[C@@H](N5)[C@@H]3CO4)c12]

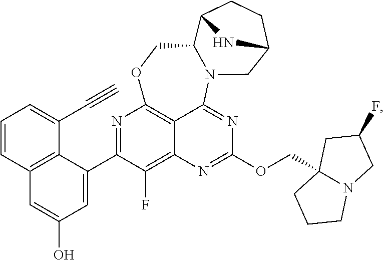 [SMILES: C#Cc1cccc2cc(O)cc(-c3nc4c5c(nc(OC[C@@]67CCCN6C[C@H](F)C7)nc5c3F)N3C[C@H]5CC[C@H](N5)[C@H]3CO4)c12]

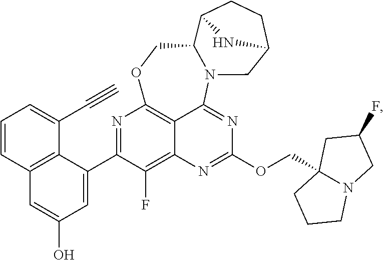 [SMILES: C#Cc1cccc2cc(O)cc(-c3nc4c5c(nc(OC[C@@]67CCCN6C[C@H](F)C7)nc5c3F)N3C[C@@H]5CC[C@@H](N5)[C@H]3CO4)c12]

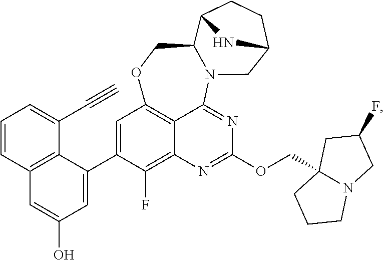 [SMILES: C#Cc1cccc2cc(O)cc(-c3cc4c5c(nc(OC[C@@]67CCCN6C[C@H](F)C7)nc5c3F)N3C[C@H]5CC[C@H](N5)[C@@H]3CO4)c12]

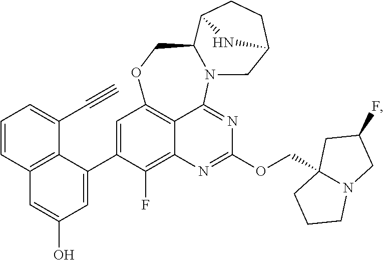 [SMILES: C#Cc1cccc2cc(O)cc(-c3cc4c5c(nc(OC[C@@]67CCCN6C[C@H](F)C7)nc5c3F)N3C[C@@H]5CC[C@@H](N5)[C@@H]3CO4)c12]

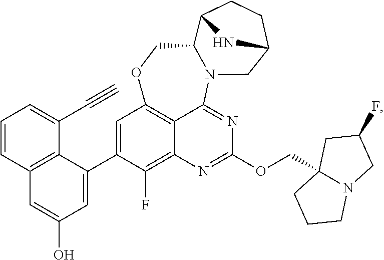 [SMILES: C#Cc1cccc2cc(O)cc(-c3cc4c5c(nc(OC[C@@]67CCCN6C[C@H](F)C7)nc5c3F)N3C[C@H]5CC[C@H](N5)[C@H]3CO4)c12]

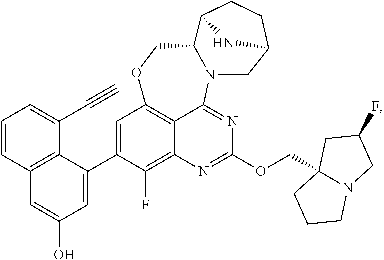 [SMILES: C#Cc1cccc2cc(O)cc(-c3cc4c5c(nc(OC[C@@]67CCCN6C[C@H](F)C7)nc5c3F)N3C[C@@H]5CC[C@@H](N5)[C@H]3CO4)c12]

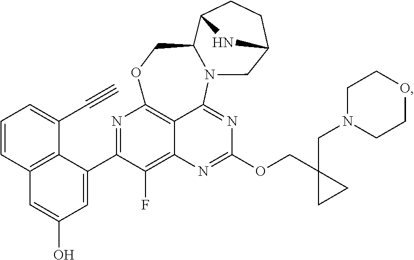 [SMILES: C#Cc1cccc2cc(O)cc(-c3nc4c5c(nc(OCC6(CN7CCOCC7)CC6)nc5c3F)N3C[C@H]5CC[C@H](N5)[C@@H]3CO4)c12]

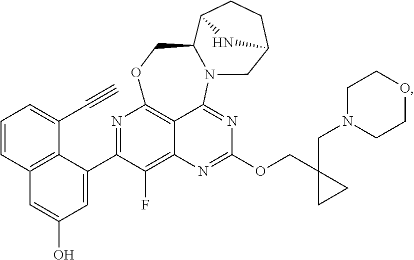 [SMILES: C#Cc1cccc2cc(O)cc(-c3nc4c5c(nc(OCC6(CN7CCOCC7)CC6)nc5c3F)N3C[C@@H]5CC[C@@H](N5)[C@@H]3CO4)c12]

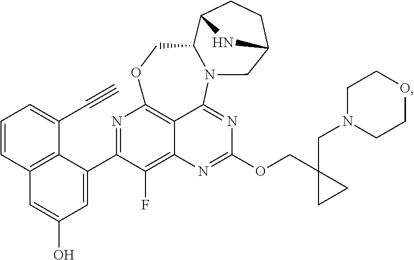 [SMILES: C#Cc1cccc2cc(O)cc(-c3nc4c5c(nc(OCC6(CN7CCOCC7)CC6)nc5c3F)N3C[C@H]5CC[C@H](N5)[C@H]3CO4)c12]

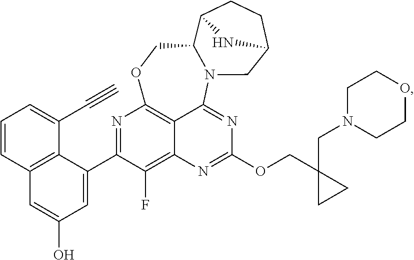 [SMILES: C#Cc1cccc2cc(O)cc(-c3nc4c5c(nc(OCC6(CN7CCOCC7)CC6)nc5c3F)N3C[C@@H]5CC[C@@H](N5)[C@H]3CO4)c12]

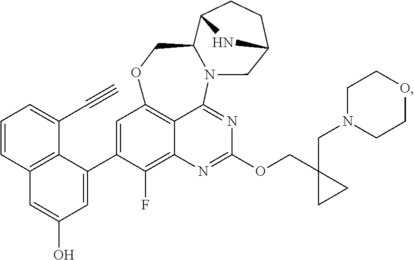 [SMILES: C#Cc1cccc2cc(O)cc(-c3cc4c5c(nc(OCC6(CN7CCOCC7)CC6)nc5c3F)N3C[C@H]5CC[C@H](N5)[C@@H]3CO4)c12]

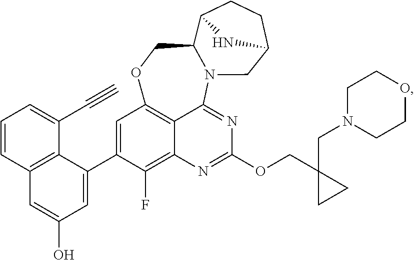 [SMILES: C#Cc1cccc2cc(O)cc(-c3cc4c5c(nc(OCC6(CN7CCOCC7)CC6)nc5c3F)N3C[C@@H]5CC[C@@H](N5)[C@@H]3CO4)c12]

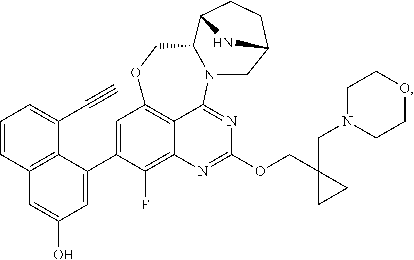 [SMILES: C#Cc1cccc2cc(O)cc(-c3cc4c5c(nc(OCC6(CN7CCOCC7)CC6)nc5c3F)N3C[C@H]5CC[C@H](N5)[C@H]3CO4)c12]

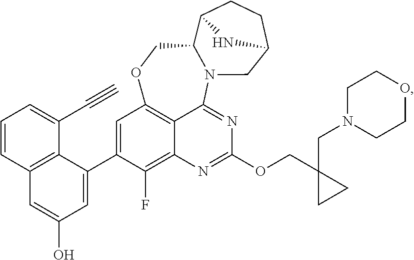 [SMILES: C#Cc1cccc2cc(O)cc(-c3cc4c5c(nc(OCC6(CN7CCOCC7)CC6)nc5c3F)N3C[C@@H]5CC[C@@H](N5)[C@H]3CO4)c12]

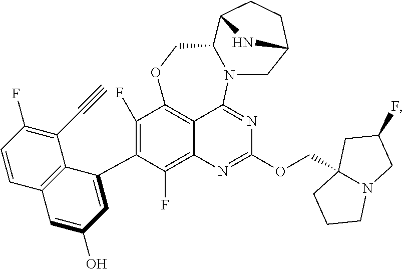

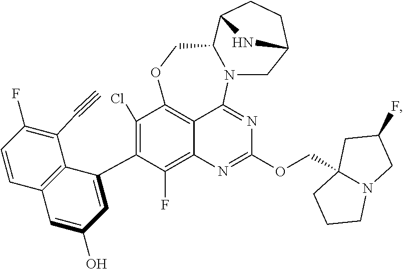

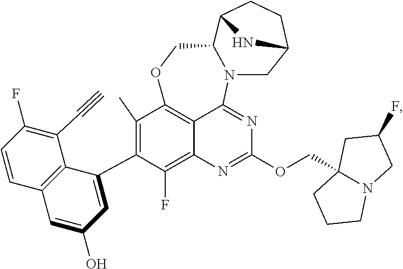

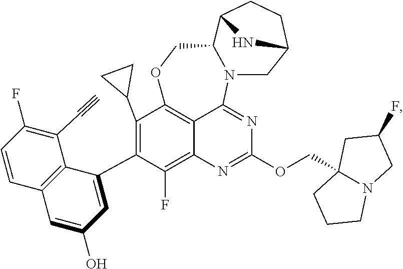

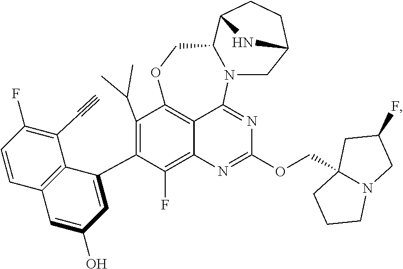

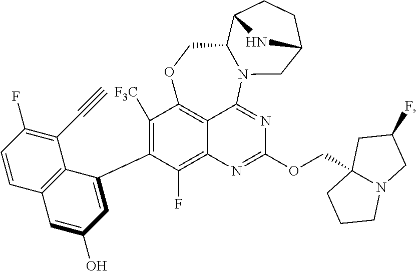

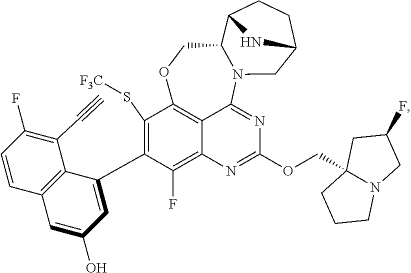

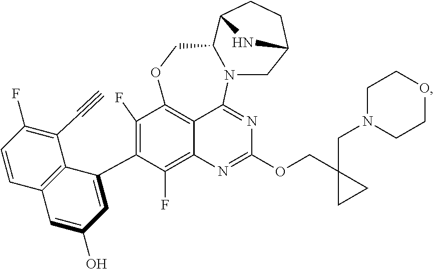

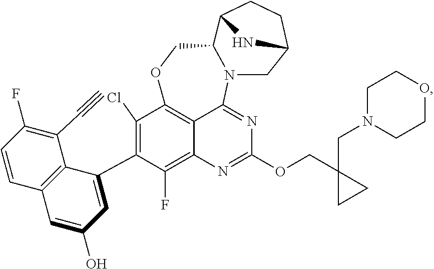

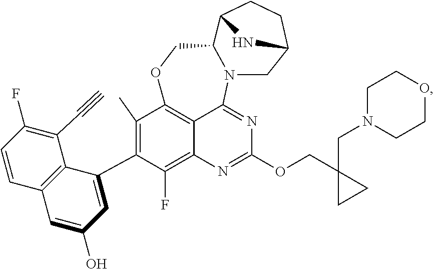

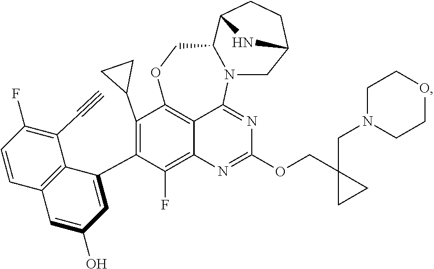

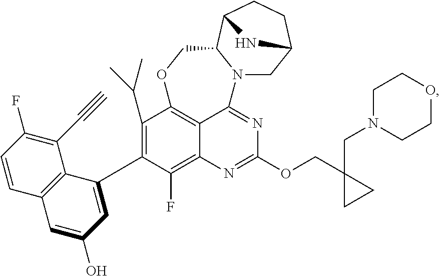

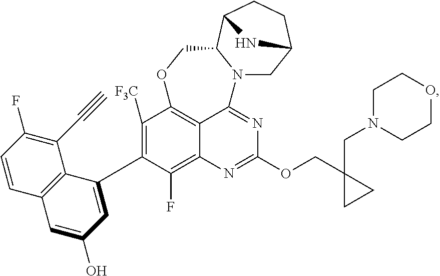

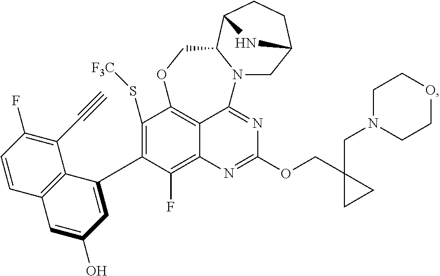

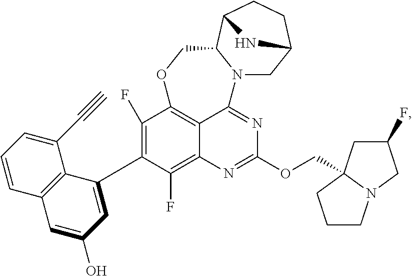

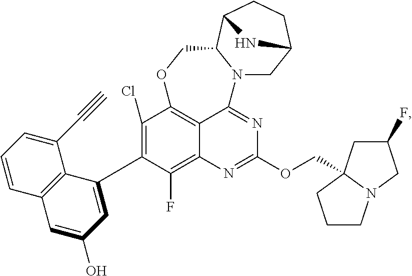

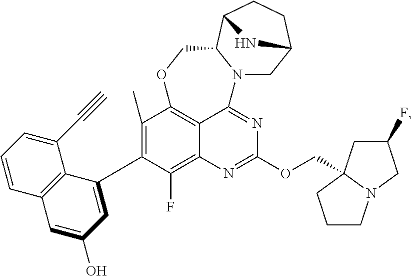

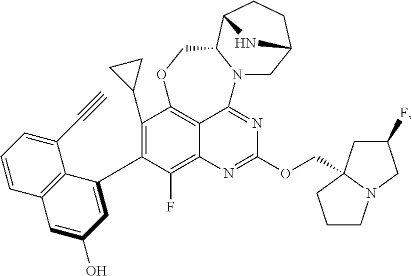

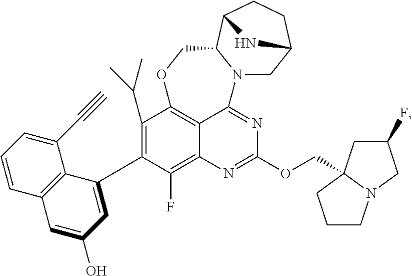

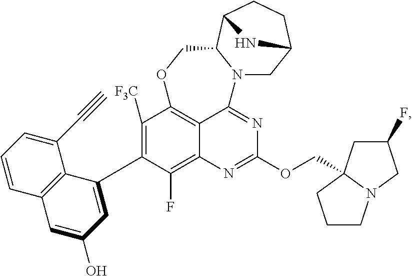

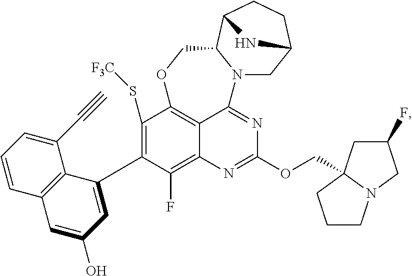

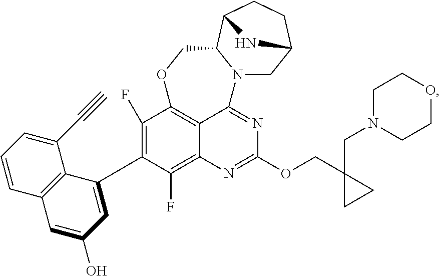

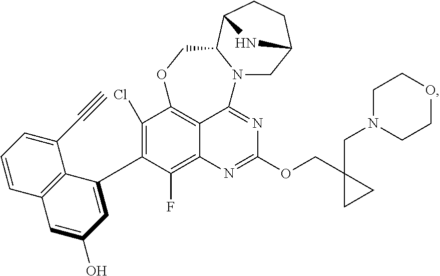

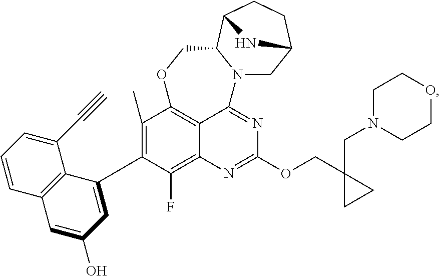

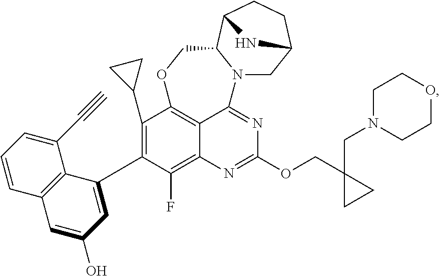

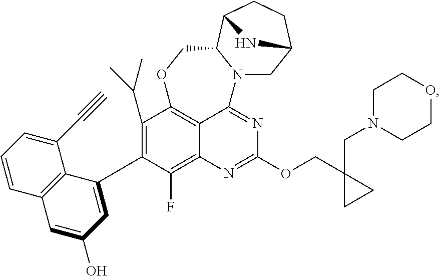

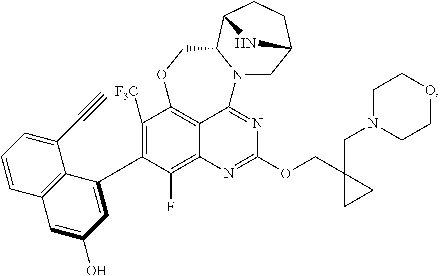

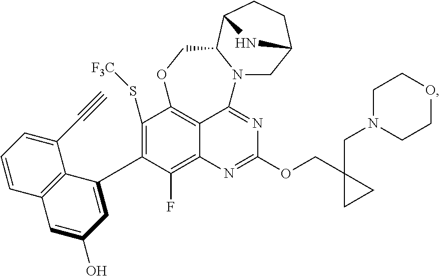

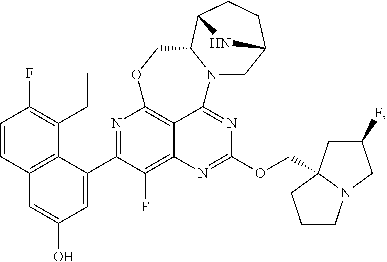 [SMILES: CCc1c(F)ccc2cc(O)cc(-c3nc4c5c(nc(OC[C@@]67CCCN6C[C@H](F)C7)nc5c3F)N3C[C@H]5CC[C@H](N5)[C@H]3CO4)c12]

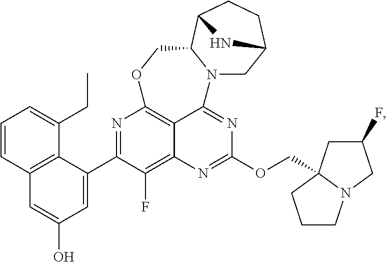 [SMILES: CCc1cccc2cc(O)cc(-c3nc4c5c(nc(OC[C@@]67CCCN6C[C@H](F)C7)nc5c3F)N3C[C@H]5CC[C@H](N5)[C@H]3CO4)c12]

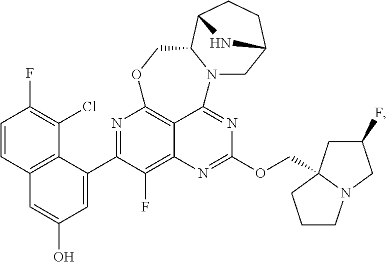 [SMILES: Oc1cc(-c2nc3c4c(nc(OC[C@@]56CCCN5C[C@H](F)C6)nc4c2F)N2C[C@H]4CC[C@H](N4)[C@H]2CO3)c2c(Cl)c(F)ccc2c1]

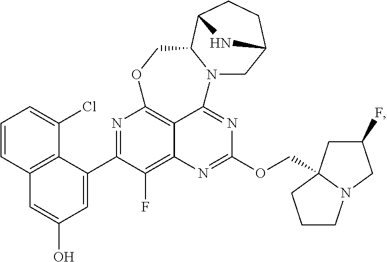 [SMILES: Oc1cc(-c2nc3c4c(nc(OC[C@@]56CCCN5C[C@H](F)C6)nc4c2F)N2C[C@H]4CC[C@H](N4)[C@H]2CO3)c2c(Cl)cccc2c1]

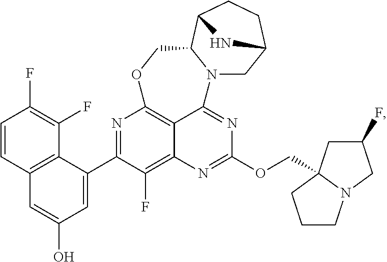 [SMILES: Oc1cc(-c2nc3c4c(nc(OC[C@@]56CCCN5C[C@H](F)C6)nc4c2F)N2C[C@H]4CC[C@H](N4)[C@H]2CO3)c2c(F)c(F)ccc2c1]

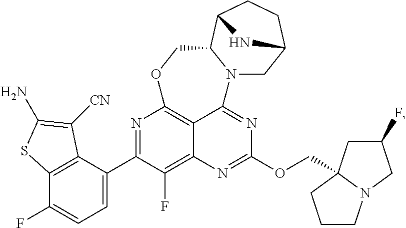 [SMILES: N#Cc1c(N)sc2c(F)ccc(-c3nc4c5c(nc(OC[C@@]67CCCN6C[C@H](F)C7)nc5c3F)N3C[C@H]5CC[C@H](N5)[C@H]3CO4)c12]

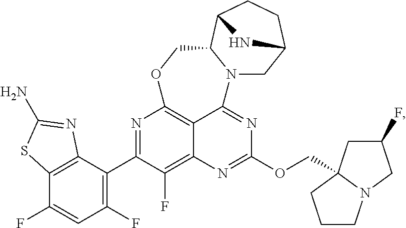 [SMILES: Nc1nc2c(-c3nc4c5c(nc(OC[C@@]67CCCN6C[C@H](F)C7)nc5c3F)N3C[C@H]5CC[C@H](N5)[C@H]3CO4)c(F)cc(F)c2s1]

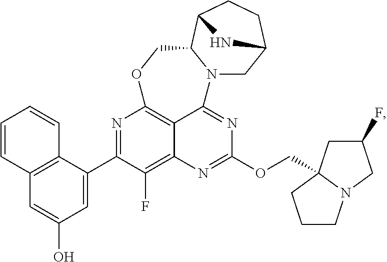 [SMILES: Oc1cc(-c2nc3c4c(nc(OC[C@@]56CCCN5C[C@H](F)C6)nc4c2F)N2C[C@H]4CC[C@H](N4)[C@H]2CO3)c2ccccc2c1]

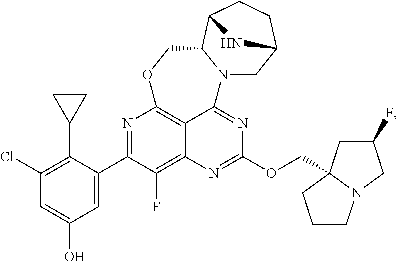 [SMILES: Oc1cc(Cl)c(C2CC2)c(-c2nc3c4c(nc(OC[C@@]56CCCN5C[C@H](F)C6)nc4c2F)N2C[C@H]4CC[C@H](N4)[C@H]2CO3)c1]

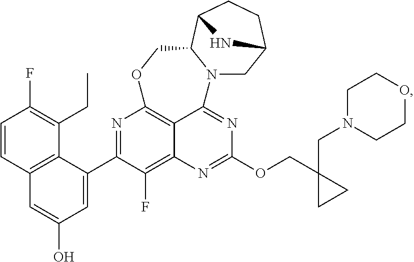 [SMILES: CCc1c(F)ccc2cc(O)cc(-c3nc4c5c(nc(OCC6(CN7CCOCC7)CC6)nc5c3F)N3C[C@H]5CC[C@H](N5)[C@H]3CO4)c12]

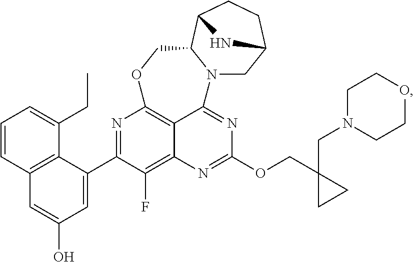 [SMILES: CCc1cccc2cc(O)cc(-c3nc4c5c(nc(OCC6(CN7CCOCC7)CC6)nc5c3F)N3C[C@H]5CC[C@H](N5)[C@H]3CO4)c12]

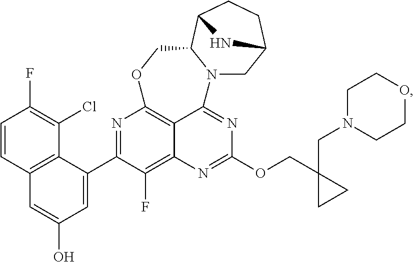 [SMILES: Oc1cc(-c2nc3c4c(nc(OCC5(CN6CCOCC6)CC5)nc4c2F)N2C[C@H]4CC[C@H](N4)[C@H]2CO3)c2c(Cl)c(F)ccc2c1]

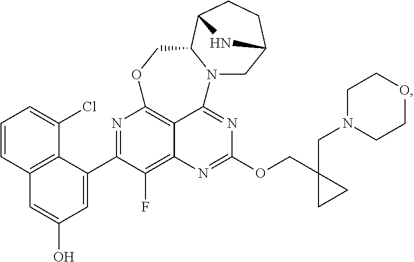 [SMILES: Oc1cc(-c2nc3c4c(nc(OCC5(CN6CCOCC6)CC5)nc4c2F)N2C[C@H]4CC[C@H](N4)[C@H]2CO3)c2c(Cl)cccc2c1]

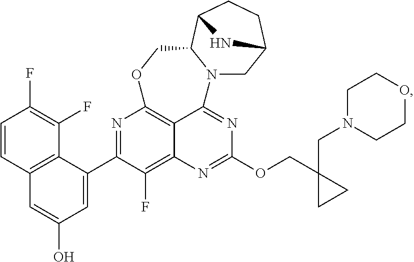 [SMILES: Oc1cc(-c2nc3c4c(nc(OCC5(CN6CCOCC6)CC5)nc4c2F)N2C[C@H]4CC[C@H](N4)[C@H]2CO3)c2c(F)c(F)ccc2c1]

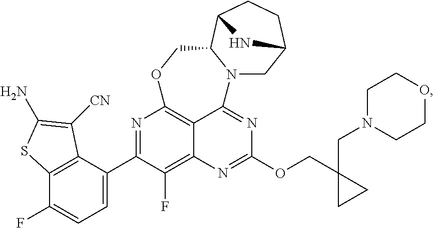 [SMILES: N#Cc1c(N)sc2c(F)ccc(-c3nc4c5c(nc(OCC6(CN7CCOCC7)CC6)nc5c3F)N3C[C@H]5CC[C@H](N5)[C@H]3CO4)c12]

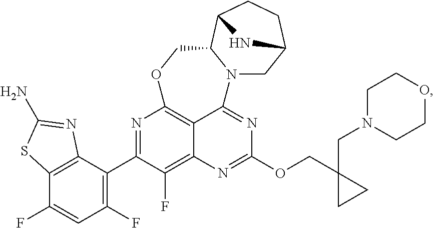 [SMILES: Nc1nc2c(-c3nc4c5c(nc(OCC6(CN7CCOCC7)CC6)nc5c3F)N3C[C@H]5CC[C@H](N5)[C@H]3CO4)c(F)cc(F)c2s1]

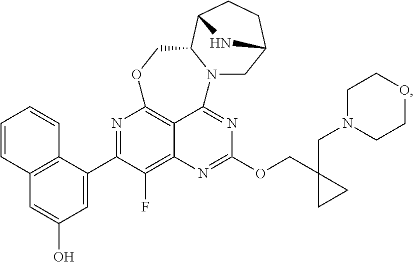 [SMILES: Oc1cc(-c2nc3c4c(nc(OCC5(CN6CCOCC6)CC5)nc4c2F)N2C[C@H]4CC[C@H](N4)[C@H]2CO3)c2ccccc2c1]

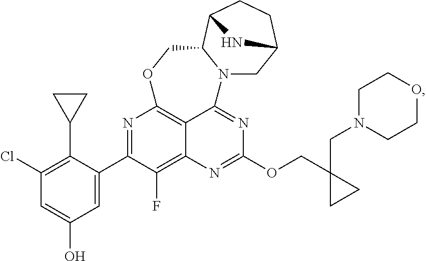 [SMILES: Oc1cc(Cl)c(C2CC2)c(-c2nc3c4c(nc(OCC5(CN6CCOCC6)CC5)nc4c2F)N2C[C@H]4CC[C@H](N4)[C@H]2CO3)c1]

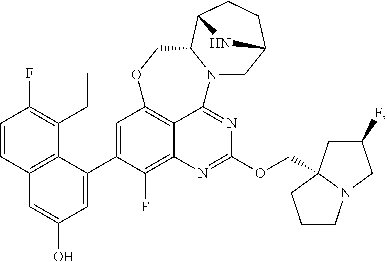 [SMILES: CCc1c(F)ccc2cc(O)cc(-c3cc4c5c(nc(OC[C@@]67CCCN6C[C@H](F)C7)nc5c3F)N3C[C@H]5CC[C@H](N5)[C@H]3CO4)c12]

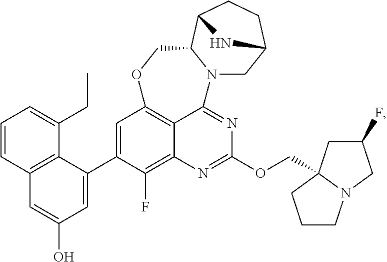 [SMILES: CCc1cccc2cc(O)cc(-c3cc4c5c(nc(OC[C@@]67CCCN6C[C@H](F)C7)nc5c3F)N3C[C@H]5CC[C@H](N5)[C@H]3CO4)c12]

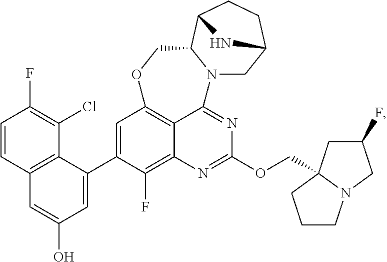 [SMILES: Oc1cc(-c2cc3c4c(nc(OC[C@@]56CCCN5C[C@H](F)C6)nc4c2F)N2C[C@H]4CC[C@H](N4)[C@H]2CO3)c2c(Cl)c(F)ccc2c1]

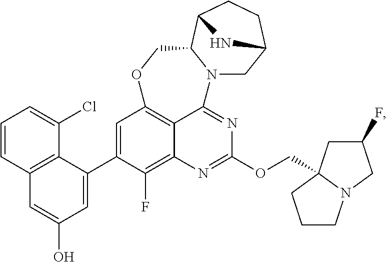 [SMILES: Oc1cc(-c2cc3c4c(nc(OC[C@@]56CCCN5C[C@H](F)C6)nc4c2F)N2C[C@H]4CC[C@H](N4)[C@H]2CO3)c2c(Cl)cccc2c1]

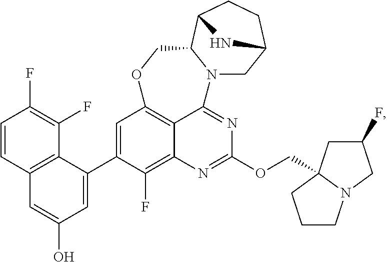 [SMILES: Oc1cc(-c2cc3c4c(nc(OC[C@@]56CCCN5C[C@H](F)C6)nc4c2F)N2C[C@H]4CC[C@H](N4)[C@H]2CO3)c2c(F)c(F)ccc2c1]

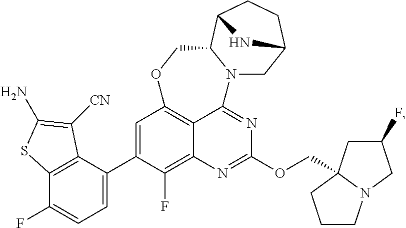 [SMILES: N#Cc1c(N)sc2c(F)ccc(-c3cc4c5c(nc(OC[C@@]67CCCN6C[C@H](F)C7)nc5c3F)N3C[C@H]5CC[C@H](N5)[C@H]3CO4)c12]

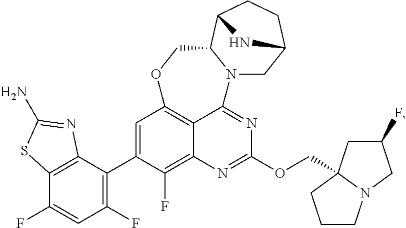 [SMILES: Nc1nc2c(-c3cc4c5c(nc(OC[C@@]67CCCN6C[C@H](F)C7)nc5c3F)N3C[C@H]5CC[C@H](N5)[C@H]3CO4)c(F)cc(F)c2s1]

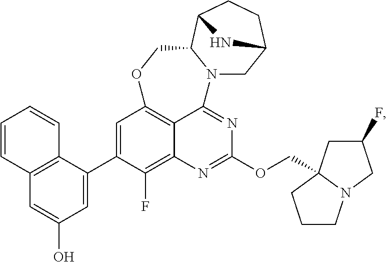 [SMILES: Oc1cc(-c2cc3c4c(nc(OC[C@@]56CCCN5C[C@H](F)C6)nc4c2F)N2C[C@H]4CC[C@H](N4)[C@H]2CO3)c2ccccc2c1]

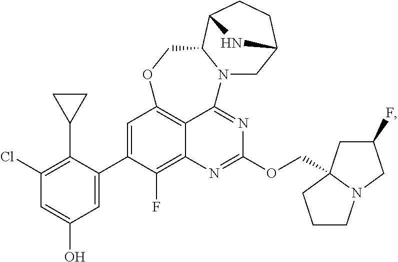 [SMILES: Oc1cc(Cl)c(C2CC2)c(-c2cc3c4c(nc(OC[C@@]56CCCN5C[C@H](F)C6)nc4c2F)N2C[C@H]4CC[C@H](N4)[C@H]2CO3)c1]

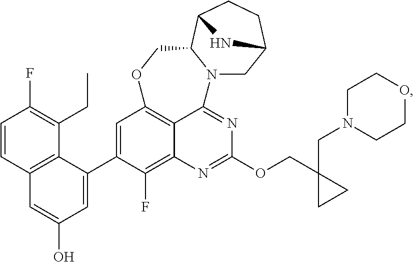 [SMILES: CCc1c(F)ccc2cc(O)cc(-c3cc4c5c(nc(OCC6(CN7CCOCC7)CC6)nc5c3F)N3C[C@H]5CC[C@H](N5)[C@H]3CO4)c12]

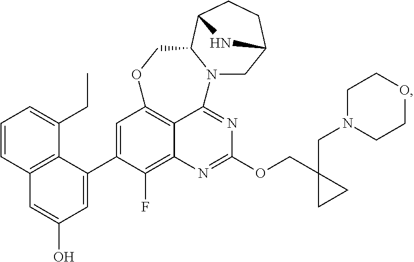 [SMILES: CCc1cccc2cc(O)cc(-c3cc4c5c(nc(OCC6(CN7CCOCC7)CC6)nc5c3F)N3C[C@H]5CC[C@H](N5)[C@H]3CO4)c12]

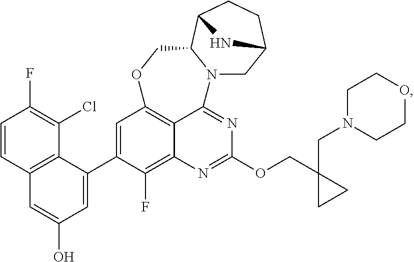 [SMILES: Oc1cc(-c2cc3c4c(nc(OCC5(CN6CCOCC6)CC5)nc4c2F)N2C[C@H]4CC[C@H](N4)[C@H]2CO3)c2c(Cl)c(F)ccc2c1]

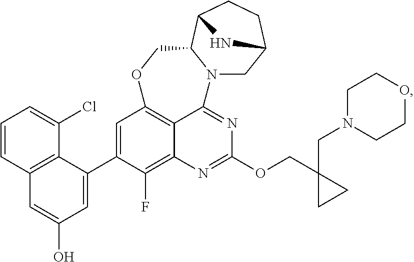 [SMILES: Oc1cc(-c2cc3c4c(nc(OCC5(CN6CCOCC6)CC5)nc4c2F)N2C[C@H]4CC[C@H](N4)[C@H]2CO3)c2c(Cl)cccc2c1]

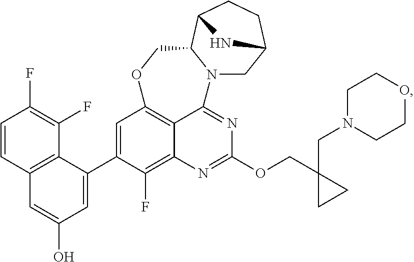 [SMILES: Oc1cc(-c2cc3c4c(nc(OCC5(CN6CCOCC6)CC5)nc4c2F)N2C[C@H]4CC[C@H](N4)[C@H]2CO3)c2c(F)c(F)ccc2c1]

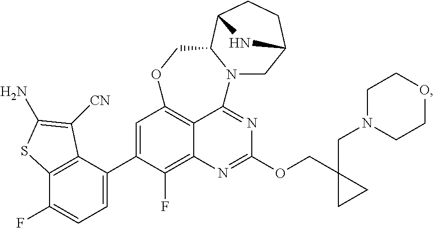 [SMILES: N#Cc1c(N)sc2c(F)ccc(-c3cc4c5c(nc(OCC6(CN7CCOCC7)CC6)nc5c3F)N3C[C@H]5CC[C@H](N5)[C@H]3CO4)c12]

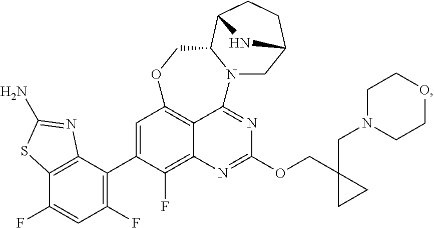 [SMILES: Nc1nc2c(-c3cc4c5c(nc(OCC6(CN7CCOCC7)CC6)nc5c3F)N3C[C@H]5CC[C@H](N5)[C@H]3CO4)c(F)cc(F)c2s1]

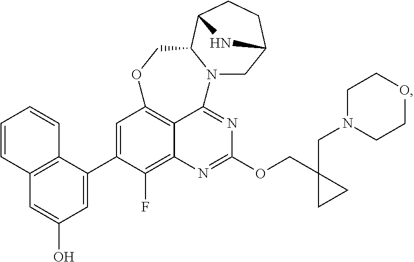 [SMILES: Oc1cc(-c2cc3c4c(nc(OCC5(CN6CCOCC6)CC5)nc4c2F)N2C[C@H]4CC[C@H](N4)[C@H]2CO3)c2ccccc2c1]

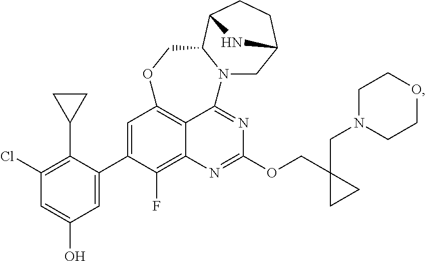 [SMILES: Oc1cc(Cl)c(C2CC2)c(-c2cc3c4c(nc(OCC5(CN6CCOCC6)CC5)nc4c2F)N2C[C@H]4CC[C@H](N4)[C@H]2CO3)c1]

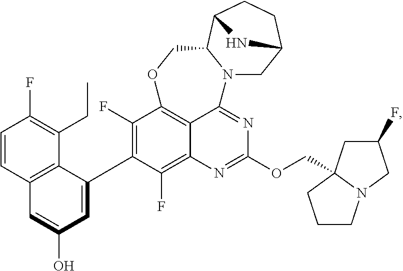

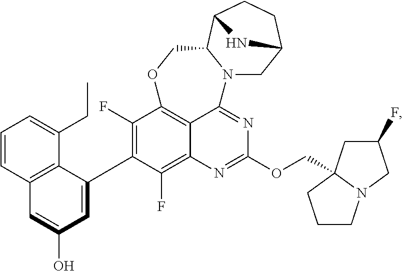

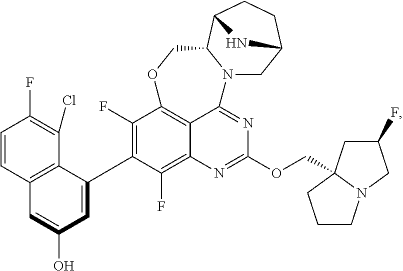

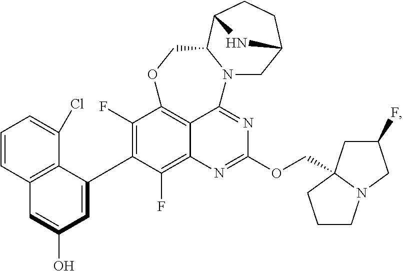

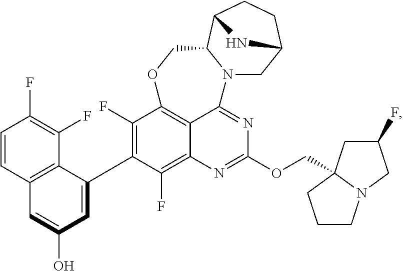

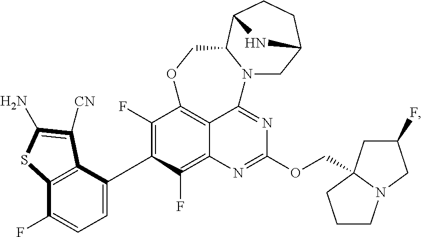

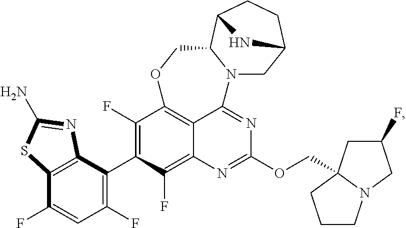

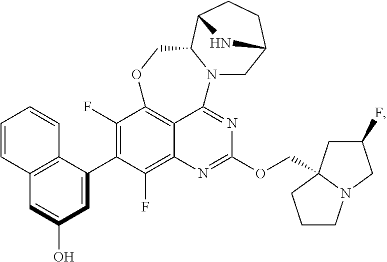

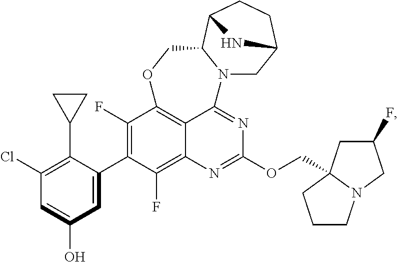

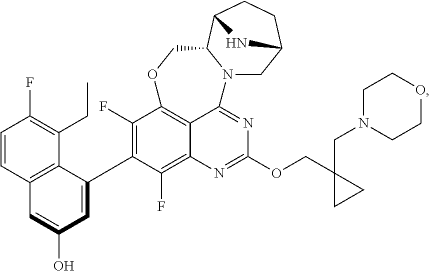

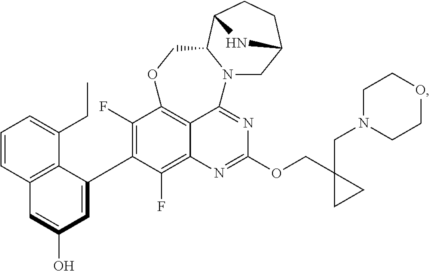

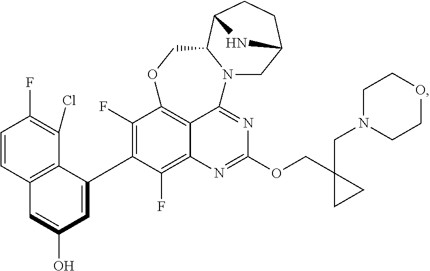

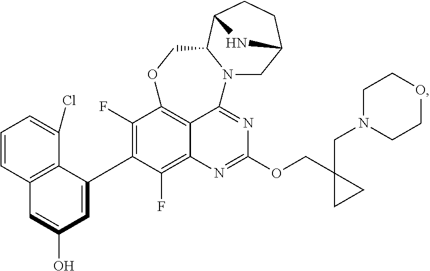

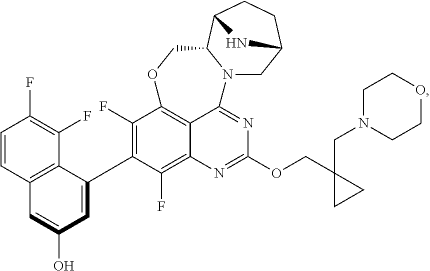

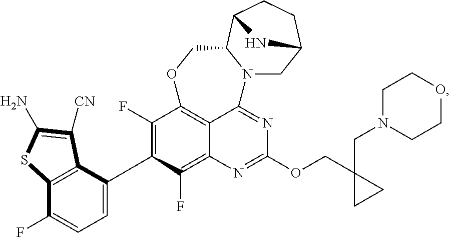

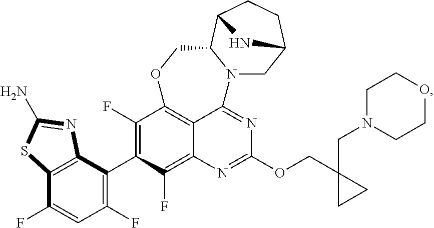

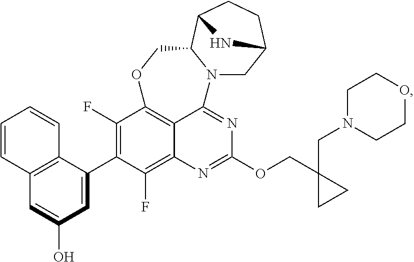

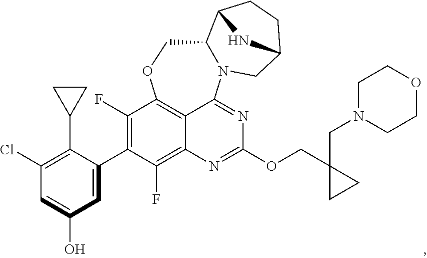

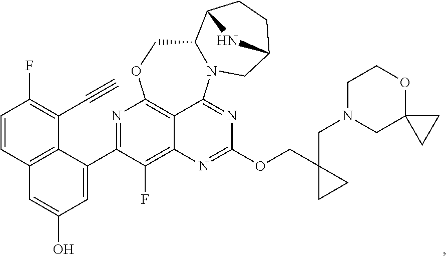 [SMILES: C#Cc1c(F)ccc2cc(O)cc(-c3nc4c5c(nc(OCC6(CN7CCOC8(CC8)C7)CC6)nc5c3F)N3C[C@H]5CC[C@H](N5)[C@H]3CO4)c12]

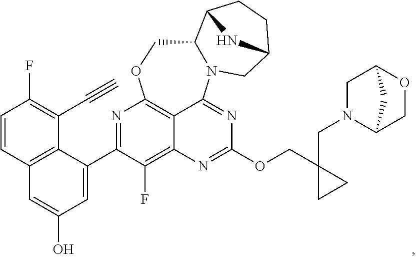 [SMILES: C#Cc1c(F)ccc2cc(O)cc(-c3nc4c5c(nc(OCC6(CN7C[C@@H]8C[C@H]7CO8)CC6)nc5c3F)N3C[C@H]5CC[C@H](N5)[C@H]3CO4)c12]

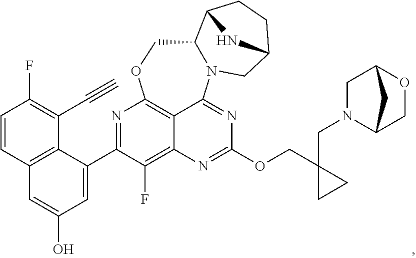 [SMILES: C#Cc1c(F)ccc2cc(O)cc(-c3nc4c5c(nc(OCC6(CN7C[C@H]8C[C@@H]7CO8)CC6)nc5c3F)N3C[C@H]5CC[C@H](N5)[C@H]3CO4)c12]

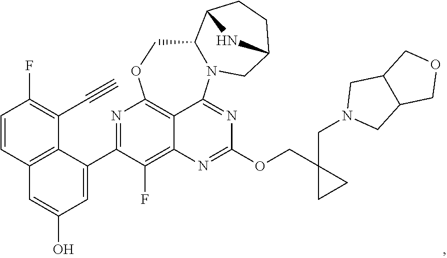 [SMILES: C#Cc1c(F)ccc2cc(O)cc(-c3nc4c5c(nc(OCC6(CN7CC8COCC8C7)CC6)nc5c3F)N3C[C@H]5CC[C@H](N5)[C@H]3CO4)c12]

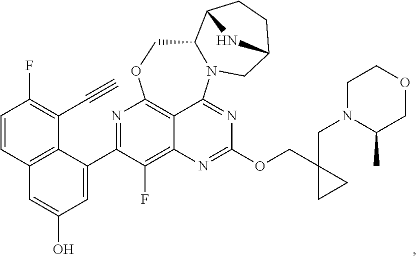 [SMILES: C#Cc1c(F)ccc2cc(O)cc(-c3nc4c5c(nc(OCC6(CN7CCOC[C@H]7C)CC6)nc5c3F)N3C[C@H]5CC[C@H](N5)[C@H]3CO4)c12]

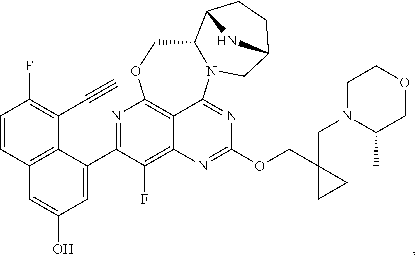 [SMILES: C#Cc1c(F)ccc2cc(O)cc(-c3nc4c5c(nc(OCC6(CN7CCOC[C@@H]7C)CC6)nc5c3F)N3C[C@H]5CC[C@H](N5)[C@H]3CO4)c12]

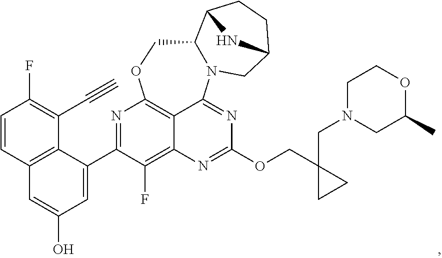 [SMILES: C#Cc1c(F)ccc2cc(O)cc(-c3nc4c5c(nc(OCC6(CN7CCO[C@@H](C)C7)CC6)nc5c3F)N3C[C@H]5CC[C@H](N5)[C@H]3CO4)c12]

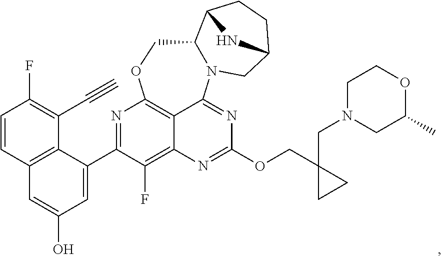 [SMILES: C#Cc1c(F)ccc2cc(O)cc(-c3nc4c5c(nc(OCC6(CN7CCO[C@H](C)C7)CC6)nc5c3F)N3C[C@H]5CC[C@H](N5)[C@H]3CO4)c12]

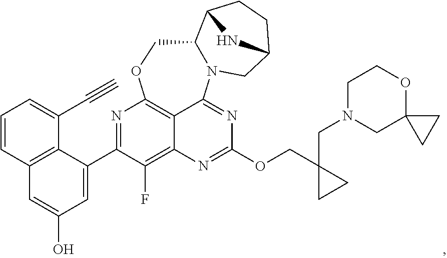 [SMILES: C#Cc1cccc2cc(O)cc(-c3nc4c5c(nc(OCC6(CN7CCOC8(CC8)C7)CC6)nc5c3F)N3C[C@H]5CC[C@H](N5)[C@H]3CO4)c12]

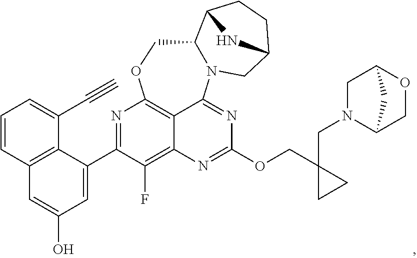 [SMILES: C#Cc1cccc2cc(O)cc(-c3nc4c5c(nc(OCC6(CN7C[C@@H]8C[C@H]7CO8)CC6)nc5c3F)N3C[C@H]5CC[C@H](N5)[C@H]3CO4)c12]

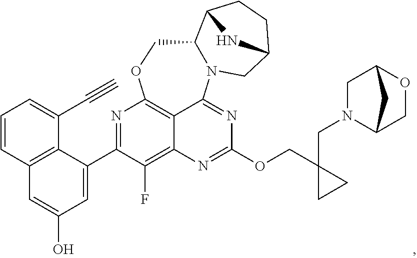 [SMILES: C#Cc1cccc2cc(O)cc(-c3nc4c5c(nc(OCC6(CN7C[C@H]8C[C@@H]7CO8)CC6)nc5c3F)N3C[C@H]5CC[C@H](N5)[C@H]3CO4)c12]

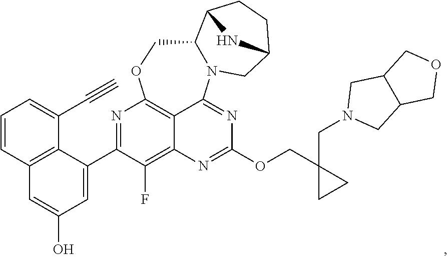 [SMILES: C#Cc1cccc2cc(O)cc(-c3nc4c5c(nc(OCC6(CN7CC8COCC8C7)CC6)nc5c3F)N3C[C@H]5CC[C@H](N5)[C@H]3CO4)c12]

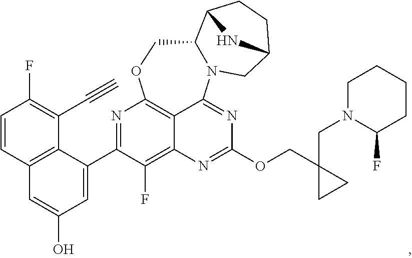 [SMILES: C#Cc1c(F)ccc2cc(O)cc(-c3nc4c5c(nc(OCC6(CN7CCCC[C@H]7F)CC6)nc5c3F)N3C[C@H]5CC[C@H](N5)[C@H]3CO4)c12]

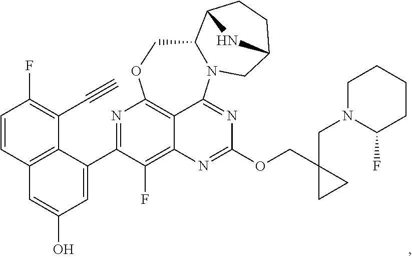 [SMILES: C#Cc1c(F)ccc2cc(O)cc(-c3nc4c5c(nc(OCC6(CN7CCCC[C@@H]7F)CC6)nc5c3F)N3C[C@H]5CC[C@H](N5)[C@H]3CO4)c12]

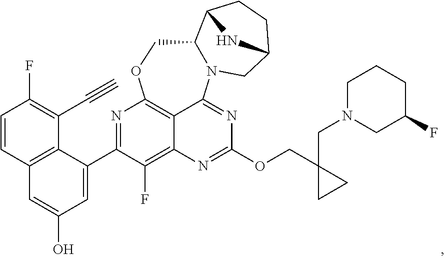 [SMILES: C#Cc1c(F)ccc2cc(O)cc(-c3nc4c5c(nc(OCC6(CN7CCC[C@@H](F)C7)CC6)nc5c3F)N3C[C@H]5CC[C@H](N5)[C@H]3CO4)c12]

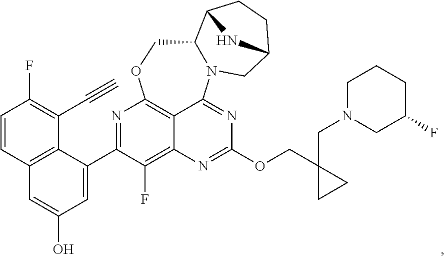 [SMILES: C#Cc1c(F)ccc2cc(O)cc(-c3nc4c5c(nc(OCC6(CN7CCC[C@H](F)C7)CC6)nc5c3F)N3C[C@H]5CC[C@H](N5)[C@H]3CO4)c12]

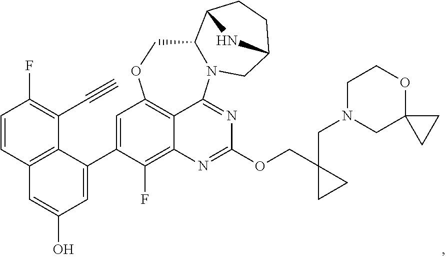 [SMILES: C#Cc1c(F)ccc2cc(O)cc(-c3cc4c5c(nc(OCC6(CN7CCOC8(CC8)C7)CC6)nc5c3F)N3C[C@H]5CC[C@H](N5)[C@H]3CO4)c12]

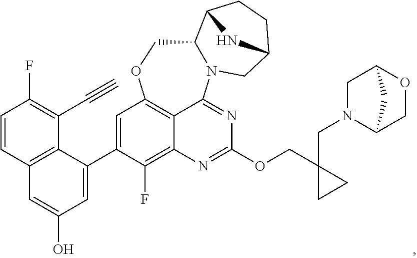 [SMILES: C#Cc1c(F)ccc2cc(O)cc(-c3cc4c5c(nc(OCC6(CN7C[C@@H]8C[C@H]7CO8)CC6)nc5c3F)N3C[C@H]5CC[C@H](N5)[C@H]3CO4)c12]

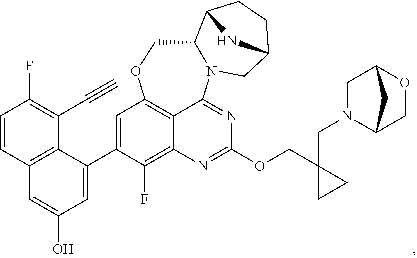 [SMILES: C#Cc1c(F)ccc2cc(O)cc(-c3cc4c5c(nc(OCC6(CN7C[C@H]8C[C@@H]7CO8)CC6)nc5c3F)N3C[C@H]5CC[C@H](N5)[C@H]3CO4)c12]

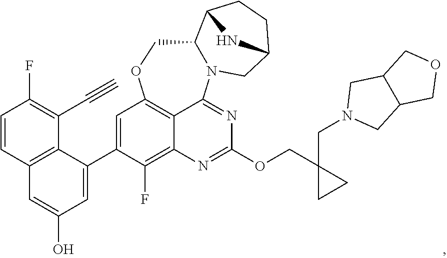 [SMILES: C#Cc1c(F)ccc2cc(O)cc(-c3cc4c5c(nc(OCC6(CN7CC8COCC8C7)CC6)nc5c3F)N3C[C@H]5CC[C@H](N5)[C@H]3CO4)c12]

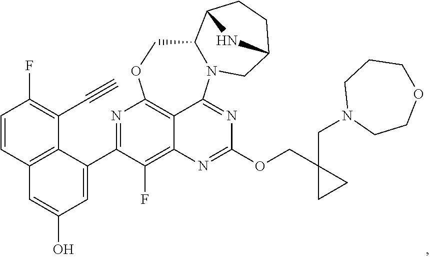 [SMILES: C#Cc1c(F)ccc2cc(O)cc(-c3nc4c5c(nc(OCC6(CN7CCCOCC7)CC6)nc5c3F)N3C[C@H]5CC[C@H](N5)[C@H]3CO4)c12]

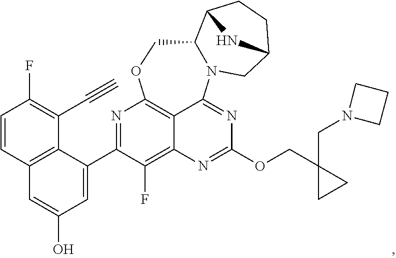 [SMILES: C#Cc1c(F)ccc2cc(O)cc(-c3nc4c5c(nc(OCC6(CN7CCC7)CC6)nc5c3F)N3C[C@H]5CC[C@H](N5)[C@H]3CO4)c12]

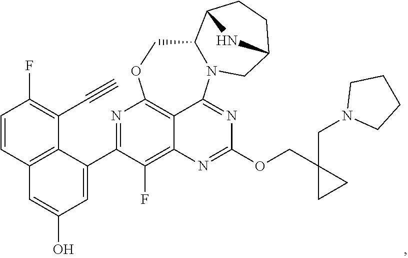 [SMILES: C#Cc1c(F)ccc2cc(O)cc(-c3nc4c5c(nc(OCC6(CN7CCCC7)CC6)nc5c3F)N3C[C@H]5CC[C@H](N5)[C@H]3CO4)c12]

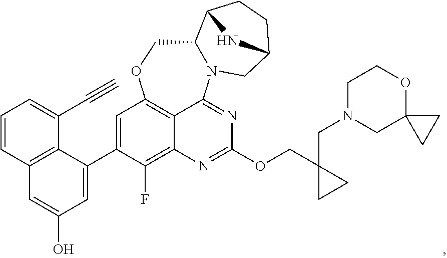 [SMILES: C#Cc1cccc2cc(O)cc(-c3cc4c5c(nc(OCC6(CN7CCOC8(CC8)C7)CC6)nc5c3F)N3C[C@H]5CC[C@H](N5)[C@H]3CO4)c12]

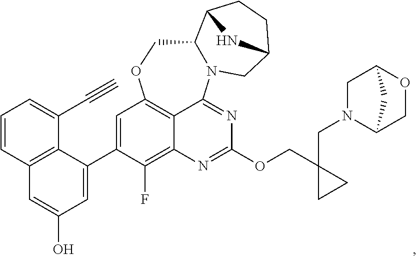 [SMILES: C#Cc1cccc2cc(O)cc(-c3cc4c5c(nc(OCC6(CN7C[C@@H]8C[C@H]7CO8)CC6)nc5c3F)N3C[C@H]5CC[C@H](N5)[C@H]3CO4)c12]

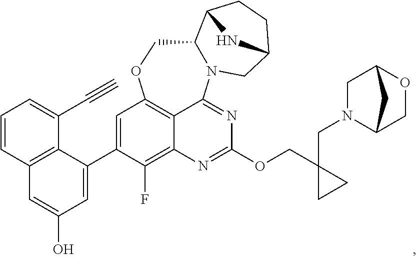 [SMILES: C#Cc1cccc2cc(O)cc(-c3cc4c5c(nc(OCC6(CN7C[C@H]8C[C@@H]7CO8)CC6)nc5c3F)N3C[C@H]5CC[C@H](N5)[C@H]3CO4)c12]

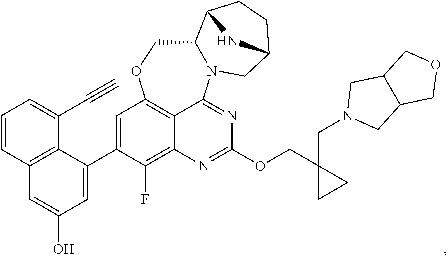 [SMILES: C#Cc1cccc2cc(O)cc(-c3cc4c5c(nc(OCC6(CN7CC8COCC8C7)CC6)nc5c3F)N3C[C@H]5CC[C@H](N5)[C@H]3CO4)c12]

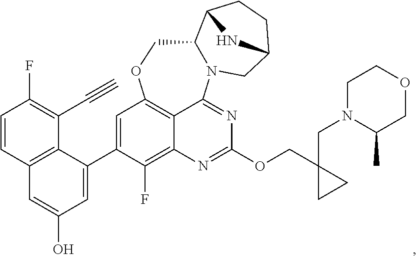 [SMILES: C#Cc1c(F)ccc2cc(O)cc(-c3cc4c5c(nc(OCC6(CN7CCOC[C@H]7C)CC6)nc5c3F)N3C[C@H]5CC[C@H](N5)[C@H]3CO4)c12]

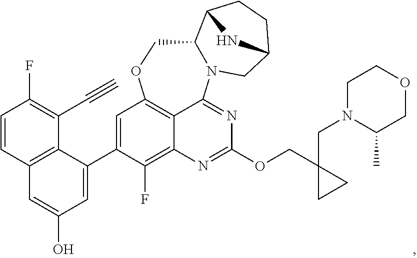 [SMILES: C#Cc1c(F)ccc2cc(O)cc(-c3cc4c5c(nc(OCC6(CN7CCOC[C@@H]7C)CC6)nc5c3F)N3C[C@H]5CC[C@H](N5)[C@H]3CO4)c12]

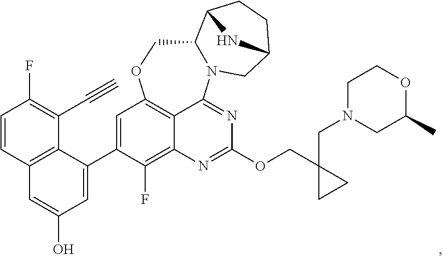 [SMILES: C#Cc1c(F)ccc2cc(O)cc(-c3cc4c5c(nc(OCC6(CN7CCO[C@@H](C)C7)CC6)nc5c3F)N3C[C@H]5CC[C@H](N5)[C@H]3CO4)c12]

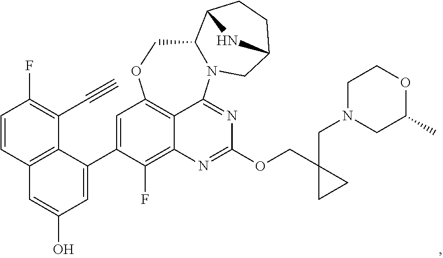 [SMILES: C#Cc1c(F)ccc2cc(O)cc(-c3cc4c5c(nc(OCC6(CN7CCO[C@H](C)C7)CC6)nc5c3F)N3C[C@H]5CC[C@H](N5)[C@H]3CO4)c12]

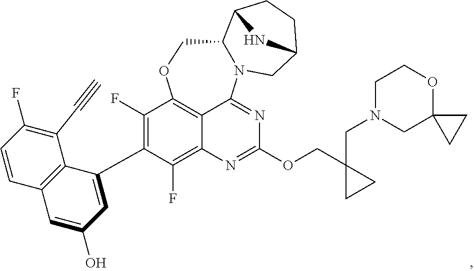

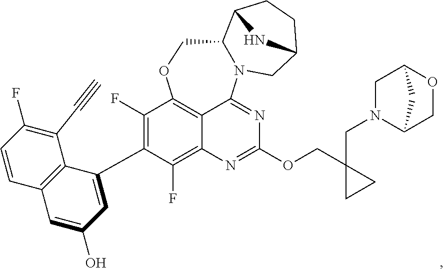

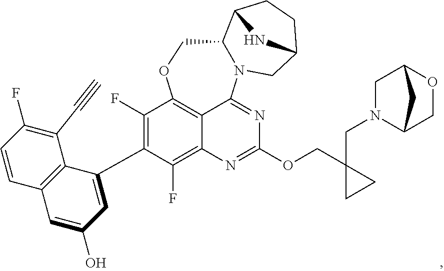

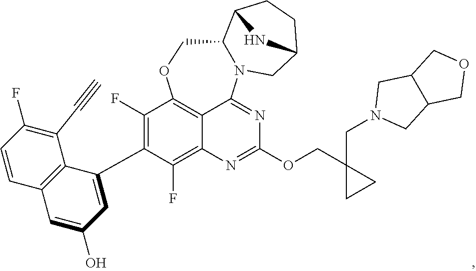

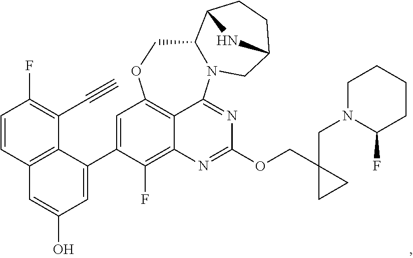 [SMILES: C#Cc1c(F)ccc2cc(O)cc(-c3cc4c5c(nc(OCC6(CN7CCCC[C@H]7F)CC6)nc5c3F)N3C[C@H]5CC[C@H](N5)[C@H]3CO4)c12]

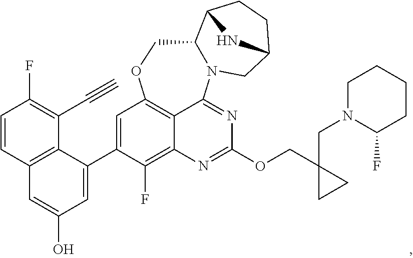 [SMILES: C#Cc1c(F)ccc2cc(O)cc(-c3cc4c5c(nc(OCC6(CN7CCCC[C@@H]7F)CC6)nc5c3F)N3C[C@H]5CC[C@H](N5)[C@H]3CO4)c12]

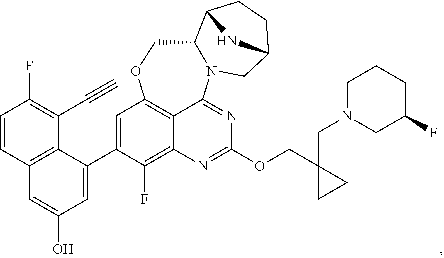 [SMILES: C#Cc1c(F)ccc2cc(O)cc(-c3cc4c5c(nc(OCC6(CN7CCC[C@@H](F)C7)CC6)nc5c3F)N3C[C@H]5CC[C@H](N5)[C@H]3CO4)c12]

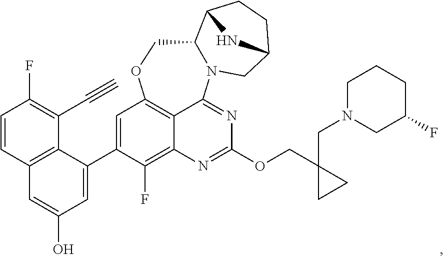 [SMILES: C#Cc1c(F)ccc2cc(O)cc(-c3cc4c5c(nc(OCC6(CN7CCC[C@H](F)C7)CC6)nc5c3F)N3C[C@H]5CC[C@H](N5)[C@H]3CO4)c12]

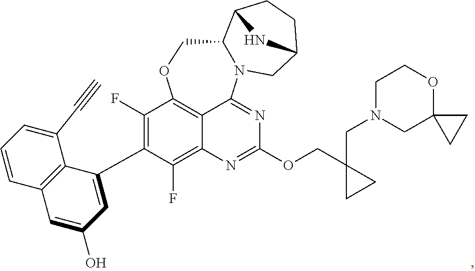

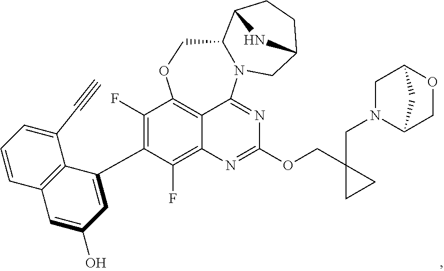

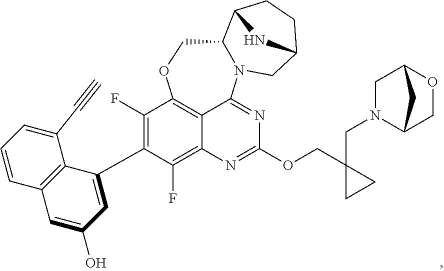

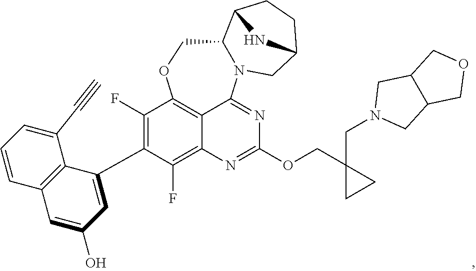

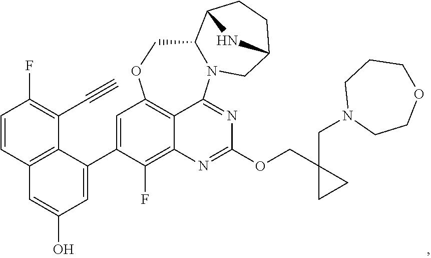 [SMILES: C#Cc1c(F)ccc2cc(O)cc(-c3cc4c5c(nc(OCC6(CN7CCCOCC7)CC6)nc5c3F)N3C[C@H]5CC[C@H](N5)[C@H]3CO4)c12]

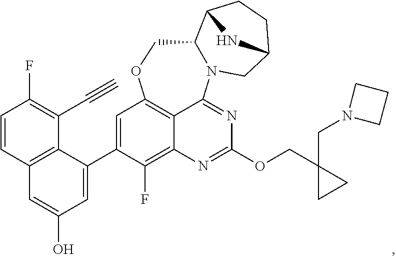 [SMILES: C#Cc1c(F)ccc2cc(O)cc(-c3cc4c5c(nc(OCC6(CN7CCC7)CC6)nc5c3F)N3C[C@H]5CC[C@H](N5)[C@H]3CO4)c12]

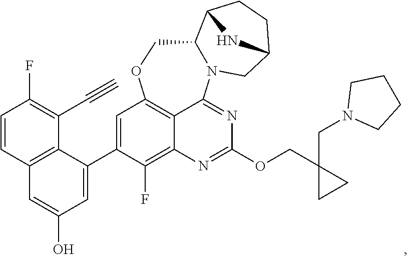 [SMILES: C#Cc1c(F)ccc2cc(O)cc(-c3cc4c5c(nc(OCC6(CN7CCCC7)CC6)nc5c3F)N3C[C@H]5CC[C@H](N5)[C@H]3CO4)c12]

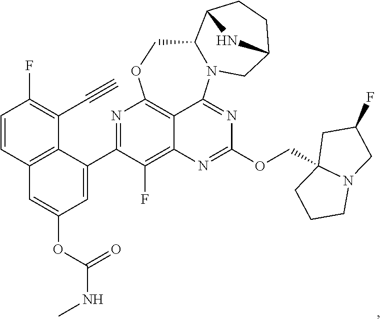 [SMILES: C#Cc1c(F)ccc2cc(OC(=O)NC)cc(-c3nc4c5c(nc(OC[C@@]67CCCN6C[C@H](F)C7)nc5c3F)N3C[C@H]5CC[C@H](N5)[C@H]3CO4)c12]

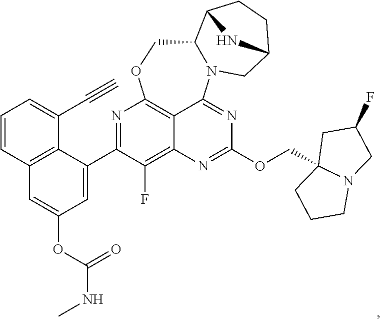 [SMILES: C#Cc1cccc2cc(OC(=O)NC)cc(-c3nc4c5c(nc(OC[C@@]67CCCN6C[C@H](F)C7)nc5c3F)N3C[C@H]5CC[C@H](N5)[C@H]3CO4)c12]

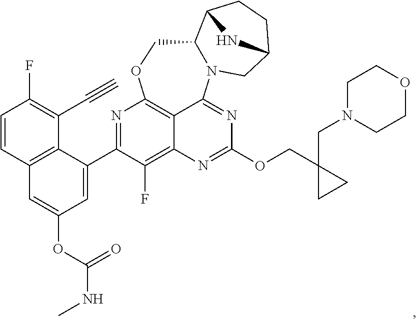 [SMILES: C#Cc1c(F)ccc2cc(OC(=O)NC)cc(-c3nc4c5c(nc(OCC6(CN7CCOCC7)CC6)nc5c3F)N3C[C@H]5CC[C@H](N5)[C@H]3CO4)c12]

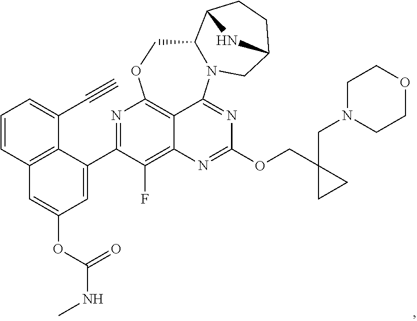 [SMILES: C#Cc1cccc2cc(OC(=O)NC)cc(-c3nc4c5c(nc(OCC6(CN7CCOCC7)CC6)nc5c3F)N3C[C@H]5CC[C@H](N5)[C@H]3CO4)c12]

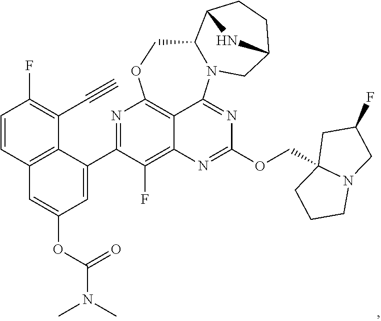 [SMILES: C#Cc1c(F)ccc2cc(OC(=O)N(C)C)cc(-c3nc4c5c(nc(OC[C@@]67CCCN6C[C@H](F)C7)nc5c3F)N3C[C@H]5CC[C@H](N5)[C@H]3CO4)c12]

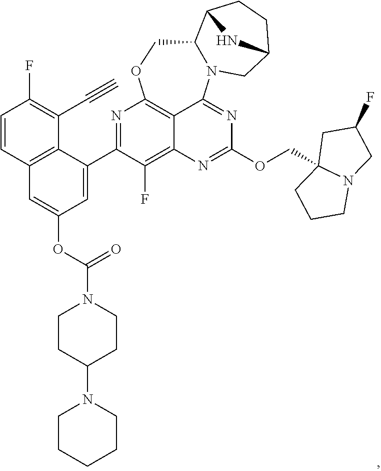 [SMILES: C#Cc1c(F)ccc2cc(OC(=O)N3CCC(N4CCCCC4)CC3)cc(-c3nc4c5c(nc(OC[C@@]67CCCN6C[C@H](F)C7)nc5c3F)N3C[C@H]5CC[C@H](N5)[C@H]3CO4)c12]

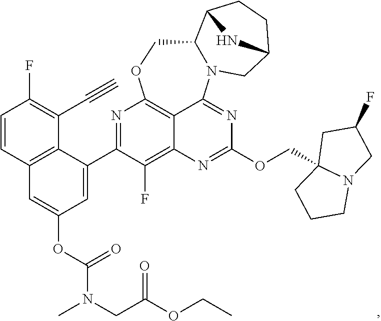 [SMILES: C#Cc1c(F)ccc2cc(OC(=O)N(C)CC(=O)OCC)cc(-c3nc4c5c(nc(OC[C@@]67CCCN6C[C@H](F)C7)nc5c3F)N3C[C@H]5CC[C@H](N5)[C@H]3CO4)c12]

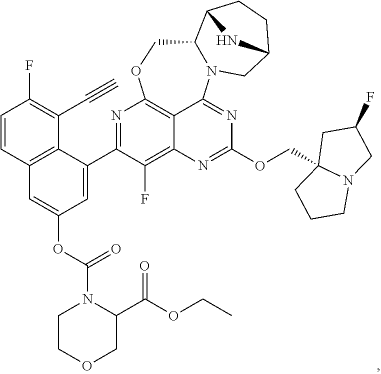 [SMILES: C#Cc1c(F)ccc2cc(OC(=O)N3CCOCC3C(=O)OCC)cc(-c3nc4c5c(nc(OC[C@@]67CCCN6C[C@H](F)C7)nc5c3F)N3C[C@H]5CC[C@H](N5)[C@H]3CO4)c12]

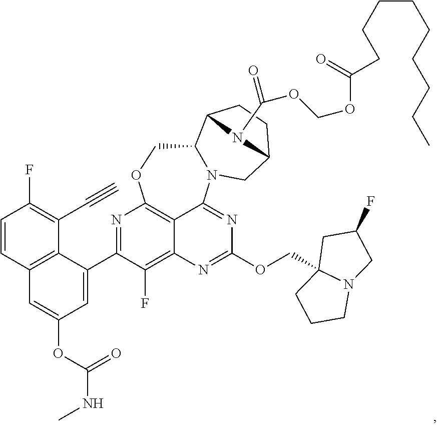 [SMILES: C#Cc1c(F)ccc2cc(OC(=O)NC)cc(-c3nc4c5c(nc(OC[C@@]67CCCN6C[C@H](F)C7)nc5c3F)N3C[C@H]5CC[C@@H]([C@H]3CO4)N5C(=O)OCOC(=O)CCCCCCCCC)c12]

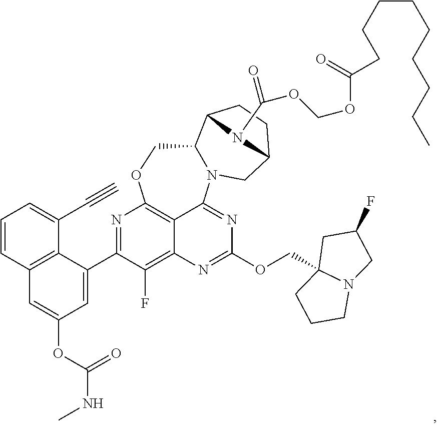 [SMILES: C#Cc1cccc2cc(OC(=O)NC)cc(-c3nc4c5c(nc(OC[C@@]67CCCN6C[C@H](F)C7)nc5c3F)N3C[C@H]5CC[C@@H]([C@H]3CO4)N5C(=O)OCOC(=O)CCCCCCCCC)c12]

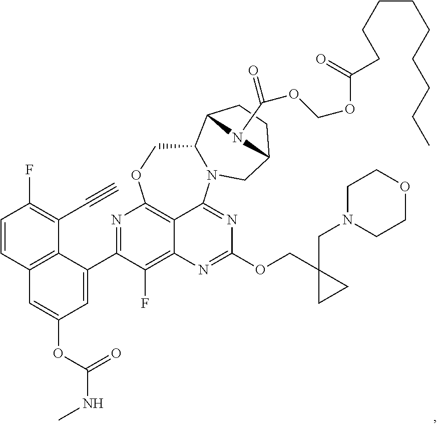 [SMILES: C#Cc1c(F)ccc2cc(OC(=O)NC)cc(-c3nc4c5c(nc(OCC6(CN7CCOCC7)CC6)nc5c3F)N3C[C@H]5CC[C@@H]([C@H]3CO4)N5C(=O)OCOC(=O)CCCCCCCCC)c12]

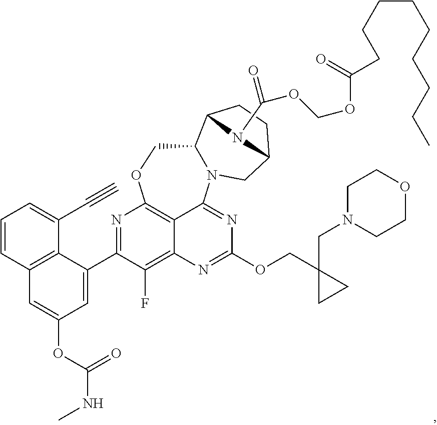 [SMILES: C#Cc1cccc2cc(OC(=O)NC)cc(-c3nc4c5c(nc(OCC6(CN7CCOCC7)CC6)nc5c3F)N3C[C@H]5CC[C@@H]([C@H]3CO4)N5C(=O)OCOC(=O)CCCCCCCCC)c12]

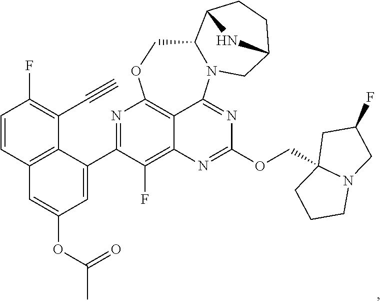 [SMILES: C#Cc1c(F)ccc2cc(OC(C)=O)cc(-c3nc4c5c(nc(OC[C@@]67CCCN6C[C@H](F)C7)nc5c3F)N3C[C@H]5CC[C@H](N5)[C@H]3CO4)c12]

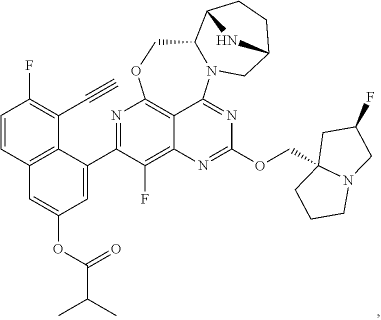 [SMILES: C#Cc1c(F)ccc2cc(OC(=O)C(C)C)cc(-c3nc4c5c(nc(OC[C@@]67CCCN6C[C@H](F)C7)nc5c3F)N3C[C@H]5CC[C@H](N5)[C@H]3CO4)c12]

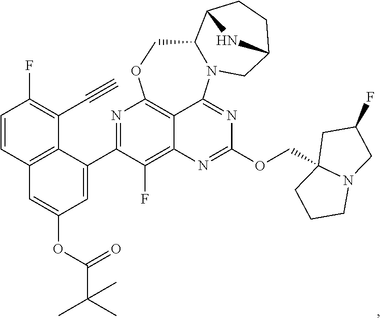 [SMILES: C#Cc1c(F)ccc2cc(OC(=O)C(C)(C)C)cc(-c3nc4c5c(nc(OC[C@@]67CCCN6C[C@H](F)C7)nc5c3F)N3C[C@H]5CC[C@H](N5)[C@H]3CO4)c12]

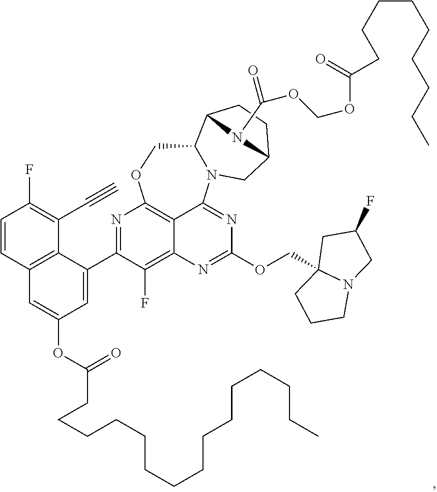 [SMILES: C#Cc1c(F)ccc2cc(OC(=O)CCCCCCCCCCCCCCC)cc(-c3nc4c5c(nc(OC[C@@]67CCCN6C[C@H](F)C7)nc5c3F)N3C[C@H]5CC[C@@H]([C@H]3CO4)N5C(=O)OCOC(=O)CCCCCCCCC)c12]

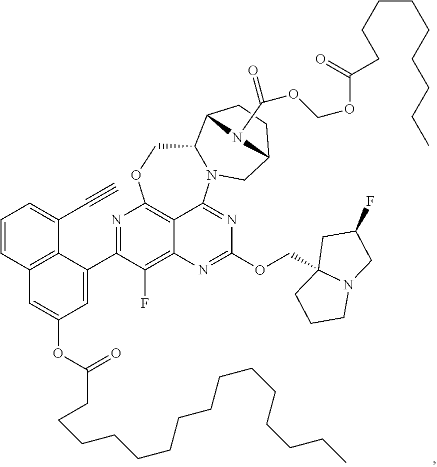 [SMILES: C#Cc1cccc2cc(OC(=O)CCCCCCCCCCCCCCC)cc(-c3nc4c5c(nc(OC[C@@]67CCCN6C[C@H](F)C7)nc5c3F)N3C[C@H]5CC[C@@H]([C@H]3CO4)N5C(=O)OCOC(=O)CCCCCCCCC)c12]

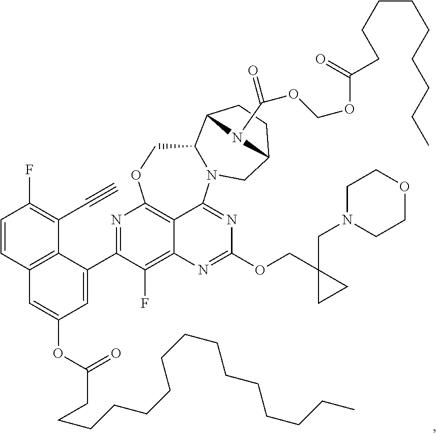 [SMILES: C#Cc1c(F)ccc2cc(OC(=O)CCCCCCCCCCCCCCC)cc(-c3nc4c5c(nc(OCC6(CN7CCOCC7)CC6)nc5c3F)N3C[C@H]5CC[C@@H]([C@H]3CO4)N5C(=O)OCOC(=O)CCCCCCCCC)c12]

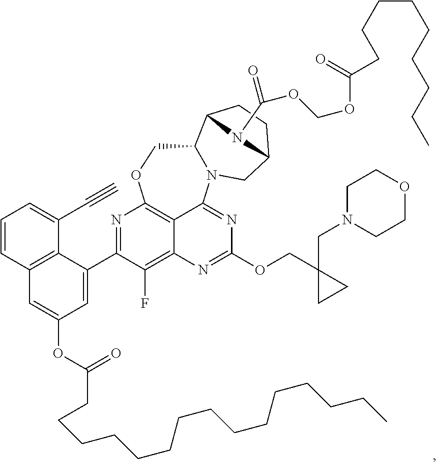 [SMILES: C#Cc1cccc2cc(OC(=O)CCCCCCCCCCCCCCC)cc(-c3nc4c5c(nc(OCC6(CN7CCOCC7)CC6)nc5c3F)N3C[C@H]5CC[C@@H]([C@H]3CO4)N5C(=O)OCOC(=O)CCCCCCCCC)c12]

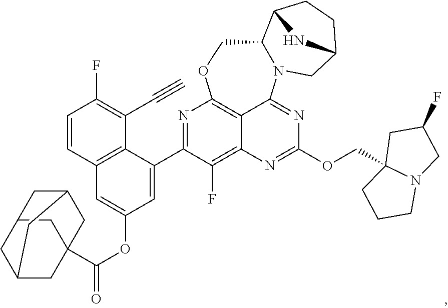 [SMILES: C#Cc1c(F)ccc2cc(OC(=O)C34CC5CC(CC(C5)C3)C4)cc(-c3nc4c5c(nc(OC[C@@]67CCCN6C[C@H](F)C7)nc5c3F)N3C[C@H]5CC[C@H](N5)[C@H]3CO4)c12]

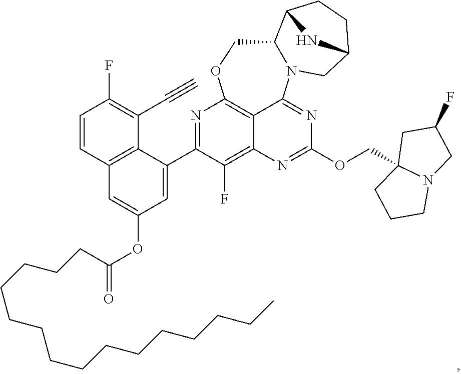 [SMILES: C#Cc1c(F)ccc2cc(OC(=O)CCCCCCCCCCCCCCC)cc(-c3nc4c5c(nc(OC[C@@]67CCCN6C[C@H](F)C7)nc5c3F)N3C[C@H]5CC[C@H](N5)[C@H]3CO4)c12]

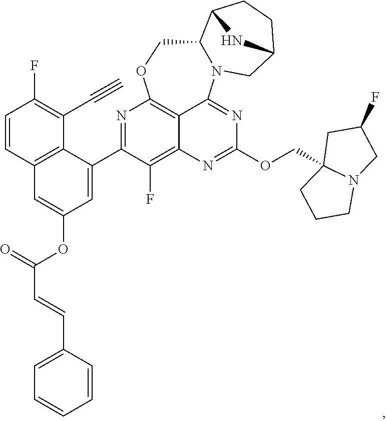 [SMILES: C#Cc1c(F)ccc2cc(OC(=O)/C=C/c3ccccc3)cc(-c3nc4c5c(nc(OC[C@@]67CCCN6C[C@H](F)C7)nc5c3F)N3C[C@H]5CC[C@H](N5)[C@H]3CO4)c12]

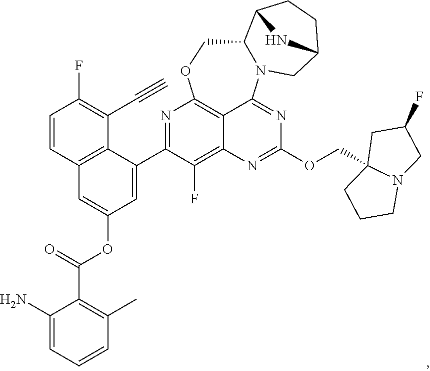 [SMILES: C#Cc1c(F)ccc2cc(OC(=O)c3c(C)cccc3N)cc(-c3nc4c5c(nc(OC[C@@]67CCCN6C[C@H](F)C7)nc5c3F)N3C[C@H]5CC[C@H](N5)[C@H]3CO4)c12]

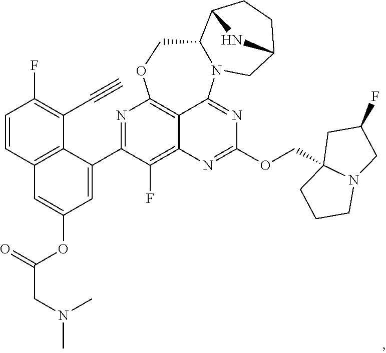 [SMILES: C#Cc1c(F)ccc2cc(OC(=O)CN(C)C)cc(-c3nc4c5c(nc(OC[C@@]67CCCN6C[C@H](F)C7)nc5c3F)N3C[C@H]5CC[C@H](N5)[C@H]3CO4)c12]

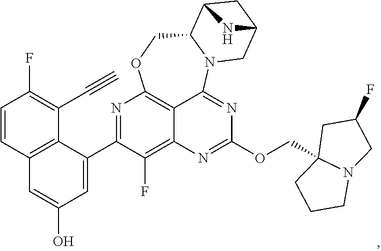 [SMILES: C#Cc1c(F)ccc2cc(O)cc(-c3nc4c5c(nc(OC[C@@]67CCCN6C[C@H](F)C7)nc5c3F)N3C[C@H]5C[C@H](N5)[C@H]3CO4)c12]

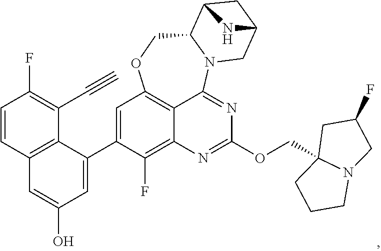 [SMILES: C#Cc1c(F)ccc2cc(O)cc(-c3cc4c5c(nc(OC[C@@]67CCCN6C[C@H](F)C7)nc5c3F)N3C[C@H]5C[C@H](N5)[C@H]3CO4)c12]

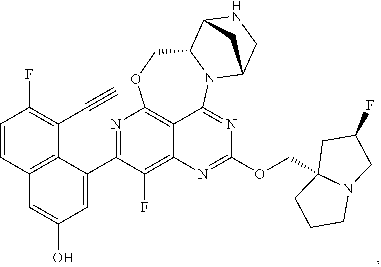 [SMILES: C#Cc1c(F)ccc2cc(O)cc(-c3nc4c5c(nc(OC[C@@]67CCCN6C[C@H](F)C7)nc5c3F)N3[C@H]5CN[C@H](C5)[C@H]3CO4)c12]

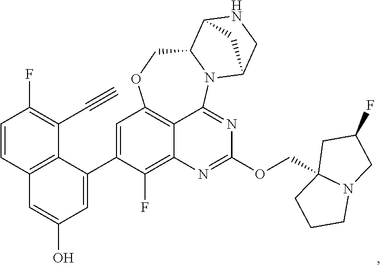 [SMILES: C#Cc1c(F)ccc2cc(O)cc(-c3cc4c5c(nc(OC[C@@]67CCCN6C[C@H](F)C7)nc5c3F)N3[C@@H]5CN[C@@H](C5)[C@H]3CO4)c12]

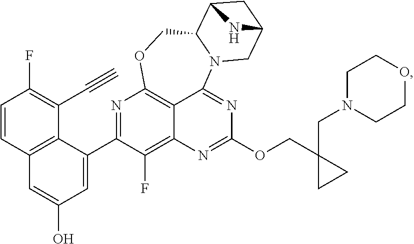 [SMILES: C#Cc1c(F)ccc2cc(O)cc(-c3nc4c5c(nc(OCC6(CN7CCOCC7)CC6)nc5c3F)N3C[C@H]5C[C@H](N5)[C@H]3CO4)c12]

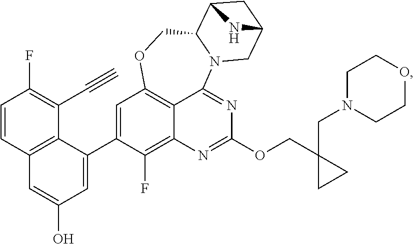 [SMILES: C#Cc1c(F)ccc2cc(O)cc(-c3cc4c5c(nc(OCC6(CN7CCOCC7)CC6)nc5c3F)N3C[C@H]5C[C@H](N5)[C@H]3CO4)c12]

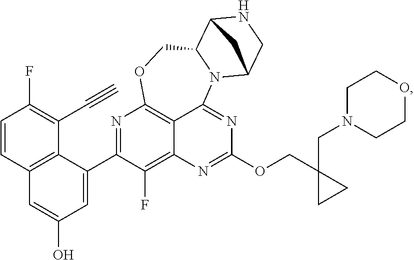 [SMILES: C#Cc1c(F)ccc2cc(O)cc(-c3nc4c5c(nc(OCC6(CN7CCOCC7)CC6)nc5c3F)N3[C@H]5CN[C@H](C5)[C@H]3CO4)c12]

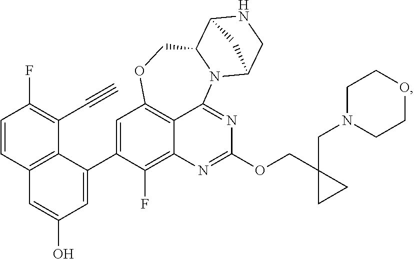 [SMILES: C#Cc1c(F)ccc2cc(O)cc(-c3cc4c5c(nc(OCC6(CN7CCOCC7)CC6)nc5c3F)N3[C@@H]5CN[C@@H](C5)[C@H]3CO4)c12]

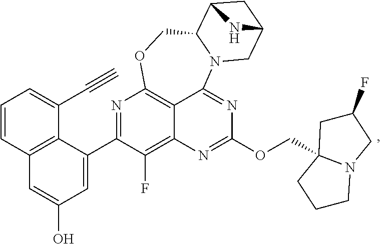 [SMILES: C#Cc1cccc2cc(O)cc(-c3nc4c5c(nc(OC[C@@]67CCCN6C[C@H](F)C7)nc5c3F)N3C[C@H]5C[C@H](N5)[C@H]3CO4)c12]

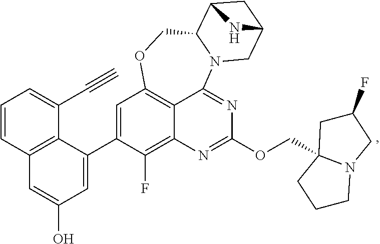 [SMILES: C#Cc1cccc2cc(O)cc(-c3cc4c5c(nc(OC[C@@]67CCCN6C[C@H](F)C7)nc5c3F)N3C[C@H]5C[C@H](N5)[C@H]3CO4)c12]

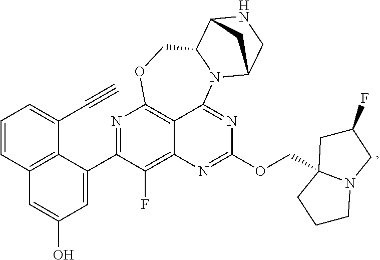 [SMILES: C#Cc1cccc2cc(O)cc(-c3nc4c5c(nc(OC[C@@]67CCCN6C[C@H](F)C7)nc5c3F)N3[C@H]5CN[C@H](C5)[C@H]3CO4)c12]

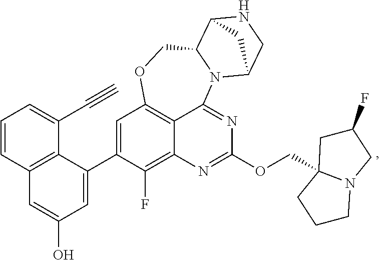 [SMILES: C#Cc1cccc2cc(O)cc(-c3cc4c5c(nc(OC[C@@]67CCCN6C[C@H](F)C7)nc5c3F)N3[C@@H]5CN[C@@H](C5)[C@H]3CO4)c12]

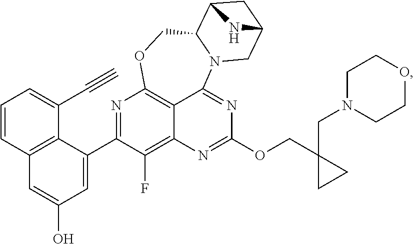 [SMILES: C#Cc1cccc2cc(O)cc(-c3nc4c5c(nc(OCC6(CN7CCOCC7)CC6)nc5c3F)N3C[C@H]5C[C@H](N5)[C@H]3CO4)c12]

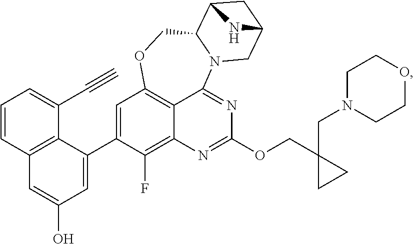 [SMILES: C#Cc1cccc2cc(O)cc(-c3cc4c5c(nc(OCC6(CN7CCOCC7)CC6)nc5c3F)N3C[C@H]5C[C@H](N5)[C@H]3CO4)c12]

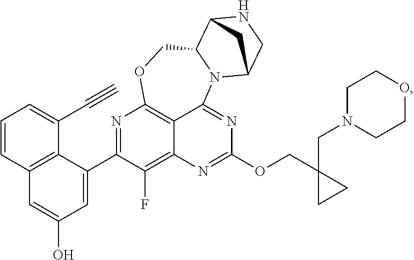 [SMILES: C#Cc1cccc2cc(O)cc(-c3nc4c5c(nc(OCC6(CN7CCOCC7)CC6)nc5c3F)N3[C@H]5CN[C@H](C5)[C@H]3CO4)c12]

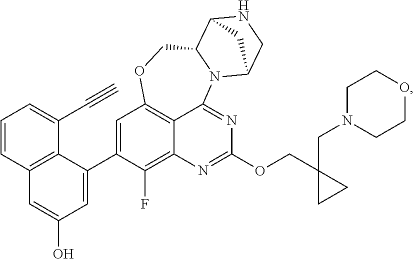 [SMILES: C#Cc1cccc2cc(O)cc(-c3cc4c5c(nc(OCC6(CN7CCOCC7)CC6)nc5c3F)N3[C@@H]5CN[C@@H](C5)[C@H]3CO4)c12]

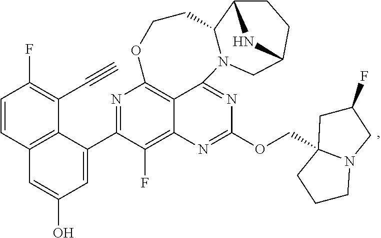 [SMILES: C#Cc1c(F)ccc2cc(O)cc(-c3nc4c5c(nc(OC[C@@]67CCCN6C[C@H](F)C7)nc5c3F)N3C[C@H]5CC[C@H](N5)[C@H]3CCO4)c12]

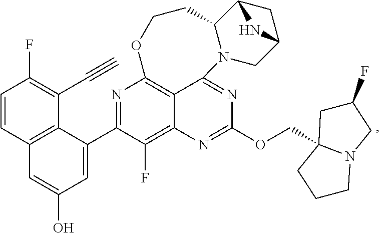 [SMILES: C#Cc1c(F)ccc2cc(O)cc(-c3nc4c5c(nc(OC[C@@]67CCCN6C[C@H](F)C7)nc5c3F)N3C[C@H]5C[C@H](N5)[C@H]3CCO4)c12]

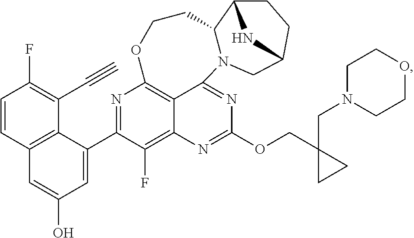 [SMILES: C#Cc1c(F)ccc2cc(O)cc(-c3nc4c5c(nc(OCC6(CN7CCOCC7)CC6)nc5c3F)N3C[C@H]5CC[C@H](N5)[C@H]3CCO4)c12]

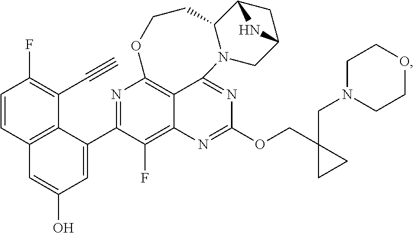 [SMILES: C#Cc1c(F)ccc2cc(O)cc(-c3nc4c5c(nc(OCC6(CN7CCOCC7)CC6)nc5c3F)N3C[C@H]5C[C@H](N5)[C@H]3CCO4)c12]

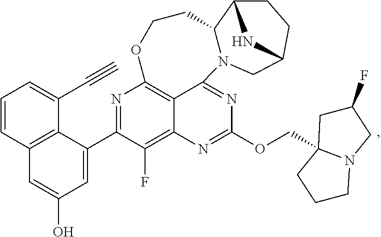 [SMILES: C#Cc1cccc2cc(O)cc(-c3nc4c5c(nc(OC[C@@]67CCCN6C[C@H](F)C7)nc5c3F)N3C[C@H]5CC[C@H](N5)[C@H]3CCO4)c12]

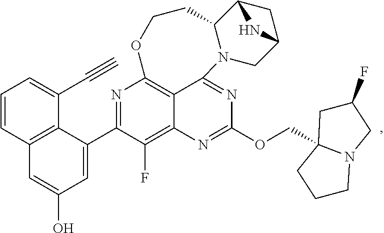 [SMILES: C#Cc1cccc2cc(O)cc(-c3nc4c5c(nc(OC[C@@]67CCCN6C[C@H](F)C7)nc5c3F)N3C[C@H]5C[C@H](N5)[C@H]3CCO4)c12]

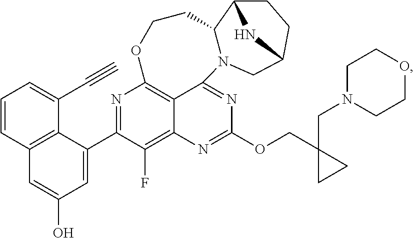 [SMILES: C#Cc1cccc2cc(O)cc(-c3nc4c5c(nc(OCC6(CN7CCOCC7)CC6)nc5c3F)N3C[C@H]5CC[C@H](N5)[C@H]3CCO4)c12]

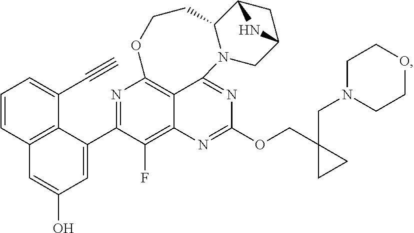 [SMILES: C#Cc1cccc2cc(O)cc(-c3nc4c5c(nc(OCC6(CN7CCOCC7)CC6)nc5c3F)N3C[C@H]5C[C@H](N5)[C@H]3CCO4)c12]

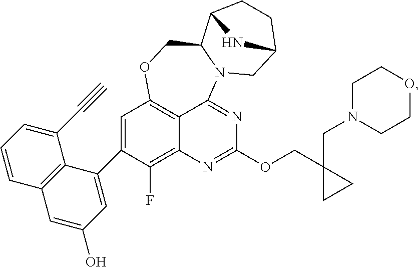 [SMILES: C#Cc1cccc2cc(O)cc(-c3cc4c5c(nc(OCC6(CN7CCOCC7)CC6)nc5c3F)N3C[C@H]5CC[C@H](N5)[C@@H]3CO4)c12]

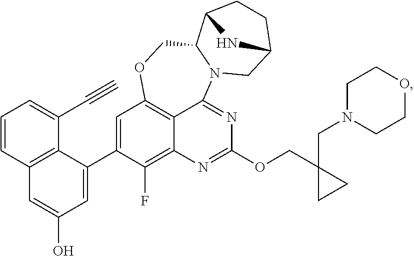 [SMILES: C#Cc1cccc2cc(O)cc(-c3cc4c5c(nc(OCC6(CN7CCOCC7)CC6)nc5c3F)N3C[C@H]5CC[C@H](N5)[C@H]3CO4)c12]

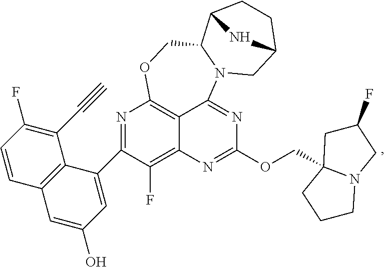 [SMILES: C#Cc1c(F)ccc2cc(O)cc(-c3nc4c5c(nc(OC[C@@]67CCCN6C[C@H](F)C7)nc5c3F)N3C[C@H]5CC[C@H](N5)[C@H]3CO4)c12]

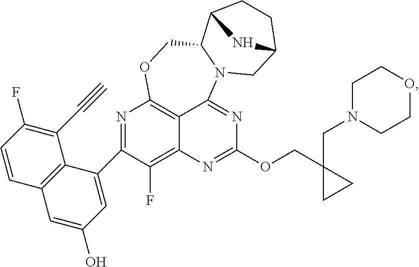 [SMILES: C#Cc1c(F)ccc2cc(O)cc(-c3nc4c5c(nc(OCC6(CN7CCOCC7)CC6)nc5c3F)N3C[C@H]5CC[C@H](N5)[C@H]3CO4)c12]

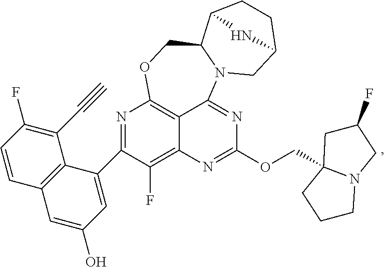 [SMILES: C#Cc1c(F)ccc2cc(O)cc(-c3nc4c5c(nc(OC[C@@]67CCCN6C[C@H](F)C7)nc5c3F)N3C[C@@H]5CC[C@@H](N5)[C@@H]3CO4)c12]

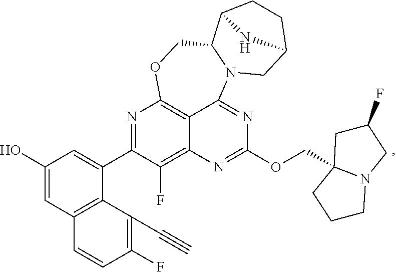 [SMILES: C#Cc1c(F)ccc2cc(O)cc(-c3nc4c5c(nc(OC[C@@]67CCCN6C[C@H](F)C7)nc5c3F)N3C[C@@H]5CC[C@@H](N5)[C@H]3CO4)c12]

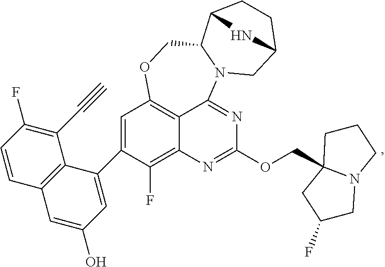 [SMILES: C#Cc1c(F)ccc2cc(O)cc(-c3cc4c5c(nc(OC[C@@]67CCCN6C[C@H](F)C7)nc5c3F)N3C[C@H]5CC[C@H](N5)[C@H]3CO4)c12]

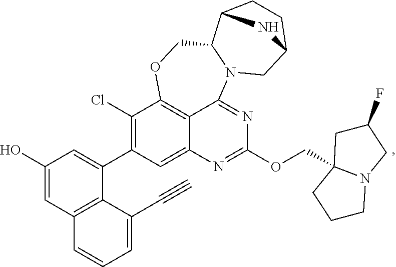 [SMILES: C#Cc1cccc2cc(O)cc(-c3cc4nc(OC[C@@]56CCCN5C[C@H](F)C6)nc5c4c(c3Cl)OC[C@@H]3[C@@H]4CC[C@H](CN53)N4)c12]

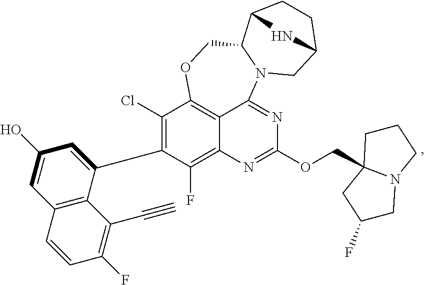

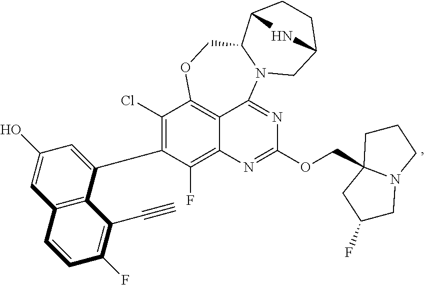

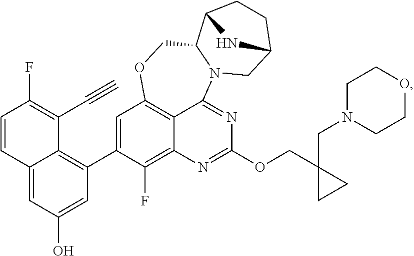 [SMILES: C#Cc1c(F)ccc2cc(O)cc(-c3cc4c5c(nc(OCC6(CN7CCOCC7)CC6)nc5c3F)N3C[C@H]5CC[C@H](N5)[C@H]3CO4)c12]

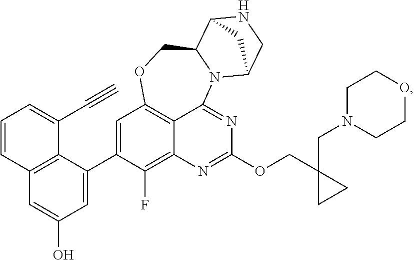 [SMILES: C#Cc1cccc2cc(O)cc(-c3cc4c5c(nc(OCC6(CN7CCOCC7)CC6)nc5c3F)N3[C@@H]5CN[C@@H](C5)[C@@H]3CO4)c12]

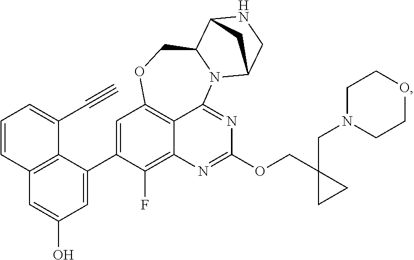 [SMILES: C#Cc1cccc2cc(O)cc(-c3cc4c5c(nc(OCC6(CN7CCOCC7)CC6)nc5c3F)N3[C@H]5CN[C@H](C5)[C@@H]3CO4)c12]

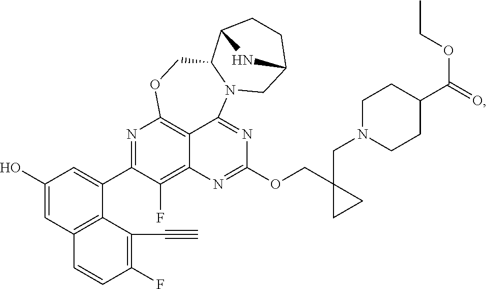 [SMILES: C#Cc1c(F)ccc2cc(O)cc(-c3nc4c5c(nc(OCC6(CN7CCC(C(=O)OCC)CC7)CC6)nc5c3F)N3C[C@H]5CC[C@H](N5)[C@H]3CO4)c12]

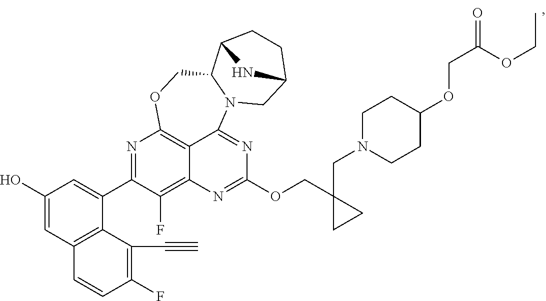 [SMILES: C#Cc1c(F)ccc2cc(O)cc(-c3nc4c5c(nc(OCC6(CN7CCC(OCC(=O)OCC)CC7)CC6)nc5c3F)N3C[C@H]5CC[C@H](N5)[C@H]3CO4)c12]

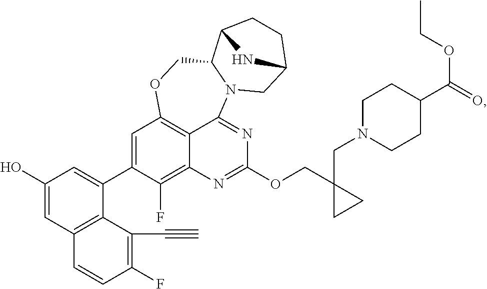 [SMILES: C#Cc1c(F)ccc2cc(O)cc(-c3cc4c5c(nc(OCC6(CN7CCC(C(=O)OCC)CC7)CC6)nc5c3F)N3C[C@H]5CC[C@H](N5)[C@H]3CO4)c12]

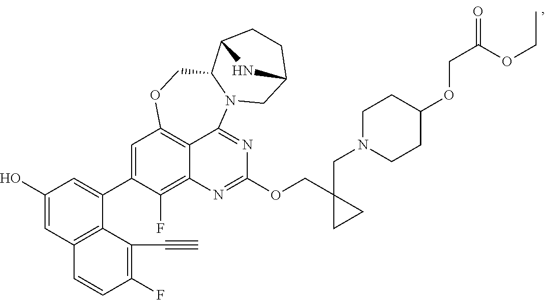 [SMILES: C#Cc1c(F)ccc2cc(O)cc(-c3cc4c5c(nc(OCC6(CN7CCC(OCC(=O)OCC)CC7)CC6)nc5c3F)N3C[C@H]5CC[C@H](N5)[C@H]3CO4)c12]

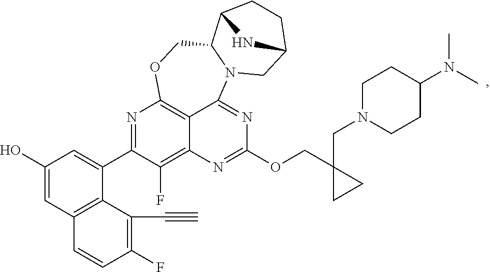 [SMILES: C#Cc1c(F)ccc2cc(O)cc(-c3nc4c5c(nc(OCC6(CN7CCC(N(C)C)CC7)CC6)nc5c3F)N3C[C@H]5CC[C@H](N5)[C@H]3CO4)c12]

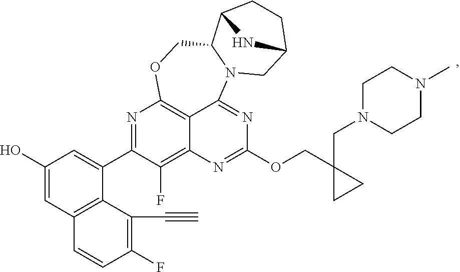 [SMILES: C#Cc1c(F)ccc2cc(O)cc(-c3nc4c5c(nc(OCC6(CN7CCN(C)CC7)CC6)nc5c3F)N3C[C@H]5CC[C@H](N5)[C@H]3CO4)c12]

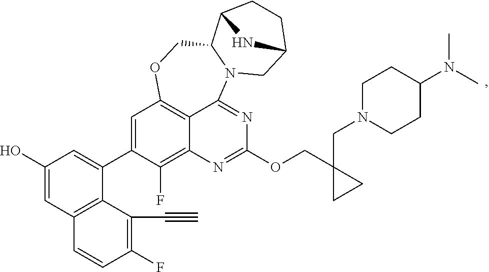 [SMILES: C#Cc1c(F)ccc2cc(O)cc(-c3cc4c5c(nc(OCC6(CN7CCC(N(C)C)CC7)CC6)nc5c3F)N3C[C@H]5CC[C@H](N5)[C@H]3CO4)c12]

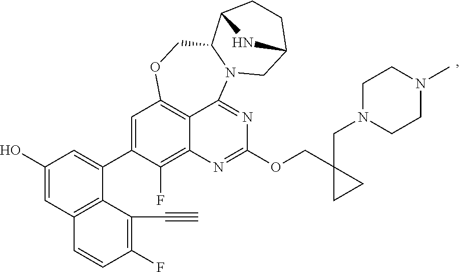 [SMILES: C#Cc1c(F)ccc2cc(O)cc(-c3cc4c5c(nc(OCC6(CN7CCN(C)CC7)CC6)nc5c3F)N3C[C@H]5CC[C@H](N5)[C@H]3CO4)c12]

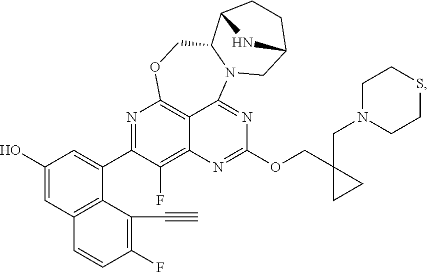 [SMILES: C#Cc1c(F)ccc2cc(O)cc(-c3nc4c5c(nc(OCC6(CN7CCSCC7)CC6)nc5c3F)N3C[C@H]5CC[C@H](N5)[C@H]3CO4)c12]

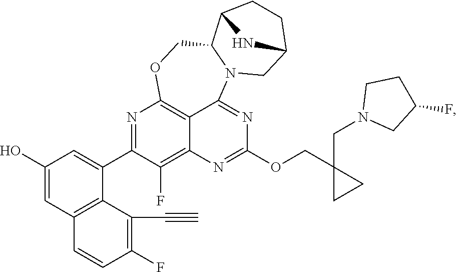 [SMILES: C#Cc1c(F)ccc2cc(O)cc(-c3nc4c5c(nc(OCC6(CN7CC[C@H](F)C7)CC6)nc5c3F)N3C[C@H]5CC[C@H](N5)[C@H]3CO4)c12]

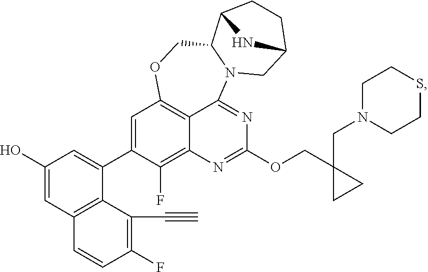 [SMILES: C#Cc1c(F)ccc2cc(O)cc(-c3cc4c5c(nc(OCC6(CN7CCSCC7)CC6)nc5c3F)N3C[C@H]5CC[C@H](N5)[C@H]3CO4)c12]

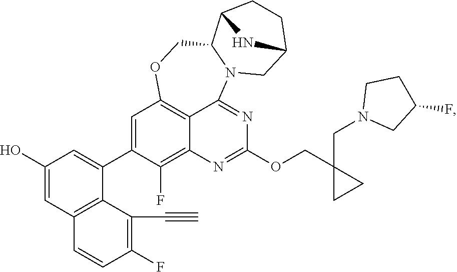 [SMILES: C#Cc1c(F)ccc2cc(O)cc(-c3cc4c5c(nc(OCC6(CN7CC[C@H](F)C7)CC6)nc5c3F)N3C[C@H]5CC[C@H](N5)[C@H]3CO4)c12]

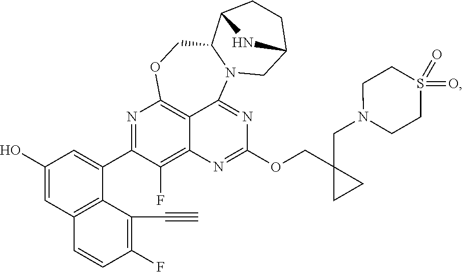 [SMILES: C#Cc1c(F)ccc2cc(O)cc(-c3nc4c5c(nc(OCC6(CN7CCS(=O)(=O)CC7)CC6)nc5c3F)N3C[C@H]5CC[C@H](N5)[C@H]3CO4)c12]

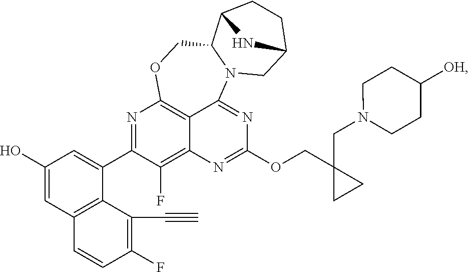 [SMILES: C#Cc1c(F)ccc2cc(O)cc(-c3nc4c5c(nc(OCC6(CN7CCC(O)CC7)CC6)nc5c3F)N3C[C@H]5CC[C@H](N5)[C@H]3CO4)c12]

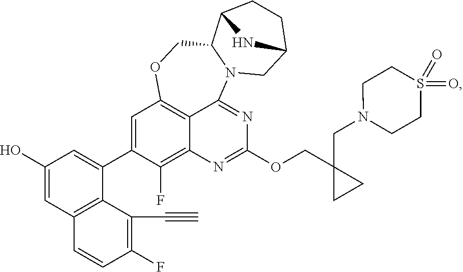 [SMILES: C#Cc1c(F)ccc2cc(O)cc(-c3cc4c5c(nc(OCC6(CN7CCS(=O)(=O)CC7)CC6)nc5c3F)N3C[C@H]5CC[C@H](N5)[C@H]3CO4)c12]

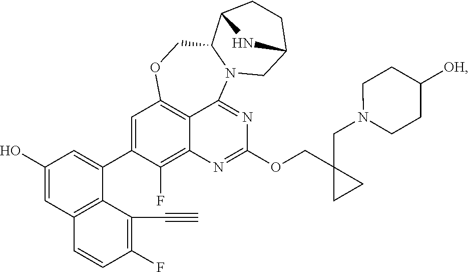 [SMILES: C#Cc1c(F)ccc2cc(O)cc(-c3cc4c5c(nc(OCC6(CN7CCC(O)CC7)CC6)nc5c3F)N3C[C@H]5CC[C@H](N5)[C@H]3CO4)c12]

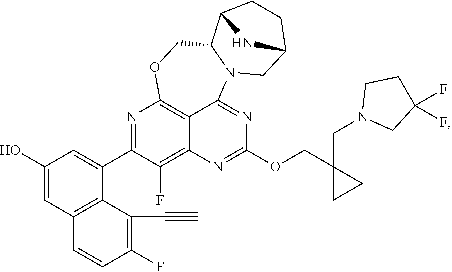 [SMILES: C#Cc1c(F)ccc2cc(O)cc(-c3nc4c5c(nc(OCC6(CN7CCC(F)(F)C7)CC6)nc5c3F)N3C[C@H]5CC[C@H](N5)[C@H]3CO4)c12]

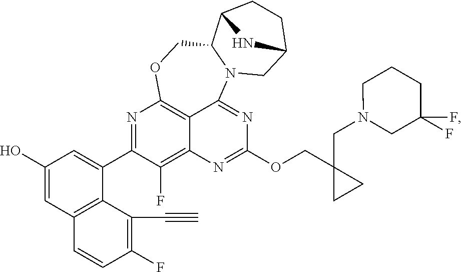 [SMILES: C#Cc1c(F)ccc2cc(O)cc(-c3nc4c5c(nc(OCC6(CN7CCCC(F)(F)C7)CC6)nc5c3F)N3C[C@H]5CC[C@H](N5)[C@H]3CO4)c12]

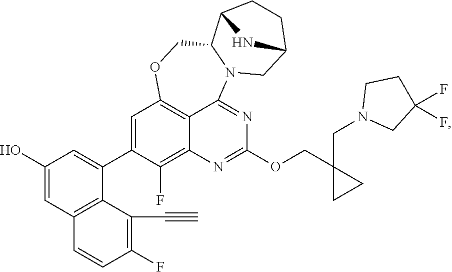 [SMILES: C#Cc1c(F)ccc2cc(O)cc(-c3cc4c5c(nc(OCC6(CN7CCC(F)(F)C7)CC6)nc5c3F)N3C[C@H]5CC[C@H](N5)[C@H]3CO4)c12]

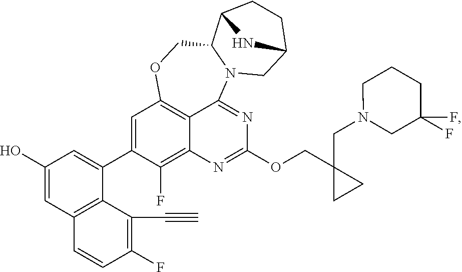 [SMILES: C#Cc1c(F)ccc2cc(O)cc(-c3cc4c5c(nc(OCC6(CN7CCCC(F)(F)C7)CC6)nc5c3F)N3C[C@H]5CC[C@H](N5)[C@H]3CO4)c12]

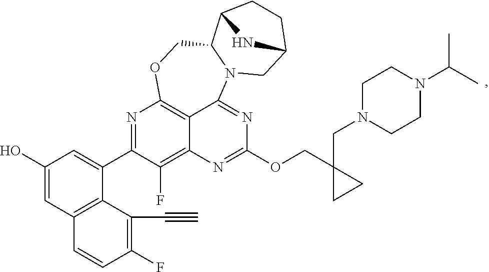 [SMILES: C#Cc1c(F)ccc2cc(O)cc(-c3nc4c5c(nc(OCC6(CN7CCN(C(C)C)CC7)CC6)nc5c3F)N3C[C@H]5CC[C@H](N5)[C@H]3CO4)c12]

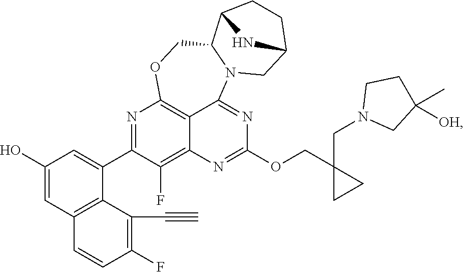 [SMILES: C#Cc1c(F)ccc2cc(O)cc(-c3nc4c5c(nc(OCC6(CN7CCC(C)(O)C7)CC6)nc5c3F)N3C[C@H]5CC[C@H](N5)[C@H]3CO4)c12]

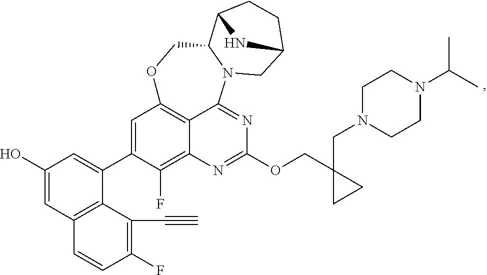 [SMILES: C#Cc1c(F)ccc2cc(O)cc(-c3cc4c5c(nc(OCC6(CN7CCN(C(C)C)CC7)CC6)nc5c3F)N3C[C@H]5CC[C@H](N5)[C@H]3CO4)c12]

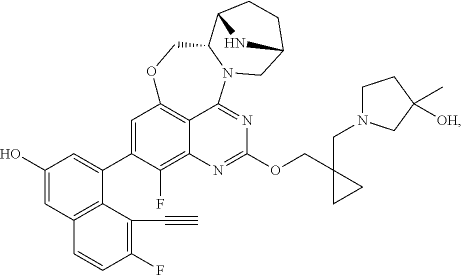 [SMILES: C#Cc1c(F)ccc2cc(O)cc(-c3cc4c5c(nc(OCC6(CN7CCC(C)(O)C7)CC6)nc5c3F)N3C[C@H]5CC[C@H](N5)[C@H]3CO4)c12]

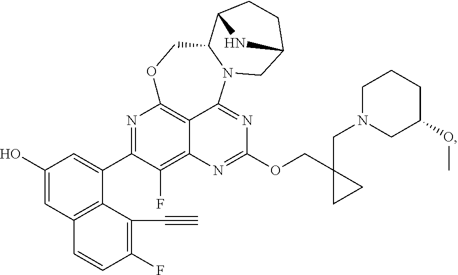 [SMILES: C#Cc1c(F)ccc2cc(O)cc(-c3nc4c5c(nc(OCC6(CN7CCC[C@H](OC)C7)CC6)nc5c3F)N3C[C@H]5CC[C@H](N5)[C@H]3CO4)c12]

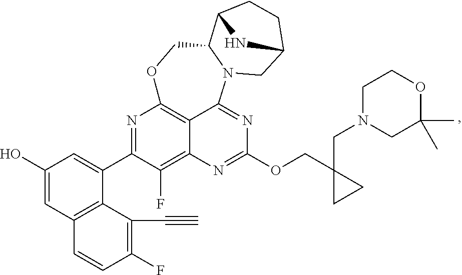 [SMILES: C#Cc1c(F)ccc2cc(O)cc(-c3nc4c5c(nc(OCC6(CN7CCOC(C)(C)C7)CC6)nc5c3F)N3C[C@H]5CC[C@H](N5)[C@H]3CO4)c12]

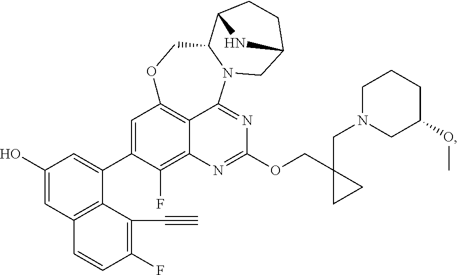 [SMILES: C#Cc1c(F)ccc2cc(O)cc(-c3cc4c5c(nc(OCC6(CN7CCC[C@H](OC)C7)CC6)nc5c3F)N3C[C@H]5CC[C@H](N5)[C@H]3CO4)c12]

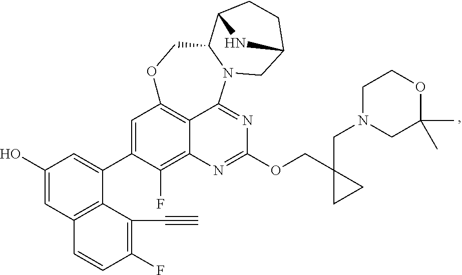 [SMILES: C#Cc1c(F)ccc2cc(O)cc(-c3cc4c5c(nc(OCC6(CN7CCOC(C)(C)C7)CC6)nc5c3F)N3C[C@H]5CC[C@H](N5)[C@H]3CO4)c12]

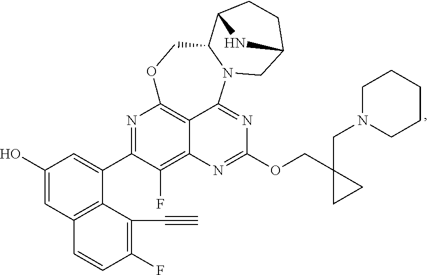 [SMILES: C#Cc1c(F)ccc2cc(O)cc(-c3nc4c5c(nc(OCC6(CN7CCCCC7)CC6)nc5c3F)N3C[C@H]5CC[C@H](N5)[C@H]3CO4)c12]

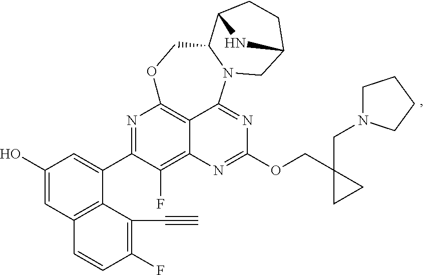 [SMILES: C#Cc1c(F)ccc2cc(O)cc(-c3nc4c5c(nc(OCC6(CN7CCCC7)CC6)nc5c3F)N3C[C@H]5CC[C@H](N5)[C@H]3CO4)c12]

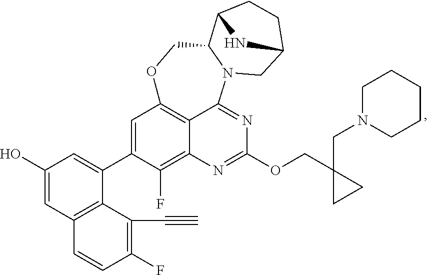 [SMILES: C#Cc1c(F)ccc2cc(O)cc(-c3cc4c5c(nc(OCC6(CN7CCCCC7)CC6)nc5c3F)N3C[C@H]5CC[C@H](N5)[C@H]3CO4)c12]

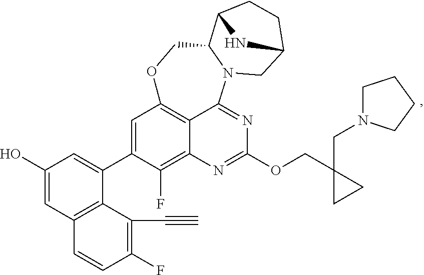 [SMILES: C#Cc1c(F)ccc2cc(O)cc(-c3cc4c5c(nc(OCC6(CN7CCCC7)CC6)nc5c3F)N3C[C@H]5CC[C@H](N5)[C@H]3CO4)c12]

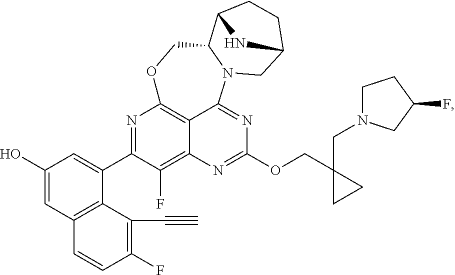 [SMILES: C#Cc1c(F)ccc2cc(O)cc(-c3nc4c5c(nc(OCC6(CN7CC[C@@H](F)C7)CC6)nc5c3F)N3C[C@H]5CC[C@H](N5)[C@H]3CO4)c12]

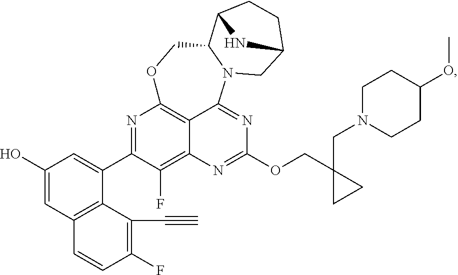 [SMILES: C#Cc1c(F)ccc2cc(O)cc(-c3nc4c5c(nc(OCC6(CN7CCC(OC)CC7)CC6)nc5c3F)N3C[C@H]5CC[C@H](N5)[C@H]3CO4)c12]

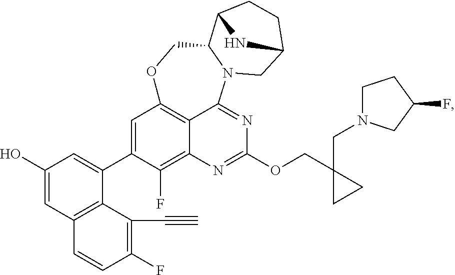 [SMILES: C#Cc1c(F)ccc2cc(O)cc(-c3cc4c5c(nc(OCC6(CN7CC[C@@H](F)C7)CC6)nc5c3F)N3C[C@H]5CC[C@H](N5)[C@H]3CO4)c12]

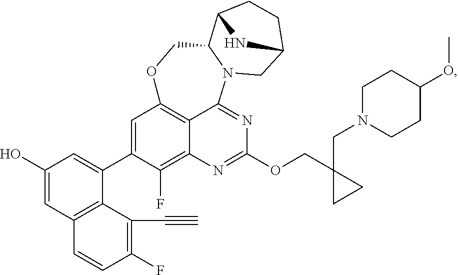 [SMILES: C#Cc1c(F)ccc2cc(O)cc(-c3cc4c5c(nc(OCC6(CN7CCC(OC)CC7)CC6)nc5c3F)N3C[C@H]5CC[C@H](N5)[C@H]3CO4)c12]

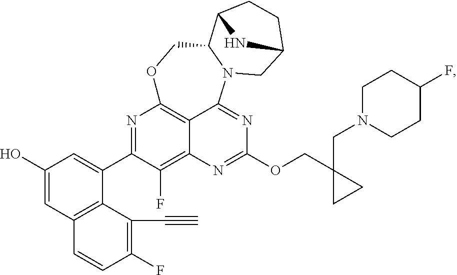 [SMILES: C#Cc1c(F)ccc2cc(O)cc(-c3nc4c5c(nc(OCC6(CN7CCC(F)CC7)CC6)nc5c3F)N3C[C@H]5CC[C@H](N5)[C@H]3CO4)c12]

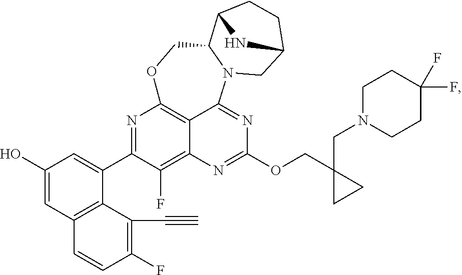 [SMILES: C#Cc1c(F)ccc2cc(O)cc(-c3nc4c5c(nc(OCC6(CN7CCC(F)(F)CC7)CC6)nc5c3F)N3C[C@H]5CC[C@H](N5)[C@H]3CO4)c12]

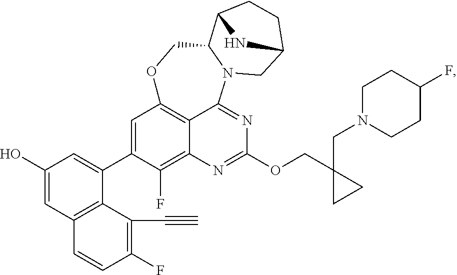 [SMILES: C#Cc1c(F)ccc2cc(O)cc(-c3cc4c5c(nc(OCC6(CN7CCC(F)CC7)CC6)nc5c3F)N3C[C@H]5CC[C@H](N5)[C@H]3CO4)c12]

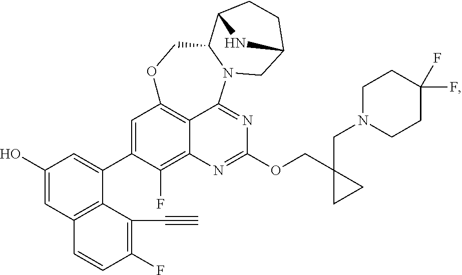 [SMILES: C#Cc1c(F)ccc2cc(O)cc(-c3cc4c5c(nc(OCC6(CN7CCC(F)(F)CC7)CC6)nc5c3F)N3C[C@H]5CC[C@H](N5)[C@H]3CO4)c12]

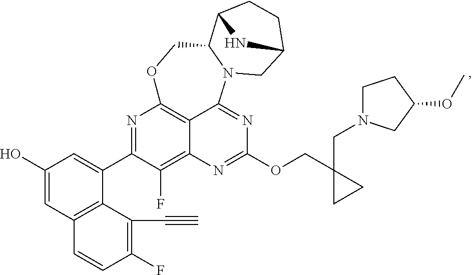 [SMILES: C#Cc1c(F)ccc2cc(O)cc(-c3nc4c5c(nc(OCC6(CN7CC[C@H](OC)C7)CC6)nc5c3F)N3C[C@H]5CC[C@H](N5)[C@H]3CO4)c12]

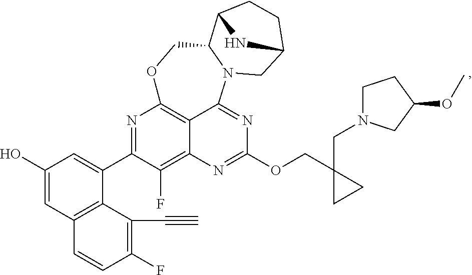 [SMILES: C#Cc1c(F)ccc2cc(O)cc(-c3nc4c5c(nc(OCC6(CN7CC[C@@H](OC)C7)CC6)nc5c3F)N3C[C@H]5CC[C@H](N5)[C@H]3CO4)c12]

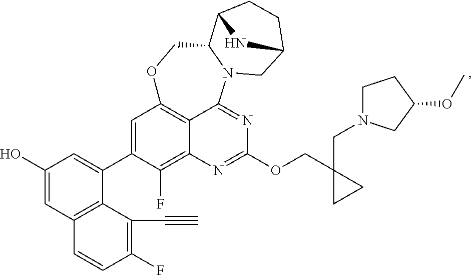 [SMILES: C#Cc1c(F)ccc2cc(O)cc(-c3cc4c5c(nc(OCC6(CN7CC[C@H](OC)C7)CC6)nc5c3F)N3C[C@H]5CC[C@H](N5)[C@H]3CO4)c12]

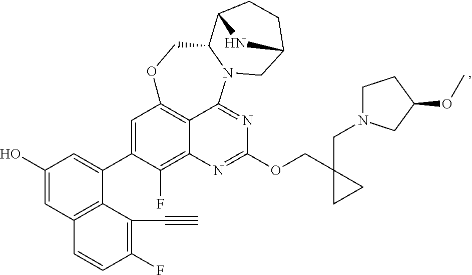 [SMILES: C#Cc1c(F)ccc2cc(O)cc(-c3cc4c5c(nc(OCC6(CN7CC[C@@H](OC)C7)CC6)nc5c3F)N3C[C@H]5CC[C@H](N5)[C@H]3CO4)c12]

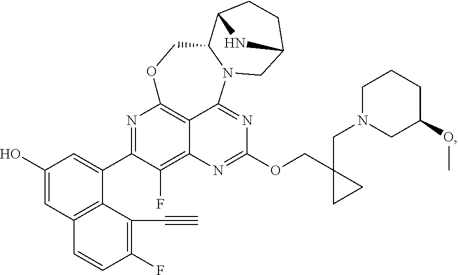 [SMILES: C#Cc1c(F)ccc2cc(O)cc(-c3nc4c5c(nc(OCC6(CN7CCC[C@@H](OC)C7)CC6)nc5c3F)N3C[C@H]5CC[C@H](N5)[C@H]3CO4)c12]

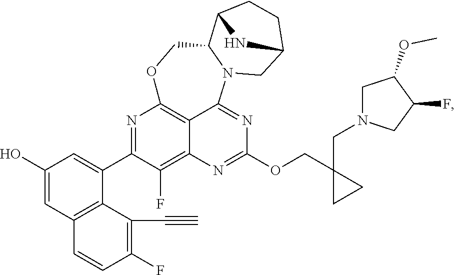 [SMILES: C#Cc1c(F)ccc2cc(O)cc(-c3nc4c5c(nc(OCC6(CN7C[C@H](OC)[C@@H](F)C7)CC6)nc5c3F)N3C[C@H]5CC[C@H](N5)[C@H]3CO4)c12]

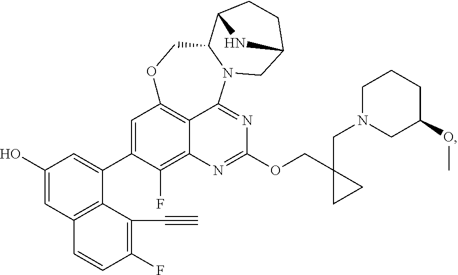 [SMILES: C#Cc1c(F)ccc2cc(O)cc(-c3cc4c5c(nc(OCC6(CN7CCC[C@@H](OC)C7)CC6)nc5c3F)N3C[C@H]5CC[C@H](N5)[C@H]3CO4)c12]

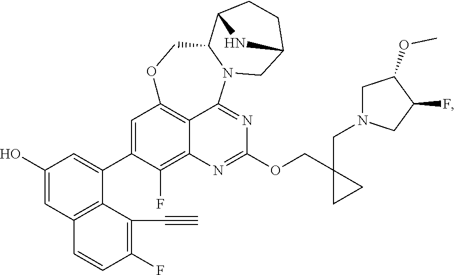 [SMILES: C#Cc1c(F)ccc2cc(O)cc(-c3cc4c5c(nc(OCC6(CN7C[C@H](OC)[C@@H](F)C7)CC6)nc5c3F)N3C[C@H]5CC[C@H](N5)[C@H]3CO4)c12]

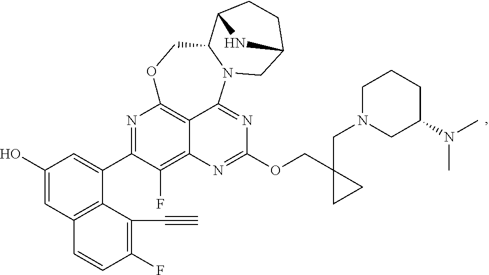 [SMILES: C#Cc1c(F)ccc2cc(O)cc(-c3nc4c5c(nc(OCC6(CN7CCC[C@H](N(C)C)C7)CC6)nc5c3F)N3C[C@H]5CC[C@H](N5)[C@H]3CO4)c12]

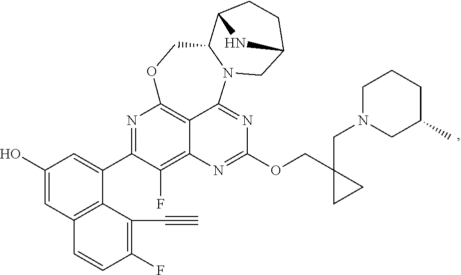 [SMILES: C#Cc1c(F)ccc2cc(O)cc(-c3nc4c5c(nc(OCC6(CN7CCC[C@H](C)C7)CC6)nc5c3F)N3C[C@H]5CC[C@H](N5)[C@H]3CO4)c12]

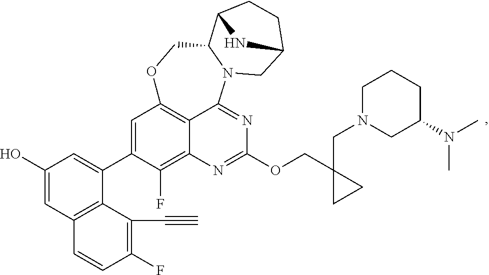 [SMILES: C#Cc1c(F)ccc2cc(O)cc(-c3cc4c5c(nc(OCC6(CN7CCC[C@H](N(C)C)C7)CC6)nc5c3F)N3C[C@H]5CC[C@H](N5)[C@H]3CO4)c12]

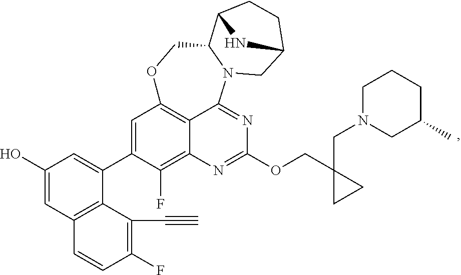 [SMILES: C#Cc1c(F)ccc2cc(O)cc(-c3cc4c5c(nc(OCC6(CN7CCC[C@H](C)C7)CC6)nc5c3F)N3C[C@H]5CC[C@H](N5)[C@H]3CO4)c12]

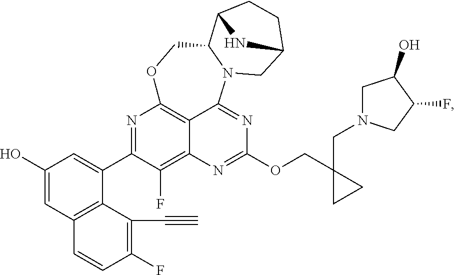 [SMILES: C#Cc1c(F)ccc2cc(O)cc(-c3nc4c5c(nc(OCC6(CN7C[C@@H](O)[C@H](F)C7)CC6)nc5c3F)N3C[C@H]5CC[C@H](N5)[C@H]3CO4)c12]

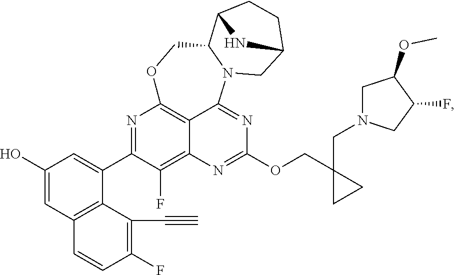 [SMILES: C#Cc1c(F)ccc2cc(O)cc(-c3nc4c5c(nc(OCC6(CN7C[C@@H](F)[C@H](OC)C7)CC6)nc5c3F)N3C[C@H]5CC[C@H](N5)[C@H]3CO4)c12]

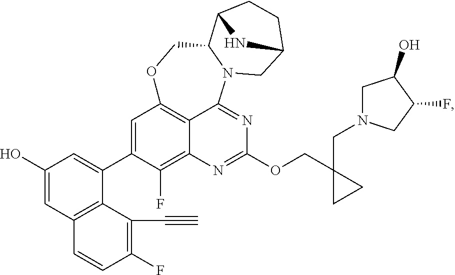 [SMILES: C#Cc1c(F)ccc2cc(O)cc(-c3cc4c5c(nc(OCC6(CN7C[C@@H](O)[C@H](F)C7)CC6)nc5c3F)N3C[C@H]5CC[C@H](N5)[C@H]3CO4)c12]

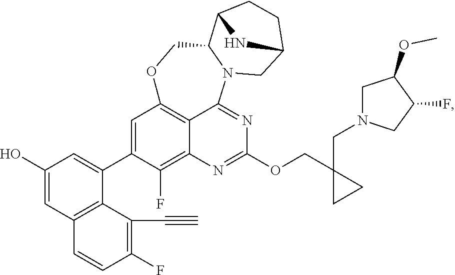 [SMILES: C#Cc1c(F)ccc2cc(O)cc(-c3cc4c5c(nc(OCC6(CN7C[C@@H](F)[C@H](OC)C7)CC6)nc5c3F)N3C[C@H]5CC[C@H](N5)[C@H]3CO4)c12]

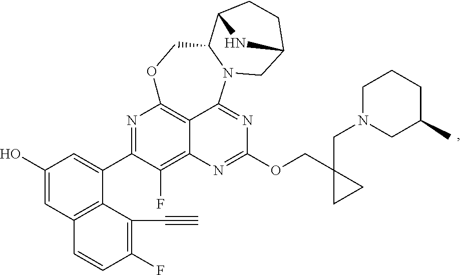 [SMILES: C#Cc1c(F)ccc2cc(O)cc(-c3nc4c5c(nc(OCC6(CN7CCC[C@@H](C)C7)CC6)nc5c3F)N3C[C@H]5CC[C@H](N5)[C@H]3CO4)c12]

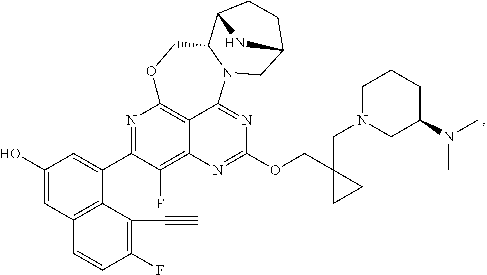 [SMILES: C#Cc1c(F)ccc2cc(O)cc(-c3nc4c5c(nc(OCC6(CN7CCC[C@@H](N(C)C)C7)CC6)nc5c3F)N3C[C@H]5CC[C@H](N5)[C@H]3CO4)c12]

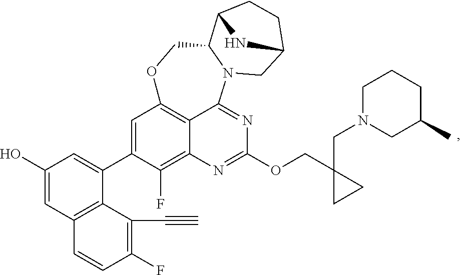 [SMILES: C#Cc1c(F)ccc2cc(O)cc(-c3cc4c5c(nc(OCC6(CN7CCC[C@@H](C)C7)CC6)nc5c3F)N3C[C@H]5CC[C@H](N5)[C@H]3CO4)c12]

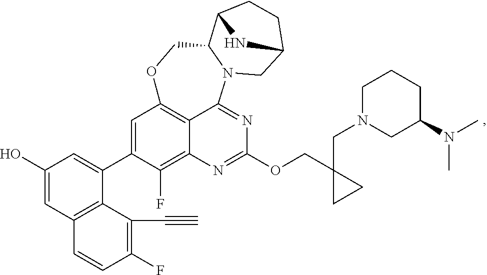 [SMILES: C#Cc1c(F)ccc2cc(O)cc(-c3cc4c5c(nc(OCC6(CN7CCC[C@@H](N(C)C)C7)CC6)nc5c3F)N3C[C@H]5CC[C@H](N5)[C@H]3CO4)c12]

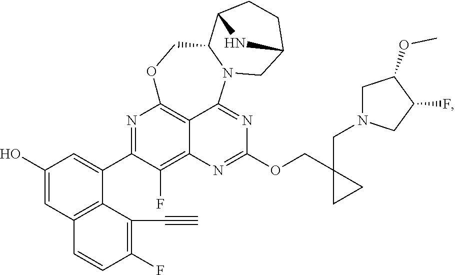 [SMILES: C#Cc1c(F)ccc2cc(O)cc(-c3nc4c5c(nc(OCC6(CN7C[C@@H](F)[C@@H](OC)C7)CC6)nc5c3F)N3C[C@H]5CC[C@H](N5)[C@H]3CO4)c12]

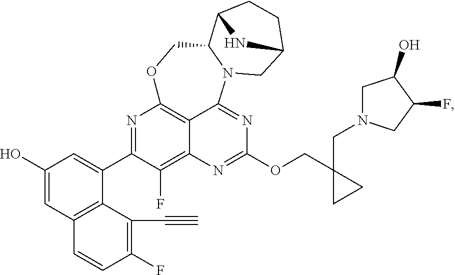 [SMILES: C#Cc1c(F)ccc2cc(O)cc(-c3nc4c5c(nc(OCC6(CN7C[C@@H](O)[C@@H](F)C7)CC6)nc5c3F)N3C[C@H]5CC[C@H](N5)[C@H]3CO4)c12]

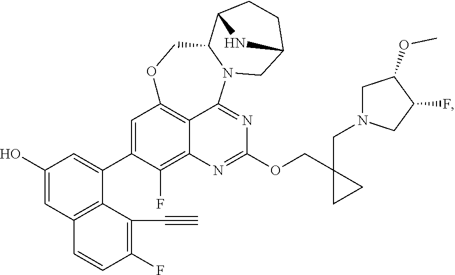 [SMILES: C#Cc1c(F)ccc2cc(O)cc(-c3cc4c5c(nc(OCC6(CN7C[C@@H](F)[C@@H](OC)C7)CC6)nc5c3F)N3C[C@H]5CC[C@H](N5)[C@H]3CO4)c12]

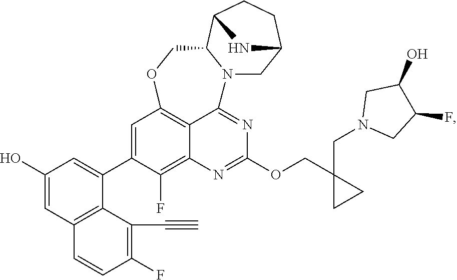 [SMILES: C#Cc1c(F)ccc2cc(O)cc(-c3cc4c5c(nc(OCC6(CN7C[C@@H](O)[C@@H](F)C7)CC6)nc5c3F)N3C[C@H]5CC[C@H](N5)[C@H]3CO4)c12]

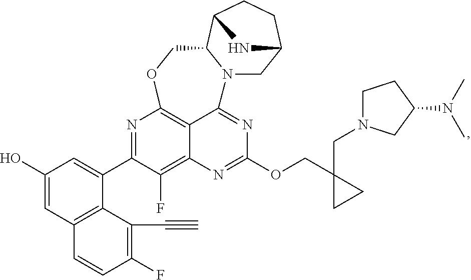 [SMILES: C#Cc1c(F)ccc2cc(O)cc(-c3nc4c5c(nc(OCC6(CN7CC[C@H](N(C)C)C7)CC6)nc5c3F)N3C[C@H]5CC[C@H](N5)[C@H]3CO4)c12]

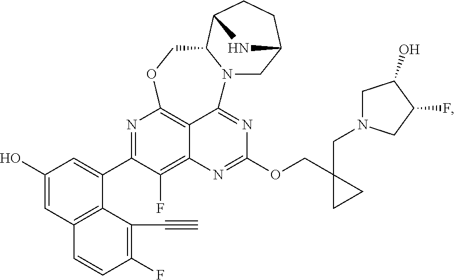 [SMILES: C#Cc1c(F)ccc2cc(O)cc(-c3nc4c5c(nc(OCC6(CN7C[C@@H](F)[C@@H](O)C7)CC6)nc5c3F)N3C[C@H]5CC[C@H](N5)[C@H]3CO4)c12]

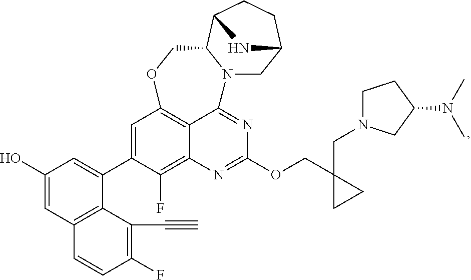 [SMILES: C#Cc1c(F)ccc2cc(O)cc(-c3cc4c5c(nc(OCC6(CN7CC[C@H](N(C)C)C7)CC6)nc5c3F)N3C[C@H]5CC[C@H](N5)[C@H]3CO4)c12]

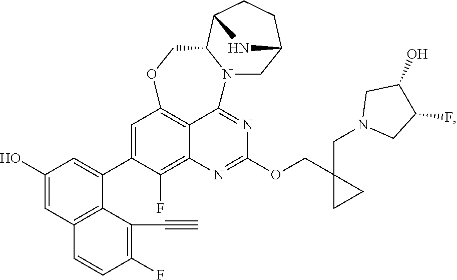 [SMILES: C#Cc1c(F)ccc2cc(O)cc(-c3cc4c5c(nc(OCC6(CN7C[C@@H](F)[C@@H](O)C7)CC6)nc5c3F)N3C[C@H]5CC[C@H](N5)[C@H]3CO4)c12]

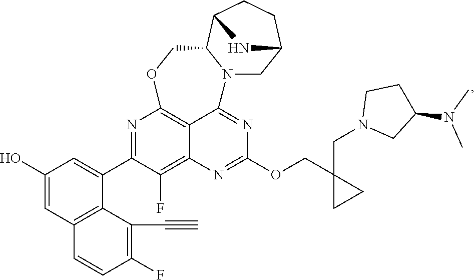 [SMILES: C#Cc1c(F)ccc2cc(O)cc(-c3nc4c5c(nc(OCC6(CN7CC[C@@H](N(C)C)C7)CC6)nc5c3F)N3C[C@H]5CC[C@H](N5)[C@H]3CO4)c12]

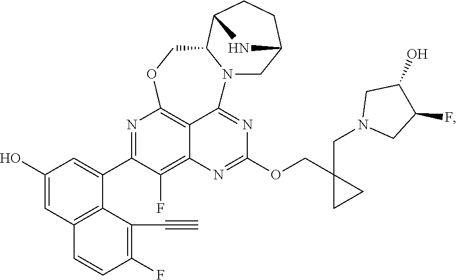 [SMILES: C#Cc1c(F)ccc2cc(O)cc(-c3nc4c5c(nc(OCC6(CN7C[C@H](O)[C@@H](F)C7)CC6)nc5c3F)N3C[C@H]5CC[C@H](N5)[C@H]3CO4)c12]

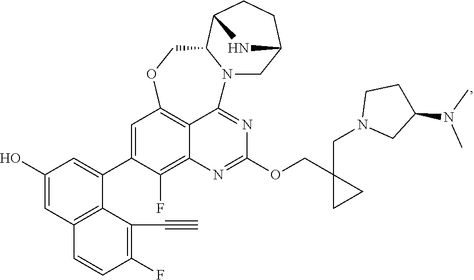 [SMILES: C#Cc1c(F)ccc2cc(O)cc(-c3cc4c5c(nc(OCC6(CN7CC[C@@H](N(C)C)C7)CC6)nc5c3F)N3C[C@H]5CC[C@H](N5)[C@H]3CO4)c12]

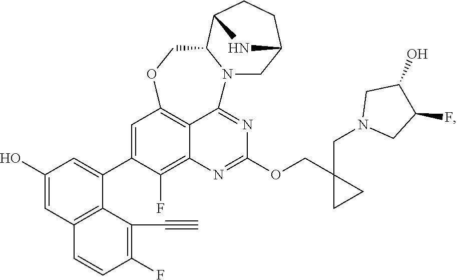 [SMILES: C#Cc1c(F)ccc2cc(O)cc(-c3cc4c5c(nc(OCC6(CN7C[C@H](O)[C@@H](F)C7)CC6)nc5c3F)N3C[C@H]5CC[C@H](N5)[C@H]3CO4)c12]

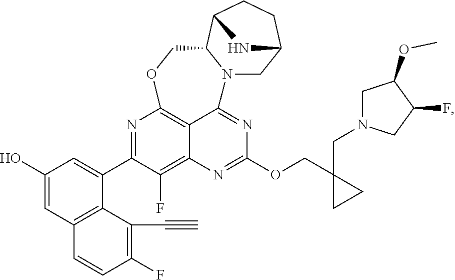 [SMILES: C#Cc1c(F)ccc2cc(O)cc(-c3nc4c5c(nc(OCC6(CN7C[C@H](F)[C@H](OC)C7)CC6)nc5c3F)N3C[C@H]5CC[C@H](N5)[C@H]3CO4)c12]

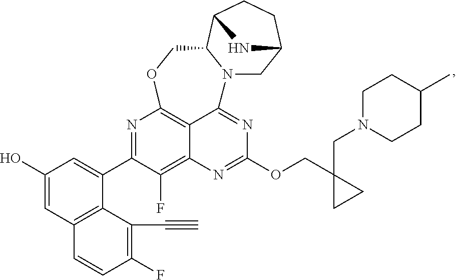 [SMILES: C#Cc1c(F)ccc2cc(O)cc(-c3nc4c5c(nc(OCC6(CN7CCC(C)CC7)CC6)nc5c3F)N3C[C@H]5CC[C@H](N5)[C@H]3CO4)c12]

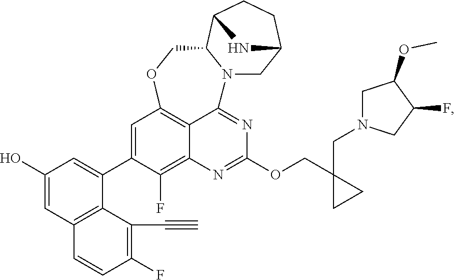 [SMILES: C#Cc1c(F)ccc2cc(O)cc(-c3cc4c5c(nc(OCC6(CN7C[C@H](F)[C@H](OC)C7)CC6)nc5c3F)N3C[C@H]5CC[C@H](N5)[C@H]3CO4)c12]

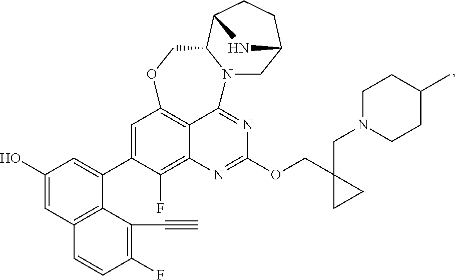 [SMILES: C#Cc1c(F)ccc2cc(O)cc(-c3cc4c5c(nc(OCC6(CN7CCC(C)CC7)CC6)nc5c3F)N3C[C@H]5CC[C@H](N5)[C@H]3CO4)c12]

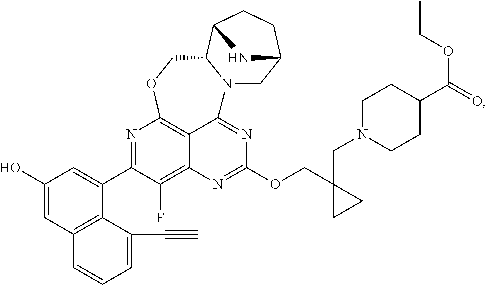 [SMILES: C#Cc1cccc2cc(O)cc(-c3nc4c5c(nc(OCC6(CN7CCC(C(=O)OCC)CC7)CC6)nc5c3F)N3C[C@H]5CC[C@H](N5)[C@H]3CO4)c12]

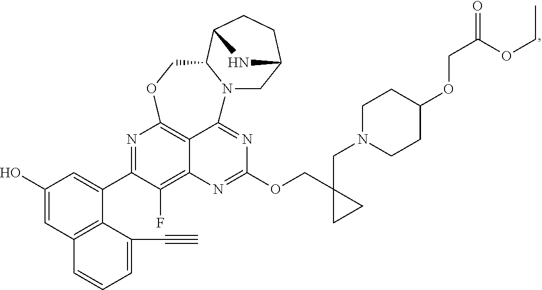 [SMILES: C#Cc1cccc2cc(O)cc(-c3nc4c5c(nc(OCC6(CN7CCC(OCC(=O)OCC)CC7)CC6)nc5c3F)N3C[C@H]5CC[C@H](N5)[C@H]3CO4)c12]

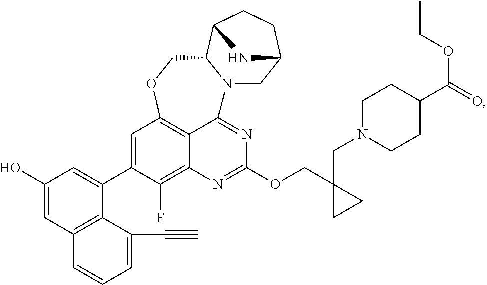 [SMILES: C#Cc1cccc2cc(O)cc(-c3cc4c5c(nc(OCC6(CN7CCC(C(=O)OCC)CC7)CC6)nc5c3F)N3C[C@H]5CC[C@H](N5)[C@H]3CO4)c12]

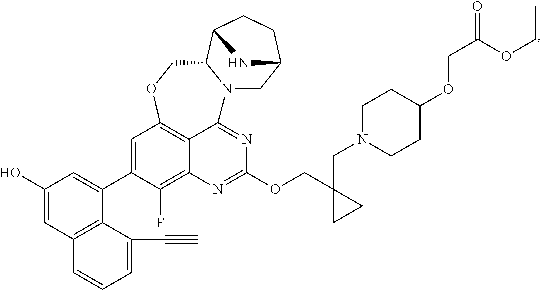 [SMILES: C#Cc1cccc2cc(O)cc(-c3cc4c5c(nc(OCC6(CN7CCC(OCC(=O)OCC)CC7)CC6)nc5c3F)N3C[C@H]5CC[C@H](N5)[C@H]3CO4)c12]

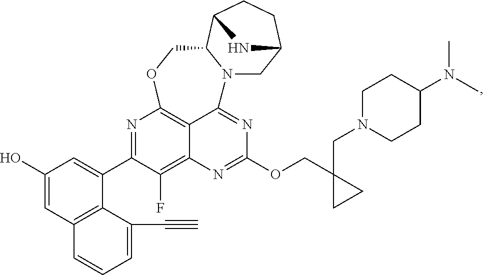 [SMILES: C#Cc1cccc2cc(O)cc(-c3nc4c5c(nc(OCC6(CN7CCC(N(C)C)CC7)CC6)nc5c3F)N3C[C@H]5CC[C@H](N5)[C@H]3CO4)c12]

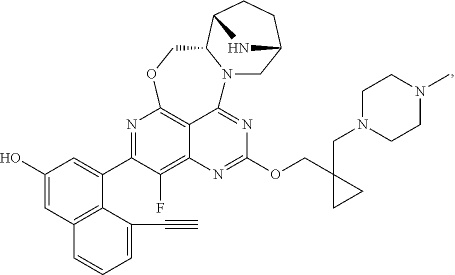 [SMILES: C#Cc1cccc2cc(O)cc(-c3nc4c5c(nc(OCC6(CN7CCN(C)CC7)CC6)nc5c3F)N3C[C@H]5CC[C@H](N5)[C@H]3CO4)c12]

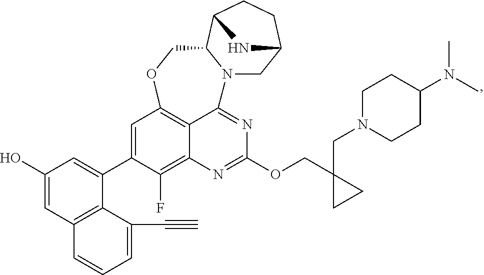 [SMILES: C#Cc1cccc2cc(O)cc(-c3cc4c5c(nc(OCC6(CN7CCC(N(C)C)CC7)CC6)nc5c3F)N3C[C@H]5CC[C@H](N5)[C@H]3CO4)c12]

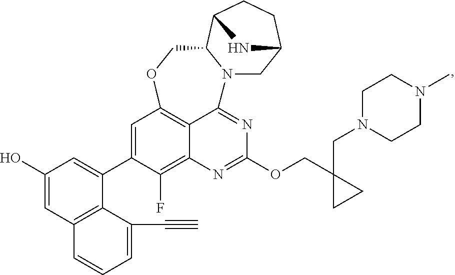 [SMILES: C#Cc1cccc2cc(O)cc(-c3cc4c5c(nc(OCC6(CN7CCN(C)CC7)CC6)nc5c3F)N3C[C@H]5CC[C@H](N5)[C@H]3CO4)c12]

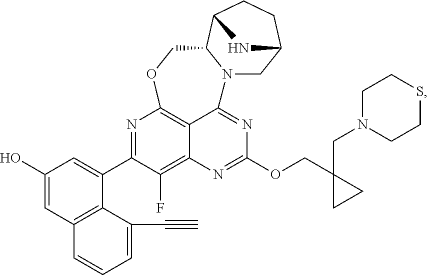 [SMILES: C#Cc1cccc2cc(O)cc(-c3nc4c5c(nc(OCC6(CN7CCSCC7)CC6)nc5c3F)N3C[C@H]5CC[C@H](N5)[C@H]3CO4)c12]

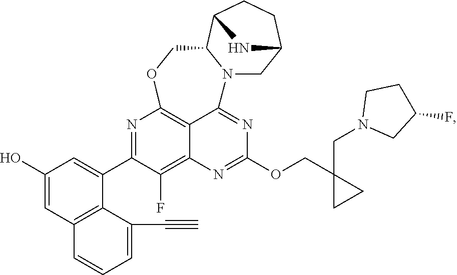 [SMILES: C#Cc1cccc2cc(O)cc(-c3nc4c5c(nc(OCC6(CN7CC[C@H](F)C7)CC6)nc5c3F)N3C[C@H]5CC[C@H](N5)[C@H]3CO4)c12]

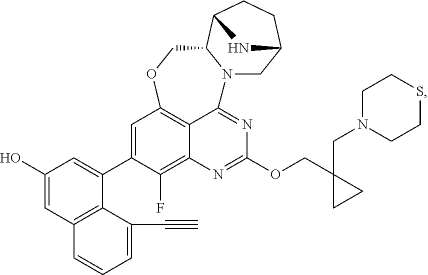 [SMILES: C#Cc1cccc2cc(O)cc(-c3cc4c5c(nc(OCC6(CN7CCSCC7)CC6)nc5c3F)N3C[C@H]5CC[C@H](N5)[C@H]3CO4)c12]

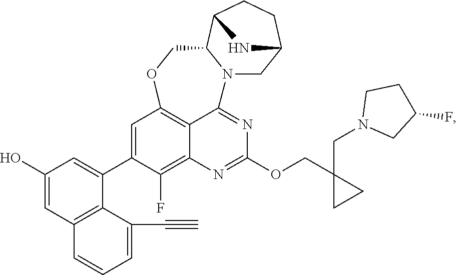 [SMILES: C#Cc1cccc2cc(O)cc(-c3cc4c5c(nc(OCC6(CN7CC[C@H](F)C7)CC6)nc5c3F)N3C[C@H]5CC[C@H](N5)[C@H]3CO4)c12]

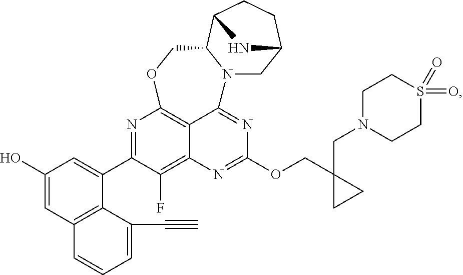 [SMILES: C#Cc1cccc2cc(O)cc(-c3nc4c5c(nc(OCC6(CN7CCS(=O)(=O)CC7)CC6)nc5c3F)N3C[C@H]5CC[C@H](N5)[C@H]3CO4)c12]

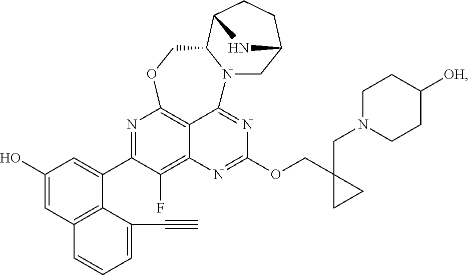 [SMILES: C#Cc1cccc2cc(O)cc(-c3nc4c5c(nc(OCC6(CN7CCC(O)CC7)CC6)nc5c3F)N3C[C@H]5CC[C@H](N5)[C@H]3CO4)c12]

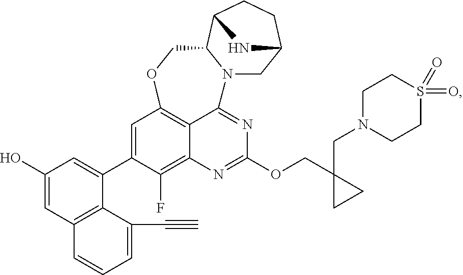 [SMILES: C#Cc1cccc2cc(O)cc(-c3cc4c5c(nc(OCC6(CN7CCS(=O)(=O)CC7)CC6)nc5c3F)N3C[C@H]5CC[C@H](N5)[C@H]3CO4)c12]

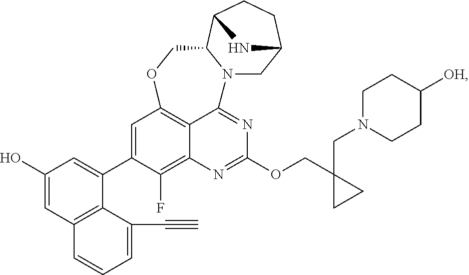 [SMILES: C#Cc1cccc2cc(O)cc(-c3cc4c5c(nc(OCC6(CN7CCC(O)CC7)CC6)nc5c3F)N3C[C@H]5CC[C@H](N5)[C@H]3CO4)c12]

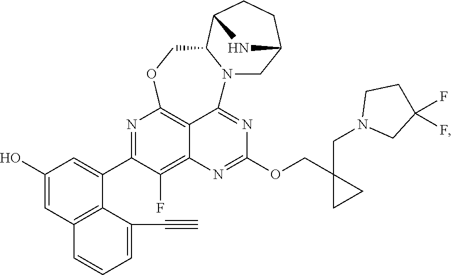 [SMILES: C#Cc1cccc2cc(O)cc(-c3nc4c5c(nc(OCC6(CN7CCC(F)(F)C7)CC6)nc5c3F)N3C[C@H]5CC[C@H](N5)[C@H]3CO4)c12]

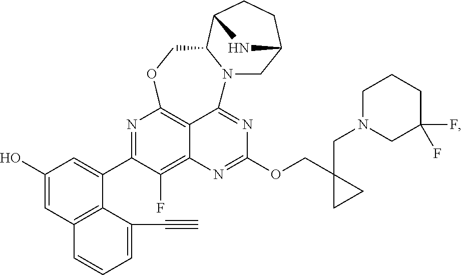 [SMILES: C#Cc1cccc2cc(O)cc(-c3nc4c5c(nc(OCC6(CN7CCCC(F)(F)C7)CC6)nc5c3F)N3C[C@H]5CC[C@H](N5)[C@H]3CO4)c12]

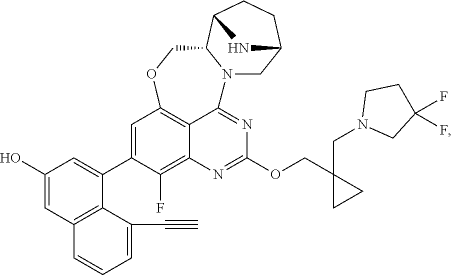 [SMILES: C#Cc1cccc2cc(O)cc(-c3cc4c5c(nc(OCC6(CN7CCC(F)(F)C7)CC6)nc5c3F)N3C[C@H]5CC[C@H](N5)[C@H]3CO4)c12]

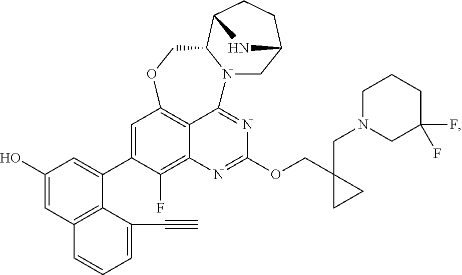 [SMILES: C#Cc1cccc2cc(O)cc(-c3cc4c5c(nc(OCC6(CN7CCCC(F)(F)C7)CC6)nc5c3F)N3C[C@H]5CC[C@H](N5)[C@H]3CO4)c12]

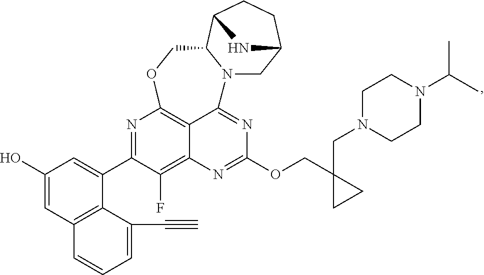 [SMILES: C#Cc1cccc2cc(O)cc(-c3nc4c5c(nc(OCC6(CN7CCN(C(C)C)CC7)CC6)nc5c3F)N3C[C@H]5CC[C@H](N5)[C@H]3CO4)c12]

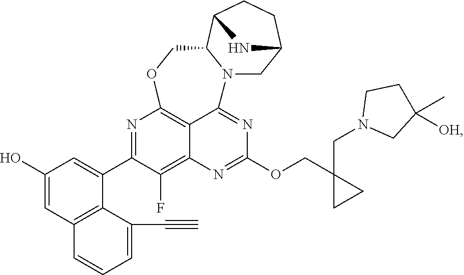 [SMILES: C#Cc1cccc2cc(O)cc(-c3nc4c5c(nc(OCC6(CN7CCC(C)(O)C7)CC6)nc5c3F)N3C[C@H]5CC[C@H](N5)[C@H]3CO4)c12]

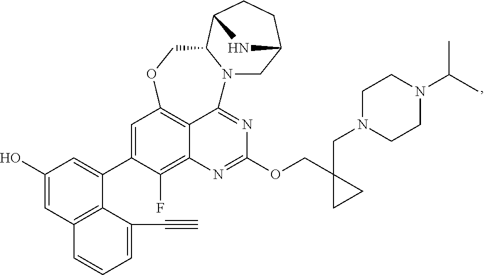 [SMILES: C#Cc1cccc2cc(O)cc(-c3cc4c5c(nc(OCC6(CN7CCN(C(C)C)CC7)CC6)nc5c3F)N3C[C@H]5CC[C@H](N5)[C@H]3CO4)c12]

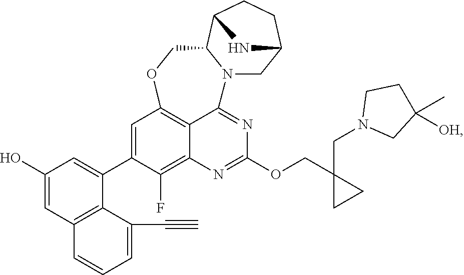 [SMILES: C#Cc1cccc2cc(O)cc(-c3cc4c5c(nc(OCC6(CN7CCC(C)(O)C7)CC6)nc5c3F)N3C[C@H]5CC[C@H](N5)[C@H]3CO4)c12]

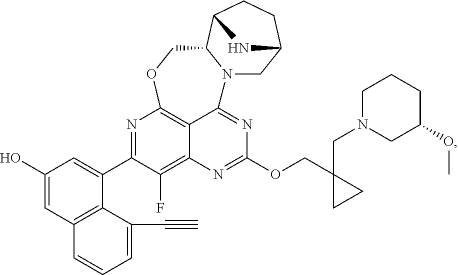 [SMILES: C#Cc1cccc2cc(O)cc(-c3nc4c5c(nc(OCC6(CN7CCC[C@H](OC)C7)CC6)nc5c3F)N3C[C@H]5CC[C@H](N5)[C@H]3CO4)c12]

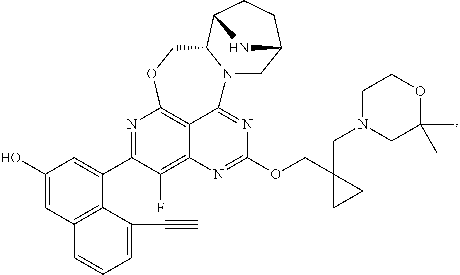 [SMILES: C#Cc1cccc2cc(O)cc(-c3nc4c5c(nc(OCC6(CN7CCOC(C)(C)C7)CC6)nc5c3F)N3C[C@H]5CC[C@H](N5)[C@H]3CO4)c12]

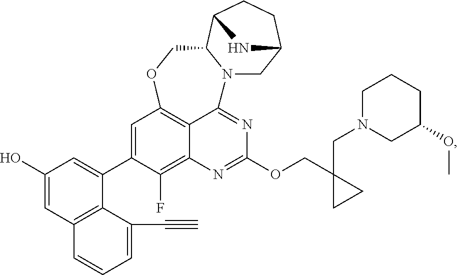 [SMILES: C#Cc1cccc2cc(O)cc(-c3cc4c5c(nc(OCC6(CN7CCC[C@H](OC)C7)CC6)nc5c3F)N3C[C@H]5CC[C@H](N5)[C@H]3CO4)c12]

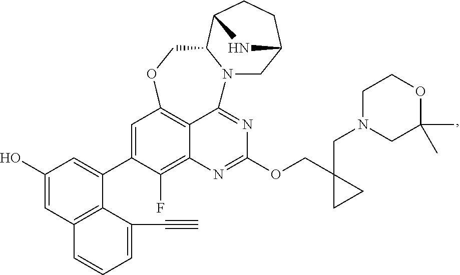 [SMILES: C#Cc1cccc2cc(O)cc(-c3cc4c5c(nc(OCC6(CN7CCOC(C)(C)C7)CC6)nc5c3F)N3C[C@H]5CC[C@H](N5)[C@H]3CO4)c12]

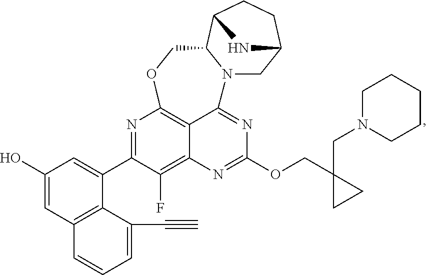 [SMILES: C#Cc1cccc2cc(O)cc(-c3nc4c5c(nc(OCC6(CN7CCCCC7)CC6)nc5c3F)N3C[C@H]5CC[C@H](N5)[C@H]3CO4)c12]

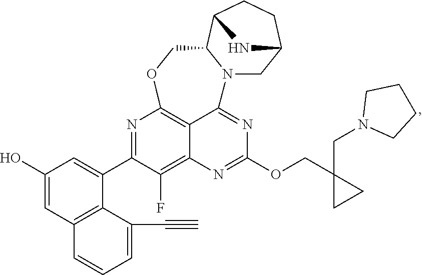 [SMILES: C#Cc1cccc2cc(O)cc(-c3nc4c5c(nc(OCC6(CN7CCCC7)CC6)nc5c3F)N3C[C@H]5CC[C@H](N5)[C@H]3CO4)c12]

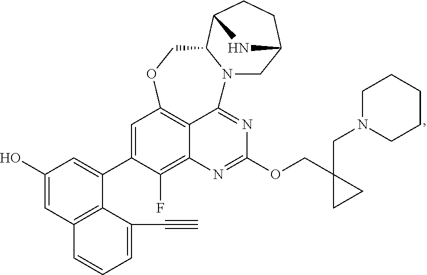 [SMILES: C#Cc1cccc2cc(O)cc(-c3cc4c5c(nc(OCC6(CN7CCCCC7)CC6)nc5c3F)N3C[C@H]5CC[C@H](N5)[C@H]3CO4)c12]

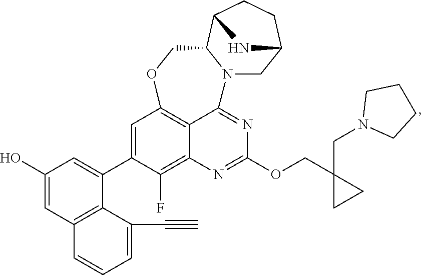 [SMILES: C#Cc1cccc2cc(O)cc(-c3cc4c5c(nc(OCC6(CN7CCCC7)CC6)nc5c3F)N3C[C@H]5CC[C@H](N5)[C@H]3CO4)c12]

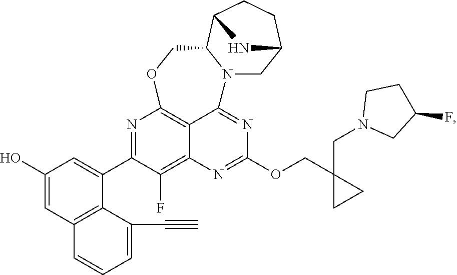 [SMILES: C#Cc1cccc2cc(O)cc(-c3nc4c5c(nc(OCC6(CN7CC[C@@H](F)C7)CC6)nc5c3F)N3C[C@H]5CC[C@H](N5)[C@H]3CO4)c12]

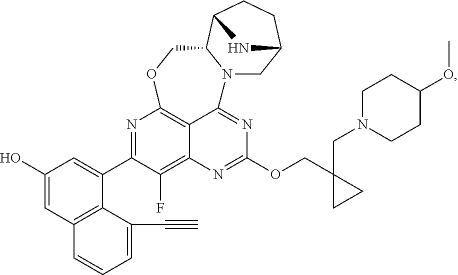 [SMILES: C#Cc1cccc2cc(O)cc(-c3nc4c5c(nc(OCC6(CN7CCC(OC)CC7)CC6)nc5c3F)N3C[C@H]5CC[C@H](N5)[C@H]3CO4)c12]

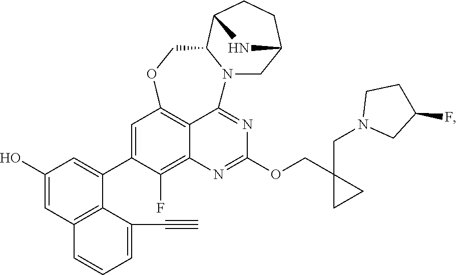 [SMILES: C#Cc1cccc2cc(O)cc(-c3cc4c5c(nc(OCC6(CN7CC[C@@H](F)C7)CC6)nc5c3F)N3C[C@H]5CC[C@H](N5)[C@H]3CO4)c12]

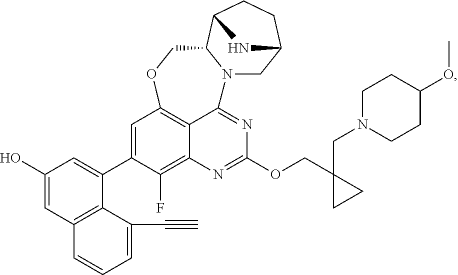 [SMILES: C#Cc1cccc2cc(O)cc(-c3cc4c5c(nc(OCC6(CN7CCC(OC)CC7)CC6)nc5c3F)N3C[C@H]5CC[C@H](N5)[C@H]3CO4)c12]

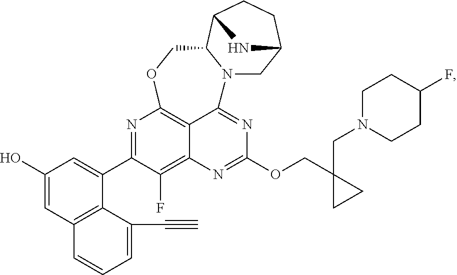 [SMILES: C#Cc1cccc2cc(O)cc(-c3nc4c5c(nc(OCC6(CN7CCC(F)CC7)CC6)nc5c3F)N3C[C@H]5CC[C@H](N5)[C@H]3CO4)c12]

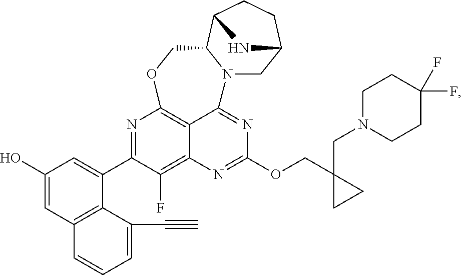 [SMILES: C#Cc1cccc2cc(O)cc(-c3nc4c5c(nc(OCC6(CN7CCC(F)(F)CC7)CC6)nc5c3F)N3C[C@H]5CC[C@H](N5)[C@H]3CO4)c12]

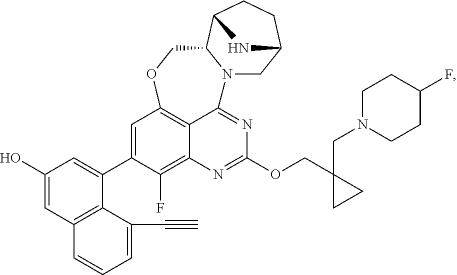 [SMILES: C#Cc1cccc2cc(O)cc(-c3cc4c5c(nc(OCC6(CN7CCC(F)CC7)CC6)nc5c3F)N3C[C@H]5CC[C@H](N5)[C@H]3CO4)c12]

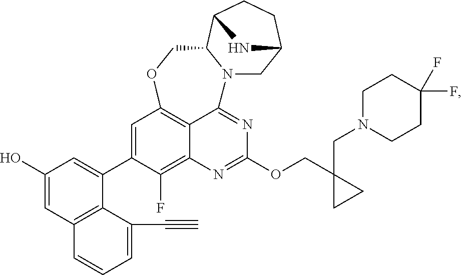 [SMILES: C#Cc1cccc2cc(O)cc(-c3cc4c5c(nc(OCC6(CN7CCC(F)(F)CC7)CC6)nc5c3F)N3C[C@H]5CC[C@H](N5)[C@H]3CO4)c12]

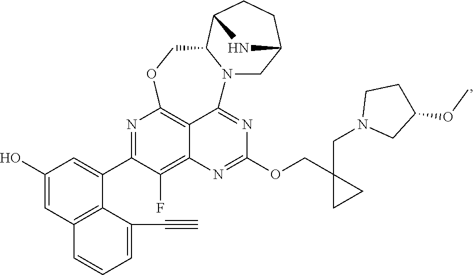 [SMILES: C#Cc1cccc2cc(O)cc(-c3nc4c5c(nc(OCC6(CN7CC[C@H](OC)C7)CC6)nc5c3F)N3C[C@H]5CC[C@H](N5)[C@H]3CO4)c12]

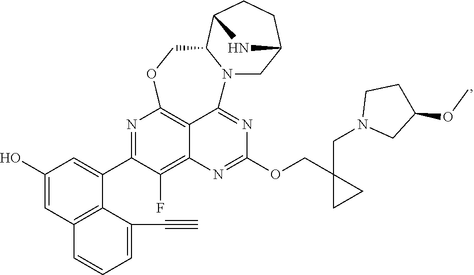 [SMILES: C#Cc1cccc2cc(O)cc(-c3nc4c5c(nc(OCC6(CN7CC[C@@H](OC)C7)CC6)nc5c3F)N3C[C@H]5CC[C@H](N5)[C@H]3CO4)c12]

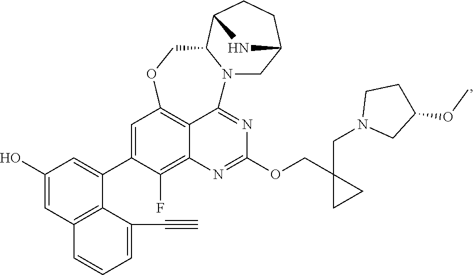 [SMILES: C#Cc1cccc2cc(O)cc(-c3cc4c5c(nc(OCC6(CN7CC[C@H](OC)C7)CC6)nc5c3F)N3C[C@H]5CC[C@H](N5)[C@H]3CO4)c12]

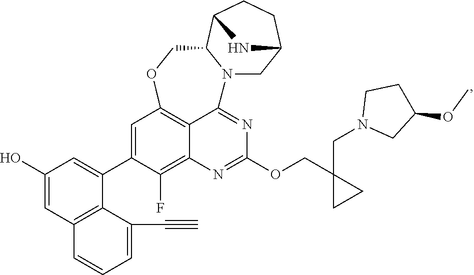 [SMILES: C#Cc1cccc2cc(O)cc(-c3cc4c5c(nc(OCC6(CN7CC[C@@H](OC)C7)CC6)nc5c3F)N3C[C@H]5CC[C@H](N5)[C@H]3CO4)c12]

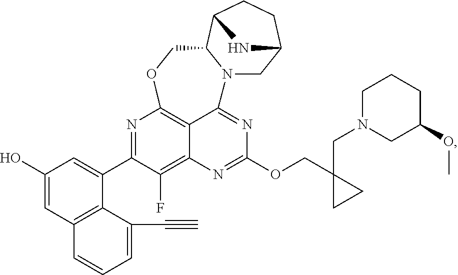 [SMILES: C#Cc1cccc2cc(O)cc(-c3nc4c5c(nc(OCC6(CN7CCC[C@@H](OC)C7)CC6)nc5c3F)N3C[C@H]5CC[C@H](N5)[C@H]3CO4)c12]

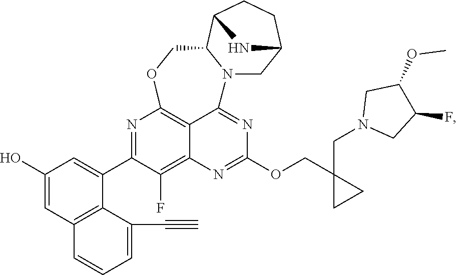 [SMILES: C#Cc1cccc2cc(O)cc(-c3nc4c5c(nc(OCC6(CN7C[C@H](OC)[C@@H](F)C7)CC6)nc5c3F)N3C[C@H]5CC[C@H](N5)[C@H]3CO4)c12]

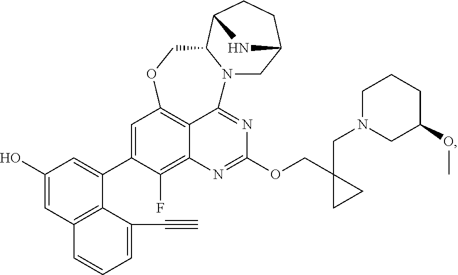 [SMILES: C#Cc1cccc2cc(O)cc(-c3cc4c5c(nc(OCC6(CN7CCC[C@@H](OC)C7)CC6)nc5c3F)N3C[C@H]5CC[C@H](N5)[C@H]3CO4)c12]

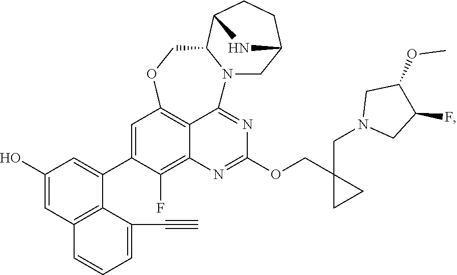 [SMILES: C#Cc1cccc2cc(O)cc(-c3cc4c5c(nc(OCC6(CN7C[C@H](OC)[C@@H](F)C7)CC6)nc5c3F)N3C[C@H]5CC[C@H](N5)[C@H]3CO4)c12]

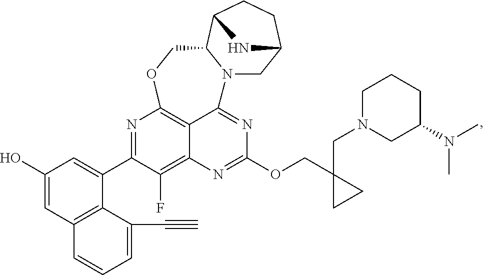 [SMILES: C#Cc1cccc2cc(O)cc(-c3nc4c5c(nc(OCC6(CN7CCC[C@H](N(C)C)C7)CC6)nc5c3F)N3C[C@H]5CC[C@H](N5)[C@H]3CO4)c12]

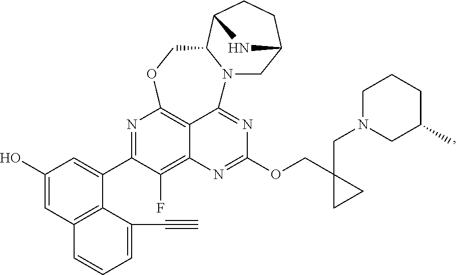 [SMILES: C#Cc1cccc2cc(O)cc(-c3nc4c5c(nc(OCC6(CN7CCC[C@H](C)C7)CC6)nc5c3F)N3C[C@H]5CC[C@H](N5)[C@H]3CO4)c12]

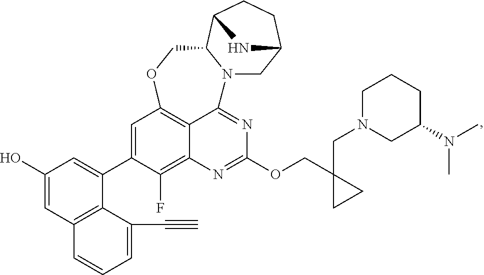 [SMILES: C#Cc1cccc2cc(O)cc(-c3cc4c5c(nc(OCC6(CN7CCC[C@H](N(C)C)C7)CC6)nc5c3F)N3C[C@H]5CC[C@H](N5)[C@H]3CO4)c12]

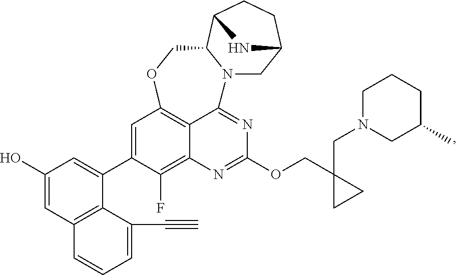 [SMILES: C#Cc1cccc2cc(O)cc(-c3cc4c5c(nc(OCC6(CN7CCC[C@H](C)C7)CC6)nc5c3F)N3C[C@H]5CC[C@H](N5)[C@H]3CO4)c12]

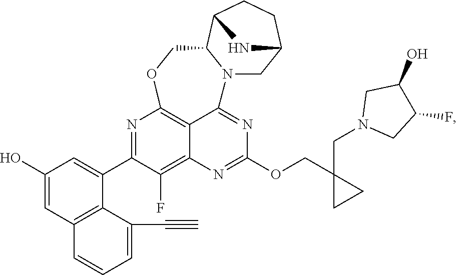 [SMILES: C#Cc1cccc2cc(O)cc(-c3nc4c5c(nc(OCC6(CN7C[C@@H](O)[C@H](F)C7)CC6)nc5c3F)N3C[C@H]5CC[C@H](N5)[C@H]3CO4)c12]

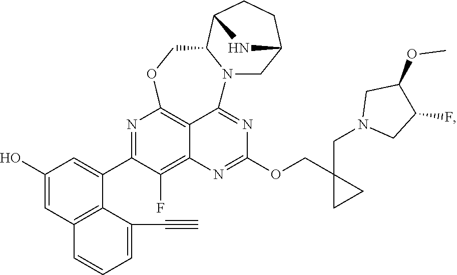 [SMILES: C#Cc1cccc2cc(O)cc(-c3nc4c5c(nc(OCC6(CN7C[C@@H](F)[C@H](OC)C7)CC6)nc5c3F)N3C[C@H]5CC[C@H](N5)[C@H]3CO4)c12]

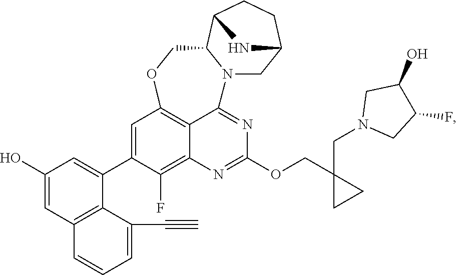 [SMILES: C#Cc1cccc2cc(O)cc(-c3cc4c5c(nc(OCC6(CN7C[C@@H](O)[C@H](F)C7)CC6)nc5c3F)N3C[C@H]5CC[C@H](N5)[C@H]3CO4)c12]

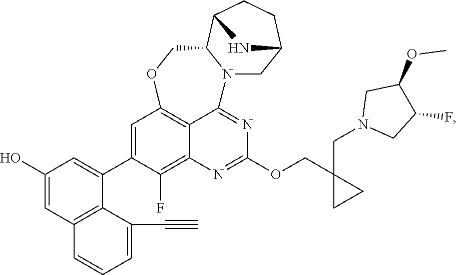 [SMILES: C#Cc1cccc2cc(O)cc(-c3cc4c5c(nc(OCC6(CN7C[C@@H](F)[C@H](OC)C7)CC6)nc5c3F)N3C[C@H]5CC[C@H](N5)[C@H]3CO4)c12]

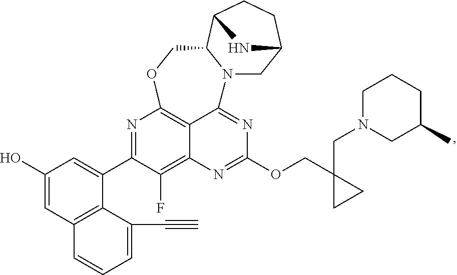 [SMILES: C#Cc1cccc2cc(O)cc(-c3nc4c5c(nc(OCC6(CN7CCC[C@@H](C)C7)CC6)nc5c3F)N3C[C@H]5CC[C@H](N5)[C@H]3CO4)c12]

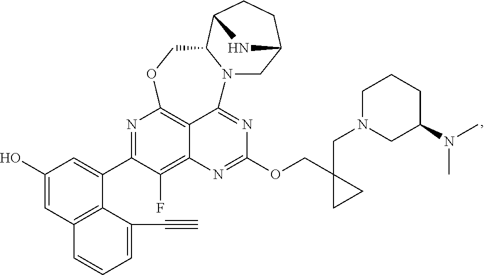 [SMILES: C#Cc1cccc2cc(O)cc(-c3nc4c5c(nc(OCC6(CN7CCC[C@@H](N(C)C)C7)CC6)nc5c3F)N3C[C@H]5CC[C@H](N5)[C@H]3CO4)c12]

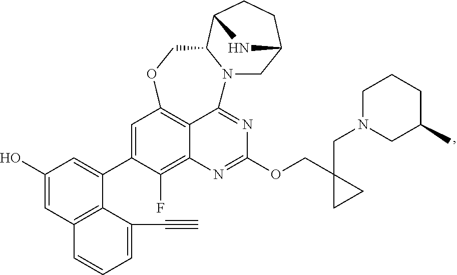 [SMILES: C#Cc1cccc2cc(O)cc(-c3cc4c5c(nc(OCC6(CN7CCC[C@@H](C)C7)CC6)nc5c3F)N3C[C@H]5CC[C@H](N5)[C@H]3CO4)c12]

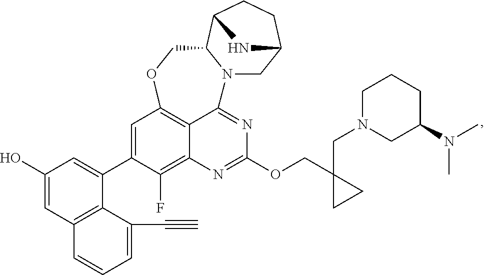 [SMILES: C#Cc1cccc2cc(O)cc(-c3cc4c5c(nc(OCC6(CN7CCC[C@@H](N(C)C)C7)CC6)nc5c3F)N3C[C@H]5CC[C@H](N5)[C@H]3CO4)c12]

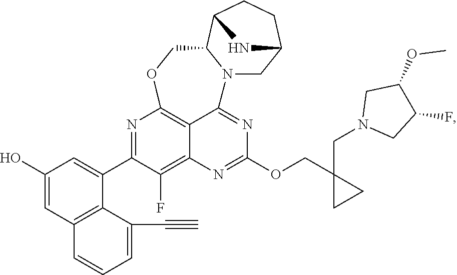 [SMILES: C#Cc1cccc2cc(O)cc(-c3nc4c5c(nc(OCC6(CN7C[C@@H](F)[C@@H](OC)C7)CC6)nc5c3F)N3C[C@H]5CC[C@H](N5)[C@H]3CO4)c12]

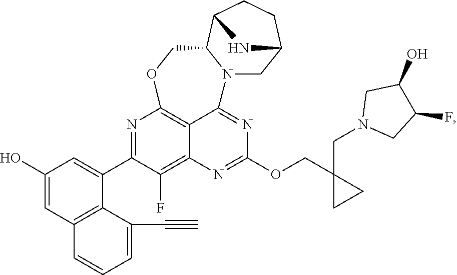 [SMILES: C#Cc1cccc2cc(O)cc(-c3nc4c5c(nc(OCC6(CN7C[C@@H](O)[C@@H](F)C7)CC6)nc5c3F)N3C[C@H]5CC[C@H](N5)[C@H]3CO4)c12]

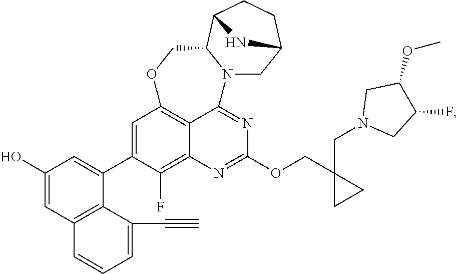 [SMILES: C#Cc1cccc2cc(O)cc(-c3cc4c5c(nc(OCC6(CN7C[C@@H](F)[C@@H](OC)C7)CC6)nc5c3F)N3C[C@H]5CC[C@H](N5)[C@H]3CO4)c12]

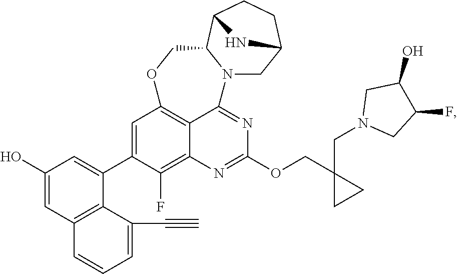 [SMILES: C#Cc1cccc2cc(O)cc(-c3cc4c5c(nc(OCC6(CN7C[C@@H](O)[C@@H](F)C7)CC6)nc5c3F)N3C[C@H]5CC[C@H](N5)[C@H]3CO4)c12]

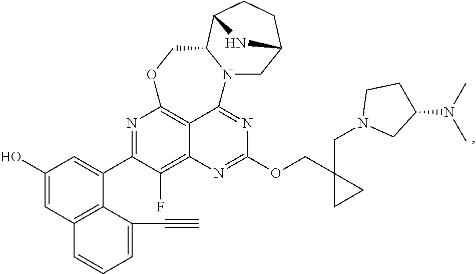 [SMILES: C#Cc1cccc2cc(O)cc(-c3nc4c5c(nc(OCC6(CN7CC[C@H](N(C)C)C7)CC6)nc5c3F)N3C[C@H]5CC[C@H](N5)[C@H]3CO4)c12]

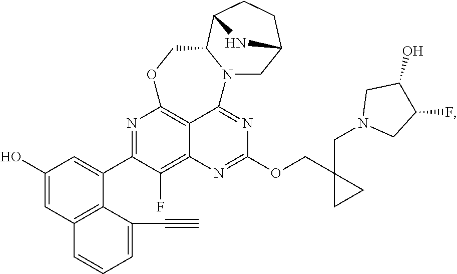 [SMILES: C#Cc1cccc2cc(O)cc(-c3nc4c5c(nc(OCC6(CN7C[C@@H](F)[C@@H](O)C7)CC6)nc5c3F)N3C[C@H]5CC[C@H](N5)[C@H]3CO4)c12]

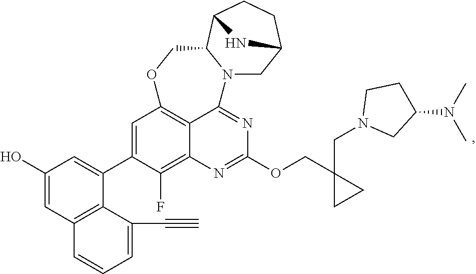 [SMILES: C#Cc1cccc2cc(O)cc(-c3cc4c5c(nc(OCC6(CN7CC[C@H](N(C)C)C7)CC6)nc5c3F)N3C[C@H]5CC[C@H](N5)[C@H]3CO4)c12]

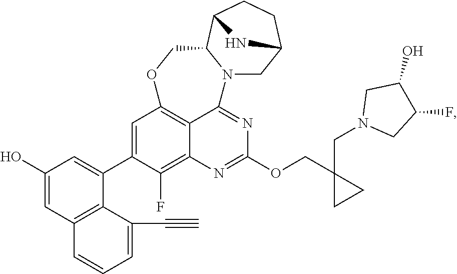 [SMILES: C#Cc1cccc2cc(O)cc(-c3cc4c5c(nc(OCC6(CN7C[C@@H](F)[C@@H](O)C7)CC6)nc5c3F)N3C[C@H]5CC[C@H](N5)[C@H]3CO4)c12]

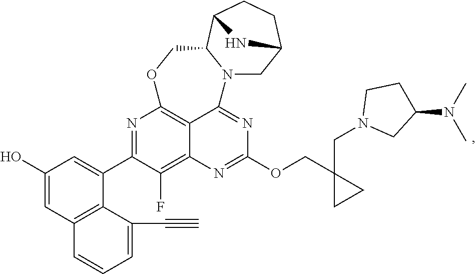 [SMILES: C#Cc1cccc2cc(O)cc(-c3nc4c5c(nc(OCC6(CN7CC[C@@H](N(C)C)C7)CC6)nc5c3F)N3C[C@H]5CC[C@H](N5)[C@H]3CO4)c12]

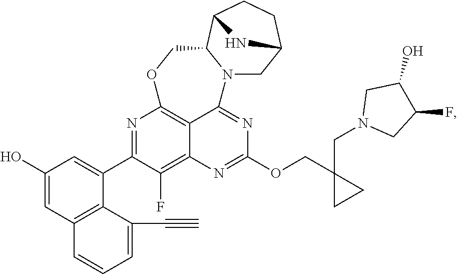 [SMILES: C#Cc1cccc2cc(O)cc(-c3nc4c5c(nc(OCC6(CN7C[C@H](O)[C@@H](F)C7)CC6)nc5c3F)N3C[C@H]5CC[C@H](N5)[C@H]3CO4)c12]

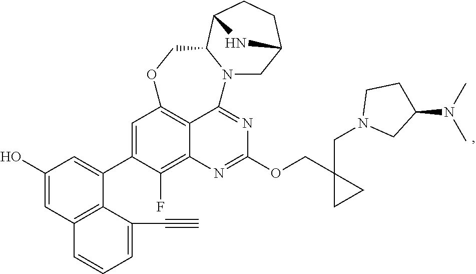 [SMILES: C#Cc1cccc2cc(O)cc(-c3cc4c5c(nc(OCC6(CN7CC[C@@H](N(C)C)C7)CC6)nc5c3F)N3C[C@H]5CC[C@H](N5)[C@H]3CO4)c12]

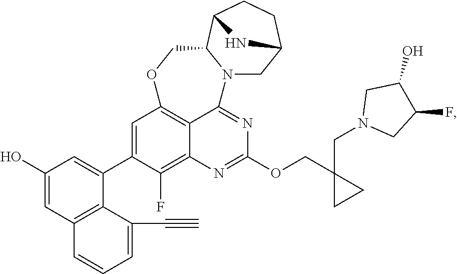 [SMILES: C#Cc1cccc2cc(O)cc(-c3cc4c5c(nc(OCC6(CN7C[C@H](O)[C@@H](F)C7)CC6)nc5c3F)N3C[C@H]5CC[C@H](N5)[C@H]3CO4)c12]

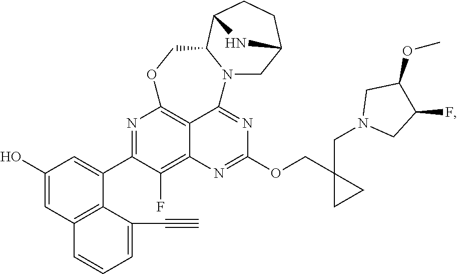 [SMILES: C#Cc1cccc2cc(O)cc(-c3nc4c5c(nc(OCC6(CN7C[C@H](F)[C@H](OC)C7)CC6)nc5c3F)N3C[C@H]5CC[C@H](N5)[C@H]3CO4)c12]

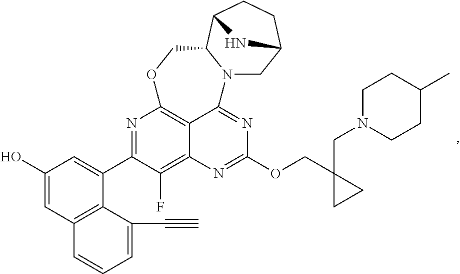 [SMILES: C#Cc1cccc2cc(O)cc(-c3nc4c5c(nc(OCC6(CN7CCC(C)CC7)CC6)nc5c3F)N3C[C@H]5CC[C@H](N5)[C@H]3CO4)c12]

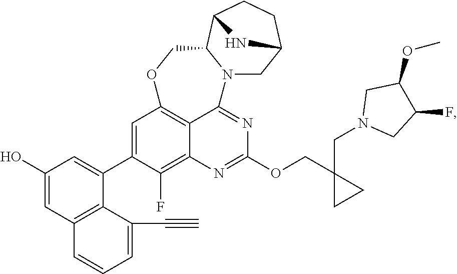 [SMILES: C#Cc1cccc2cc(O)cc(-c3cc4c5c(nc(OCC6(CN7C[C@H](F)[C@H](OC)C7)CC6)nc5c3F)N3C[C@H]5CC[C@H](N5)[C@H]3CO4)c12]

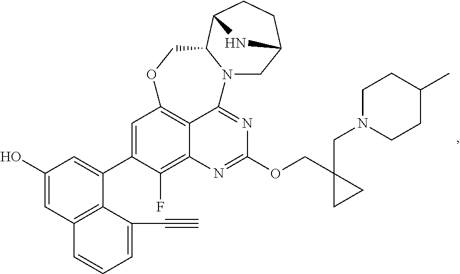 [SMILES: C#Cc1cccc2cc(O)cc(-c3cc4c5c(nc(OCC6(CN7CCC(C)CC7)CC6)nc5c3F)N3C[C@H]5CC[C@H](N5)[C@H]3CO4)c12]

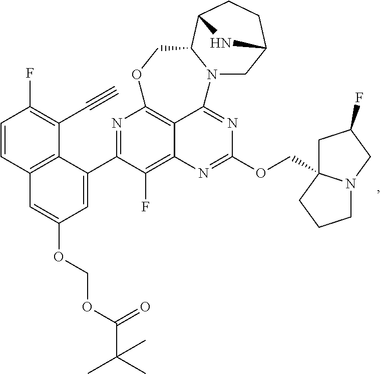 [SMILES: C#Cc1c(F)ccc2cc(OCOC(=O)C(C)(C)C)cc(-c3nc4c5c(nc(OC[C@@]67CCCN6C[C@H](F)C7)nc5c3F)N3C[C@H]5CC[C@H](N5)[C@H]3CO4)c12]

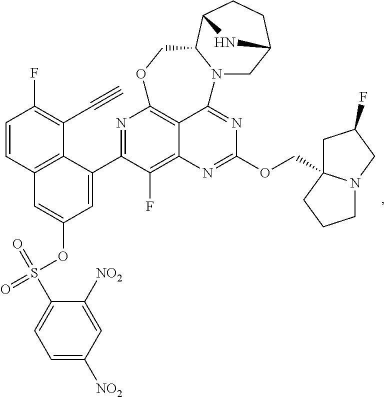 [SMILES: C#Cc1c(F)ccc2cc(OS(=O)(=O)c3ccc([N+](=O)[O-])cc3[N+](=O)[O-])cc(-c3nc4c5c(nc(OC[C@@]67CCCN6C[C@H](F)C7)nc5c3F)N3C[C@H]5CC[C@H](N5)[C@H]3CO4)c12]

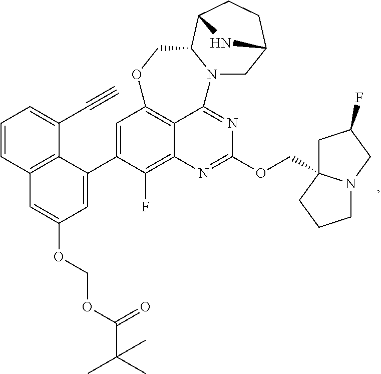 [SMILES: C#Cc1cccc2cc(OCOC(=O)C(C)(C)C)cc(-c3cc4c5c(nc(OC[C@@]67CCCN6C[C@H](F)C7)nc5c3F)N3C[C@H]5CC[C@H](N5)[C@H]3CO4)c12]

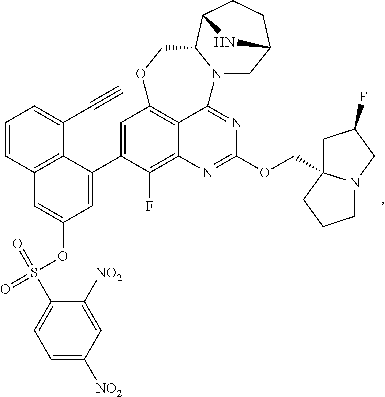 [SMILES: C#Cc1cccc2cc(OS(=O)(=O)c3ccc([N+](=O)[O-])cc3[N+](=O)[O-])cc(-c3cc4c5c(nc(OC[C@@]67CCCN6C[C@H](F)C7)nc5c3F)N3C[C@H]5CC[C@H](N5)[C@H]3CO4)c12]

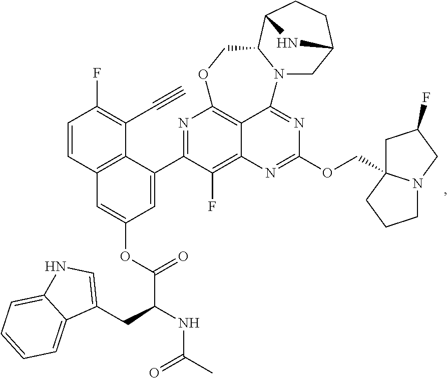 [SMILES: C#Cc1c(F)ccc2cc(OC(=O)[C@H](Cc3c[nH]c4ccccc34)NC(C)=O)cc(-c3nc4c5c(nc(OC[C@@]67CCCN6C[C@H](F)C7)nc5c3F)N3C[C@H]5CC[C@H](N5)[C@H]3CO4)c12]

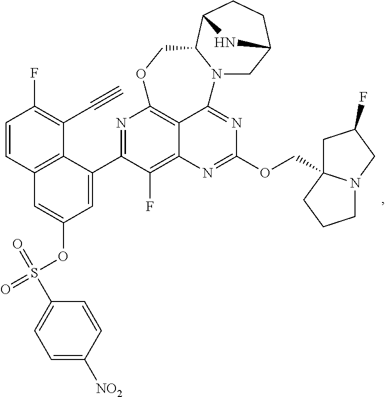 [SMILES: C#Cc1c(F)ccc2cc(OS(=O)(=O)c3ccc([N+](=O)[O-])cc3)cc(-c3nc4c5c(nc(OC[C@@]67CCCN6C[C@H](F)C7)nc5c3F)N3C[C@H]5CC[C@H](N5)[C@H]3CO4)c12]

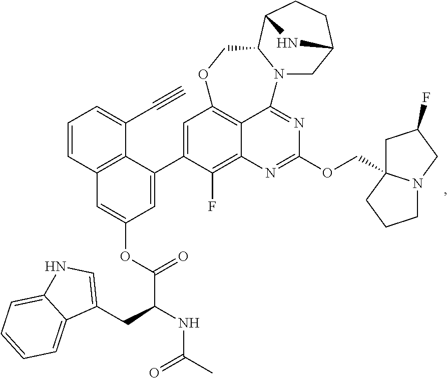 [SMILES: C#Cc1cccc2cc(OC(=O)[C@H](Cc3c[nH]c4ccccc34)NC(C)=O)cc(-c3cc4c5c(nc(OC[C@@]67CCCN6C[C@H](F)C7)nc5c3F)N3C[C@H]5CC[C@H](N5)[C@H]3CO4)c12]

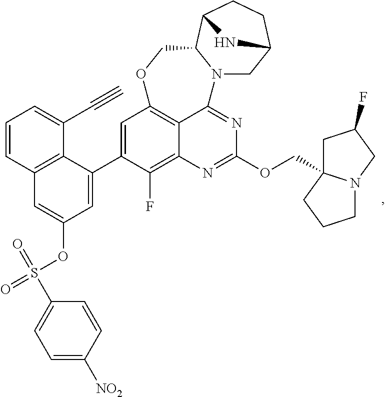 [SMILES: C#Cc1cccc2cc(OS(=O)(=O)c3ccc([N+](=O)[O-])cc3)cc(-c3cc4c5c(nc(OC[C@@]67CCCN6C[C@H](F)C7)nc5c3F)N3C[C@H]5CC[C@H](N5)[C@H]3CO4)c12]

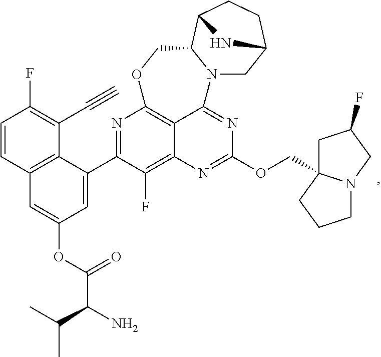 [SMILES: C#Cc1c(F)ccc2cc(OC(=O)[C@@H](N)C(C)C)cc(-c3nc4c5c(nc(OC[C@@]67CCCN6C[C@H](F)C7)nc5c3F)N3C[C@H]5CC[C@H](N5)[C@H]3CO4)c12]

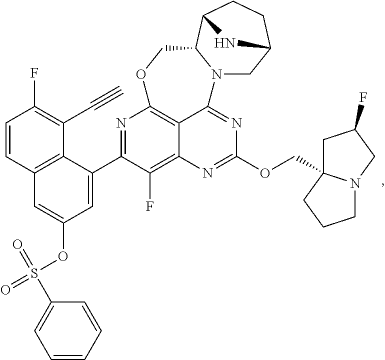 [SMILES: C#Cc1c(F)ccc2cc(OS(=O)(=O)c3ccccc3)cc(-c3nc4c5c(nc(OC[C@@]67CCCN6C[C@H](F)C7)nc5c3F)N3C[C@H]5CC[C@H](N5)[C@H]3CO4)c12]

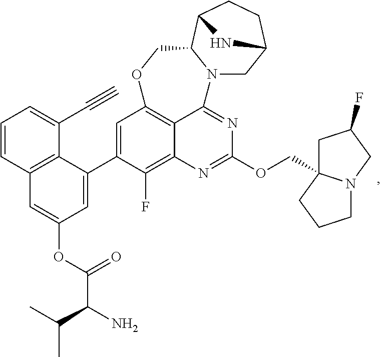 [SMILES: C#Cc1cccc2cc(OC(=O)[C@@H](N)C(C)C)cc(-c3cc4c5c(nc(OC[C@@]67CCCN6C[C@H](F)C7)nc5c3F)N3C[C@H]5CC[C@H](N5)[C@H]3CO4)c12]

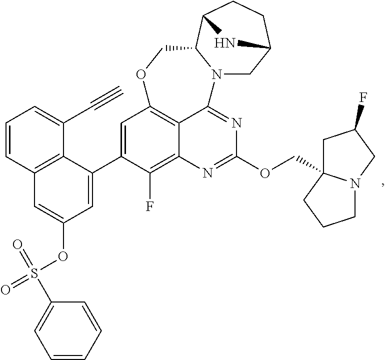 [SMILES: C#Cc1cccc2cc(OS(=O)(=O)c3ccccc3)cc(-c3cc4c5c(nc(OC[C@@]67CCCN6C[C@H](F)C7)nc5c3F)N3C[C@H]5CC[C@H](N5)[C@H]3CO4)c12]

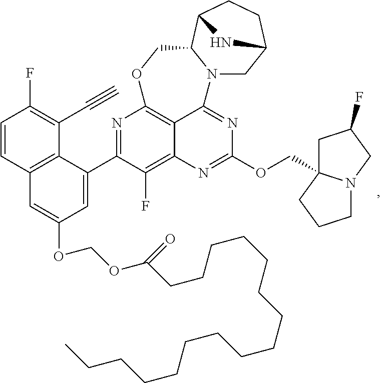 [SMILES: C#Cc1c(F)ccc2cc(OCOC(=O)CCCCCCCCCCCCCCCC)cc(-c3nc4c5c(nc(OC[C@@]67CCCN6C[C@H](F)C7)nc5c3F)N3C[C@H]5CC[C@H](N5)[C@H]3CO4)c12]

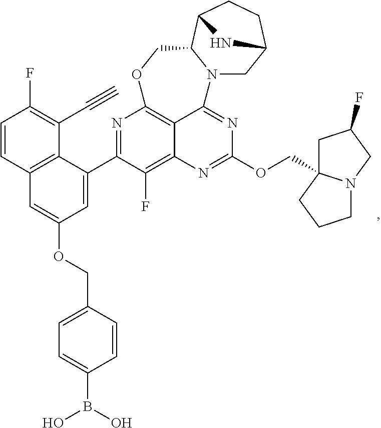 [SMILES: C#Cc1c(F)ccc2cc(OCc3ccc(B(O)O)cc3)cc(-c3nc4c5c(nc(OC[C@@]67CCCN6C[C@H](F)C7)nc5c3F)N3C[C@H]5CC[C@H](N5)[C@H]3CO4)c12]

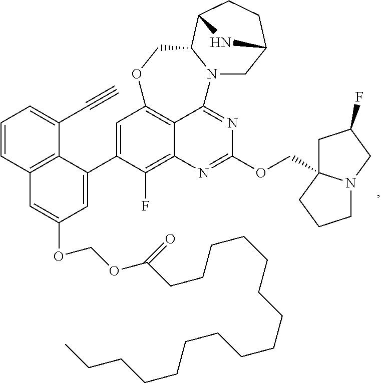 [SMILES: C#Cc1cccc2cc(OCOC(=O)CCCCCCCCCCCCCCCC)cc(-c3cc4c5c(nc(OC[C@@]67CCCN6C[C@H](F)C7)nc5c3F)N3C[C@H]5CC[C@H](N5)[C@H]3CO4)c12]

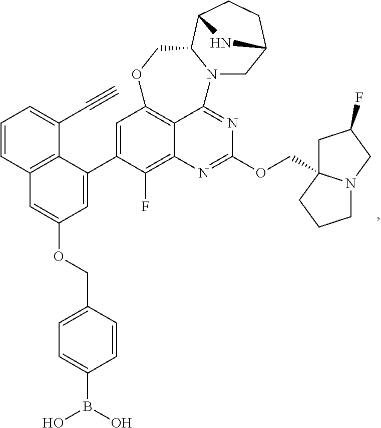 [SMILES: C#Cc1cccc2cc(OCc3ccc(B(O)O)cc3)cc(-c3cc4c5c(nc(OC[C@@]67CCCN6C[C@H](F)C7)nc5c3F)N3C[C@H]5CC[C@H](N5)[C@H]3CO4)c12]

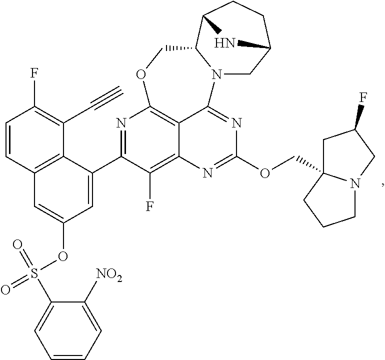 [SMILES: C#Cc1c(F)ccc2cc(OS(=O)(=O)c3ccccc3[N+](=O)[O-])cc(-c3nc4c5c(nc(OC[C@@]67CCCN6C[C@H](F)C7)nc5c3F)N3C[C@H]5CC[C@H](N5)[C@H]3CO4)c12]

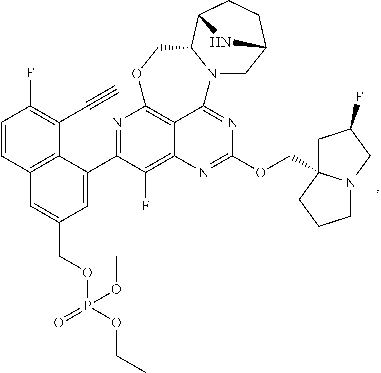 [SMILES: C#Cc1c(F)ccc2cc(COP(=O)(OC)OCC)cc(-c3nc4c5c(nc(OC[C@@]67CCCN6C[C@H](F)C7)nc5c3F)N3C[C@H]5CC[C@H](N5)[C@H]3CO4)c12]

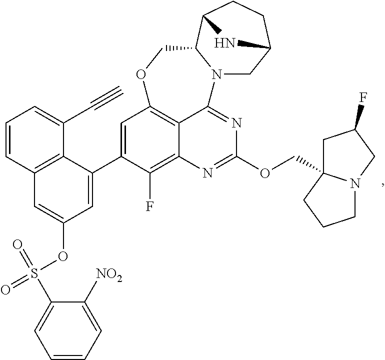 [SMILES: C#Cc1cccc2cc(OS(=O)(=O)c3ccccc3[N+](=O)[O-])cc(-c3cc4c5c(nc(OC[C@@]67CCCN6C[C@H](F)C7)nc5c3F)N3C[C@H]5CC[C@H](N5)[C@H]3CO4)c12]

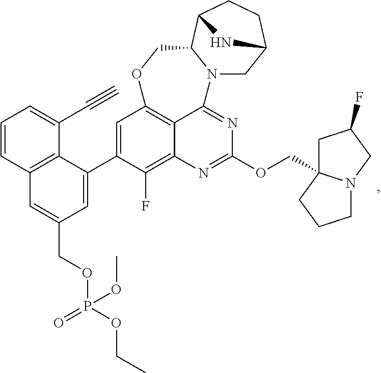 [SMILES: C#Cc1cccc2cc(COP(=O)(OC)OCC)cc(-c3cc4c5c(nc(OC[C@@]67CCCN6C[C@H](F)C7)nc5c3F)N3C[C@H]5CC[C@H](N5)[C@H]3CO4)c12]

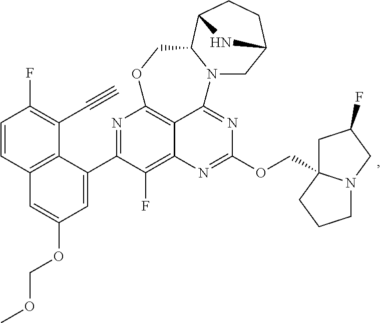 [SMILES: C#Cc1c(F)ccc2cc(OCOC)cc(-c3nc4c5c(nc(OC[C@@]67CCCN6C[C@H](F)C7)nc5c3F)N3C[C@H]5CC[C@H](N5)[C@H]3CO4)c12]

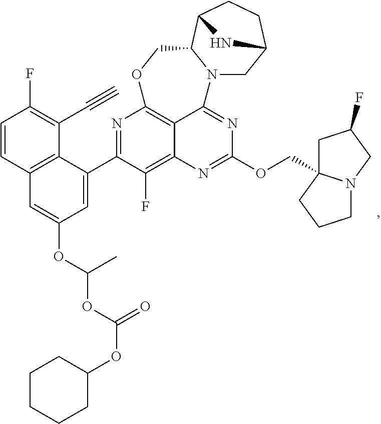 [SMILES: C#Cc1c(F)ccc2cc(OC(C)OC(=O)OC3CCCCC3)cc(-c3nc4c5c(nc(OC[C@@]67CCCN6C[C@H](F)C7)nc5c3F)N3C[C@H]5CC[C@H](N5)[C@H]3CO4)c12]

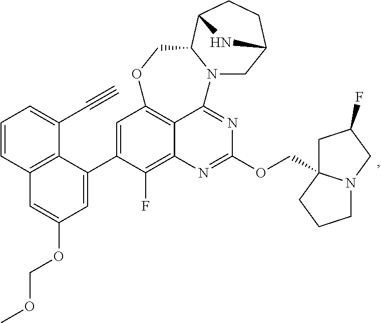 [SMILES: C#Cc1cccc2cc(OCOC)cc(-c3cc4c5c(nc(OC[C@@]67CCCN6C[C@H](F)C7)nc5c3F)N3C[C@H]5CC[C@H](N5)[C@H]3CO4)c12]

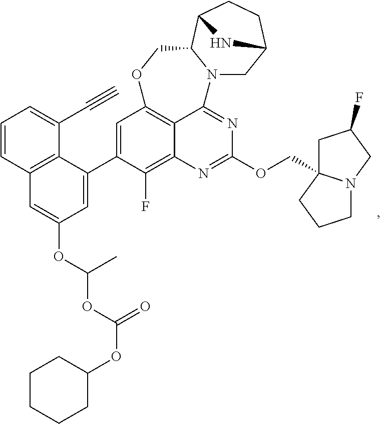 [SMILES: C#Cc1cccc2cc(OC(C)OC(=O)OC3CCCCC3)cc(-c3cc4c5c(nc(OC[C@@]67CCCN6C[C@H](F)C7)nc5c3F)N3C[C@H]5CC[C@H](N5)[C@H]3CO4)c12]

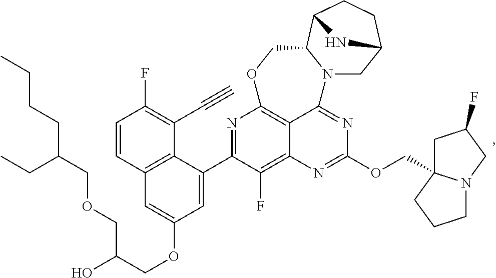 [SMILES: C#Cc1c(F)ccc2cc(OCC(O)COCC(CC)CCCC)cc(-c3nc4c5c(nc(OC[C@@]67CCCN6C[C@H](F)C7)nc5c3F)N3C[C@H]5CC[C@H](N5)[C@H]3CO4)c12]

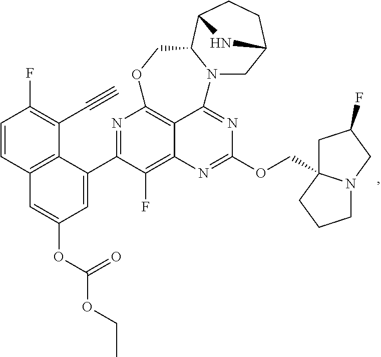 [SMILES: C#Cc1c(F)ccc2cc(OC(=O)OCC)cc(-c3nc4c5c(nc(OC[C@@]67CCCN6C[C@H](F)C7)nc5c3F)N3C[C@H]5CC[C@H](N5)[C@H]3CO4)c12]

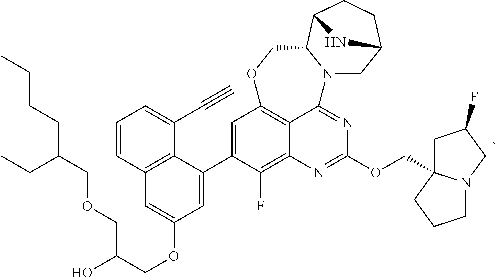 [SMILES: C#Cc1cccc2cc(OCC(O)COCC(CC)CCCC)cc(-c3cc4c5c(nc(OC[C@@]67CCCN6C[C@H](F)C7)nc5c3F)N3C[C@H]5CC[C@H](N5)[C@H]3CO4)c12]

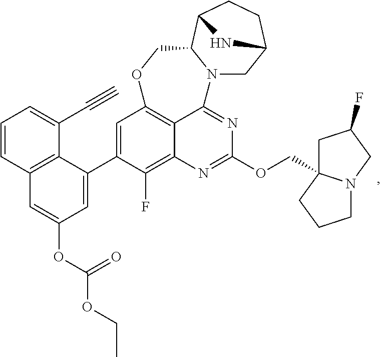 [SMILES: C#Cc1cccc2cc(OC(=O)OCC)cc(-c3cc4c5c(nc(OC[C@@]67CCCN6C[C@H](F)C7)nc5c3F)N3C[C@H]5CC[C@H](N5)[C@H]3CO4)c12]

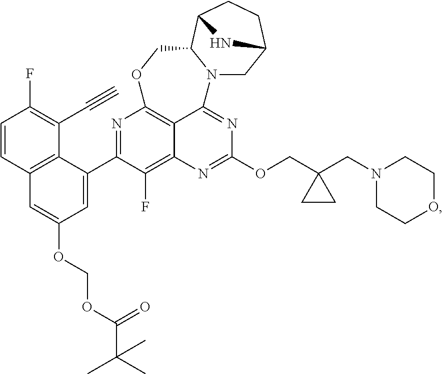 [SMILES: C#Cc1c(F)ccc2cc(OCOC(=O)C(C)(C)C)cc(-c3nc4c5c(nc(OCC6(CN7CCOCC7)CC6)nc5c3F)N3C[C@H]5CC[C@H](N5)[C@H]3CO4)c12]

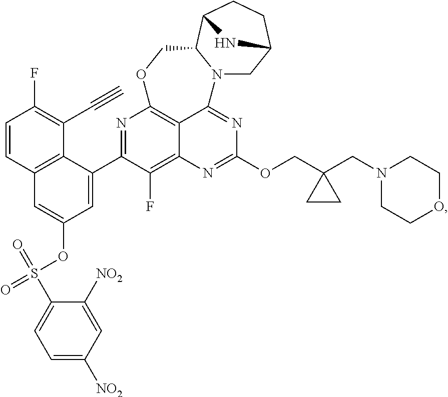 [SMILES: C#Cc1c(F)ccc2cc(OS(=O)(=O)c3ccc([N+](=O)[O-])cc3[N+](=O)[O-])cc(-c3nc4c5c(nc(OCC6(CN7CCOCC7)CC6)nc5c3F)N3C[C@H]5CC[C@H](N5)[C@H]3CO4)c12]

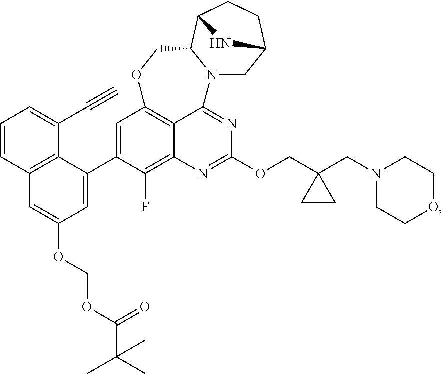 [SMILES: C#Cc1cccc2cc(OCOC(=O)C(C)(C)C)cc(-c3cc4c5c(nc(OCC6(CN7CCOCC7)CC6)nc5c3F)N3C[C@H]5CC[C@H](N5)[C@H]3CO4)c12]

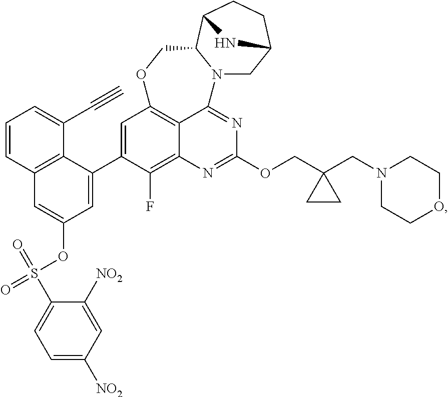 [SMILES: C#Cc1cccc2cc(OS(=O)(=O)c3ccc([N+](=O)[O-])cc3[N+](=O)[O-])cc(-c3cc4c5c(nc(OCC6(CN7CCOCC7)CC6)nc5c3F)N3C[C@H]5CC[C@H](N5)[C@H]3CO4)c12]

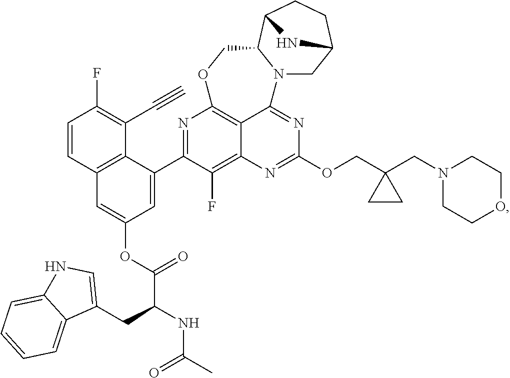 [SMILES: C#Cc1c(F)ccc2cc(OC(=O)[C@H](Cc3c[nH]c4ccccc34)NC(C)=O)cc(-c3nc4c5c(nc(OCC6(CN7CCOCC7)CC6)nc5c3F)N3C[C@H]5CC[C@H](N5)[C@H]3CO4)c12]

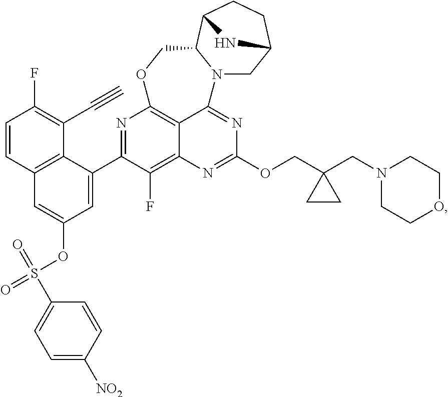 [SMILES: C#Cc1c(F)ccc2cc(OS(=O)(=O)c3ccc([N+](=O)[O-])cc3)cc(-c3nc4c5c(nc(OCC6(CN7CCOCC7)CC6)nc5c3F)N3C[C@H]5CC[C@H](N5)[C@H]3CO4)c12]

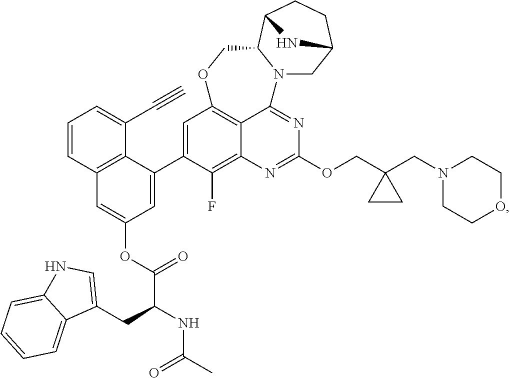 [SMILES: C#Cc1cccc2cc(OC(=O)[C@H](Cc3c[nH]c4ccccc34)NC(C)=O)cc(-c3cc4c5c(nc(OCC6(CN7CCOCC7)CC6)nc5c3F)N3C[C@H]5CC[C@H](N5)[C@H]3CO4)c12]

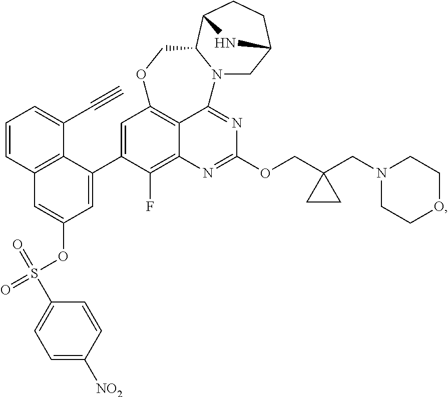 [SMILES: C#Cc1cccc2cc(OS(=O)(=O)c3ccc([N+](=O)[O-])cc3)cc(-c3cc4c5c(nc(OCC6(CN7CCOCC7)CC6)nc5c3F)N3C[C@H]5CC[C@H](N5)[C@H]3CO4)c12]

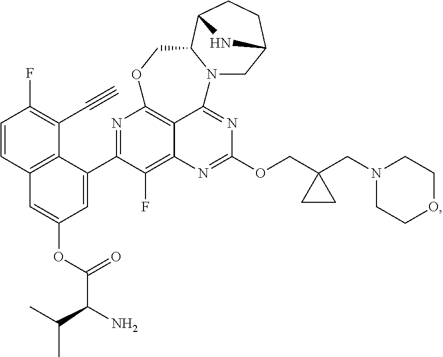 [SMILES: C#Cc1c(F)ccc2cc(OC(=O)[C@@H](N)C(C)C)cc(-c3nc4c5c(nc(OCC6(CN7CCOCC7)CC6)nc5c3F)N3C[C@H]5CC[C@H](N5)[C@H]3CO4)c12]

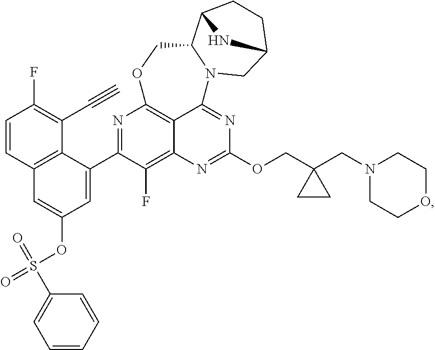 [SMILES: C#Cc1c(F)ccc2cc(OS(=O)(=O)c3ccccc3)cc(-c3nc4c5c(nc(OCC6(CN7CCOCC7)CC6)nc5c3F)N3C[C@H]5CC[C@H](N5)[C@H]3CO4)c12]

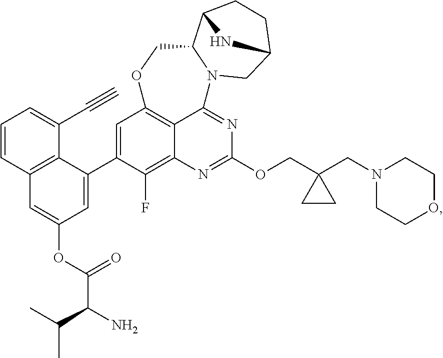 [SMILES: C#Cc1cccc2cc(OC(=O)[C@@H](N)C(C)C)cc(-c3cc4c5c(nc(OCC6(CN7CCOCC7)CC6)nc5c3F)N3C[C@H]5CC[C@H](N5)[C@H]3CO4)c12]

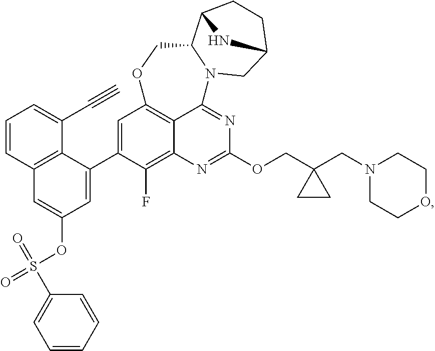 [SMILES: C#Cc1cccc2cc(OS(=O)(=O)c3ccccc3)cc(-c3cc4c5c(nc(OCC6(CN7CCOCC7)CC6)nc5c3F)N3C[C@H]5CC[C@H](N5)[C@H]3CO4)c12]

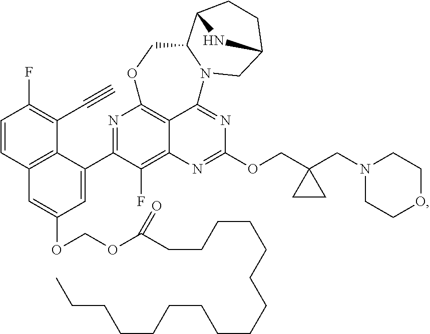 [SMILES: C#Cc1c(F)ccc2cc(OCOC(=O)CCCCCCCCCCCCCCCC)cc(-c3nc4c5c(nc(OCC6(CN7CCOCC7)CC6)nc5c3F)N3C[C@H]5CC[C@H](N5)[C@H]3CO4)c12]

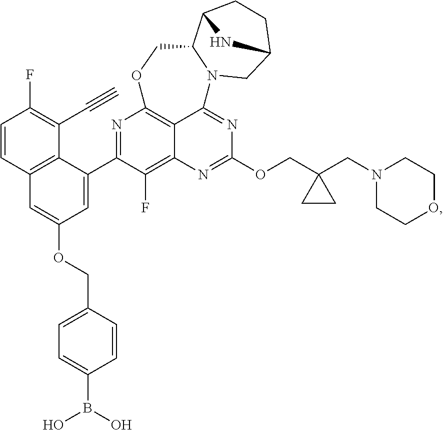 [SMILES: C#Cc1c(F)ccc2cc(OCc3ccc(B(O)O)cc3)cc(-c3nc4c5c(nc(OCC6(CN7CCOCC7)CC6)nc5c3F)N3C[C@H]5CC[C@H](N5)[C@H]3CO4)c12]

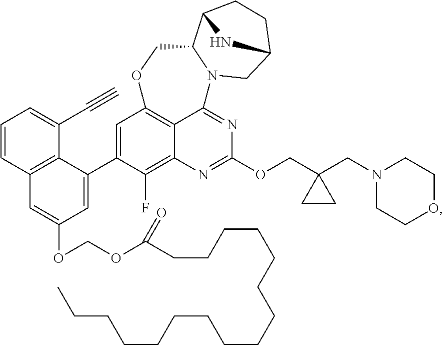 [SMILES: C#Cc1cccc2cc(OCOC(=O)CCCCCCCCCCCCCCCC)cc(-c3cc4c5c(nc(OCC6(CN7CCOCC7)CC6)nc5c3F)N3C[C@H]5CC[C@H](N5)[C@H]3CO4)c12]

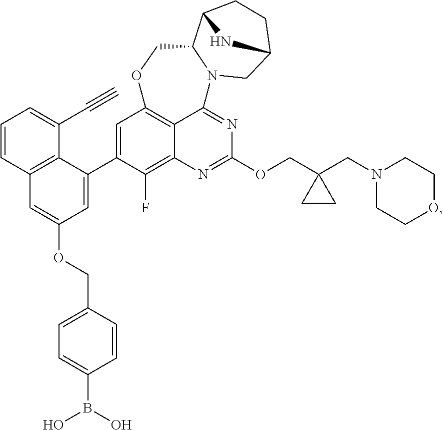 [SMILES: C#Cc1cccc2cc(OCc3ccc(B(O)O)cc3)cc(-c3cc4c5c(nc(OCC6(CN7CCOCC7)CC6)nc5c3F)N3C[C@H]5CC[C@H](N5)[C@H]3CO4)c12]

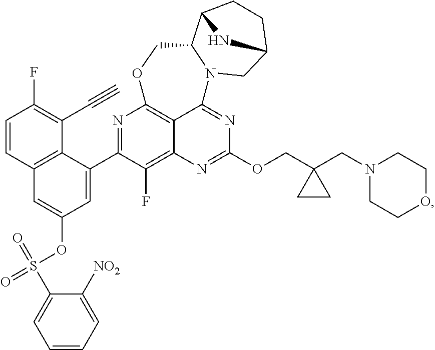 [SMILES: C#Cc1c(F)ccc2cc(OS(=O)(=O)c3ccccc3[N+](=O)[O-])cc(-c3nc4c5c(nc(OCC6(CN7CCOCC7)CC6)nc5c3F)N3C[C@H]5CC[C@H](N5)[C@H]3CO4)c12]

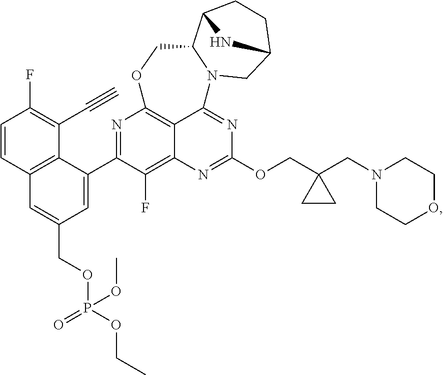 [SMILES: C#Cc1c(F)ccc2cc(COP(=O)(OC)OCC)cc(-c3nc4c5c(nc(OCC6(CN7CCOCC7)CC6)nc5c3F)N3C[C@H]5CC[C@H](N5)[C@H]3CO4)c12]

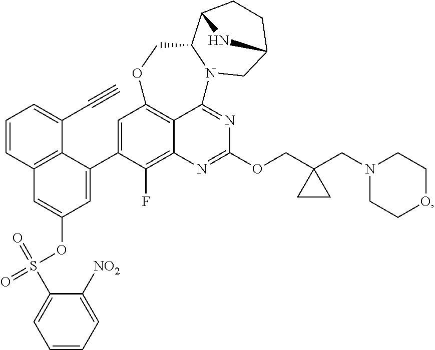 [SMILES: C#Cc1cccc2cc(OS(=O)(=O)c3ccccc3[N+](=O)[O-])cc(-c3cc4c5c(nc(OCC6(CN7CCOCC7)CC6)nc5c3F)N3C[C@H]5CC[C@H](N5)[C@H]3CO4)c12]

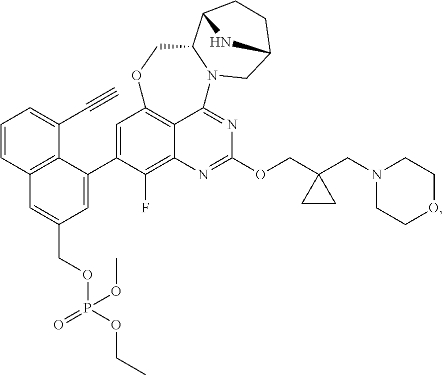 [SMILES: C#Cc1cccc2cc(COP(=O)(OC)OCC)cc(-c3cc4c5c(nc(OCC6(CN7CCOCC7)CC6)nc5c3F)N3C[C@H]5CC[C@H](N5)[C@H]3CO4)c12]

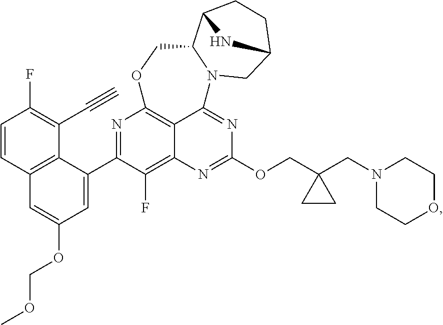 [SMILES: C#Cc1c(F)ccc2cc(OCOC)cc(-c3nc4c5c(nc(OCC6(CN7CCOCC7)CC6)nc5c3F)N3C[C@H]5CC[C@H](N5)[C@H]3CO4)c12]

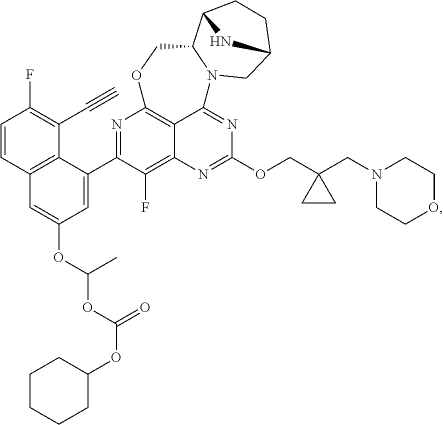 [SMILES: C#Cc1c(F)ccc2cc(OC(C)OC(=O)OC3CCCCC3)cc(-c3nc4c5c(nc(OCC6(CN7CCOCC7)CC6)nc5c3F)N3C[C@H]5CC[C@H](N5)[C@H]3CO4)c12]

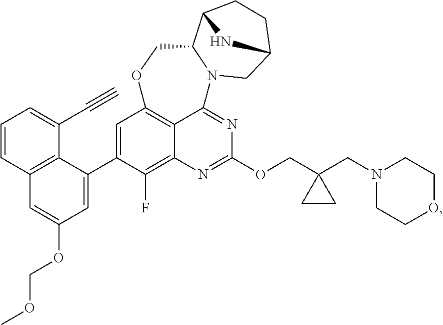 [SMILES: C#Cc1cccc2cc(OCOC)cc(-c3cc4c5c(nc(OCC6(CN7CCOCC7)CC6)nc5c3F)N3C[C@H]5CC[C@H](N5)[C@H]3CO4)c12]

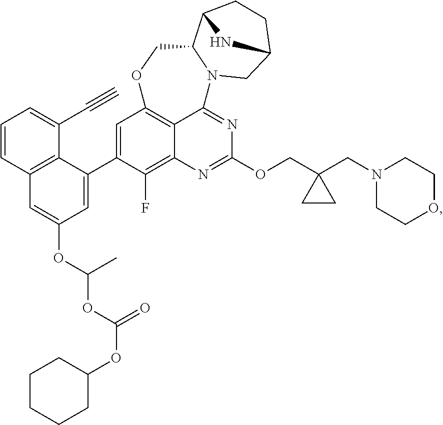 [SMILES: C#Cc1cccc2cc(OC(C)OC(=O)OC3CCCCC3)cc(-c3cc4c5c(nc(OCC6(CN7CCOCC7)CC6)nc5c3F)N3C[C@H]5CC[C@H](N5)[C@H]3CO4)c12]

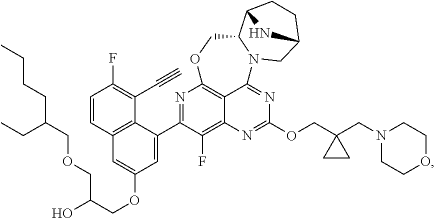 [SMILES: C#Cc1c(F)ccc2cc(OCC(O)COCC(CC)CCCC)cc(-c3nc4c5c(nc(OCC6(CN7CCOCC7)CC6)nc5c3F)N3C[C@H]5CC[C@H](N5)[C@H]3CO4)c12]

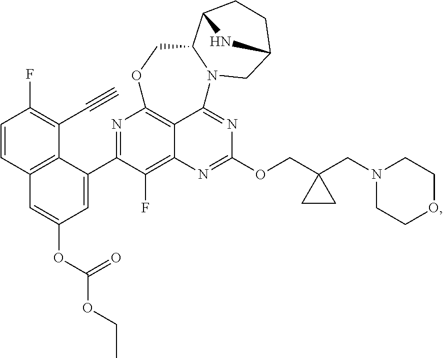 [SMILES: C#Cc1c(F)ccc2cc(OC(=O)OCC)cc(-c3nc4c5c(nc(OCC6(CN7CCOCC7)CC6)nc5c3F)N3C[C@H]5CC[C@H](N5)[C@H]3CO4)c12]

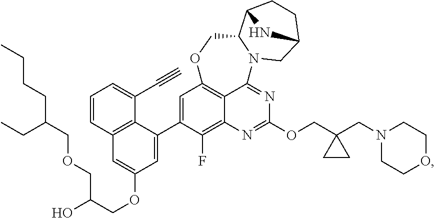 [SMILES: C#Cc1cccc2cc(OCC(O)COCC(CC)CCCC)cc(-c3cc4c5c(nc(OCC6(CN7CCOCC7)CC6)nc5c3F)N3C[C@H]5CC[C@H](N5)[C@H]3CO4)c12]

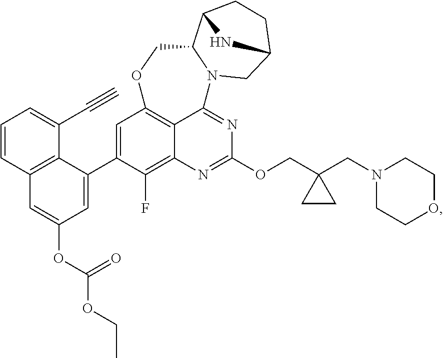 [SMILES: C#Cc1cccc2cc(OC(=O)OCC)cc(-c3cc4c5c(nc(OCC6(CN7CCOCC7)CC6)nc5c3F)N3C[C@H]5CC[C@H](N5)[C@H]3CO4)c12]

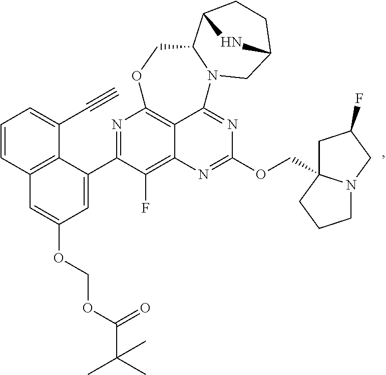 [SMILES: C#Cc1cccc2cc(OCOC(=O)C(C)(C)C)cc(-c3nc4c5c(nc(OC[C@@]67CCCN6C[C@H](F)C7)nc5c3F)N3C[C@H]5CC[C@H](N5)[C@H]3CO4)c12]

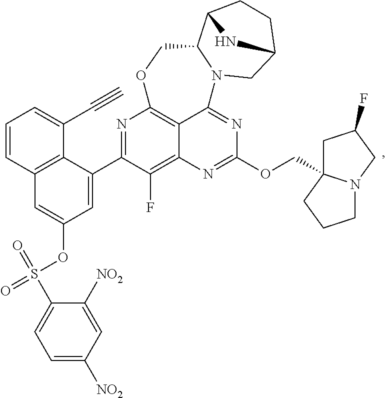 [SMILES: C#Cc1cccc2cc(OS(=O)(=O)c3ccc([N+](=O)[O-])cc3[N+](=O)[O-])cc(-c3nc4c5c(nc(OC[C@@]67CCCN6C[C@H](F)C7)nc5c3F)N3C[C@H]5CC[C@H](N5)[C@H]3CO4)c12]

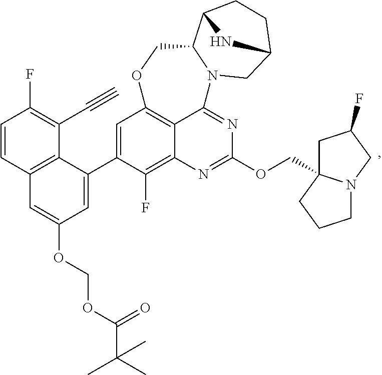 [SMILES: C#Cc1c(F)ccc2cc(OCOC(=O)C(C)(C)C)cc(-c3cc4c5c(nc(OC[C@@]67CCCN6C[C@H](F)C7)nc5c3F)N3C[C@H]5CC[C@H](N5)[C@H]3CO4)c12]

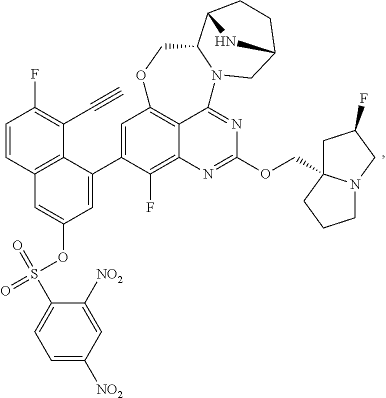 [SMILES: C#Cc1c(F)ccc2cc(OS(=O)(=O)c3ccc([N+](=O)[O-])cc3[N+](=O)[O-])cc(-c3cc4c5c(nc(OC[C@@]67CCCN6C[C@H](F)C7)nc5c3F)N3C[C@H]5CC[C@H](N5)[C@H]3CO4)c12]

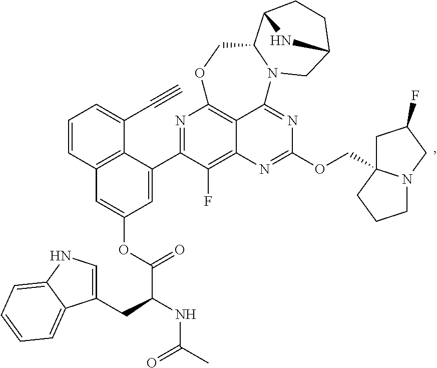 [SMILES: C#Cc1cccc2cc(OC(=O)[C@H](Cc3c[nH]c4ccccc34)NC(C)=O)cc(-c3nc4c5c(nc(OC[C@@]67CCCN6C[C@H](F)C7)nc5c3F)N3C[C@H]5CC[C@H](N5)[C@H]3CO4)c12]

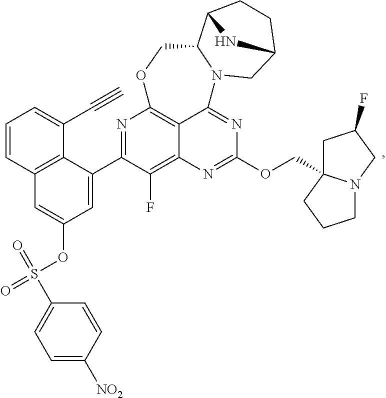 [SMILES: C#Cc1cccc2cc(OS(=O)(=O)c3ccc([N+](=O)[O-])cc3)cc(-c3nc4c5c(nc(OC[C@@]67CCCN6C[C@H](F)C7)nc5c3F)N3C[C@H]5CC[C@H](N5)[C@H]3CO4)c12]

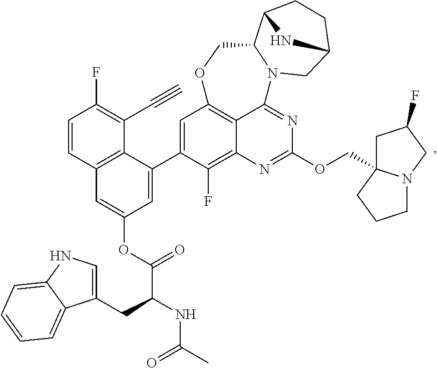 [SMILES: C#Cc1c(F)ccc2cc(OC(=O)[C@H](Cc3c[nH]c4ccccc34)NC(C)=O)cc(-c3cc4c5c(nc(OC[C@@]67CCCN6C[C@H](F)C7)nc5c3F)N3C[C@H]5CC[C@H](N5)[C@H]3CO4)c12]

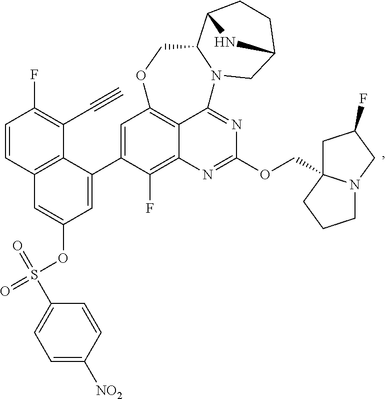 [SMILES: C#Cc1c(F)ccc2cc(OS(=O)(=O)c3ccc([N+](=O)[O-])cc3)cc(-c3cc4c5c(nc(OC[C@@]67CCCN6C[C@H](F)C7)nc5c3F)N3C[C@H]5CC[C@H](N5)[C@H]3CO4)c12]

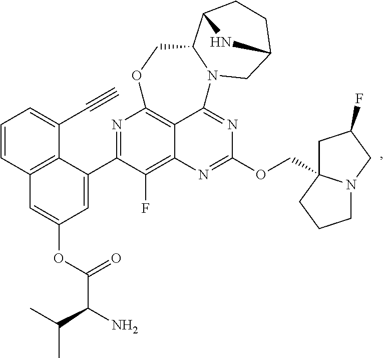 [SMILES: C#Cc1cccc2cc(OC(=O)[C@@H](N)C(C)C)cc(-c3nc4c5c(nc(OC[C@@]67CCCN6C[C@H](F)C7)nc5c3F)N3C[C@H]5CC[C@H](N5)[C@H]3CO4)c12]

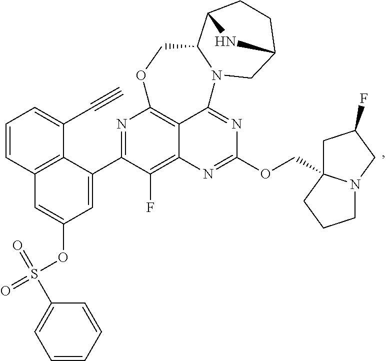 [SMILES: C#Cc1cccc2cc(OS(=O)(=O)c3ccccc3)cc(-c3nc4c5c(nc(OC[C@@]67CCCN6C[C@H](F)C7)nc5c3F)N3C[C@H]5CC[C@H](N5)[C@H]3CO4)c12]

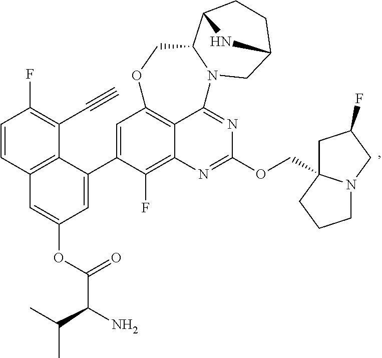 [SMILES: C#Cc1c(F)ccc2cc(OC(=O)[C@@H](N)C(C)C)cc(-c3cc4c5c(nc(OC[C@@]67CCCN6C[C@H](F)C7)nc5c3F)N3C[C@H]5CC[C@H](N5)[C@H]3CO4)c12]

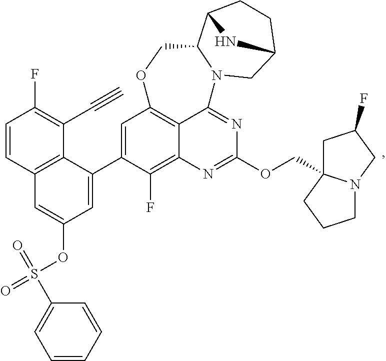 [SMILES: C#Cc1c(F)ccc2cc(OS(=O)(=O)c3ccccc3)cc(-c3cc4c5c(nc(OC[C@@]67CCCN6C[C@H](F)C7)nc5c3F)N3C[C@H]5CC[C@H](N5)[C@H]3CO4)c12]

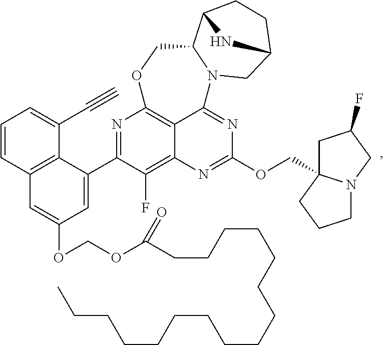 [SMILES: C#Cc1cccc2cc(OCOC(=O)CCCCCCCCCCCCCCCC)cc(-c3nc4c5c(nc(OC[C@@]67CCCN6C[C@H](F)C7)nc5c3F)N3C[C@H]5CC[C@H](N5)[C@H]3CO4)c12]

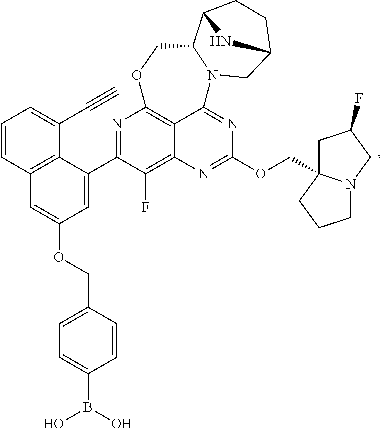 [SMILES: C#Cc1cccc2cc(OCc3ccc(B(O)O)cc3)cc(-c3nc4c5c(nc(OC[C@@]67CCCN6C[C@H](F)C7)nc5c3F)N3C[C@H]5CC[C@H](N5)[C@H]3CO4)c12]

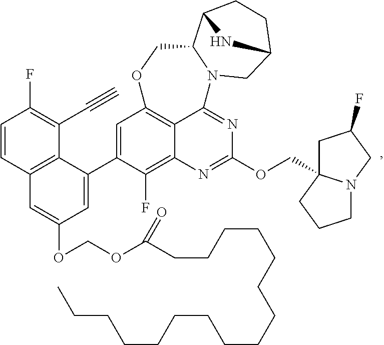 [SMILES: C#Cc1c(F)ccc2cc(OCOC(=O)CCCCCCCCCCCCCCCC)cc(-c3cc4c5c(nc(OC[C@@]67CCCN6C[C@H](F)C7)nc5c3F)N3C[C@H]5CC[C@H](N5)[C@H]3CO4)c12]

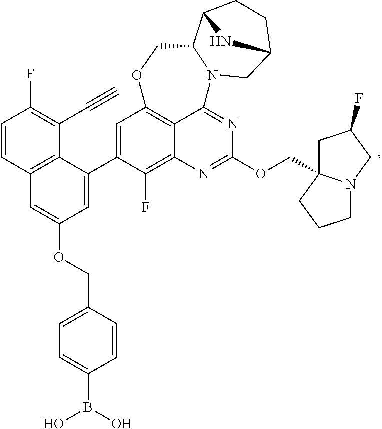 [SMILES: C#Cc1c(F)ccc2cc(OCc3ccc(B(O)O)cc3)cc(-c3cc4c5c(nc(OC[C@@]67CCCN6C[C@H](F)C7)nc5c3F)N3C[C@H]5CC[C@H](N5)[C@H]3CO4)c12]

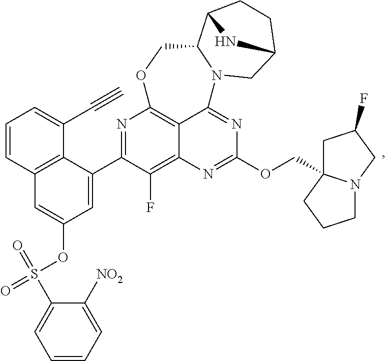 [SMILES: C#Cc1cccc2cc(OS(=O)(=O)c3ccccc3[N+](=O)[O-])cc(-c3nc4c5c(nc(OC[C@@]67CCCN6C[C@H](F)C7)nc5c3F)N3C[C@H]5CC[C@H](N5)[C@H]3CO4)c12]

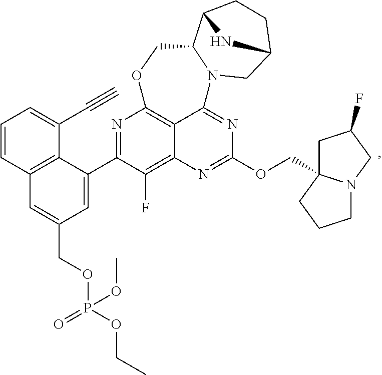 [SMILES: C#Cc1cccc2cc(COP(=O)(OC)OCC)cc(-c3nc4c5c(nc(OC[C@@]67CCCN6C[C@H](F)C7)nc5c3F)N3C[C@H]5CC[C@H](N5)[C@H]3CO4)c12]

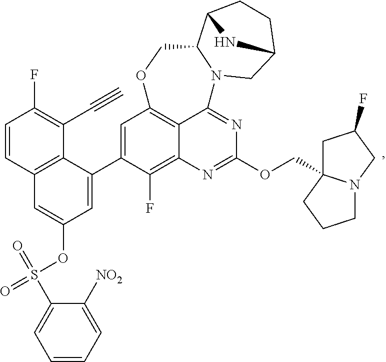 [SMILES: C#Cc1c(F)ccc2cc(OS(=O)(=O)c3ccccc3[N+](=O)[O-])cc(-c3cc4c5c(nc(OC[C@@]67CCCN6C[C@H](F)C7)nc5c3F)N3C[C@H]5CC[C@H](N5)[C@H]3CO4)c12]

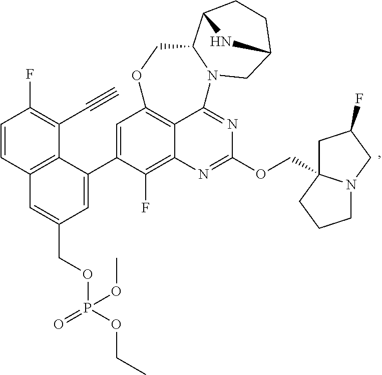 [SMILES: C#Cc1c(F)ccc2cc(COP(=O)(OC)OCC)cc(-c3cc4c5c(nc(OC[C@@]67CCCN6C[C@H](F)C7)nc5c3F)N3C[C@H]5CC[C@H](N5)[C@H]3CO4)c12]

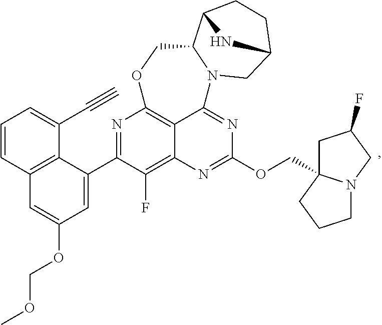 [SMILES: C#Cc1cccc2cc(OCOC)cc(-c3nc4c5c(nc(OC[C@@]67CCCN6C[C@H](F)C7)nc5c3F)N3C[C@H]5CC[C@H](N5)[C@H]3CO4)c12]

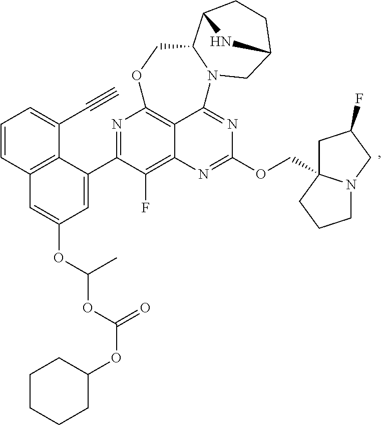 [SMILES: C#Cc1cccc2cc(OC(C)OC(=O)OC3CCCCC3)cc(-c3nc4c5c(nc(OC[C@@]67CCCN6C[C@H](F)C7)nc5c3F)N3C[C@H]5CC[C@H](N5)[C@H]3CO4)c12]

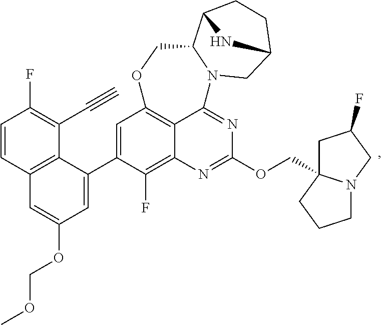 [SMILES: C#Cc1c(F)ccc2cc(OCOC)cc(-c3cc4c5c(nc(OC[C@@]67CCCN6C[C@H](F)C7)nc5c3F)N3C[C@H]5CC[C@H](N5)[C@H]3CO4)c12]

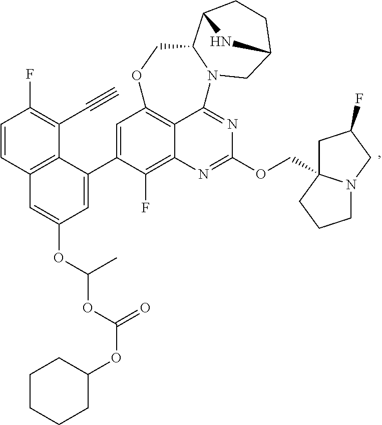 [SMILES: C#Cc1c(F)ccc2cc(OC(C)OC(=O)OC3CCCCC3)cc(-c3cc4c5c(nc(OC[C@@]67CCCN6C[C@H](F)C7)nc5c3F)N3C[C@H]5CC[C@H](N5)[C@H]3CO4)c12]

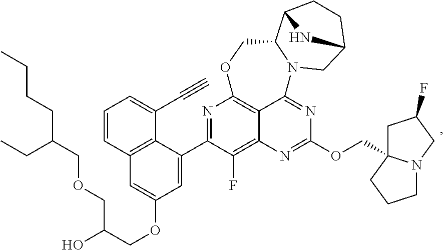 [SMILES: C#Cc1cccc2cc(OCC(O)COCC(CC)CCCC)cc(-c3nc4c5c(nc(OC[C@@]67CCCN6C[C@H](F)C7)nc5c3F)N3C[C@H]5CC[C@H](N5)[C@H]3CO4)c12]

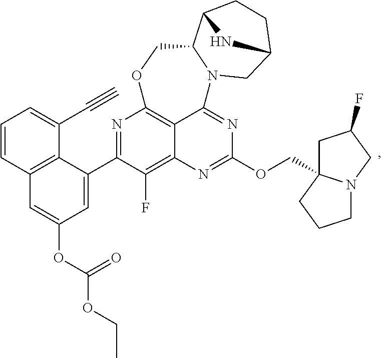 [SMILES: C#Cc1cccc2cc(OC(=O)OCC)cc(-c3nc4c5c(nc(OC[C@@]67CCCN6C[C@H](F)C7)nc5c3F)N3C[C@H]5CC[C@H](N5)[C@H]3CO4)c12]

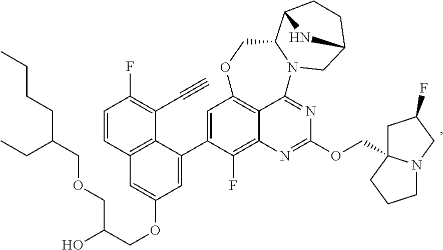 [SMILES: C#Cc1c(F)ccc2cc(OCC(O)COCC(CC)CCCC)cc(-c3cc4c5c(nc(OC[C@@]67CCCN6C[C@H](F)C7)nc5c3F)N3C[C@H]5CC[C@H](N5)[C@H]3CO4)c12]

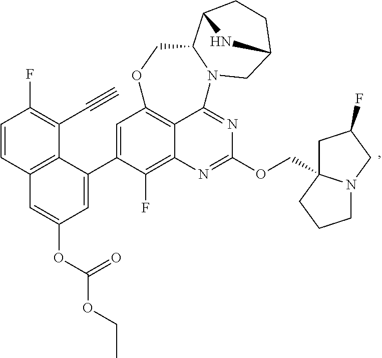 [SMILES: C#Cc1c(F)ccc2cc(OC(=O)OCC)cc(-c3cc4c5c(nc(OC[C@@]67CCCN6C[C@H](F)C7)nc5c3F)N3C[C@H]5CC[C@H](N5)[C@H]3CO4)c12]

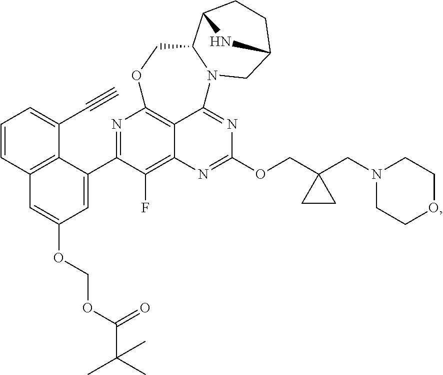 [SMILES: C#Cc1cccc2cc(OCOC(=O)C(C)(C)C)cc(-c3nc4c5c(nc(OCC6(CN7CCOCC7)CC6)nc5c3F)N3C[C@H]5CC[C@H](N5)[C@H]3CO4)c12]

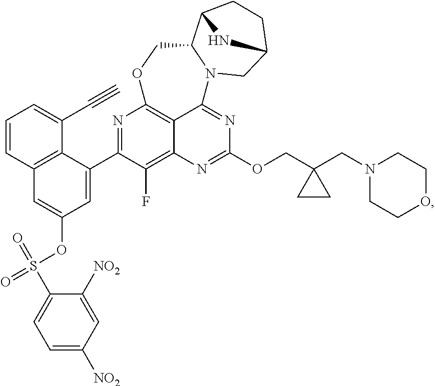 [SMILES: C#Cc1cccc2cc(OS(=O)(=O)c3ccc([N+](=O)[O-])cc3[N+](=O)[O-])cc(-c3nc4c5c(nc(OCC6(CN7CCOCC7)CC6)nc5c3F)N3C[C@H]5CC[C@H](N5)[C@H]3CO4)c12]

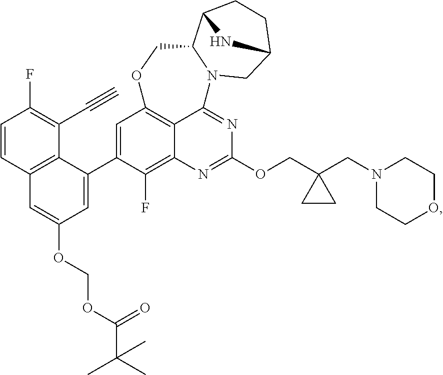 [SMILES: C#Cc1c(F)ccc2cc(OCOC(=O)C(C)(C)C)cc(-c3cc4c5c(nc(OCC6(CN7CCOCC7)CC6)nc5c3F)N3C[C@H]5CC[C@H](N5)[C@H]3CO4)c12]

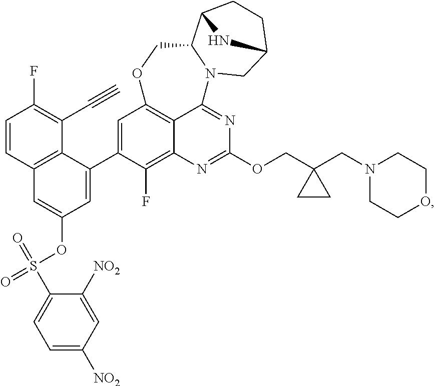 [SMILES: C#Cc1c(F)ccc2cc(OS(=O)(=O)c3ccc([N+](=O)[O-])cc3[N+](=O)[O-])cc(-c3cc4c5c(nc(OCC6(CN7CCOCC7)CC6)nc5c3F)N3C[C@H]5CC[C@H](N5)[C@H]3CO4)c12]

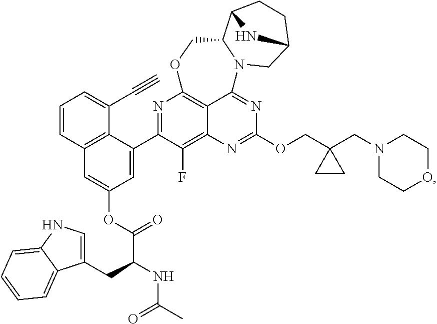 [SMILES: C#Cc1cccc2cc(OC(=O)[C@H](Cc3c[nH]c4ccccc34)NC(C)=O)cc(-c3nc4c5c(nc(OCC6(CN7CCOCC7)CC6)nc5c3F)N3C[C@H]5CC[C@H](N5)[C@H]3CO4)c12]

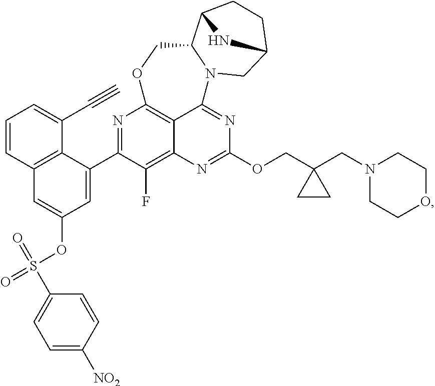 [SMILES: C#Cc1cccc2cc(OS(=O)(=O)c3ccc([N+](=O)[O-])cc3)cc(-c3nc4c5c(nc(OCC6(CN7CCOCC7)CC6)nc5c3F)N3C[C@H]5CC[C@H](N5)[C@H]3CO4)c12]

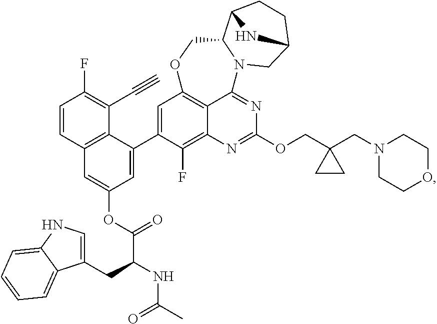 [SMILES: C#Cc1c(F)ccc2cc(OC(=O)[C@H](Cc3c[nH]c4ccccc34)NC(C)=O)cc(-c3cc4c5c(nc(OCC6(CN7CCOCC7)CC6)nc5c3F)N3C[C@H]5CC[C@H](N5)[C@H]3CO4)c12]

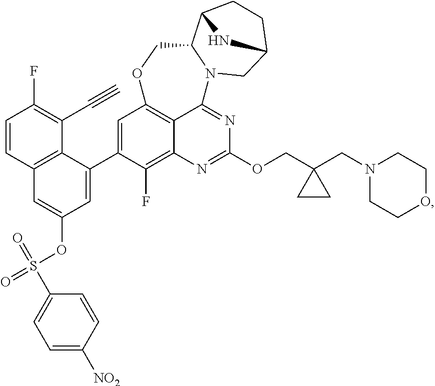 [SMILES: C#Cc1c(F)ccc2cc(OS(=O)(=O)c3ccc([N+](=O)[O-])cc3)cc(-c3cc4c5c(nc(OCC6(CN7CCOCC7)CC6)nc5c3F)N3C[C@H]5CC[C@H](N5)[C@H]3CO4)c12]

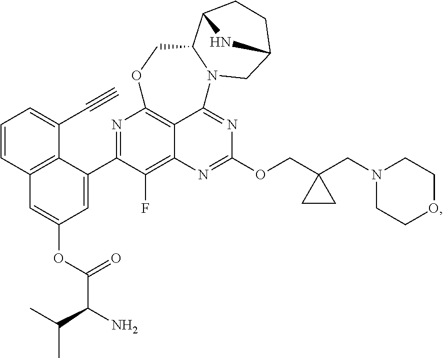 [SMILES: C#Cc1cccc2cc(OC(=O)[C@@H](N)C(C)C)cc(-c3nc4c5c(nc(OCC6(CN7CCOCC7)CC6)nc5c3F)N3C[C@H]5CC[C@H](N5)[C@H]3CO4)c12]

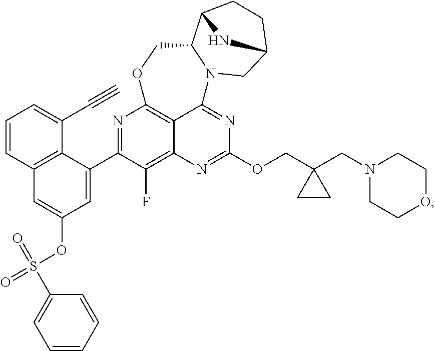 [SMILES: C#Cc1cccc2cc(OS(=O)(=O)c3ccccc3)cc(-c3nc4c5c(nc(OCC6(CN7CCOCC7)CC6)nc5c3F)N3C[C@H]5CC[C@H](N5)[C@H]3CO4)c12]

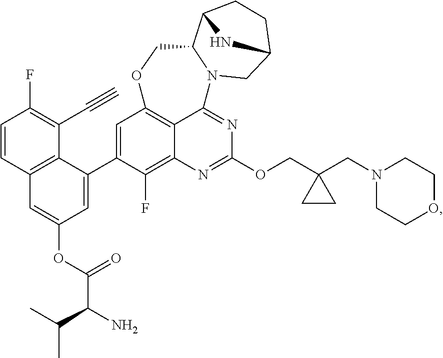 [SMILES: C#Cc1c(F)ccc2cc(OC(=O)[C@@H](N)C(C)C)cc(-c3cc4c5c(nc(OCC6(CN7CCOCC7)CC6)nc5c3F)N3C[C@H]5CC[C@H](N5)[C@H]3CO4)c12]

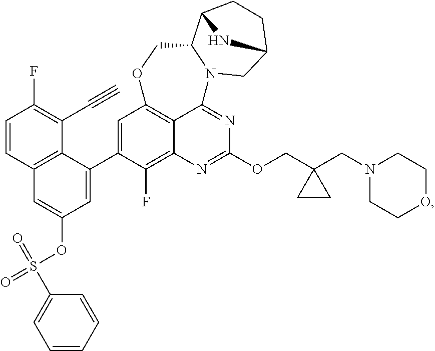 [SMILES: C#Cc1c(F)ccc2cc(OS(=O)(=O)c3ccccc3)cc(-c3cc4c5c(nc(OCC6(CN7CCOCC7)CC6)nc5c3F)N3C[C@H]5CC[C@H](N5)[C@H]3CO4)c12]

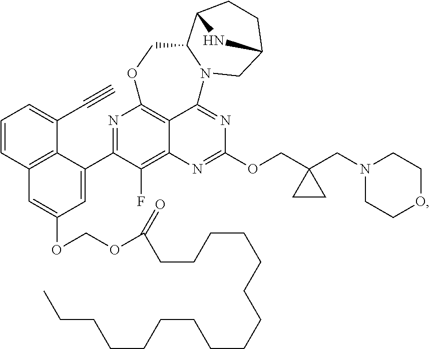 [SMILES: C#Cc1cccc2cc(OCOC(=O)CCCCCCCCCCCCCCCC)cc(-c3nc4c5c(nc(OCC6(CN7CCOCC7)CC6)nc5c3F)N3C[C@H]5CC[C@H](N5)[C@H]3CO4)c12]

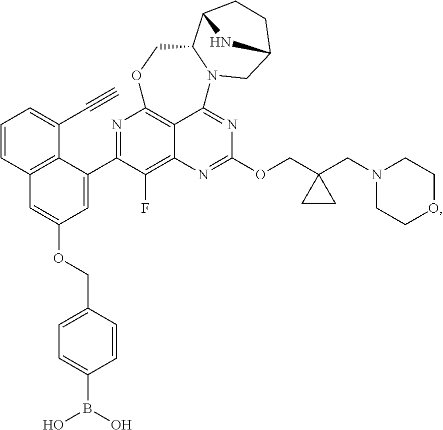 [SMILES: C#Cc1cccc2cc(OCc3ccc(B(O)O)cc3)cc(-c3nc4c5c(nc(OCC6(CN7CCOCC7)CC6)nc5c3F)N3C[C@H]5CC[C@H](N5)[C@H]3CO4)c12]

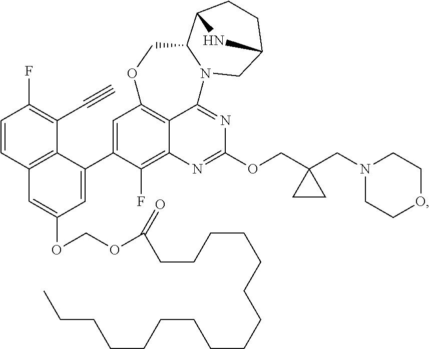 [SMILES: C#Cc1c(F)ccc2cc(OCOC(=O)CCCCCCCCCCCCCCCC)cc(-c3cc4c5c(nc(OCC6(CN7CCOCC7)CC6)nc5c3F)N3C[C@H]5CC[C@H](N5)[C@H]3CO4)c12]

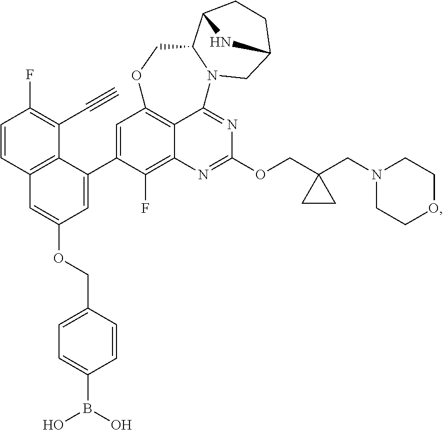 [SMILES: C#Cc1c(F)ccc2cc(OCc3ccc(B(O)O)cc3)cc(-c3cc4c5c(nc(OCC6(CN7CCOCC7)CC6)nc5c3F)N3C[C@H]5CC[C@H](N5)[C@H]3CO4)c12]

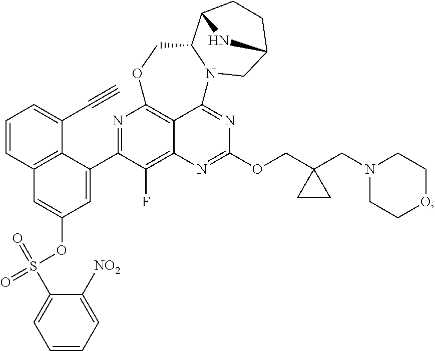 [SMILES: C#Cc1cccc2cc(OS(=O)(=O)c3ccccc3[N+](=O)[O-])cc(-c3nc4c5c(nc(OCC6(CN7CCOCC7)CC6)nc5c3F)N3C[C@H]5CC[C@H](N5)[C@H]3CO4)c12]

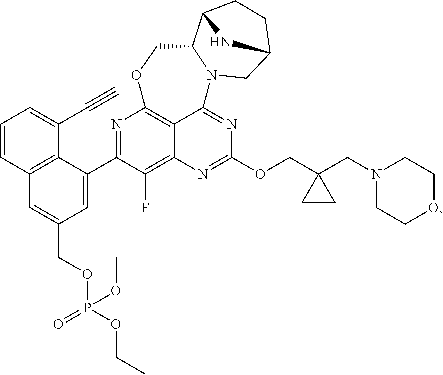 [SMILES: C#Cc1cccc2cc(COP(=O)(OC)OCC)cc(-c3nc4c5c(nc(OCC6(CN7CCOCC7)CC6)nc5c3F)N3C[C@H]5CC[C@H](N5)[C@H]3CO4)c12]

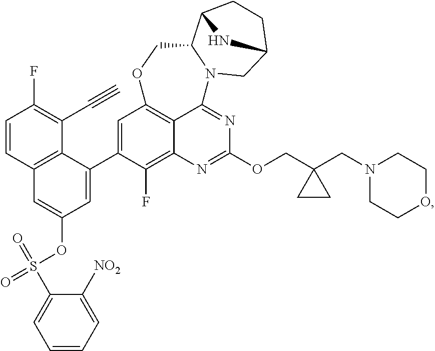 [SMILES: C#Cc1c(F)ccc2cc(OS(=O)(=O)c3ccccc3[N+](=O)[O-])cc(-c3cc4c5c(nc(OCC6(CN7CCOCC7)CC6)nc5c3F)N3C[C@H]5CC[C@H](N5)[C@H]3CO4)c12]

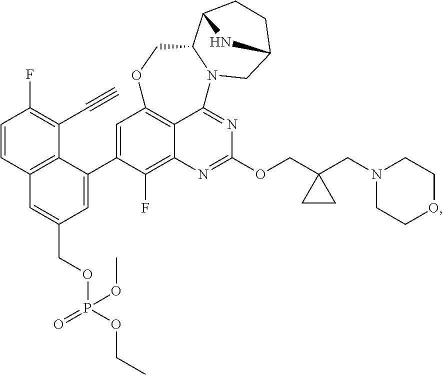 [SMILES: C#Cc1c(F)ccc2cc(COP(=O)(OC)OCC)cc(-c3cc4c5c(nc(OCC6(CN7CCOCC7)CC6)nc5c3F)N3C[C@H]5CC[C@H](N5)[C@H]3CO4)c12]

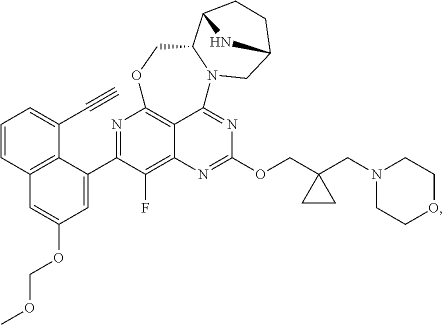 [SMILES: C#Cc1cccc2cc(OCOC)cc(-c3nc4c5c(nc(OCC6(CN7CCOCC7)CC6)nc5c3F)N3C[C@H]5CC[C@H](N5)[C@H]3CO4)c12]

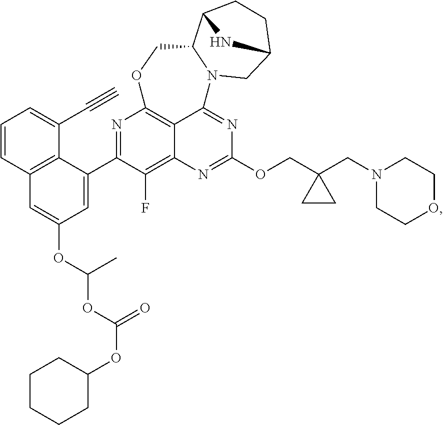 [SMILES: C#Cc1cccc2cc(OC(C)OC(=O)OC3CCCCC3)cc(-c3nc4c5c(nc(OCC6(CN7CCOCC7)CC6)nc5c3F)N3C[C@H]5CC[C@H](N5)[C@H]3CO4)c12]

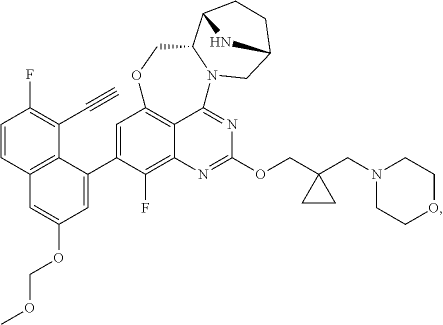 [SMILES: C#Cc1c(F)ccc2cc(OCOC)cc(-c3cc4c5c(nc(OCC6(CN7CCOCC7)CC6)nc5c3F)N3C[C@H]5CC[C@H](N5)[C@H]3CO4)c12]

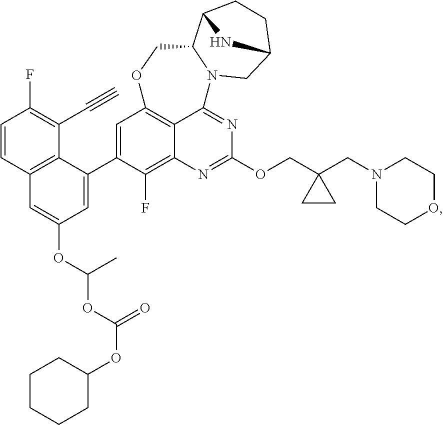 [SMILES: C#Cc1c(F)ccc2cc(OC(C)OC(=O)OC3CCCCC3)cc(-c3cc4c5c(nc(OCC6(CN7CCOCC7)CC6)nc5c3F)N3C[C@H]5CC[C@H](N5)[C@H]3CO4)c12]

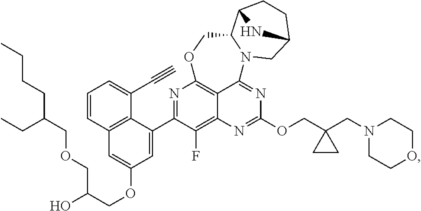 [SMILES: C#Cc1cccc2cc(OCC(O)COCC(CC)CCCC)cc(-c3nc4c5c(nc(OCC6(CN7CCOCC7)CC6)nc5c3F)N3C[C@H]5CC[C@H](N5)[C@H]3CO4)c12]

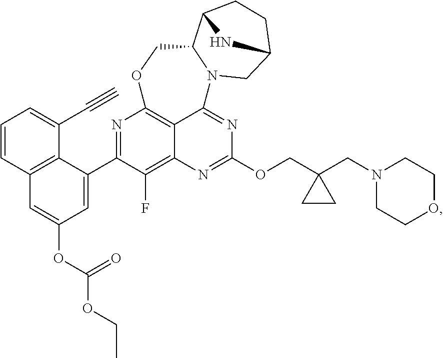 [SMILES: C#Cc1cccc2cc(OC(=O)OCC)cc(-c3nc4c5c(nc(OCC6(CN7CCOCC7)CC6)nc5c3F)N3C[C@H]5CC[C@H](N5)[C@H]3CO4)c12]

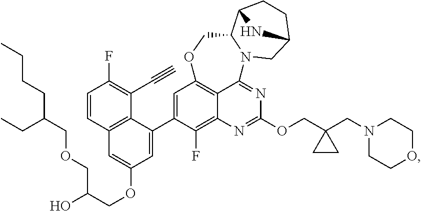 [SMILES: C#Cc1c(F)ccc2cc(OCC(O)COCC(CC)CCCC)cc(-c3cc4c5c(nc(OCC6(CN7CCOCC7)CC6)nc5c3F)N3C[C@H]5CC[C@H](N5)[C@H]3CO4)c12]

or

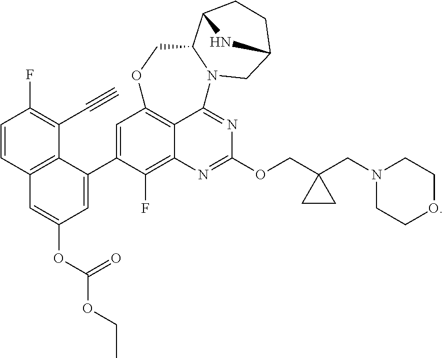 [SMILES: C#Cc1c(F)ccc2cc(OC(=O)OCC)cc(-c3cc4c5c(nc(OCC6(CN7CCOCC7)CC6)nc5c3F)N3C[C@H]5CC[C@H](N5)[C@H]3CO4)c12]

[30]. An intermediate selected from any one of intermediates in the following Table 11:

TABLE 11

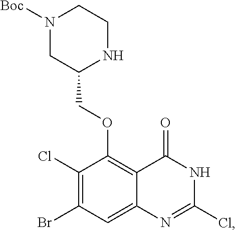 [SMILES: CC(C)(C)OC(=O)N1CCN[C@H](COc2c(Cl)c(Br)cc3nc(Cl)[nH]c(=O)c23)C1]

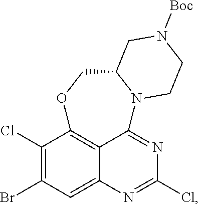 [SMILES: CC(C)(C)OC(=O)N1CCN2c3nc(Cl)nc4cc(Br)c(Cl)c(c34)OC[C@@H]2C1]

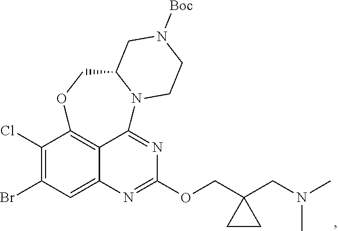

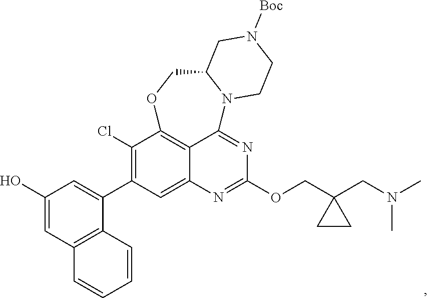

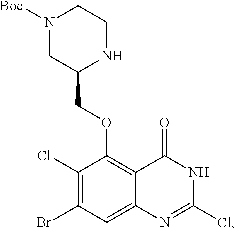 [SMILES: CC(C)(C)OC(=O)N1CCN[C@@H](COc2c(Cl)c(Br)cc3nc(Cl)[nH]c(=O)c23)C1]

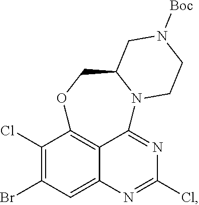 [SMILES: CC(C)(C)OC(=O)N1CCN2c3nc(Cl)nc4cc(Br)c(Cl)c(c34)OC[C@H]2C1]

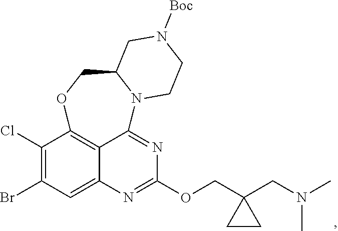 [SMILES: CN(C)CC1(COc2nc3c4c(c(Cl)c(Br)cc4n2)OC[C@H]2CN(C(=O)OC(C)(C)C)CCN32)CC1]

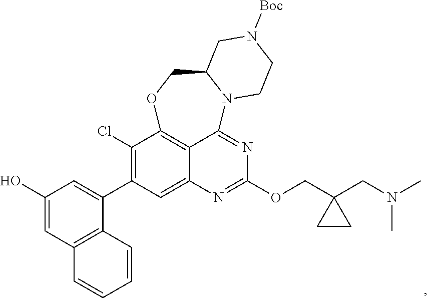

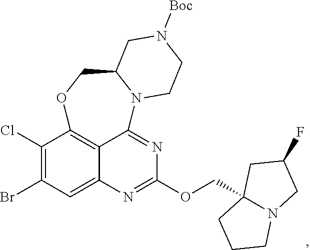 [SMILES: CC(C)(C)OC(=O)N1CCN2c3nc(OC[C@@]45CCCN4C[C@H](F)C5)nc4cc(Br)c(Cl)c(c34)OC[C@H]2C1]

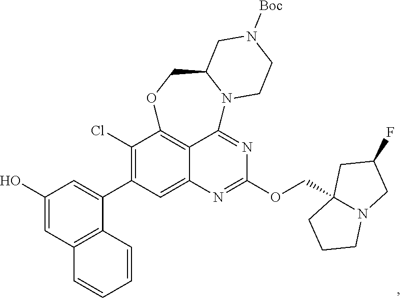 [SMILES: CC(C)(C)OC(=O)N1CCN2c3nc(OC[C@@]45CCCN4C[C@H](F)C5)nc4cc(-c5cc(O)cc6ccccc56)c(Cl)c(c34)OC[C@H]2C1]

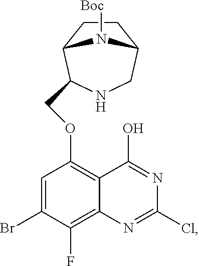 [SMILES: CC(C)(C)OC(=O)N1[C@@H]2CC[C@H]1[C@H](COc1cc(Br)c(F)c3nc(Cl)nc(O)c13)NC2]

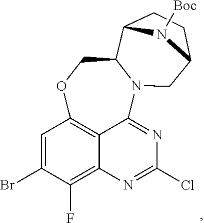 [SMILES: CC(C)(C)OC(=O)N1[C@@H]2CC[C@H]1[C@@H]1COc3cc(Br)c(F)c4nc(Cl)nc(c34)N1C2]

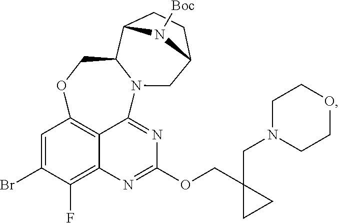 [SMILES: CC(C)(C)OC(=O)N1[C@@H]2CC[C@H]1[C@@H]1COc3cc(Br)c(F)c4nc(OCC5(CN6CCOCC6)CC5)nc(c34)N1C2]

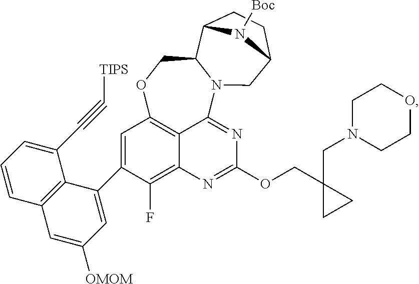

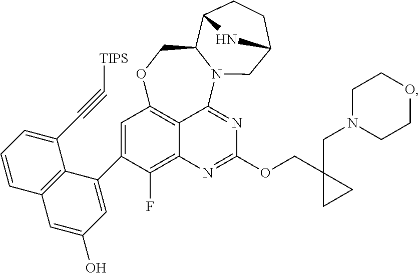

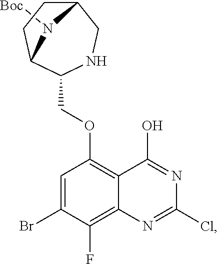 [SMILES: CC(C)(C)OC(=O)N1[C@@H]2CC[C@H]1[C@@H](COc1cc(Br)c(F)c3nc(Cl)nc(O)c13)NC2]

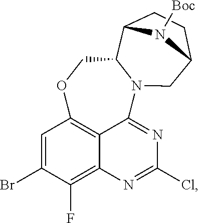 [SMILES: CC(C)(C)OC(=O)N1[C@@H]2CC[C@H]1[C@H]1COc3cc(Br)c(F)c4nc(Cl)nc(c34)N1C2]

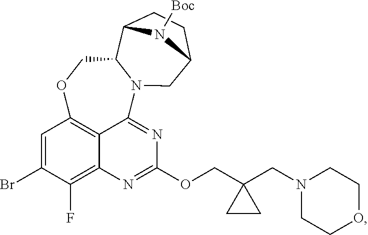 [SMILES: CC(C)(C)OC(=O)N1[C@@H]2CC[C@H]1[C@H]1COc3cc(Br)c(F)c4nc(OCC5(CN6CCOCC6)CC5)nc(c34)N1C2]

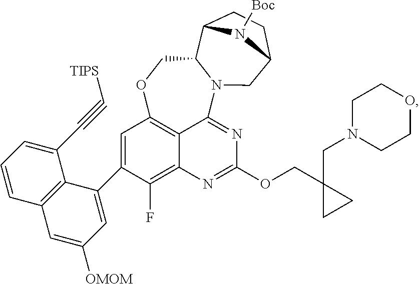

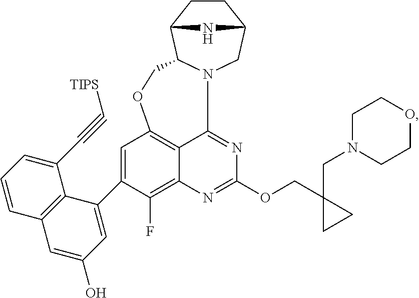

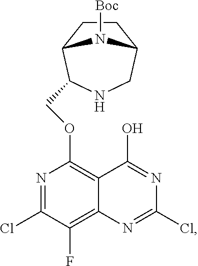 [SMILES: CC(C)(C)OC(=O)N1[C@@H]2CC[C@H]1[C@@H](COc1nc(Cl)c(F)c3nc(Cl)nc(O)c13)NC2]

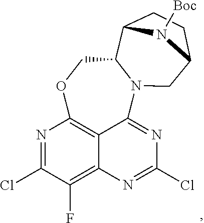 [SMILES: CC(C)(C)OC(=O)N1[C@@H]2CC[C@H]1[C@H]1COc3nc(Cl)c(F)c4nc(Cl)nc(c34)N1C2]

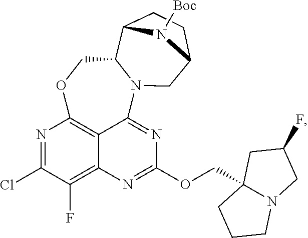 [SMILES: CC(C)(C)OC(=O)N1[C@@H]2CC[C@H]1[C@H]1COc3nc(Cl)c(F)c4nc(OC[C@@]56CCCN5C[C@H](F)C6)nc(c34)N1C2]

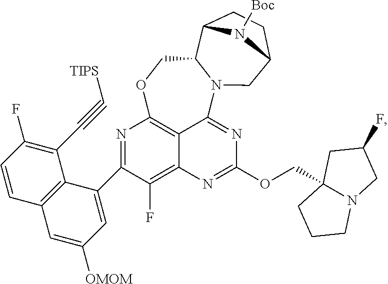

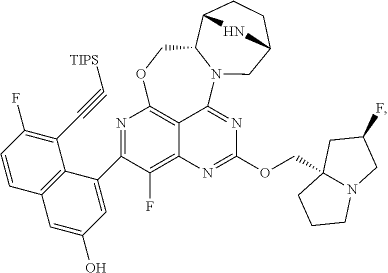

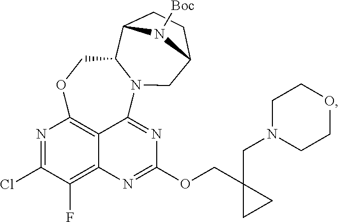 [SMILES: CC(C)(C)OC(=O)N1[C@@H]2CC[C@H]1[C@H]1COc3nc(Cl)c(F)c4nc(OCC5(CN6CCOCC6)CC5)nc(c34)N1C2]

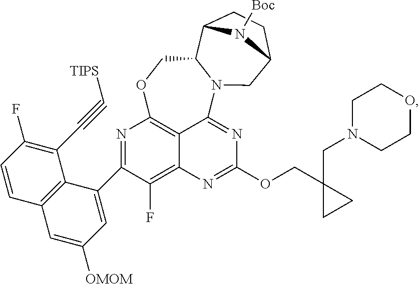

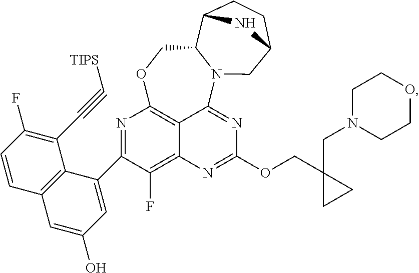

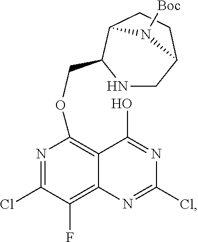 [SMILES: CC(C)(C)OC(=O)N1[C@H]2CC[C@@H]1[C@H](COc1nc(Cl)c(F)c3nc(Cl)nc(O)c13)NC2]

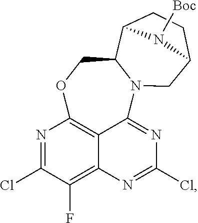 [SMILES: CC(C)(C)OC(=O)N1[C@H]2CC[C@@H]1[C@@H]1COc3nc(Cl)c(F)c4nc(Cl)nc(c34)N1C2]

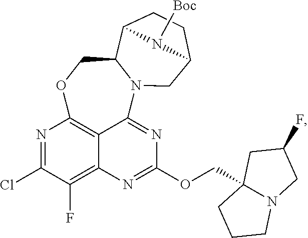 [SMILES: CC(C)(C)OC(=O)N1[C@H]2CC[C@@H]1[C@@H]1COc3nc(Cl)c(F)c4nc(OC[C@@]56CCCN5C[C@H](F)C6)nc(c34)N1C2]

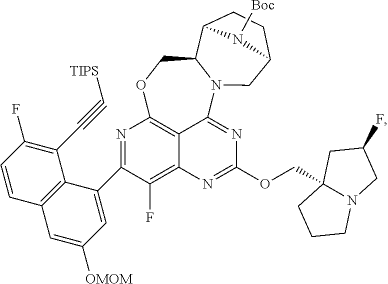

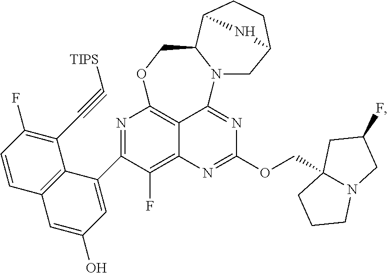

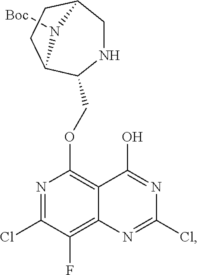 [SMILES: CC(C)(C)OC(=O)N1[C@H]2CC[C@@H]1[C@@H](COc1nc(Cl)c(F)c3nc(Cl)nc(O)c13)NC2]

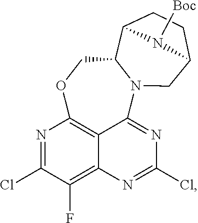 [SMILES: CC(C)(C)OC(=O)N1[C@H]2CC[C@@H]1[C@H]1COc3nc(Cl)c(F)c4nc(Cl)nc(c34)N1C2]

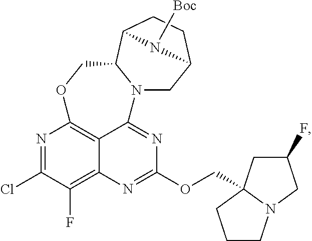 [SMILES: CC(C)(C)OC(=O)N1[C@H]2CC[C@@H]1[C@H]1COc3nc(Cl)c(F)c4nc(OC[C@@]56CCCN5C[C@H](F)C6)nc(c34)N1C2]

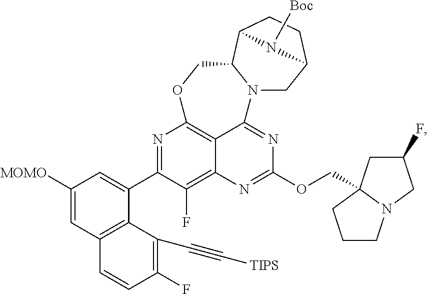

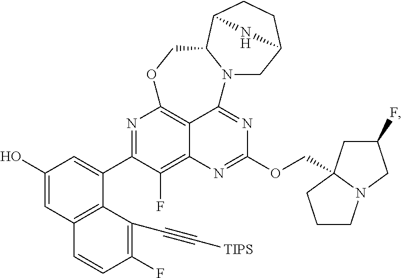

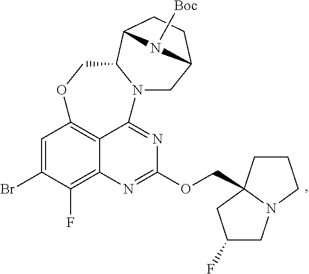 [SMILES: CC(C)(C)OC(=O)N1[C@@H]2CC[C@H]1[C@H]1COc3cc(Br)c(F)c4nc(OC[C@@]56CCCN5C[C@H](F)C6)nc(c34)N1C2]

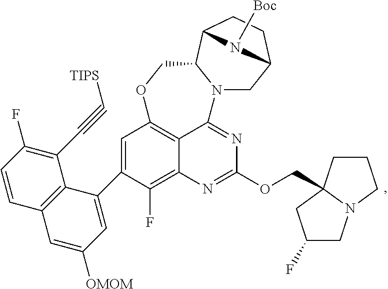

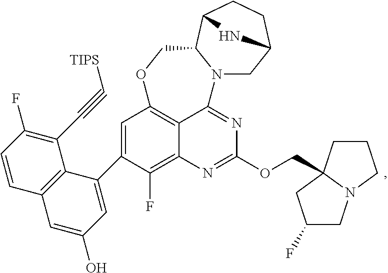

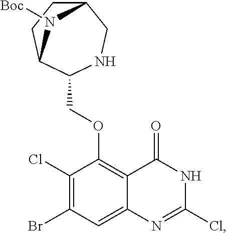 [SMILES: CC(C)(C)OC(=O)N1[C@@H]2CC[C@H]1[C@@H](COc1c(Cl)c(Br)cc3nc(Cl)[nH]c(=O)c13)NC2]

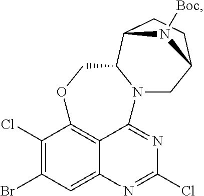 [SMILES: CC(C)(C)OC(=O)N1[C@@H]2CC[C@H]1[C@H]1COc3c(Cl)c(Br)cc4nc(Cl)nc(c34)N1C2]

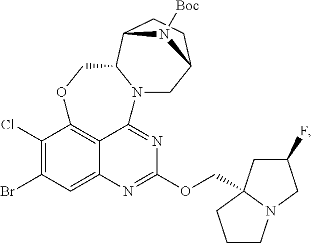 [SMILES: CC(C)(C)OC(=O)N1[C@@H]2CC[C@H]1[C@H]1COc3c(Cl)c(Br)cc4nc(OC[C@@]56CCCN5C[C@H](F)C6)nc(c34)N1C2]

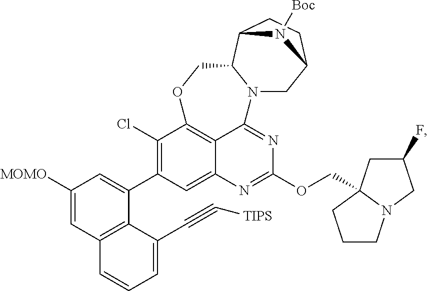

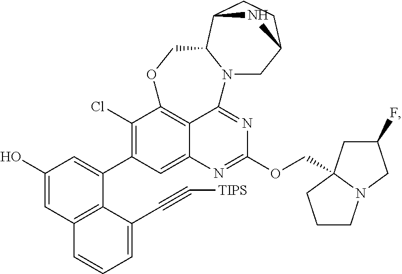

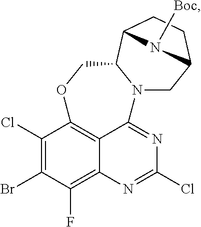

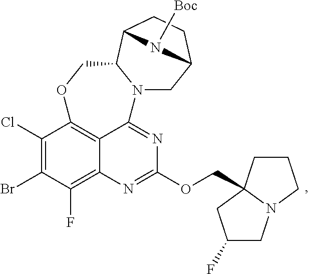 [SMILES: CC(C)(C)OC(=O)N1[C@@H]2CC[C@H]1[C@H]1COc3c(Cl)c(Br)c(F)c4nc(OC[C@@]56CCCN5C[C@H](F)C6)nc(c34)N1C2]

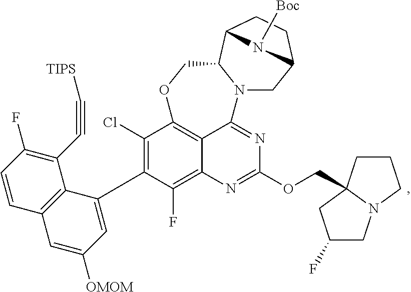

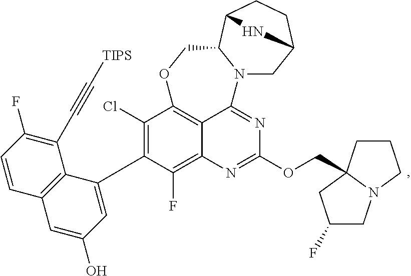

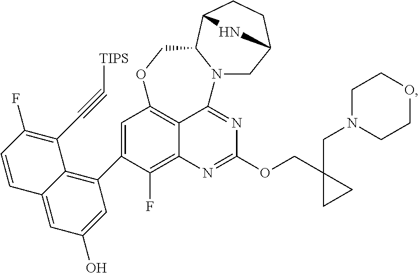

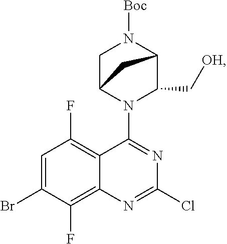 [SMILES: CC(C)(C)OC(=O)N1C[C@@H]2C[C@H]1[C@H](CO)N2c1nc(Cl)nc2c(F)c(Br)cc(F)c12]

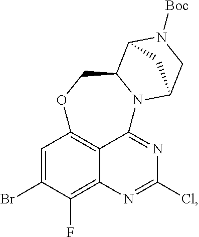 [SMILES: CC(C)(C)OC(=O)N1C[C@@H]2C[C@H]1[C@@H]1COc3cc(Br)c(F)c4nc(Cl)nc(c34)N21]

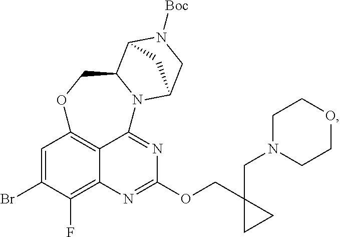 [SMILES: CC(C)(C)OC(=O)N1C[C@@H]2C[C@H]1[C@@H]1COc3cc(Br)c(F)c4nc(OCC5(CN6CCOCC6)CC5)nc(c34)N21]

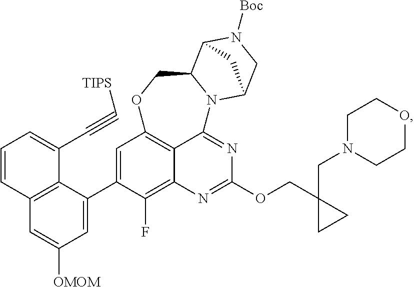

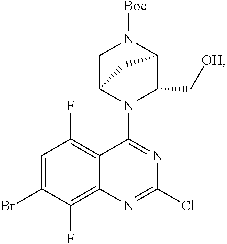 [SMILES: CC(C)(C)OC(=O)N1C[C@H]2C[C@@H]1[C@H](CO)N2c1nc(Cl)nc2c(F)c(Br)cc(F)c12]

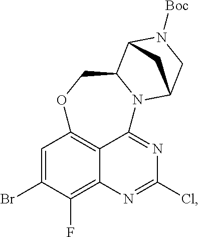 [SMILES: CC(C)(C)OC(=O)N1C[C@H]2C[C@@H]1[C@@H]1COc3cc(Br)c(F)c4nc(Cl)nc(c34)N21]

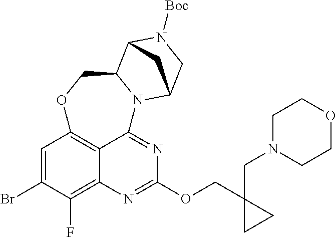 [SMILES: CC(C)(C)OC(=O)N1C[C@H]2C[C@@H]1[C@@H]1COc3cc(Br)c(F)c4nc(OCC5(CN6CCOCC6)CC5)nc(c34)N21]

or

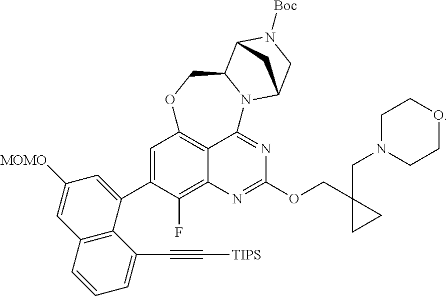

[31]. A pharmaceutical composition comprising a compound, a stereoisomer thereof, an atropisomer thereof, a pharmaceutically acceptable salt thereof, a pharmaceutically acceptable salt of the stereoisomer

thereof or a pharmaceutically acceptable salt of the atropisomer thereof of any one of aspects [1] to [29], and at least one pharmaceutically acceptable excipient.

[32]. Use of a compound, a stereoisomer thereof, an atropisomer thereof, a pharmaceutically acceptable salt thereof, a pharmaceutically acceptable salt of the stereoisomer thereof or a pharmaceutically acceptable salt of the atropisomer thereof of any one of aspects [1] to [29]; or the pharmaceutical composition of aspect [31] for the manufacture of a medicament for the treatment of cancer related to KRAS G12D mutant protein. In some embodiments, the cancer is selected from pancreatic cancer, colorectal cancer, endometrial cancer or lung cancer. In some embodiments, the lung cancer is selected from non-small cell lung cancer or small cell lung cancer.

[33]. A method of treating a subject having a cancer related to KRAS G12D mutant protein, said method comprising administering to the subject a therapeutically effective amount of a compound, a stereoisomer thereof, an atropisomer thereof, a pharmaceutically acceptable salt thereof, a pharmaceutically acceptable salt of the stereoisomer thereof or a pharmaceutically acceptable salt of the atropisomer thereof of any one of aspects [1] to [29]; or the pharmaceutical composition of aspect [31]. In some embodiments, the cancer is selected from pancreatic cancer, colorectal cancer, endometrial cancer or lung cancer. In some embodiments, the lung cancer is selected from non-small cell lung cancer or small cell lung cancer.

[34]. A compound, a stereoisomer thereof, an atropisomer thereof, a pharmaceutically acceptable salt thereof, a pharmaceutically acceptable salt of the stereoisomer thereof or a pharmaceutically acceptable salt of the atropisomer thereof of any one of aspects [1] to [29]; or the pharmaceutical composition of aspect [31] for use in the treatment of cancer related to KRAS G12D mutant protein. In some embodiments, the cancer is selected from pancreatic cancer, colorectal cancer, endometrial cancer or lung cancer. In some embodiments, the lung cancer is selected from non-small cell lung cancer or small cell lung cancer.

### Definition

Unless defined otherwise, all technical and scientific terms used herein have the same meaning as is commonly understood by one of skill in the art to which this invention belongs. All patents, patent applications, and publications referred to herein are incorporated by reference.

As used herein, “KRAS G12D” refers to a mutant form of a mammalian KRAS protein that contains an amino acid substitution of an aspartic acid for a glycine at amino acid position 12. The assignment of amino acid codon and residue positions for human KRas is based on the amino acid sequence identified by UniProtKB/Swiss-Prot P01116: Variantp . Gly12Asp.

As used herein, a “KRAS G12D inhibitor” refers to compounds of the present invention that are represented by Formula (I), as described herein. These compounds are capable of negatively modulating or inhibiting all or a portion of the enzymatic activity of KRAS G12D.

A “KRAS G12D-associated disease or disorder” as used herein refers to diseases or disorders associated with or mediated by or having a KRAS G12D mutation. A non-limiting example of a KRAS G12D-associated disease or disorder is a KRAS G12D-associated cancer.

As used herein, the term “subject,” “individual” or “patient” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject has been identified or diagnosed as having a cancer having a KRAS G12D mutation (e.g., as determined using a regulatory agency-approved, e.g., FDA-approved, assay or kit). In some embodiments, the subject has a tumor that is positive for a KRAS G12D mutation (e.g., as determined using a regulatory agency-approved assay or kit). The subject can be a subject with a tumor(s) that is positive for a KRas G12D mutation (e.g., identified as positive using a regulatory agency-approved, e.g., FDA-approved, assay or kit). The subject can be a subject whose tumors have a KRAS G12D mutation (e.g., where the tumor is identified as such using a regulatory agency-approved, e.g., FDA-approved, kit or assay). In some embodiments, the subject is suspected of having a KRAS G12D gene-associated cancer. In some embodiments, the subject has a clinical record indicating that the subject has a tumor that has a KRAS G12D mutation (and optionally the clinical record indicates that the subject should be treated with any of the compositions provided herein).

The term “halogen” or “halo”, as used herein, unless otherwise indicated, means fluoro, chloro, bromo or iodo. The preferred halogen groups include —F, —Cl and —Br.

The term “alkyl”, as used herein, unless otherwise indicated, includes saturated monovalent hydrocarbon radicals having straight or branched. For example, —C₁₋₆alkyl radicals include methyl, ethyl, propyl, isopropyl, n-butyl, isobutyl, sec-butyl, t-butyl, n-pentyl, 3-(2-methyl)butyl, 2-pentyl, 2-methylbutyl, neopentyl, n-hexyl, 2-hexyl and 2-methylpentyl. Similary, C₁₋₃, as in C₁₋₃alkyl is defined to identify the group as having 1,2,3,4,5 or 6 carbon atoms in a linear or branched arrangement.

The term “haloalkyl” (such as —C₁₋₆haloalkyl, —C₁₋₄haloalkyl or —C₁₋₃haloalkyl) as used herein, unless otherwise indicated, an alkyl chain (such as —C₁₋₆alkyl, —C₁₋₄alkyl or —C₁₋₃alkyl) as defined herein in which one or more (such as one, two or three) hydrogen has been replaced by a halogen. Examples include trifluoromethyl, difluoromethyl and fluoromethyl.

The term “alkylene” means a difunctional group obtained by removal of a hydrogen atom from an alkyl group defined above. For example, methylene (i.e., —CH₂—), ethylene (i.e., —CH₂—CH₂— or —CH(CH₃)—) and propylene (i.e., —CH₂—CH₂—CH₂—, —CH(—CH₂—CH3)— or —CH₂—CH(CH₃)—).

The term “alkenyl” means a straight or branch-chained hydrocarbon radical containing one or more double bonds and typically from 2 to 20 carbon atoms in length. For example, “C₂₋₆alkenyl” contains from 2 to 6 carbon atoms. Alkenyl group include, but are not limited to, for example, ethenyl, propenyl, butenyl, 2-methyl-2-buten-1-yl, hepetenyl, octenyl and the like.

The term “alkynyl” contains a straight or branch-chained hydrocarbon radical containing one or more triple bonds and typically from 2 to 20 carbon atoms in length. For example, “C₂₋₆alkynyl” contains from 2 to 6 carbon atoms. Representative alkynyl groups include, but are not limited to, for example, ethynyl, 1-propynyl, 1-butynyl, heptynyl, octynyl and the like.

The term “alkoxy” radicals are oxygen ethers formed from the previously described alkyl groups.

The term “aryl”, as used herein, unless otherwise indicated, refers to an unsubstituted or substituted mono or polycyclic aromatic ring system containing carbon ring atoms. The preferred aryls are mono cyclic or bicyclic 6-10 membered aromatic ring systems. Phenyl and naphthyl are preferred aryls.

The term “heterocyclic”, as used herein, unless otherwise indicated, refers to unsubstituted and substituted mono or polycyclic non-aromatic ring system containing one or more heteroatoms, which comprising moncyclic heterocyclic ring, bicyclic heterocyclic ring, bridged heterocyclic ring, fused heterocyclic ring or sipro heterocyclic ring. Preferred heteroatoms include N, O, and S, including N-oxides, sulfur oxides, and dioxides. Preferably, the ring is three to ten membered and is either fully saturated or has one or more degrees of unsaturation. Multiple degrees of substitution, preferably one, two or three, are included within the present definition. Examples of such heterocyclic groups include, but are not limited to azetidinyl, pyrrolidinyl,piperidinyl, piperazinyl, oxopiperazinyl, oxopiperidinyl, oxoazepinyl, azepinyl, tetrahydrofuranyl, dioxolanyl, tetrahydroimidazolyl, tetrahydrothiazolyl, tetrahydrooxazolyl, tetrahydropyranyl, morpholinyl, thiomorpholinyl, thiamorpholinylsulfoxide, thiamorpholinylsulfone and oxadiazolyl.

The term “heteroaryl”, as used herein, unless otherwise indicated, represents an aromatic ring system containing carbon(s) and at least one heteroatom. Heteroaryl may be monocyclic or polycyclic, substituted or unsubstituted. A monocyclic heteroaryl group may have 1 to 4 heteroatoms in the ring, while a polycyclic heteroaryl may contain 1 to 10 hetero atoms. A polycyclic heteroaryl ring may contain fused, spiro or bridged ring junction, for example, bycyclicheteroaryl is a polycyclic heteroaryl. Bicyclic heteroaryl rings may contain from 8 to 12 member atoms. Monocyclic heteroaryl rings may contain from 5 to 8 member atoms (cabons and heteroatoms). Examples of heteroaryl groups include, but are not limited to thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl,benzotriazolyladeninyl, quinolinyl or isoquinolinyl.

The term “carbocyclic” refers to a substituted or unsubstituted monocyclic ring, bicyclic ring, bridged ring, fused ring, sipiro ring non-aromatic ring system only containing carbon atoms. Examplary “cycloalkyl” groups includes but not limited to, cyclopropyl, cyclobutyl, cyclopentyl, cyclohexyl and so on.

The term “oxo” refers to oxygen atom together with the attached carbon atom forms the group

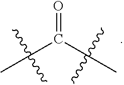 [SMILES: *C(*)=O]

The term “composition”, as used herein, is intended to encompass a product comprising the specified ingredients in the specified amounts, as well as any product which results, directly or indirectly, from combinations of the specified ingredients in the specified amounts. Accordingly, pharmaceutical compositions containing the compounds of the present invention as the active ingredient as well as methods of preparing the instan

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. When the compound of the present invention is basic, its corresponding salt can be conveniently prepared from pharmaceutically acceptable non-toxic acids, including inorganic and organic acids. Since the compounds in the present invention are intended for pharmaceutical use they are preferably provided in substantially pure form, for example at least 60% pure, more suitably at least 75% pure, especially at least 98% pure (% are on a weight for weight basis).

The present invention includes within its scope the prodrugs of the compounds of this invention. In general, such prodrugs will be functional derivatives of the compounds that are readily converted in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques know in the art as well as those methods set forth herein.

The present invention includes compounds described can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof.

The present invention includes all stereoisomers of the compound and pharmaceutically acceptable salts thereof. Further, mixtures of stereoisomers as well as isolated specific stereoisomers are also included. During the course of the synthetic procedures used to prepare such compounds or in using racemization or epimerization procedures known to those skilled in the art, the products of such procedures can be a mixture of stereoisomers.

The term “stereoisomer” as used in the present invention refers to an isomer in which atoms or groups of atoms in the molecule are connected to each other in the same order but differ in spatial arrangement, including conformational isomers and conformational isomers. The configuration isomers include geometric isomers and optical isomers, and optical isomers mainly include enantiomers and diastereomers. The invention includes all possible stereoisomers of the compound.

Certain of the compounds provided herein may exist as atropisomers, which are conformational stereoisomers that occur when rotation about a single bond in the molecule is prevented, or greatly slowed, as a result of steric interactions with other parts of the molecule. The compounds provided herein include all atropisomers, both as pure individual atropisomer preparations, enriched preparations of each, or a non-specific mixture of each. Where the rotational barrier about the single bond is high enough, and interconversion between conformations is slow enough, separation and isolation of the isomeric species may be permitted.

The present invention is intended to include all isotopes of atoms occurring in the present compounds. Isotopes include those atoms having the same atomic number but different mass numbers. By way of general example and without limitation, isotopes of hydrogen include deuterium and tritium. The isotopes of hydrogen can be denoted as ¹H(hydrogen), ²H(deuterium) and ³H(tritium). They are also commonly denoted as D for deuterium and T for tritium. In the application, CD₃ denotes a methyl group wherein all of the hydrogen atoms are deuterium. Isotopes of carbon include ¹³C and ¹⁴C. Isotopically-labeled compounds of the invention can generally be prepared by conventional techniques known to those skilled in the art or by prcesses analogous to those described herein, using an appropriate isotopically-labeled reagent in place of the non-labeled reagent.

When a tautomer of the compound in the present inventon exists, the present invention includes any possible tautomers and pharmaceutically acceptable salts thereof, and mixtures thereof, except where specifically stated otherwise.

When the compound in present invention and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable.

The pharmaceutical compositions of the present invention comprise a compound in present invention (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds in present invention or a prodrug or a metabolite or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in- oil liquid emulsion. In addition to the common dosage forms set out above, the compound represented by Formula I or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt. The compounds of Formula I or pharmaceutically acceptable salts thereof, can also be included in pharmaceutical compositions in combination with one or more other therapeutically active compounds.

The pharmaceutical carrier employed can be, for example, a solid, liquid or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient. For example, a formulation intended for the oral administration to humans may contain from about 0.5 mg to about 5 g of active agent, compounded with an appropriate and convenient amount of carrier material which may vary from about 0.05 to about 95 percent of the total composition. Unit dosage forms will generally contain between from about 0.01 mg to about 2 g of the active ingredient, typically 0.01 mg, 0.02 mg, 1 mg, 2 mg, 3 mg, 4 mg, 5 mg, 6 mg, 7 mg, 8 mg, 9 mg, 10 mg, 25 mg, 50 mg, 100 mg, 200 mg, 300 mg, 400 mg, 500 mg, 600 mg, 800 mg, 1000 mg, 1500 mg or 2000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder or the like. Further, the compositions can be in a form suitable for use in transdermal devices. These formulations may be prepared, utilizing a compound represented by Formula I of this invention or a pharmaceutically acceptable salt thereof, via conventional processing methods. As an example, a cream or ointment is prepared by admixing hydrophilic material and water, together with about 0.05 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound described by Formula I or pharmaceutically acceptable salts thereof, may also be prepared in powder or liquid concentrate form.

Generally, dosage levels on the order of from about 0.001 mg/kg to about 150 mg/kg of body weight per day are useful in the treatment of the above-indicated conditions or alternatively about 0.05 mg to about 7 g per patient per day. For example, inflammation, cancer, psoriasis, allergy/asthma, disease and conditions of the immune system, disease and conditions of the central nervous system (CNS), may be effectively treated by the administration of from about 0.001 to 50 mg of the compound per kilogram of body weight per day or alternatively about 0.05 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

These and other aspects will become apparent from the following written description of the invention.

It is to be understood that, if any prior art publication is referred to herein; such reference does not constitute an admission that the publication forms a part of the common general knowledge in the art in any country.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and Examples should not be construed as limiting the scope of the invention.

### Methods of Prepration

Compounds of the present invention can be synthesized from commercially available reagents using the synthetic methods and reaction schemes described herein. The examples which outline specific synthetic route, and the generic schemes below are meant to provide guidance to the ordinarily skilled synthetic chemist, who will readily appreciate that the solvent, concentration, reagent, protecting group, order of synthetic steps, time, temperature, and the like can be modified as necessary, well within the skill and judgment of the ordinarily skilled artisan.

### EXAMPLES

The following examples are provided to better illustrate the present invention. All parts and percentages are by weight and all temperatures are degrees celsius, unless explicitly stated otherwise. The abbreviations in following Table 12 have been used in the examples:

TABLE 12

| | | | |
|---|---|---|---|
| DMF | N,N-Dimethylformamide | TEA | Triethylamine |
| EtOAc, EA | Ethyl acetate | DABCO | Triethylenediamine |
| Hex | Hexane | aq. | aqueous |
| MeOH | Methanol | sat. | saturated |
| DCM | Dichloromethane | TLC | Thin layer |
| | | | chromatography |
| DCE | 1,2-Dichloroethane | Prep - TLC | Preparative thin layer |
| | | | chromatography |
| EtOH | Ethanol | MOMO | Methoxymethoxy |
| THF | Tetrahydrofuran | NCS | N-Chlorosuccinimide |
| DIPEA, DIEA | N,N-Diisopropylethylamine | TIPS | Triisopropylsilyl |
| Pd(PPh₃)₄ | Tetrakis(triphenylphosphine)palladium | IPA.M, MIPA, | Isopropylamine |
| | | or IPAMine | |
| Pd(dppf)Cl₂ | [1,1′-Bis(diphenylphosphino)ferro- | DEA | Diethylamine |
| | cene]dichloropalladium(II) | | |
| cataCXium A Pd G₃ | [(Di(1-adamantyl)-butylphosphine)- | MTBE | Methyl tert-butyl ether |
| | 2-(2′-amino-1,1′-biphenyl)]palladium(II) | | |
| | methanesulfonate | | |
| TFA | 2,2,2-Trifluoroacetic acid | | |

### Preparation of INT 1

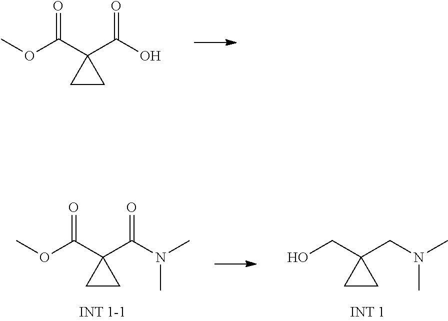

Oxalyl chloride (22.65 g, 178.45 mmol) was added to a mixed solution of 1-(methoxycarbonyl)cyclopropane-1-carboxylic acid (20.06 g, 139.18 mmol) in DCM (100 mL) and DMF (100 mg, 1.37 mmol) at 0° C. under nitrogen atmosphere. The solution was stirred for 3 h at room temperature. The solution was concentrated to give a yellow semi-solid. The semi-solid was taken up in THF (50 mL) and the solution was cooled to 0° C., and dimethylamine/THF (60 mL, 120 mmol, 2M) was added dropwise slowly and the resulting suspension was stirred for 2 hours. EtOAc (200 mL) was added and the mixture was washed with brine (2×200 mL). The organic layer was dried over anhydrous Na₂SO₄ and concentrated to yield Intermediate 1-1 (INT 1-1, 7.37 g, 43.05 mmol) as a yellow/brown oil. MS m/z: 172 [M+H]⁺.

Lithium aluminium hydride (3.21 g, 84.59 mmol) was added portion-wise to a solution of Intermediate 1-1 (INT 1-1, 7.37 g, 43.05 mmol) in THF at 0° C., and the resulting solution was stirred for 2 h at room temperature. The solution was cooled to 0° C. Water (3.5 mL), NaOH solution (15%, 3.5 mL), water (10 mL) were added portion-wise to give a white suspension. The resulting suspension was filtered through Celite, and the solids were washed with THF (150 mL). The combined filtrates were concentrated and purified by silica gel chromatography (eluting with DCM: MeOH=1:10, v/v) to give Intermediate 1 (INT 1, 3.79 g, 29.33 mmol) as a pale yellow oil. MS m/z: 130 [M+H]⁺.

### Preparation of INT 2

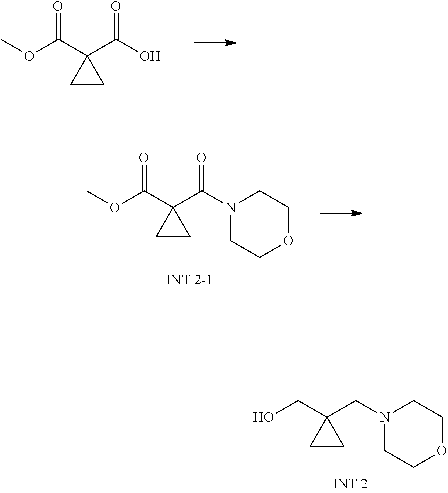

Oxalyl chloride (53.55 g, 0.42 mmol) was added to a mixed solution of 1-(methoxycarbonypcyclopropane-1-carboxylic acid (50.45 g, 350.04 mmol) in DCM (500 mL) and DMF (5.16 g, 70.59 mmol) at 0° C. under nitrogen atmosphere. The solution was stirred for about 3 hours at room temperature. The solution was concentrated under reduced pressure to give a yellow semi-solid. The semi-solid was dissolved in DCM (100 mL) and the resulting solution was added dropwise to a solution of morpholine (34.00 g, 390.27 mmol) and TEA (52.93 g, 523.08 mmol) in DCM (500 mL) at 0° C. The resulting suspension was stirred for 18 hours. Water (500 mL) was added and the mixture was extracted with EA. The organic layer was washed with aq. citric acid (5%, 500 mL) followed by brine (500 mL), then dried over anhydrous Na₂SO₄ and concentrated under reduced pressure. The residue was purified by silica gel chromatography (eluted with EtOAc: Hex) to yield INT 2-1 (70.05 g, 328.52 mmol, 93.85% yield) as a brown oil. MS m/z: 214 [M+H]⁺.

Lithium aluminium hydride (30 g, 790.51 mmol) was added portion-wise to a solution of INT 2-1 (70.05 g, 328.52 mmol) in THF (700 mL) at 0° C., and the resulting solution was stirred for 3 h at room temperature. The solution was cooled to 0° C. and water (30 mL), NaOH solution (15%, 30 mL), water (90 mL) were added portion-wise to give a white suspension. The resulting suspension was filtered through Celite, and the solids were washed with THF (150 mL). The combined filtrates were concentrated and purified by silica gel chromatography (eluting with DCM:MeOH=1:50, v/v) to give INT 2 (40.61 g, 237.16 mmol, 72.19 yield) as a colorless oil. MS m/z: 172 [M+H]⁺.

### Preparation of INT 3

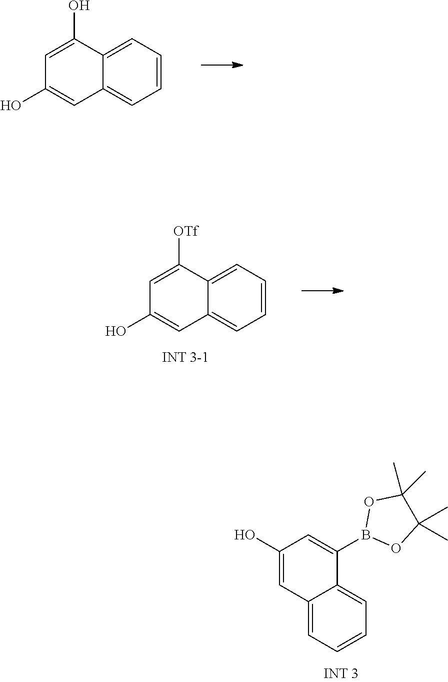 [SMILES: Oc1cc(O)c2ccccc2c1 ; O=S(=O)(Oc1cc(O)cc2ccccc12)C(F)(F)F ; CC1(C)OB(c2cc(O)cc3ccccc23)OC1(C)C]

Trifluoromethanesulfonic anhydride (15.55 g, 55.12 mmol) and DIEA (7.37 g, 57.03 mmol) were added dropwise simultaneously to a solution of 1,3-dihydroxynaphthalene (8.82 g, 55.07 mmol) in DCM (300 mL) at 10° C. After stirring for 1 h, the solution was partitioned between water and DCM. The organic phase was dried over anhydrous Na₂SO₄, filtered and concentrated under reduced pressure. The residue was purified by silica gel chromatography (eluting with hexane/EA) to give intermediate 3-1 (INT 3-1, 6.92 g, 23.68 mmol).

To a solution of Intermediate 3-1 (INT 3-1, 6.87 g, 23.51 mmol) and 4,4,4′,4′,5,5,5′,5′-octamethyl-2,2′-bi(1,3,2-dioxaborolane) (11.66 g, 45.92 mmol) in 1.4-dioxane (100 mL) were added KOAc (8.39 g, 85.49 mmol) and Pd(dppf)Cl₂ (1.73 g, 2.36 mmol). The reaction mixture was stirred at 85° C. for 4.5 hours under nitrogen atmosphere. The reaction mixture was diluted with water (100 mL) and extracted with EtOAc (150 mL, 100 mL). The combined organic layers were washed with brine (150 mL), dried over anhydrous Na₂SO₄ and concentrated under reduced pressure. The residue was purified by silica gel chromatography (eluting with Hex:EA=30:1-15:1, v/v) to give Intermediate 3 (INT 3, 7.14 g, 26.43 mmol). MS m/z: 271 [M+H]⁺.

### Preparation of INT 4

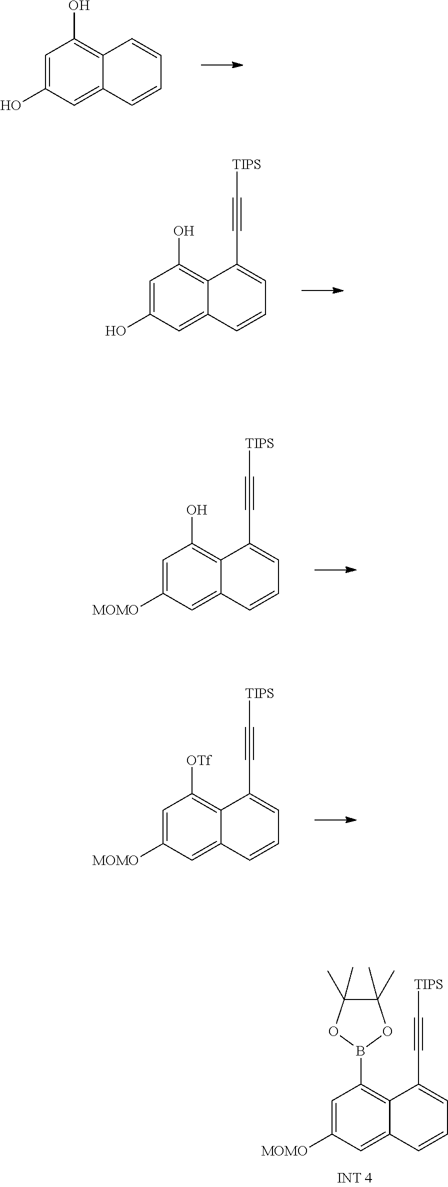

Following the procedure described in WO2021041671, INT 4 (Intermediate 4) was synthesized with naphthalene-1,3-diol as starting material.

### Preparation of INT 5

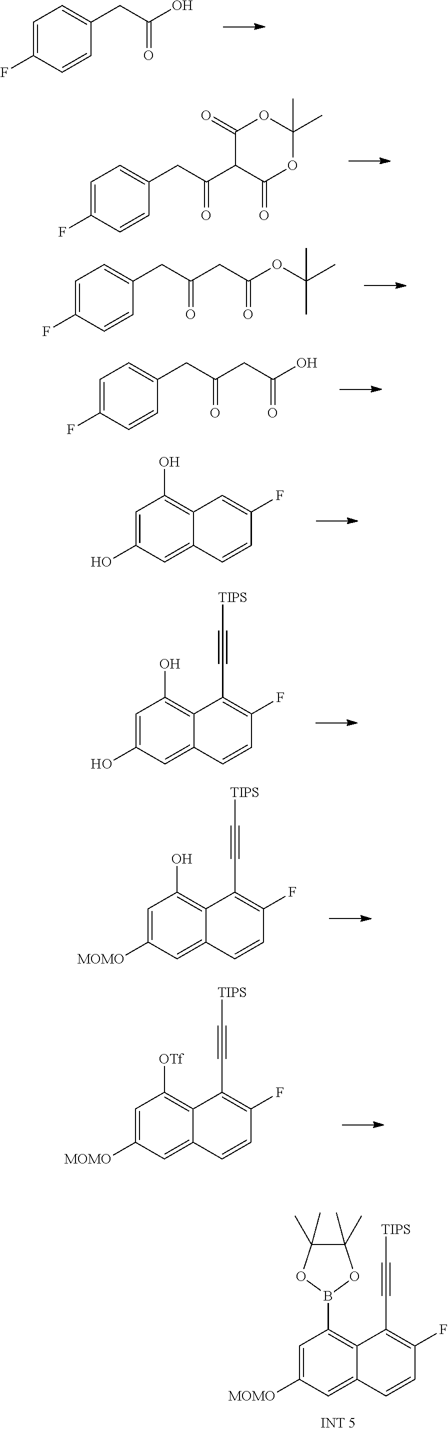

Following the procedure of WO2021041671, INT 5 (Intermediate 5) was synthesized with 2-(4-fluorophenyl) acetic acid as starting material.

### Preparation of INT 6A and INT 6B

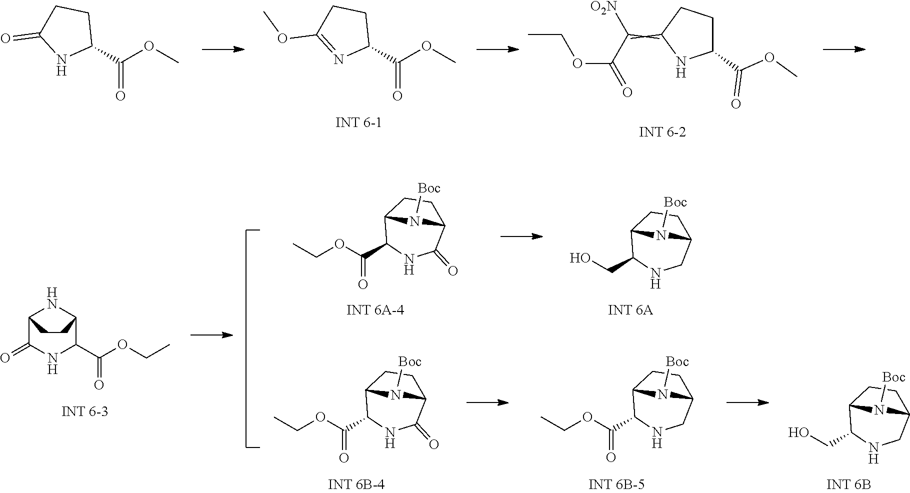

A mixture of methyl (R)-5-oxopyrrolidine-2-carboxylate (49.34 g, 344.70 mmol) and dimethyl sulfate (58.60 g, 464.59 mmol) was stirred for 22 hours at 60° C. under nitrogen atmosphere. The reaction mixture was added dropwise to a mixture of MTBE (200 mL) and TEA (30 mL) at 0° C. The resulting mixture was diluted with sat. aq. K₂CO₃ (150 mL) and extracted with MTBE (5×100 mL). The combined organic phases were dried over anhydrous Na₂SO₄, filtered and concentrated under reduced pressure to give the product INT 6-1 (47.37 g, 87.44%). MS (ESI, m/z): 158 [M+H]⁺.

A solution of INT 6-1 (47.37 g, 301.39 mmol) in ethyl 2-nitroacetate (121.31 g, 911.40 mmol) was stirred at 60° C. for 24 hours. The resulting mixture was concentrated under reduced pressure and purified by silica gel column chromatography, eluted with 0- 30% EA in Hex to afford INT 6-2 (33.22 g, 128.64 mmol, 42.6% yield). MS (ESI, m/z): 259 [M+H]⁺.

To a solution of INT 6-2 (38.66 g, 149.71 mmol) in ethanol (1000 mL) was added Pd/C (30.33 g, 28.50 mmol). The reaction mixture was stirred at room temperature under hydrogen atmosphere for 60 hours. The resulting mixture was filtered and the filtrate was concentrated under reduced pressure. The residue was purified by silica gel column chromatography, eluted with 0-10% methanol in DCM to afford INT 6-3 (16.24 g, 81.92 mmol, 54.7% yield). MS (ESI, m/z): 199 [M+H]⁺.

To a solution of INT 6-3 (16.24 g, 81.92 mmol) in DCM (200 mL) were added di-tert-butyl dicarbonate (17.78 g, 81.46 mmol) and triethylamine (24.75 g, 244.61 mmol) at 0° C. The reaction mixture was stirred at room temperature for 16 hours. The resulting mixture was quenched with water (100 mL) and extracted with DCM (2×100 mL). The resulting organic layers were dried over anhydrous sodium sulphate, concentrated under reduced pressure. The residue was purified by silica gel column chromatography, eluted with 0-50% EtOAc in Hex to afford INT 6B-4 (16.22 g, 54.36 mmol, 66.3% yield) MS (ESI, m/z): 299 [M+H]⁺, and eluted subsequently with 50-70% EtOAc in Hex to afford INT 6A-4 (5.23 g, 17.53 mmol, 21.3% yield). MS (ESI, m/z): 299 [M+H]⁺.

A solution of INT 6B-4 (16.22 g, 54.36 mmol) in Borane-dimethyl sulfide complex (2 M in THF, 140 mL) was stirred at room temperature for 16 hours under nitrogen atmosphere. The resulting mixture was quenched with methanol (50 mL) and stirred at 50° C. for 16 hours. The mixture was concentrated and the residue was purified by silica gel column chromatography, eluted with 0-50% EA in Hex to afford INT 6B-5 (9.36 g, 32.91 mmol, 60.5% yield). MS (ESI, m/z): 285 [M+H]⁺.

To a 0° C. solution of INT 6B-5 (4.08 g, 14.35 mmol) in THF (30 mL) was added Lithium Aluminum Hydride (1 M in THF, 20 mL, 20.00 mmol). The reaction mixture was stirred at room temperature for 2 h, quenched with ice water (5 mL), and filtered. The filtrate was concentrated under reduced pressure. The residue was purified by silica chromatography (eluting with DCM:MeOH=30:1˜15:1, v/v) to give INT 6B (2.55 g, 10.52 mmol). MS: m/z 243 [M+1]⁺.

A solution of INT 6A-4 (1.02 g, 3.42 mmol) in Borane-dimethyl sulfide complex (2 M in THF, 8 mL) was stirred at room temperature for 16 hours under nitrogen atmosphere. The resulting mixture was quenched with methanol (50 mL) and stirred at 50° C. for 16 hours. The mixture was concentrated and the residue was purified by silica gel chromatography (eluting with from Hex:EA=1:1 to DCM:MeOH=15:1, v/v) to give INT 6A (453 mg, 1.87 mmol). MS: m/z 243 [M+H]⁺.

### Preparation of INT 7A and INT 7B

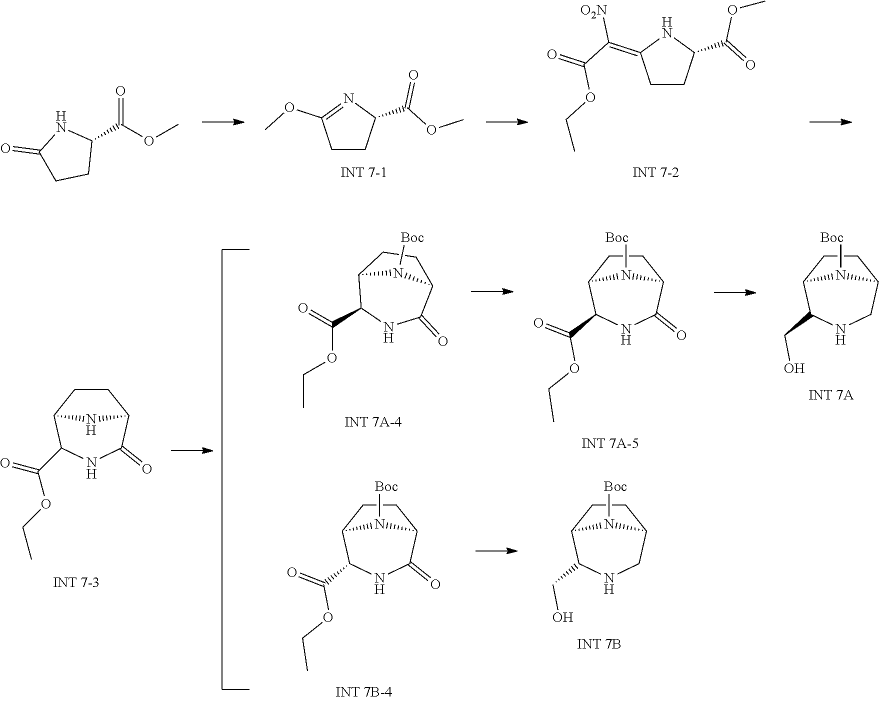

A solution of methyl (S)-5-oxopyrrolidine-2-carboxylate (40.04 g, 279.72 mmol) in dimethyl sulfate (44.80 g, 355.18 mmol) was stirred at 60° C. for 18 hours. The resulting mixture was added to a solution of TEA (40 g) in MTBE (200 mL) at 0° C. The resulting mixture was added aq. Na₂CO₃ (sat., 200 mL), extracted with MTBE (2×100 mL), the organic layers was dried over anhydrous sodium sulphate, concentrated under reduced pressure. The residue was purified by silica gel column chromatography (eluted with 0-10% MeOH in DCM) to afford INT 7-1 (35.47 g, 225.68 mmol, 80.6% yield). MS (ESI, m/z): 158 [M+H]⁺.

A solution of INT 7-1 (35.47 g, 225.68 mmol) in ethyl 2-nitroacetate (91.00 g, 683.68 mmol) was stirred at 60° C. for 24 hours. The resulting mixture was concentrated under reduced pressure and purified by silica gel column chromatography (eluted with 0-30% EtOAc in Hex) to afford INT 7-2 (24.48 g, 94.80 mmol, 42.0% yield). MS (ESI, m/z): 259 [M+H]⁺.

To a solution of INT 7-2 (32.98 g, 127.71 mmol) in ethanol (1000 mL) was added Pd/C (32.55 g, 30.58 mmol) at hydrogen atmosphere. The reaction mixture was stirred at room temperature for 60 hours. The resulting mixture was filtered and the filtrate was concentrated under reduced pressure. The residue was purified by silica gel column chromatography (eluted with 0-10% methanol in DCM) to afford INT 7-3 (10.19 g, 51.40 mmol, 40.3% yield). MS (ESI, m/z): 199 [M+H]⁺.

To a solution of INT 7-3 (10.19 g, 51.40 mmol) in DCM (100 mL) were added di-tert-butyl dicarbonate (11.67 g, 53.47 mmol) and triethylamine (15.62 g, 154.36 mmol) at 0° C. The reaction mixture was stirred at room temperature for 16 hours. The resulting mixture was quenched by water (100 mL), extracted with DCM (2×100 mL), the organic layers was dried over anhydrous sodium sulphate, concentrated under reduced pressure. The residue was purified by silica gel column chromatography, eluted with 0-50% EtOAc in Hex to afford INT 7A-4 (8.67 g, 29.06 mmol, 56.5% yield) MS (ESI, m/z): 299 [M+H]⁺. And eluted with 50-70% EtOAc in Hex to afford INT 7B-4 (3.67 g, 12.30 mmol, 23.9% yield). MS (ESI, m/z): 299 [M+H]⁺.

A solution of INT 7A-4 (8.67 g, 29.06 mmol) in borane-methyl sulfide complex (2 M in THF, 60 mL) was stirred at room temperature for 16 hours under nitrogen atmosphere. The resulting mixture was quenched by methanol (50 mL) and stirred at 50° C. for 16 hours. The mixture was concentrated and the residue was purified by silica gel column chromatography, eluted with 0-50% EtOAc in Hex to afford INT 7A-5 (4.73 g, 16.63 mmol, 57.2% yield). MS (ESI, m/z): 285 [M+H]⁺.

To a solution of INT 7A-5 (4.2 g, 14.77 mmol) in THF (60 mL) was added LAH (1 M in THF, 25 mL) at 0° C. The reaction mixture was stirred at room temperature for 2 hours. The resulting mixture was quenched by water (1 mL), 15% NaOH (1 mL), water (3 mL). The solution was filtered, the filtrate was concentrated under reduced pressure and the residue was purified by silica gel column chromatography, eluted with 0-10% methanol in DCM to afford INT 7A (1.93 g, 7.96 mmol, 53.9% yield). MS (ESI, m/z): 243 [M+H]⁺.

A solution of INT 7B-4 (3.67 g, 12.30 mmol) in borane-methyl sulfide complex (2 M in THF, 30 mL) was stirred at room temperature for 16 hours under nitrogen atmosphere. The resulting mixture was quenched by methanol (30 mL) and stirred at 50° C. for 16 hours. The mixture was concentrated and the residue was purified by silica gel column chromatography, eluted with 0-10% methanol in DCM to afford INT 7B (857 mg, 3.54 mmol, 28.8% yield). MS (ESI, m/z): 243 [M+H]⁺.

### Preparation of INT 8

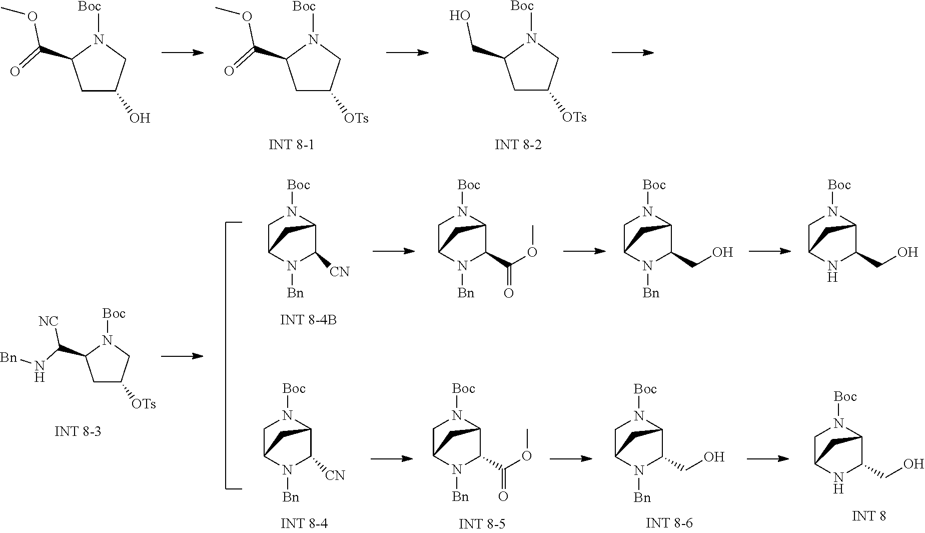

A solution of 1-(tert-butyl) 2-methyl (2S,4R)-4-hydroxypyrrolidine-1,2-dicarboxylate (11.10 g, 45.26 mmol) in pyridine (100 mL) was cooled to 0° C., tosyl chloride (22.49 g, 117.97 mmol) was added in portions. The mixture was stirred at room temperature for 24 hours. The solution was concentrated under reduced pressure. The residue was diluted with water and extracted with EtOAc (50 mL), the organic layer was washed with sat. aq. NH₄Cl (3×100 mL). The organic layer was dried over Na₂SO₄ and concentrated under reduced pressure to afford the desired product INT 8-1 (25.79 g, 64.56 mmol) as a yellow semi-solid. MS m/z: 400 [M+H]⁺.

To a solution of INT 8-1 (25.79 g, 64.56 mmol) in THF (120 mL) was added LiBH₄ (75 mL, 2 M in THF) dropwise at 0° C. The mixture was stirred at room temperature for 3 hours. The solution was quenched with water (20 mL) at 0° C. The mixture was extracted with EtOAc (150 mL), and washed with sat. aq. NaHCO₃ (150 mL). The organic layer was dried over Na₂SO₄ and concentrated under reduced pressure. The residue was purified by silica gel chromatography (eluted with DCM:MeOH=100:1, v/v) to afford the desired product INT 8-2 (14.82 g, 39.90 mmol) as an off-white solid. MS m/z: 372 [M+H]⁺.

A solution of oxalyl chloride (6.86 g, 54.05 mmol) in DCM (250 mL) at −78° C. was added anhydrous DMSO (4.58 g, 58.62 mmol). The mixture was stirred for 15 min, after which a solution of INT 8-2 (13.33 g, 35.89 mmol) in DCM (30 mL) was added slowly. After an additional 30 min, TEA (10.92 g, 107.92 mmol) was added and the reaction mixture was allowed warm to 0° C. The mixture was then quenched with water (50 mL) and diluted with DCM (100 mL). The mixture was then washed with hydrochloric acid (100 mL, 0.5 N), saturated aqueous NaHCO₃ (100 mL) and brine (100 mL) sequentially. The organic layer was dried over anhydrous sodium sulfate and concentrated under reduced pressure. The crude aldehyde intermediate was dissolved in THF (150 mL) and diethyl cyanophosphonate (90% wt %, 7.86 g, 48.19 mmol) was added followed by benzyl amine (10.15 g, 94.72 mmol). Upon completion, the reaction mixture was concentrated under reduced pressure. The residue was purified by silica gel chromatography (eluted with Hex:EtOAc=10:1, v/v) to afford the desired product INT 8-3 (6.25 g, 12.87 mmol) as a light yellow solid. MS m/z: 486 [M+H]⁺.

To a solution of INT 8-3 (6.25 g, 12.87 mmol) in DCE (80 mL) was added DIEA (6.5 mL). The mixture was stirred at 100° C. overnight. The solution was concentrated under reduced pressure. The residue was extracted with EtOAc (30 mL), and washed with brine (3×30 mL). The organic layer was dried over Na₂SO₄ and concentrated under reduced pressure. The residue was purified by silica gel chromatography (eluted with Hex:EtOAc=10:1, v/v) to afford the desired product INT 8-4 (1.48 g, the first elution) as a oil. MS m/z: 314 [M+H]⁺, and INT 8-4B (2.36 g, the second elution) as a yellow solid. MS m/z: 314 [M+H]⁺.

INT 8-4 (2.10 g, 6.70 mmol) was dissolved in NaOMe (sodium: 1.82 g, 33.61 mmol, dissolved in 67 mL MeOH) and heated to 60° C. for 5 hours under nitrogen atmosphere. The reaction mixture was cooled to 0° C. and hydrochloric acid (20 mL, 3 N) was added slowly. After stirred for 2 hours at 0° C., the reaction mixture was poured into saturated aqueous NaHCO₃ (50 mL). The mixture was extracted with DCM (2×30 mL) and the combined organic layers were dried over anhydrous sodium sulfate. The residue was purified by silica gel chromatography (eluted with Hex:EtOAc=8:1, v/v) to afford the desired product INT 8-5 (1.65 g, 4.76 mmol) as oil. MS m/z: 347 [M+H]⁺.

A solution of INT 8-5 (1.65 g, 4.76 mmol) in THF (20 mL) was cooled to 0° C. LiBH₄ (6 mL, 2 M in THF) was added dropwise. The mixture was stirred at room temperature overnight. The solution was quenched with water (10 mL) at 0° C. The mixture was extracted with EtOAc (50 mL), and the organic layer was washed with sat. aq. NaHCO₃ (50 mL). The organic layer was dried over Na₂SO₄ and concentrated under reduced pressure. The residue was purified by silica gel chromatography (eluted with DCM:MeOH=100:1, v/v) to afford the desired product INT 8-6 (1.44 g, 4.52 mmol) as oil. MS m/z: 319 [M+H]⁺.

A solution of INT 8-6 (411 mg, 1.29 mmol), Pd/C (559 mg, 0.53 mmol, 10% wt) in MeOH (15 mL) was stirred at 75° C. for 1.5 hours under hydrogen atmosphere. The reaction mixture was allowed to cool to room temperature and filtered, the filtration was concentrated under reduced pressure to afford the desired product INT 8 (334 mg, 1.46 mmol). MS m/z: 229 [M+H]⁺.

### Preparation of INT 9

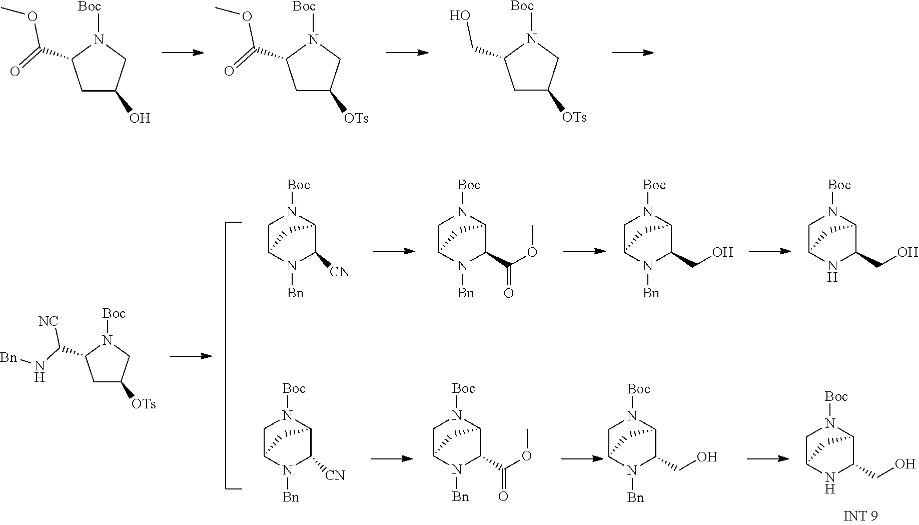

Following the procedure of INT, INT 9 (Intermediate 9) was synthesized with 1-(tert-butyl) 2-methyl (2R,4S)-4-hydroxypyrrolidine-1,2-dicarboxylate as starting material.

### Example 1

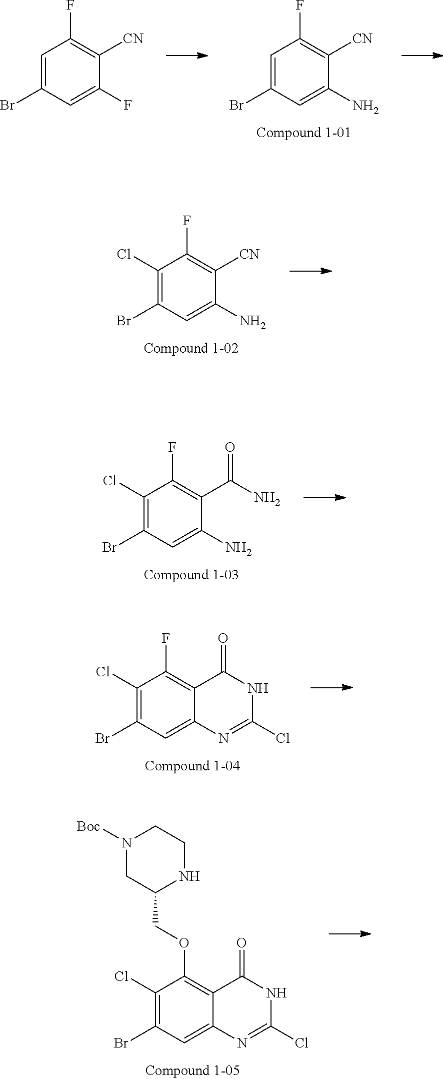 [SMILES: N#Cc1c(F)cc(Br)cc1F ; N#Cc1c(N)cc(Br)cc1F ; N#Cc1c(N)cc(Br)c(Cl)c1F ; NC(=O)c1c(N)cc(Br)c(Cl)c1F ; O=c1[nH]c(Cl)nc2cc(Br)c(Cl)c(F)c12 ; CC(C)(C)OC(=O)N1CCN[C@H](COc2c(Cl)c(Br)cc3nc(Cl)[nH]c(=O)c23)C1]

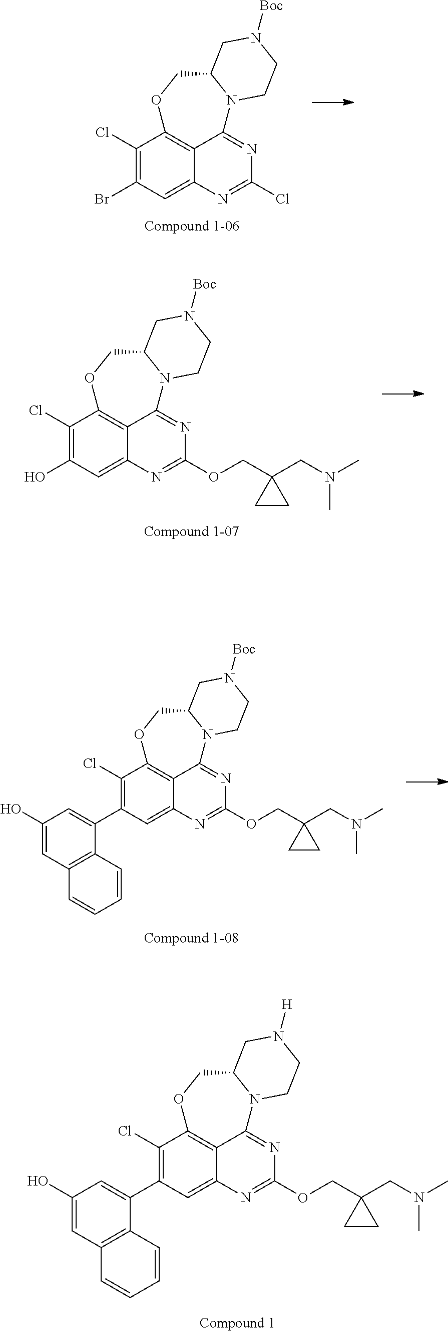 [SMILES: CC(C)(C)OC(=O)N1CCN2c3nc(Cl)nc4cc(Br)c(Cl)c(c34)OC[C@@H]2C1 ; CN(C)CC1(COc2nc3c4c(c(Cl)c(O)cc4n2)OC[C@@H]2CN(C(=O)OC(C)(C)C)CCN32)CC1 ; CN(C)CC1(COc2nc3c4c(c(Cl)c(-c5cc(O)cc6ccccc56)cc4n2)OC[C@@H]2CN(C(=O)OC(C)(C)C)CCN32)CC1 ; CN(C)CC1(COc2nc3c4c(c(Cl)c(-c5cc(O)cc6ccccc56)cc4n2)OC[C@@H]2CNCCN32)CC1]

28% Ammonium hydroxide (15 mL) was added to a microwave vial containing 4-bromo-2,6-difluorobenzonitrile (5.04 g, 23.12 mmol) in i-PrOH (10 mL). The mixture was capped and stirred at 80° C. for 4 hours. This reaction was repeated one more time, then the reaction mixtures were combined and poured into water (300 mL) and stirred for 15 minutes. A white solid was filtered which was washed with water and dried under reduced pressure to give Compound 1-01 (7.95 g, 36.97 mol). MS: m/z 215 [M+1]⁺, 217 [M+3]⁺.

To a solution of Compound 1-01 (3.05 g, 14.18 mmol) in acetonitrile (20 mL) was added NCS (2.10 g, 15.73 mmol). The mixture was stirred at 65° C. for 18 hours. Upon completion, the reaction mixture was evaporated, the residue was dissolved in DCM (100 mL), washed with water (60 mL) and separated. The collected organic layer was concentrated under reduced pressure. The residue was purified by silica chromatography (eluting with EA:Hex=6:1, v/v) to give Compound 1-02 (1.93 g, 7.74 mol). MS: m/z 249 [M+1]⁺, 251 [M+3]⁺.

To a solution of Compound 1-02 (1.93 g, 7.74 mmol) and potassium carbonate (2.21 g, 15.99 mmol) in DMSO (20 mL) was added 30% Hydrogen peroxide (3 mL) dropwise and the mixture was stirred at room temperature for 1 hour. The reaction mixture was poured into water (200 mL), stirred for one hour and the solid was filtered off, washed thoroughly with water and dried to afford Compound 1-03 (1.66 g, 6.21 mol). MS: m/z 267 [M+1]⁺, 269 [M+3]⁺.

To a solution of Compound 1-03 (1.03 g, 3.85 mmol) in 1,4-Dioxane (10 mL) was added thiophosgene (0.7 mL) and the mixture was stirred at room temperature for 1 h then at 105° C. for 1 hour. The mixture was allowed to cool, then the solvent was concentrated under reduced pressure to afford Compound 1-04 (1275 mg, 4.09 mol). MS: m/z 311[M+1]⁺, 313 [M+3]⁺.

To a solution of (S)-4-N-Boc-2-(hydroxymethyl)piperazine (350 mg, 1.62 mmol) in dry THF (6 mL) was added NaH (262 mg, 6.55 mmol) under nitrogen at 0° C. and then stirred at room temperature for 30 minutes. Then the solution of Compound 1-04 (504 mg, 1.62 mmol) in dry THF (4 mL) was added and the reaction mixture was stirred at room temperature for 30 minutes then at 65° C. for 30 minutes and allowed to cool. The reaction mixture was diluted with EtOAc (30 mL), water (30 mL),and acidified by 5% Citric acid until pH=4˜5. The suspension was filtered and the filter cake was dried under reduced pressure to give Compound 1-05 (550 mg, 1.08 mol) as a light brown solid. MS: m/z507[M+1]⁺, 509 [M+3]⁺.

To a solution of Compound 1-05 (464 mg, 0.91 mol) in phosphorus oxychloride (5 mL) was added DIEA (0.5 mL) under nitrogen and then stirred at 90° C. for 3 hours. Upon completion, the resulting was concentrated under reduced pressure. The residue was diluted with DCM (30 mL), and then quenched by saturated NaHCO₃ (50 mL) and separated. The collected organic layer was washed with water (20 mL), dried over anhydrous Na₂SO₄, filtered and concentrated under reduced pressure to give Compound 1-06 (447 mg, 0.91 mol). MS: m/z 489[M+1]⁺, 491 [M+3]⁺.

To a solution of Compound 1-06 (221 mg, 0.45 mol) and Intermediate 1 (INT 1, 121 mg, 0.94 mmol) in DMSO (5 mL) was added KF (83 mg, 1.43 mmol) and the mixture was purged with nitrogen, followed by stirring at 120° C. for 22 hours. The mixture was diluted with EtOAc (50 mL) and water (40 mL) and the organic layer was separated. The organic layer was washed with brine (40 mL) and dried over anhydrous Na₂SO₄, filtered and concentrated under reduced pressure. The residue was purified by Pre-TLC (DCM:MeOH=10:1, v/v) to give Compound 1-07 (103 mg, 0.18 mmol). MS: m/z 582 [M+1]⁺, 584 [M+3]⁺.

To a solution of Compound 1-07 (103 mg, 0.18 mmol), Intermediate 3 (INT 3, 94 mg, 0.35 mmol) and Na₂CO₃ (64 mg, 060 mmol) in 1,4-dioxane (8 mL) and water (2 mL) was added Pd(PPh₃)₄ (25 mg, 0.021 mmol) and the mixture was purged with N₂ followed by stirring at 70° C. for 3 hours. Upon completion, the reaction mixture was concentrated under reduced pressure. Then, the residue was diluted with EtOAc (50 mL) and water (40 mL) and the organic layer was separated. The combined organic layers were concentrated under reduced pressure. The residue was purified by silica chromatography (eluting with DCM:MeOH=8:1, v/v) to give Compound 1-08 (104 mg, 0.16 mmol). MS: m/z 646 [M+1]⁺, 648[M+3]⁺.

To a solution of Compound 1-08 (104 mg, 0.16 mmol) in DCM (5 mL) was added TFA (1 mL). The reaction mixture was stirred at room temperature for 1 hour. Upon completion, the reaction mixture was concentrated under reduced pressure. The residue was purified by Pre-HPLC (0.1% TFA in water/acetonitrile) to give Compound 1 (93.2 mg). MS: m/z 546 [M+1]⁺, 548 [M+1]⁺.

¹H NMR (400 MHz, MeOD-d4) δ7.74 (d, 1H), 7.44-7.37 (m, 1H), 7.34-7.31 (m, 1H), 7.30-7.14 (m, 3H), 7.02-6.95 (m, 1H), 5.45 (d, 1H), 4.85-4.67 (m, 3H), 4.64-4.44 (m, 3H), 3.90-3.47 (m, 4H), 3.38-3.24 (m, 8H), 1.09-0.87 (m, 4H).

### Example 2

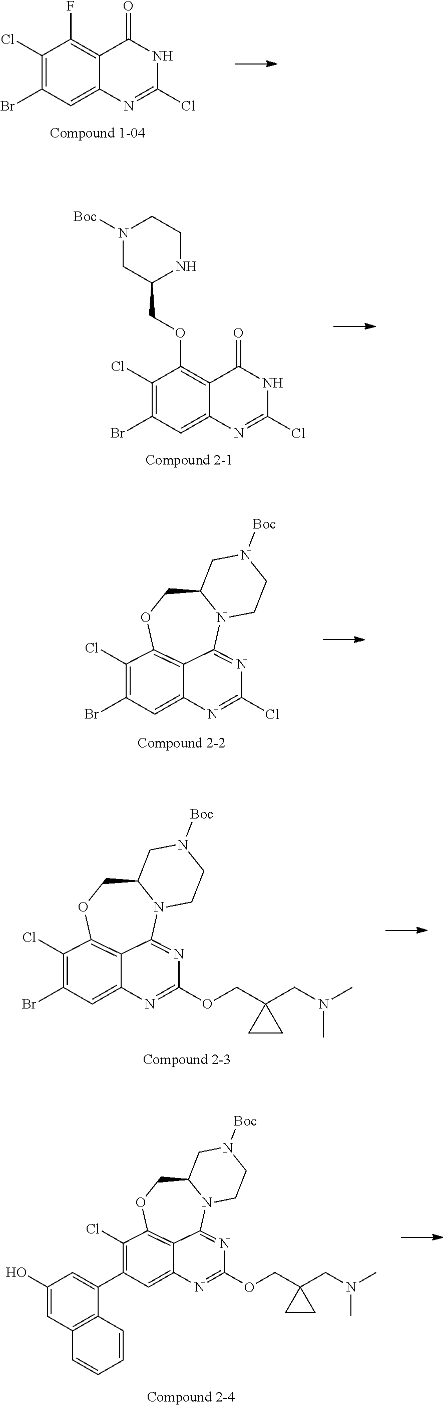 [SMILES: O=c1[nH]c(Cl)nc2cc(Br)c(Cl)c(F)c12 ; CC(C)(C)OC(=O)N1CCN[C@@H](COc2c(Cl)c(Br)cc3nc(Cl)[nH]c(=O)c23)C1 ; CC(C)(C)OC(=O)N1CCN2c3nc(Cl)nc4cc(Br)c(Cl)c(c34)OC[C@H]2C1 ; CN(C)CC1(COc2nc3c4c(c(Cl)c(Br)cc4n2)OC[C@H]2CN(C(=O)OC(C)(C)C)CCN32)CC1 ; CN(C)CC1(COc2nc3c4c(c(Cl)c(-c5cc(O)cc6ccccc56)cc4n2)OC[C@H]2CN(C(=O)OC(C)(C)C)CCN32)CC1]

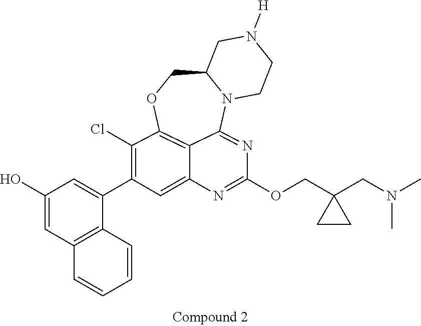 [SMILES: CN(C)CC1(COc2nc3c4c(c(Cl)c(-c5cc(O)cc6ccccc56)cc4n2)OC[C@H]2CNCCN32)CC1]

To a solution of (R)-1-Boc-3-hydroxymethyl-piperazine (325 mg, 1.51 mmol) in dry THF (10 mL) was added NaH (84 mg, 2.10 mmol) under nitrogen atmosphere at 0° C. and the mixture was stirred at room temperature for 30 minutes. Then a solution of Compound 1-04 (470 mg, 1.51 mmol) in dry THF (5 mL) was added to the reaction mixture and stirred at room temperature for 30 minutes and at 65° C. for 30 minutes and allowed to cool to room temperature. The reaction mixture was diluted with EtOAc (30 mL), water (30 mL), and acidified by 5% citric acid until pH=4˜5. The suspension was filtered and the filter cake was dried under reduced pressure to give Compound 2-1 (390 mg, 0.77 mmol) as a light brown solid. MS: m/z 507 [M+1]⁺, 509 [M+3]⁺.

DIEA (0.2 mL) and phosphorus oxychloride (0.5 mL) were added to a solution of Compound 2-1 (366.3 mg, 0.72 mmol) in toluene (5 mL) and then stirred at 80° C. for 2 hours under nitrogen atmosphere. Upon completion, the resulting mixture was concentrated under reduced pressure. The residue was diluted with DCM (30 mL), and then quenched by saturated NaHCO₃ (50 mL) and separated. The organic layer was washed with water (20 mL), dried over anhydrous Na₂SO₄, filtered and concentrated under reduced pressure. The residue was purified by silica gel chromatography (eluted with Hex:EtOAc=10:1, v/v) to give Compound 2-2 (279 mg, 0.57 mmol). MS: m/z489 [M+1]⁺, 491 [M+3]⁺.

To a solution of Compound 2-2 (137 mg, 0.28 mol) and INT 1 (80 mg, 0.62 mmol) in DMSO (4 mL) was added KF (53 mg, 0.91 mmol) and the mixture was purged with nitrogen followed by stirring at 120° C. for 24 hours. The mixture was diluted with EtOAc (50 mL) and water (40 mL) and the organic layer was separated. The organic layer was washed with brine (40 mL) and dried over anhydrous Na₂SO₄, filtered and concentrated under reduced pressure. The residue was purified by Pre-TLC (eluted with DCM:MeOH=8:1, v/v) to give Compound 2-3 (94 mg, 0.16 mmol). MS: m/z 582 [M+1]⁺, 584 [M+3]⁺.

To a solution of Compound 2-3 (94 mg, 0.16 mmol), INT 3 (70 mg, 0.26 mmol) and Na₂CO₃ (52 mg, 0.49 mmol) in 1,4-dioxane (8 mL) and water (2 mL) was added Pd(PPh₃)₄ (20 mg, 0.017 mmol) and the mixture was purged with nitrogen followed by stirring at 70° C. for 3 hours. Upon completion, the reaction mixture was concentrated under reduced pressure, the residue was diluted with EtOAc (40 mL) and water (25 mL) and the organic layer was separated. The organic layer was concentrated under reduced pressure. The residue was purified by silica gel chromatography (eluted with DCM:MeOH=8:1, v/v) to give Compound 2-4 (94 mg, 0.15 mmol). MS: m/z 646 [M+H]⁺.

To a solution of Compound 2-4 (94 mg, 0.15 mmol) in DCM (5 mL) was added TFA (1 mL). The reaction mixture was stirred at room temperature for 1 hour. Upon completion, the reaction mixture was concentrated under reduced pressure. The residue was purified by Pre-HPLC (C18 column, A: 0.1% TFA in water, B: MeOH, Gradient: 20% B to 55% B in 40 min at a flow rate of 40 mL/min, 290 nm) to give Compound 2 (78 mg, 0.10 mmol). MS: m/z 546 [M+H]⁺.

### Example 3

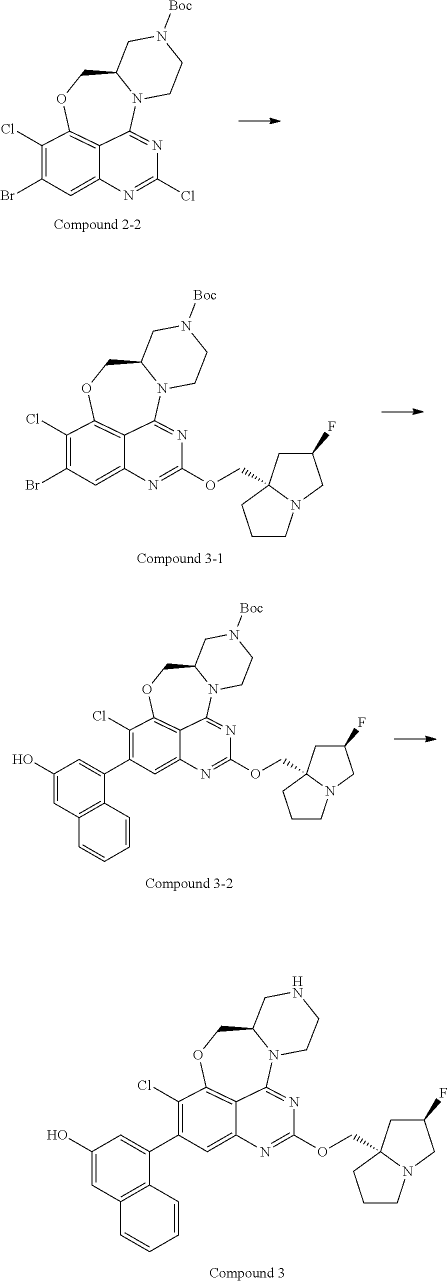 [SMILES: CC(C)(C)OC(=O)N1CCN2c3nc(Cl)nc4cc(Br)c(Cl)c(c34)OC[C@H]2C1 ; CC(C)(C)OC(=O)N1CCN2c3nc(OC[C@@]45CCCN4C[C@H](F)C5)nc4cc(Br)c(Cl)c(c34)OC[C@H]2C1 ; CC(C)(C)OC(=O)N1CCN2c3nc(OC[C@@]45CCCN4C[C@H](F)C5)nc4cc(-c5cc(O)cc6ccccc56)c(Cl)c(c34)OC[C@H]2C1 ; Oc1cc(-c2cc3nc(OC[C@@]45CCCN4C[C@H](F)C5)nc4c3c(c2Cl)OC[C@H]2CNCCN42)c2ccccc2c1]

To a solution of Compound 2-2 (142 mg, 0.29 mol) and ((2R,7aS)-2-fluorotetrahydro-1H-pyrrolizin-7a(5H)-yl)methanol (117 mg, 0.73 mmol) in DMSO (4 mL) was added KF (53 mg, 0.91 mmol) and the mixture was purged with nitrogen followed by stirring at 120° C. for 24 hours. The mixture was diluted with EtOAc (50 mL) and water (40 mL) and the organic layer was separated. The organic layer was washed with brine (40 mL) and dried over anhydrous Na₂SO₄, filtered and concentrated under reduced pressure. The residue was purified by Pre-TLC (DCM:MeOH=20:1, v/v) to give Compound 3-1 (67 mg, 0.11 mmol). MS: m/z 612 [M+1]⁺, 614 [M+3]⁺.

To a solution of Compound 3-1 (67 mg, 0.11 mmol), INT 3 (62 mg, 0.23 mmol) and Na₂CO₃ (35 mg, 0.33 mmol) in 1,4-dioxane (4 mL) and water (1 mL) was added Pd(PPh₃)₄ (15 mg, 0.013 mmol) and the mixture was purged with nitrogen followed by stirring at 70° C. for 3 hours. Upon completion, the reaction mixture was concentrated under reduced pressure, diluted with EtOAc (40 mL) and water (25 mL) and the organic layer was separated. The organic layer was concentrated under reduced pressure. The residue was purified by silica gel chromatography (eluting with DCM:MeOH=15:1, v/v) to give Compound 3-2 (36 mg, 0.053 mmol). MS: m/z 676 [M+H]⁺.

To a solution of Compound 3-2 (36 mg, 0.053 mmol) in DCM (5 mL) was added TFA (1 mL). The reaction mixture was stirred at room temperature for 3 hours. Upon completion, the reaction mixture was concentrated under reduced pressure. The residue was purified by Pre-HPLC (C18 column, A: 0.1% TFA in water, B: MeOH, Gradient: 20% B to 55% B in 45 min at a flow rate of 60 mL/min, 290 nm) to give Compound 3 (27.2 mg, 0.034 mmol). MS: m/z 576 [M+H]⁺.

### Example 4

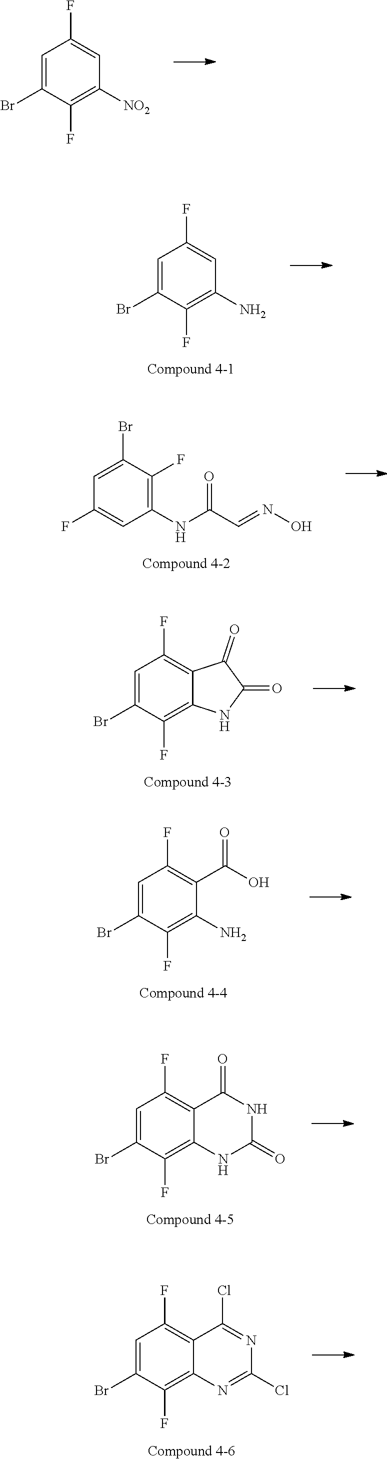

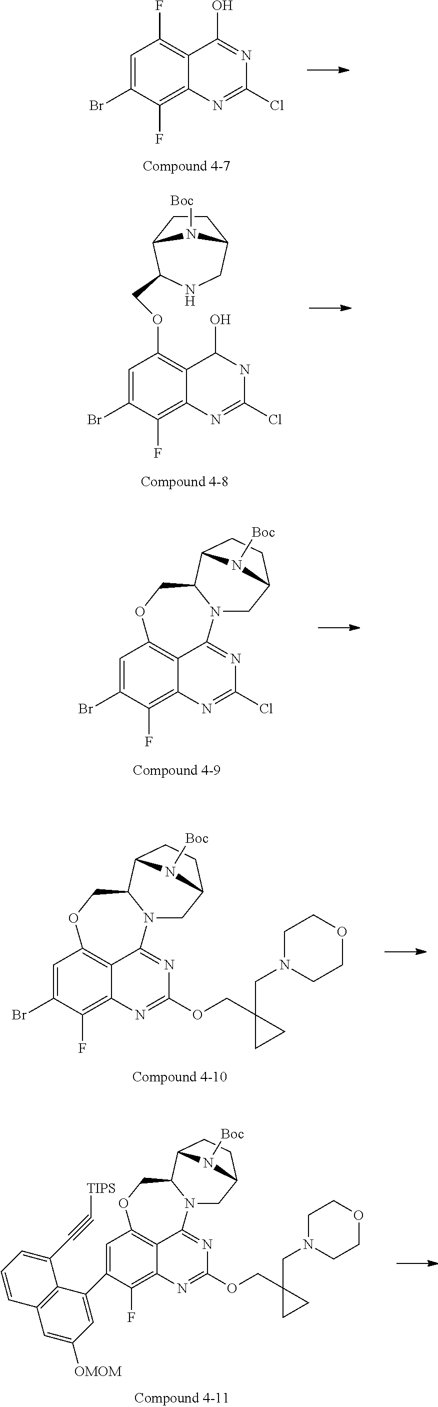

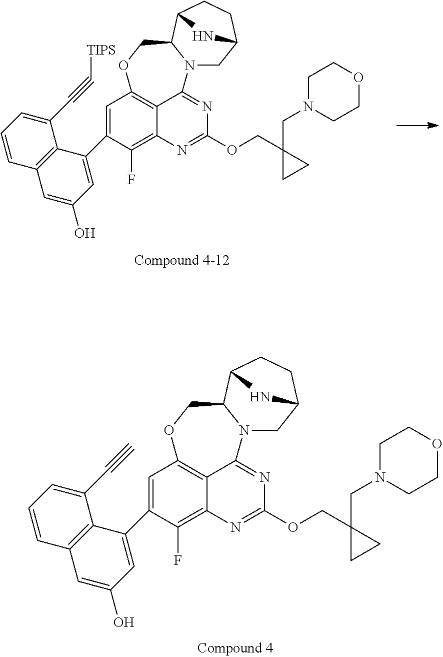

A solution of 1-bromo-2,5-difluoro-3-nitrobenzene (3.11 g, 13.06 mmol), iron (2.12 g, 37.96 mmol) and NH₄ Cl (3.49 g, 65.24 mmol) in ethanol (60 mL) and water (12 mL) was stirred at 80° C. for 2 hours. The resulting mixture was filtered and the filtrate was concentrated under reduced pressure. The residue was dissolved in DCM (100 mL), washed with brine (2×30 mL), dried over Na₂SO₄ and concentrated under reduced pressure to afford Compound 4-1 (2.49 g, 11.97 mmol, 91.6% yield). MS (ESI, m/z): 208 [M+1]⁺, 210 [M+3]⁺.

To a solution of Compound 4-1 (2.49 g, 11.97 mmol), hydroxylammoniumchlorid (2.49 g, 35.83 mmol), Na₂SO₄ (11.64 g, 95.76 mmol), chloral hydrate (2.56 g, 17.95 mmol) in water (50 mL) and ethanol (7 mL) was added hydrochloric acid (1.75 mL). The reaction mixture was stirred at 60° C. for 16 hours. The resulting mixture was cooled to room temperature and filtered, the filter cake was dried to afford Compound 4-2 (3.295 g, 11.80 mmol, 98.6% yield). MS (ESI, m/z): 279 [M+1]⁺, 281 [M+3]⁺.

Compound 4-2 (3.295 g, 11.80 mmol) was added portion-wise to sulfuric acid (29.5 mL) at 60° C. The reaction was stirred at 90° C. for 1 hour. The resulting mixture was cooled to room temperature and added slowly to ice water. The resulting precipitate was collected by filtration, washed with water and dried under reduced pressure to afford Compound 4-3 (2.173 g, 8.29 mmol, 70.2% yield). MS (ESI, m/z): 262[M+1]⁺, 264[M+3]⁺.

To a solution of Compound 4-3 (2.173 g, 8.29 mmol) in aq. NaOH (2 M, 46 mL, 93.50 mmol) was added dropwise hydrogen dioxide (5.2 mL) at 0° C. The reaction mixture was stirred at room temperature for 16 hours. Excess hydrogen dioxide was quenched with excess sodium sulfite and the mixture was neutralized to pH=7. The mixture was filtered and the filtrate was acidified to pH=2 with concentrated hydrochloric acid, the resulting precipitate was collected by filtration, washed with water and dried under reduced pressure to afford Compound 4-4 (1.782 g, 7.07 mmol, 69.8% yield). MS (ESI, m/z): 252 [M+1]⁺, 254 [M+3]⁺.

To a solution of Compound 4-4 (1.782 g, 7.07 mmol) in dichloromethane (20 mL) was added dropwise chlorosulfonyl isocyanate (1.33 g, 9.39 mmol) at 0° C. The reaction mixture was stirred at room temperature for 6 hours and concentrated under reduced pressure. Then concentrated hydrochloric acid (20 mL) was added and stirred at 100° C. for 16 hours. The resulting mixture was cooled to room temperature and filtered, the filter cake was washed by water and dried under reduced pressure to afford Compound 4-5 (0.83 g, 2.99 mmol, 75.5% yield). MS (ESI, m/z): 275 [M-1]⁻. To a solution of Compound 4-5 (0.83 g, 2.99 mmol) in POCl₃ (15 mL) was added N,N-diisopropylethylamine (2 mL). The reaction mixture was stirred at 105° C. for 2 hours. The resulting mixture was concentrated under reduced pressure and the residue was diluted with DCM (50 mL), washed with water (2×30 mL), dried over anhydrous sodium sulphate, concentrated under reduced pressure to afford Compound 4-6 (1.87 g, 5.95 mmol, 113.8% yield).

A suspension of Compound 4-6 (4.96 g, 15.80 mmol) in 5% sodium hydroxide solution (150 mL) was stirred at room temperature for 4 hours. Upon completion, the mixture was adjusted to pH 9-10 with 5% NaHCO₃ and extracted with EA twice. The combined organic phases were washed with brine, dried over anhydrous Na₂SO₄, filtered and concentrated under reduced pressure. The residue was triturated with EA:Hex=1:8 (35 mL). The suspension was filtered and the filter cake was dried under reduced pressure to give Compound 4-7 (3.76 g, 12.73 mmol). MS: m/z 295 [M+1]⁺, 297 [M+3]⁺.

To a solution of Compound 4-7 (453 mg, 1.87 mmol) in dry THF (15 mL) was added sodium hydride (60% in oil, 293 mg, 7.33 mmol) under nitrogen at 0° C. After stirring at room temperature for 30 minutes, a solution of INT 6A (556 mg, 1.88 mmol) in dry THF (5 mL) was added to the reaction mixture and stirred at 0° C. for 24 hours. After completion, the reaction mixture was diluted with EtOAc (50 mL), water (40 mL), and acidified with 5% citric acid until pH=5-6. The organic layer was separated and concentrated under reduced pressure. The residue was purified by silica gel chromatography (eluting with from Hex:EA=1:1 to DCM:MeOH=15:1, v/v) to give Compound 4-8 (481 mg, 0.93 mmol). MS: m/z 517 [M+1]⁺, 519 [M+3]⁺.

To a solution of Compound 4-8 (481 mg, 0.93 mmol) and DIEA (0.5 mL) in DCM (20 mL) was added phosphorus oxychloride (0.5 mL) under nitrogen and then stirred at room temperature for 3 hours. Upon completion, the residue was diluted with DCM (30 mL), and then quenched with saturated NaHCO₃ (50 mL) and separated. The collected organic layer was concentrated under reduced pressure. The residue was purified by silica gel chromatography (eluting with Hex:EA=8:1, v/v) to give Compound 4-9 (233 mg, 0.45mmol). MS: m/z 499 [M+1]⁺, 501 [M+3]⁺.

To a solution of Compound 4-9 (233 mg, 0.45 mmol) and INT 2 (162 mg, 0.95 mmol) in THF (4 mL) and DMF (4 mL) were added DABCO (28 mg, 0.25 mmol) and Cs₂CO₃ (302 mg, 0.93 mmol), then mixture was purged with nitrogen followed by stirring at room temperature for 20 hours. The mixture was diluted with EtOAc (50 mL) and water (30 mL) and the organic layer was separated. The organic layer was washed with brine (30 mL) and dried over anhydrous Na₂SO₄, filtered and concentrated under reduced pressure. The residue was purified by Pre-TLC (Hex:EA=2:1) to give Compound 4-10 (207 mg, 0.33 mmol). MS: m/z 634 [M+1]⁺, 636 [M+3]⁺.

To a solution of Compound 4-10 (207 mg, 0.33 mmol), INT 4 (227 mg, 0.46 mmol) and Cs₂CO₃ (403 mg, 1.24 mmol) in toluene (8 mL) and water (2 mL) was added cataCXium A Pd G₃ (30 mg, 0.041 mmol) and the mixture was purged with nitrogen followed by stirring at 100° C. for 3 hours. Upon completion, the mixture was concentrated under reduced pressure, the residue was diluted with EtOAc (40 mL) and water (30 mL) and the organic layer was separated. The organic layer was concentrated under reduced pressure. The residue was purified by Pre-TLC (Hex:EA=2:1) to give Compound 4-11 (256mg, 0.28 mmol). MS: m/z 922 [M+1]⁺.

To a solution of Compound 4-11 (256 mg, 0.28 mmol) in CH₃CN (5 mL) was added HCl/1,4-dioxane solution (4 M, 2 mL). The reaction mixture was stirred at room temperature for 1 hour. After completion, the reaction mixture was concentrated under reduced pressure, the residue was diluted with EtOAc (40 mL) and water (30 mL) and the mixture was adjusted to pH 8-9 with saturated NaHCO₃. The organic layer was separated and concentrated under reduced pressure to give Compound 4-12 (243 mg, 0.31 mmol). MS: m/z 778 [M+H]⁺.

To a mixture of Compound 4-12 (243 mg, 0.31 mmol) in DMF (5 mL) was added CsF (843 mg, 5.55 mmol). The mixture was stirred at room temperature for 20 hours. After completion, the mixture was diluted with EtOAc (40 mL) and water (30 mL) and the mixture was adjusted to pH 8-9 with saturated NaHCO₃. The organic layer was separated and concentrated under reduced pressure. The residue was purified by Pre-HPLC (C18 column, A: 0.1% TFA in water, B: CH₃ CN, Gradient: 15% B to 40% B in 30 min at a flow rate of 60 mL/min, 230 nm) to give Compound 4 (113.0 mg, 0.18 mmol). MS: m/z 622 [M+H]⁺.

### Example 5

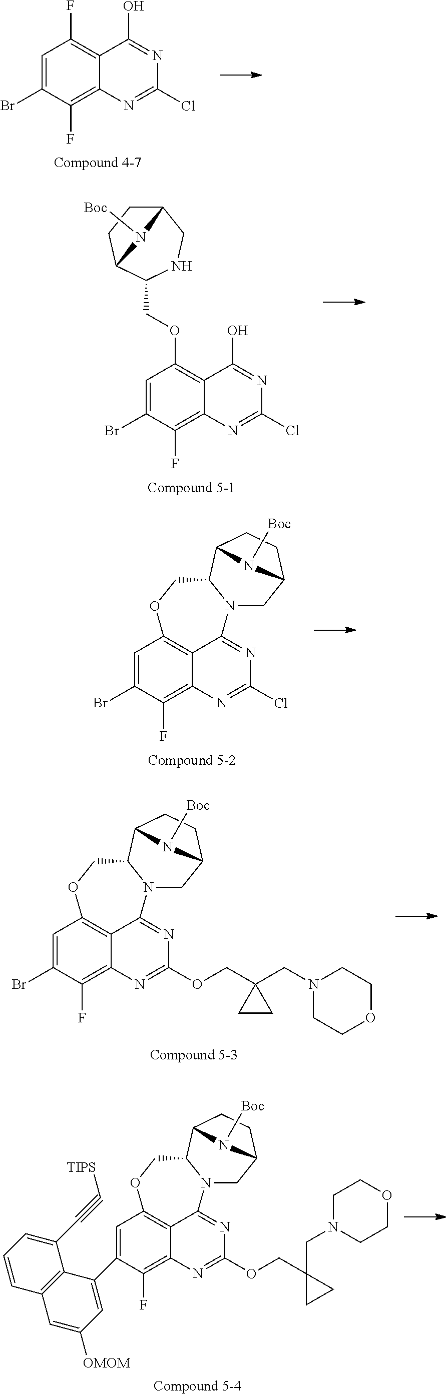

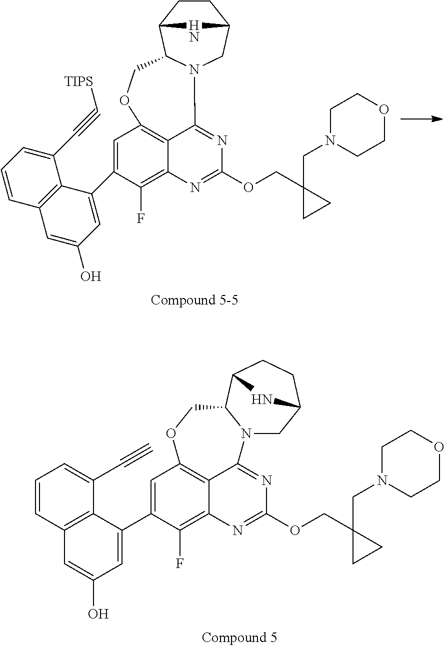

To a solution of Compound 4-7 (548 mg, 2.26 mmol) in dry THF (15 mL) was added sodium hydride (60% in oil, 376 mg, 9.40 mmol) under nitrogen at 0° C. and then stirred at room temperature for 30 minutes. A solution of INT 6B (622 mg, 2.11 mmol) in dry THF (5 mL) was added to the reaction mixture and stirred at 0° C. for 17 hours. After completion, the reaction mixture was diluted with EtOAc (50 mL) and water (40 mL), and acidified with 5% citric acid until pH=5˜6. The organic layer was washed with brine, dried over anhydrous Na₂SO₄, filtered and concentrated under reduced pressure to give Compound 5-1 (crude, 980 mg, 1.89 mmol). MS: m/z 517 [M+1]⁺, 519 [M+3]⁺.

To a solution of Compound 5-1 (crude, 980 mg, 1.89 mmol) and DIEA (0.5 mL) in DCM (20 mL) was added phosphorus oxychloride (0.5 mL) under nitrogen and then stirred at room temperature for 3 hours. Upon completion, the residue was diluted with DCM (30 mL), and then quenched by saturated NaHCO₃ (50 mL). The organic layer was collected and concentrated under reduced pressure. The residue was purified by silica gel chromatography (eluting with from Hex:EA=5:1, v/v) to give Compound 5-2 (257 mg, 0.51 mmol). MS: m/z 499 [M+1]⁺, 501 [M+3]⁺.

To a solution of Compound 5-2 (289 mg, 0.58 mmol) and INT 2 (187 mg, 1.09 mmol) in THF (5 mL) and DMF (5 mL) were added DABCO (32 mg, 0.29 mmol) and Cs₂CO₃ (360 mg, 1.10 mmol), then mixture was purged with N₂ followed by stirring at room temperature for 16 hours. The mixture was diluted with EtOAc (40 mL) and water (30 mL). The organic layer was separated, washed with brine (30 mL), dried over anhydrous Na₂SO₄, filtered and concentrated under reduced pressure. The residue was purified by Pre-TLC (Hex:EA=2:1) to give Compound 5-3 (153 mg, 0.24 mmol). MS: m/z 634 [M+1]⁺, 636 [M+3]⁺.

To a solution of Compound 5-3 (153 mg, 0.24 mmol), INT 4 (160 mg, 0.32 mmol) and Cs₂CO₃ (258 mg, 0.79 mmol) in toluene (8 mL) and water (2 mL) was added CataCXium A Pd G₃ (20 mg, 0.027 mmol) and the mixture was purged with N₂ followed by stirring at 100° C. for 3 hours. Upon completion, the mixture was concentrated under reduced pressure. The residue was diluted with EtOAc (40 mL) and water (30 mL), and the organic layer was separated. Then the organic layer was concentrated under reduced pressure. The residue was purified by Pre-TLC (Hex:EA=2:1) to give Compound 5-4 (187 mg, 0.20 mmol). MS: m/z 922 [M+1]⁺.

To a solution of Compound 5-4 (187 mg, 0.20 mmol) in CH₃CN (5 mL) was added HCl/1,4-dioxane solution (4 M, 2 mL). The reaction mixture was stirred at room temperature for 2 hours. After completion, the reaction mixture was concentrated under reduced pressure, the residue was diluted with EtOAc (40 mL) and water (30 mL), and the mixture was adjusted to pH 8-9 with saturated NaHCO₃. The organic layer was separated and concentrated under reduced pressure to give Compound 5-5 (169 mg, 0.22 mmol). MS: m/z 778 [M+H]⁺.

To a mixture of Compound 5-5 (169 mg, 0.22 mmol) in DMF (5 mL) was added CsF (742 mg, 4.88 mmol). The mixture was stirred at 45° C. for 3 hours. After completion, the mixture was diluted with EtOAc (40 mL) and water (30 mL) and the mixture was adjusted to pH 8-9 with saturated NaHCO₃. The organic layer was separated and concentrated under reduced pressure. The residue was purified by Pre-HPLC (C18 column, A: 0.1% TFA in water, B: CH3CN, Gradient: 15% B to 40% B in 30 min at a flow rate of 70 mL/min, 230 nm) to give Compound 5 (82.0 mg, 0.13 mmol). MS: m/z 622 [M+H]⁺.

### Example 6

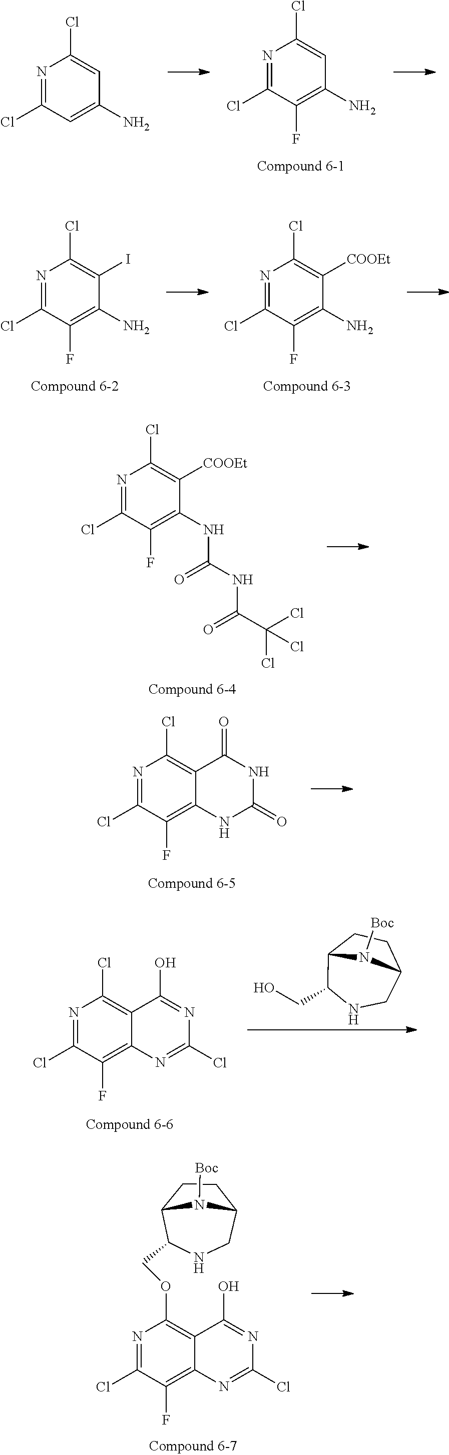

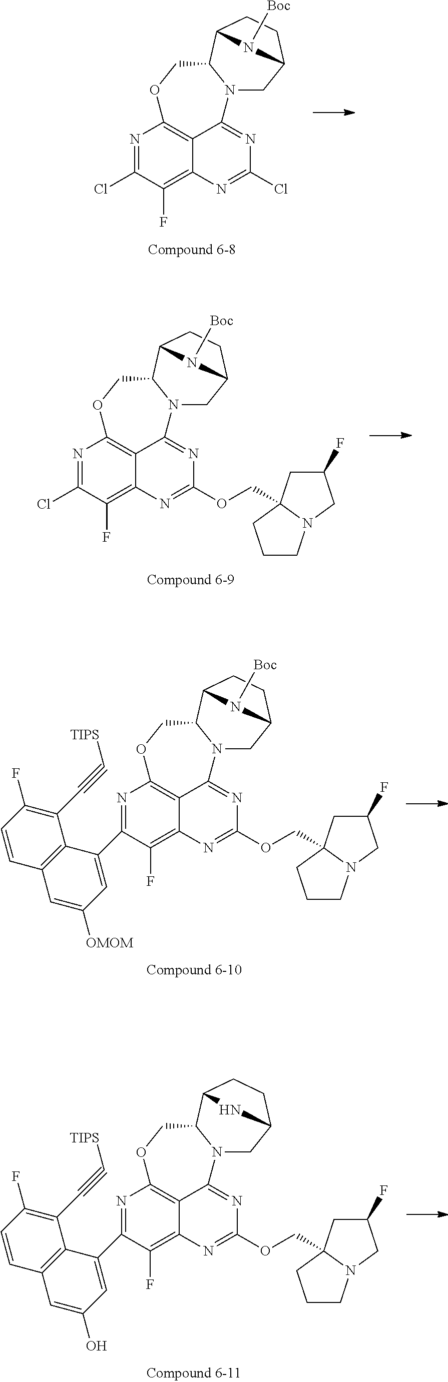

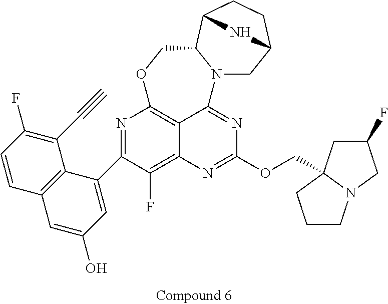 [SMILES: C#Cc1c(F)ccc2cc(O)cc(-c3nc4c5c(nc(OC[C@@]67CCCN6C[C@H](F)C7)nc5c3F)N3C[C@H]5CC[C@H](N5)[C@H]3CO4)c12]

A mixture of 2,6-dichloropyridin-4-amine (35.7 g, 219.0 mmol), 1-(Chloromethyl)-4-fluoro-1,4-diazoniabicyclo[2.2.2]octane ditetrafluoroborate (93.1 g, 262.8 mmol) in DMF (357 mL) and CH₃CN (357 mL) was stirred at 80° C. for 6 hours. The reaction mixture was quenched by water (400 mL), extracted with DCM (400 mL×3), the organic layers were combined, dried over anhydrous Na₂SO₄, filtered and concentrated under reduced pressure. The residue was purified by silica gel column (eluting with petroleum ether:EtOAc=30:1, v/v) to give Compound 6-1 (12.6 g, purity: about 50%). MS (ESI, m/z): 181 [M+H]⁺.

A mixture of Compound 6-1 (2.0 g, 11.05 mmol), NIS (2.98 g, 13.26 mmol) and p-Toluenesulfonic acid monohydrate (105 mg, 0.55 mmol) in CH₃CN (8.4 mL) was stirred at 70° C. for 4 hours under nitrogen atmosphere. The reaction mixture was quenched with water (20 mL), extracted with EtOAc (20 mL×3), and the organic layers were combined, dried over anhydrous Na₂SO₄, filtered and concentrated under reduced pressure. The residue was purified by silica gel column (eluting with petroleum ether:EtOAc=50:1˜20:1, v/v) to give Compound 6-2 (3.6 g). MS (ESI, m/z): 307 [M+H]⁺.

A mixture of Compound 6-2 (1.0 g, 3.26 mmol), Pd(PPh₃)₂Cl₂ (229 mg, 0.33 mmol) and Et₃N (1.19 g, 11.77 mmol) in EtOH (17.0 mL) was stirred at 80° C. for 20 hours under carbon monoxide atmosphere (1.5 MPa) in a sealed tube. The reaction mixture was filtered and the filtrate was concentrated under reduced pressure. The residue was purified by silica gel column to give Compound 6-3 (1.2 g). MS (ESI, m/z): 253 [M+H]⁺.

A mixture of Compound 6-3 (800 mg, 3.16 mmol), trichloroacetyl isocyanate (714 mg, 3.79 mmol) in THF (8 mL) was stirred at room temperature for 1 hour. The reaction mixture was concentrated under reduced pressure. The residue was triturated with MTBE to give Compound 6-4 (880 mg). MS (ESI, m/z): 440 [M+1]⁺, 442 [M+3]⁺.

A mixture of Compound 6-4 (780 mg, 1.77 mmol), NH₃/MeOH (1.26 mL, 7M, 8.85 mmol) and MeOH (7.8 mL) was stirred at room temperature for 1 hour. The reaction mixture was concentrated under reduced pressure. The residue was triturated with MTBE to give Compound 6-5 (550 mg). MS (ESI, m/z): 250 [M+H]⁺.

A mixture of Compound 6-5 (375 mg, 1.50 mmol), DIPEA (595 mg, 4.60 mmol) and POCl₃ (15 mL) was stirred at 105° C. for 17 hours. The reaction mixture was concentrated under reduced pressure. The residue was diluted with 1,4-dioxane (5 mL) and the resulting solution was added dropwise to aq, K₂CO₃ (20%, 30 mL). The mixture was stirred for 2 hours at RT, adjust pH to 2˜3, filtered and the filter cake was collected and dried to give Compound 6-6 (344 mg). MS (ESI, m/z): 268 [M+1]⁺, 270 [M+3]⁺.

To a solution of INT 6B (136 mg, 561.25 μmol) in THF (10 mL) was added NaH (84 mg, 2.10 mmol, 60%) at 0° C. The reaction mixture was stirred for 30 min, then a solution of Compound 6-6 (139 mg, 517.76 μmol) in THF (3 mL) was added. The mixture was stirred at room temperature for 2 hours. The resulting mixture was quenched by water (1 mL) and concentrated under reduced pressure. The residue was purified by Pre-HPLC to afford Compound 6-7 (153 mg, 322.57 μmol, 57.4% yield). MS (ESI, m/z): 474 [M+H]⁺.

To a solution of Compound 6-7 (153 mg, 322.57 μmol) in DCM (10 mL) was added DIPEA (135 mg, 1.04 mmol) and POCl₃ (249 mg, 1.62 mmol) at 0° C. The reaction mixture was stirred for 2 hours. The resulting mixture was quenched by aq. NaHCO₃ (sat., 10 mL) and extracted with DCM (2×20 mL), the organic layers were combined, dried over Na₂SO₄ and concentrated under reduced pressure to afford Compound 6-8 (65 mg, 142.45 μmol), MS (ESI, m/z): 456 [M+H]⁺.

To a solution of Compound 6-8 (65 mg, 142.45 μmol) and ((2R,7aS)-2-fluorotetrahydro-1H-pyrrolizin-7a(5H)-yl)methanol (55 mg, 345.47 μmol) in DMSO (5 mL) was added KF (37 mg, 636.86 μmol) at room temperature. The reaction mixture was stirred at 100° C. for 16 hours. The resulting mixture was quenched by water (20 mL) and extracted with EtOAc (2×30 mL). The organic layers were washed with brine (50 mL), dried over Na₂SO₄ and concentrated under reduced pressure. The residue was purified by Pre-TLC (DCM:MeOH=25:1, v/v) to afford Compound 6-9 (80 mg, 142.45 μmol), MS (ESI, m/z): 579 [M+H]⁺.

A solution of Compound 6-9 (80 mg, 142.45 μmol), INT 5 (104 mg, 202.91 μmol), cataCXium A Pd G₃ (18 mg, 24.71 μmol), Cs₂CO₃ (175 mg, 537.10 μmol) in toluene (4 mL) and water (1 mL) was stirred at 100° C. for 16 hours under nitrogen atmosphere. The reaction was diluted with EtOAc (30 mL) and washed with water (3×20 mL). The organic layer was dried over Na₂SO₄ and concentrated under reduced pressure. The residue was purified by Pre-TLC (DCM:MeOH=20:1, v/v) to afford Compound 6-10 (85 mg, 91.48 mol, 64.2% yield). MS (ESI, m/z): 929 [M+H]⁺.

To a solution of Compound 6-10 (85 mg, 91.48 μmol) in CH₃CN (6 mL) was added HCl (2 mL, 4 M in 1,4-dioxane). The reaction mixture was stirred at room temperature for 1 hour. The resulting mixture was quenched by aq.NaHCO₃ (sat., 20 mL), extracted with DCM (2×30 mL). The organic layers were combined, dried over Na₂SO₄ and concentrated under reduced pressure to afford Compound 6-11 (80 mg, 101.91 mol, 111.4% yield). MS (ESI, m/z): 785 [M+H]⁺.

To a solution of Compound 6-11 (80 mg, 101.91 μmol) in DMF (3 mL) was added CsF (0.30 g, 1.97 mmol) and the reaction mixture was stirred at 40° C. for 16 hours. The resulting mixture was concentrated under reduced pressure and the residue was purified by Pre-HPLC (Daisogel-C18, 50*250 mm, 10 μm, A: 0.1% TFA in water, B: CH₃CN, Gradient: 15% B to 40% B in 34 min at a flow rate of 60 mL/min, 230 nm) to afford Compound 6 (38.6 mg, 45.05 mol, 44.2% yield, 2 TFA salt). MS (ESI, m/z): 629 [M+H]⁺.

### Example 7

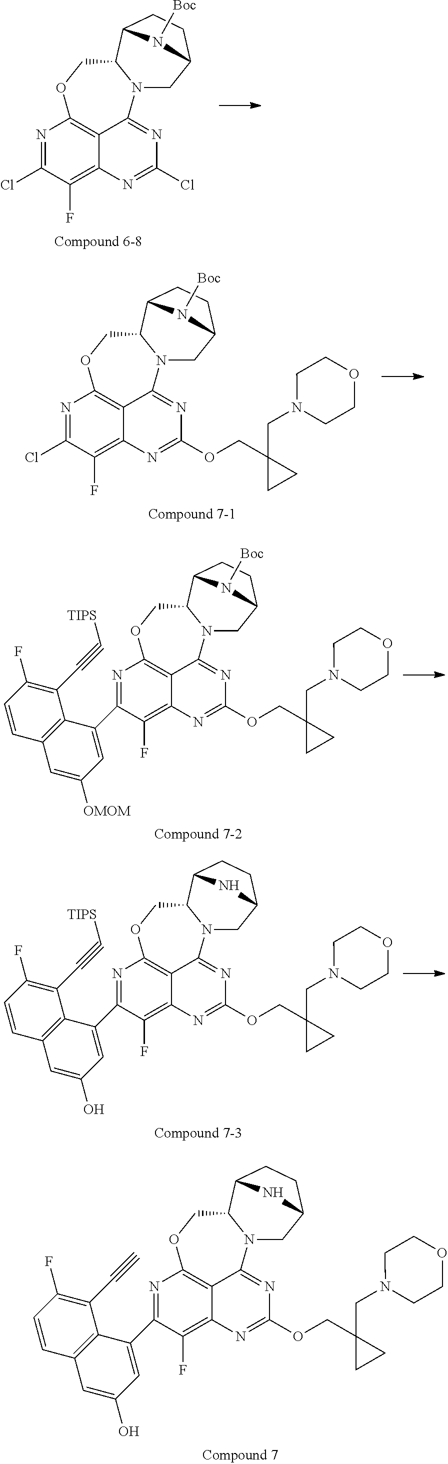

To a solution of Compound 6-8 (100 mg, 0.22 mol) and INT 2 (78 mg, 0.46 mmol) in DMSO (3 mL) was added KF (51 mg, 0.88 mmol) and the mixture was purged with nitrogen followed by stirring at 125° C. for 18 hours. The mixture was diluted with EtOAc (30 mL) and water (30 mL) and the organic layer was separated. The organic layer was washed with brine (40 mL) and dried over anhydrous Na₂SO₄, filtered and concentrated under reduced pressure. The residue was purified by Pre-TLC (Hex:EtOAc=1:1, v/v) to give Compound 7-1 (89 mg, 0.15 mmol). MS: m/z 591 [M+H]⁺.

To a solution of Compound 7-1 (89 mg, 0.15 mmol), INT 5 (133 mg, 0.26 mmol) and Cs₂CO₃ (150 mg, 1.24 mmol) in toluene (10 mL) and water (2.5 mL) was added cataCXium A Pd G₃ (13 mg, 0.018 mmol), the mixture was purged with nitrogen followed by stirring at 100° C. for 18 hours. Upon completion, the mixture was concentrated under reduced pressure, the residue was diluted with EtOAc (40 mL) and water (30 mL) and the organic layer was separated. The combined organic layer was concentrated under reduced pressure. The residue was purified by Pre-TLC (Hex:EtOAc=1:1, v/v) to give Compound 7-2 (116mg, 0.12 mmol). MS: m/z 941[M+1]⁺.

To a solution of Compound 7-2 (116 mg, 0.12 mmol) in CH₃CN (5 mL) was added HCl/1,4-dioxnae (2 mL, 4 M). The reaction mixture was stirred at room temperature for 1.5 hours. After completion, the reaction mixture was concentrated under reduced pressure, the residue was diluted with EtOAc (40 mL) and water (30 mL), the mixture was adjusted to pH 8-9 with saturated NaHCO₃. The organic layer was separated and concentrated under reduced pressure to give Compound 7-3 (94 mg, 0.12 mmol). MS: m/z 797 [M+H]⁺.

To a mixture of Compound 7-3 (94 mg, 0.12 mmol) in DMF (5 mL) was added CsF (306 mg, 2.01 mmol). The mixture was stirred at room temperature for 16 hours. After completion, the mixture was diluted with EtOAc (40 mL) and water (30 mL), the mixture was adjusted to pH 8-9 with saturated NaHCO₃. The organic layer was separated and concentrated under reduced pressure. The residue was purified by Pre-HPLC (C18 column, A: 0.1% NH₄OH in water, B: CH₃CN, Gradient: 20% B to 60% B in 30 min at a flow rate of 60 mL/min, 230 nm) to give Compound 7 (15.4 mg, 0.024 mmol). MS: m/z 641 [M+1]⁺, 643 [M+3]⁺.

### Example 8

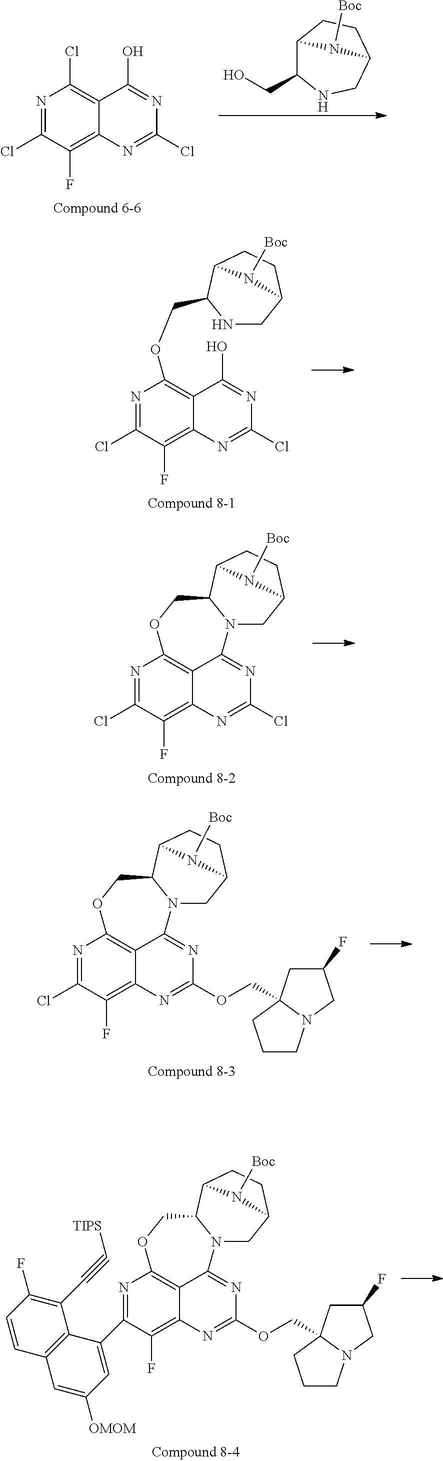

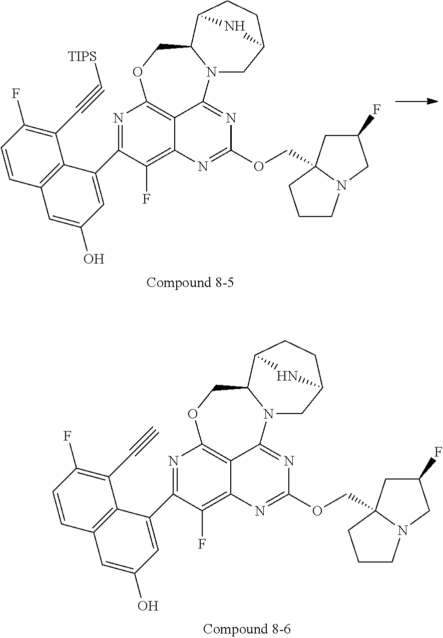

To a solution of INT 7A (197 mg, 812.99 μmol) in THF (5 mL) was added NaH (124 mg, 60%, 3.10 mmol) at 0° C. and stirred at the same temperature for 30 mins, then a solution of Compound 6-6 (231 mg, 860.46 μmol) in THF (3 mL) was added. The mixture was stirred at room temperature for 2 hours. The resulting mixture was quenched by water (20 mL) and extracted with EtOAc (2×40 mL), the organic layer was dried over Na₂SO₄ and concentrated under reduced pressure to afford Compound 8-1 (454 mg, 957.17 μmol, 117.7% yield). MS (ESI, m/z): 474 [M+H]⁺.

To a solution of Compound 8-1 (625 mg, 1.31 mmol) in DCM (10 mL) was added N,N-diisopropylethylamine (0.6 mL, 3.63 mmol) and POCl₃ (612 mg, 3.99 mmol) at 0° C. and stirred for 2 hours. The resulting mixture was quenched by sat. aq. NaHCO₃ (10 mL) and extracted with DCM (2×20 mL), the organic layer was dried over Na₂SO₄ and concentrated under reduced pressure, the residue was purified by Pre-TLC (EtOAc:Hex=1:2, v/v) to afford Compound 8-2 (155 mg, 339.69 μmol, 25.7% yield). MS (ESI, m/z): 456 [M+H]⁺.

To a solution of Compound 8-2 (155 mg, 339.69 μmol) and ((2R,7aS)-2-fluorotetrahydro-1H-pyrrolizin-7a(5H)-yl)methanol (117 mg, 734.92 μmol) in DMSO (6 mL) was added KF (63 mg, 1.08 mmol) at room temperature. The reaction mixture was stirred at 100° C. for 16 hours. The resulting mixture was quenched by water (20 mL) and extracted with EtOAc (2×30 mL). The organic layers were washed with brine (50 mL), dried over Na₂SO₄ and concentrated in reduced pressure. The residue was purified by Pre- TLC (DCM:MeOH=25:1, v/v) to afford Compound 8-3 (155 mg, 78.8% yield). MS (ESI, m/z): 579 [M +H]⁺.

A solution of Compound 8-3 (155 mg, 267.68 μmol), INT 5 (207 mg, 403.87 μmol), cataCXium A Pd G₃ (31 mg, 42.56 μmol), Cs₂CO₃ (281 mg, 862.44 μmol) in toluene (6 mL) and water (1.5 mL) was stirred at 100° C. for 16 hours under nitrogen atmosphere. The reaction mixture was diluted with EtOAc (30 mL) and washed with water (3×20 mL). The organic layer was dried over Na₂SO₄ and concentrated under reduced pressure. The residue was purified by Pre-TLC (DCM:MeOH=20:1, v/v) to afford Compound 8-4 (117 mg, 125.92 μmol, 47.0% yield). MS (ESI, m/z): 929 [M +H]⁺.

To a solution of Compound 8-4 (117 mg, 125.92 μmol) in acetonitrile (6 mL) was added HCl (2 mL, 4 M in 1,4-dioxane). The reaction mixture was stirred at room temperature for 1 hour. The resulting mixture was quenched by sat. aq. NaHCO₃ (20 mL), extracted with DCM (2×30 mL). The organic layers were dried over Na₂SO₄ and concentrated under reduced pressure to afford Compound 8-5 (115 mg, 116.3% yield). MS (ESI, m/z): 785 [M+H]⁺.

To a solution of Compound 8-5 (115 mg, 146.50 μmol) in DMF (3 mL) was added CsF (0.47 g, 3.09 mmol) and the reaction mixture was stirred at 40° C. for 16 hours. The resulting mixture was concentrated under reduced pressure and the residue was purified by Pre-HPLC (C18 column, A: 0.1% TFA in water, B: CH₃CN, Gradient: 15% B to 35% B in 32 min at a flow rate of 60 mL/min, 230 nm) to afford Compound 8 (58.3 mg, TFA salt, 46.45% yield). MS (ESI, m/z): 629 [M+H]⁺.

### Example 9

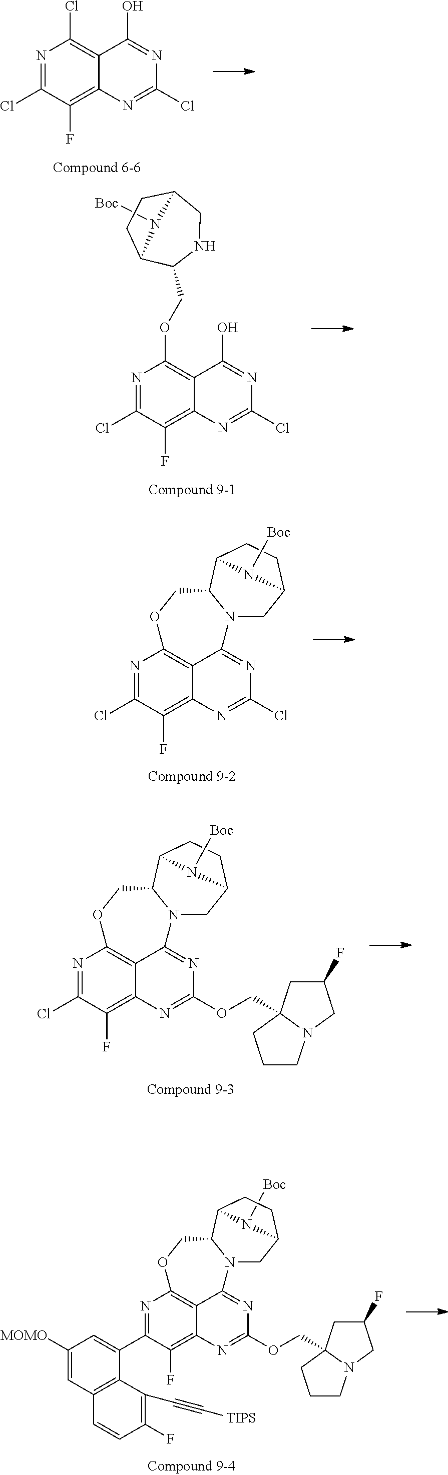

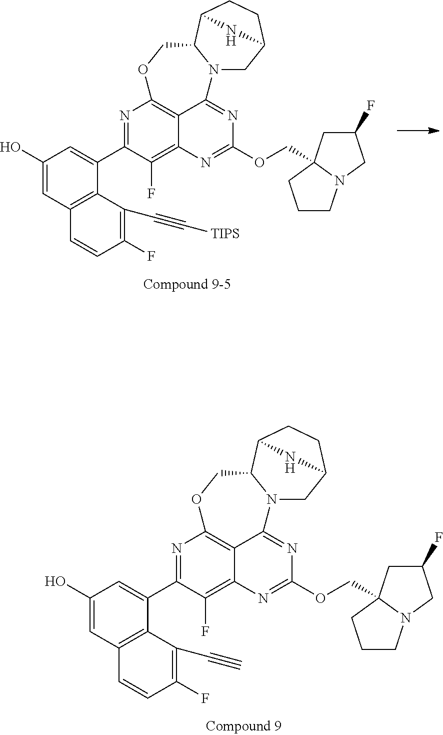

To a solution of INT 7B (208 mg, 0.86 μmol) in THF (5 mL) was added NaH (133 mg, 3.33 mmol, 60%) at −5° C. and stirred for 30 mins, then a solution of Compound 6-6 (191 mg, 711.47 μmol) was added. The mixture was stirred at room temperature for 1 hour. The resulting mixture was quenched by water (100 mL) and extracted with EtOAc (2×50 mL). The organic layers combined, dried over Na₂SO₄ and concentrated under reduced pressure to give the desired product Compound 9-1 (364 mg, 0.77 mmol). MS (ESI, m/z): 474 [M+H]⁺.

To a solution of Compound 9-1 (364 mg, 0.77 μmol) in DCM (10 mL) was added N,N-diisopropylethylamine (0.5 ml) and POCl₃ (0.5 ml) at −20° C. and stirred for 2 hours. The resulting mixture was added to aq. NaHCO₃ (sat., 20 mL) and extracted with DCM (2×20 mL). The organic layer was dried over Na₂SO₄ and concentrated under reduced pressure. The residue was purified by Pre-TLC to afford Compound 9-2 (49 mg, 0.11 mmol). MS (ESI, m/z): 456 [M+H]⁺.

To a solution of Compound 9-2 (66 mg, 144.64 μmol) and ((2R,7aS)-2-fluorotetrahydro-1H-pyrrolizin-7a(5H)-yl)methanol (41 mg, 257.54 μmol) in DMSO (5 mL) was added KF (31 mg, 533.59 μmol). The reaction mixture was stirred at 95° C. for 16 hours under nitrogen atmosphere. The resulting mixture was quenched by water (20 mL) and extracted with EtOAc (2×30 mL). The organic layers were washed with brine (50 mL), dried over Na₂SO₄ and concentrated in reduced pressure. The residue was purified by Pre-TLC to afford Compound 9-3 (64 mg,110.53 μmol). MS (ESI, m/z): 579 [M+H]⁺.

A solution of Compound 9-3 (64 mg, 110.53 μmol), INT 5 (75 mg, 146.33 μmoL), cataCXium A Pd G₃ (16 mg, 21.97 μmol), Cs₂CO₃ (66 mg, 202.57 μmol) in toluene (4 mL) and water (1 mL) was stirred at 100° C. for 16 hours under nitrogen atmosphere. The reaction was diluted with EtOAc (30 mL) and washed with water (3×20 mL). The organic layer was dried over Na₂SO₄ and concentrated under reduced pressure. The residue was purified by Pre-TLC to afford Compound 9-4 (85 mg, 91.48 μmol). MS (ESI, m/z): 929 [M+H]⁺.

To a solution of Compound 9-4 (85 mg, 91.48 μmol) in acetonitrile (3 mL) was added HCl (1 mL, 4 M in 1,4-dioxane). The reaction mixture was stirred at room temperature for 1 hour. The resulting mixture was quenched by aq.NaHCO₃ (sat., 20 mL), extracted with DCM (2×30 mL). The organic layers were dried over Na₂SO₄ and concentrated under reduced pressure to afford Compound 9-5 (84 mg, 107.01μmol). MS (ESI, m/z): 785 [M+H]⁺.

To a solution of Compound 9-5 (84 mg, 107.01 μmol) in DMF (3 mL) was added CsF (0.54 g, 3.55 mmol) and the reaction mixture was stirred at 40° C. for 20 hours. The resulting mixture was concentrated under reduced pressure and the residue was purified by Pre-HPLC (C18 column, A: 10 mmol/L NH₄HCO₃ in water, B: CH₃CN, Gradient: 20% B to 54% B in 40 min at a flow rate of 60 mL/min, 240 nm) to afford Compound 9 (7.4 mg, 11.77 μmop. MS (ESI, m/z): 629 [M+H]⁺.

### Example 10

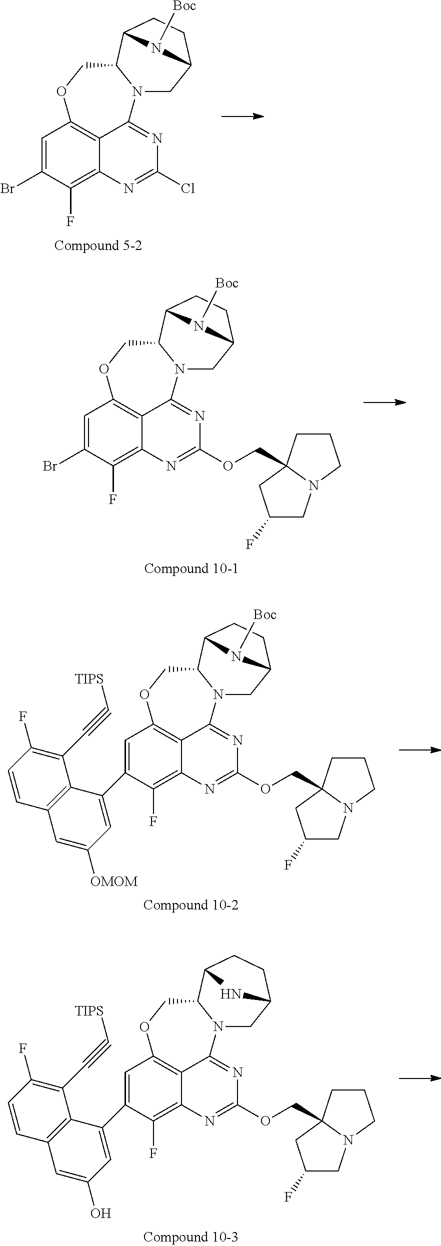

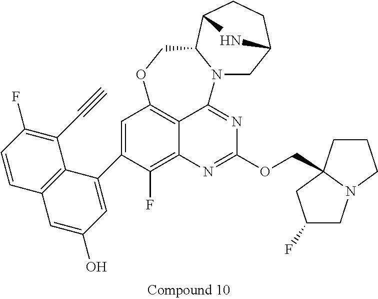 [SMILES: C#Cc1c(F)ccc2cc(O)cc(-c3cc4c5c(nc(OC[C@@]67CCCN6C[C@H](F)C7)nc5c3F)N3C[C@H]5CC[C@H](N5)[C@H]3CO4)c12]

To a solution of Compound 5-2 (39.5 mg, 0.079 mmol) and ((2R,7aS)-2-fluorotetrahydro-1H-pyrrolizin-7a(5H)-yl)methanol (19.7 mg, 0.12 mmol) in THF (0.5 mL) and DMF (0.5 mL) were added DABCO (1.7 mg, 0.015 mmol) and Cs₂CO₃ (77.9 mg, 0.24 mmol), then the mixture was purged with nitrogen followed by stirring at room temperature for 16 hours. The mixture was diluted with EtOAc (20 mL) and water (10 mL). The organic layer was collected, washed with brine (20 mL) and dried over anhydrous Na₂SO₄, filtered and concentrated under reduced pressure. The residue was purified by Pre-TLC (eluting with EtOAc) to give Compound 10-1 (38 mg, 0.061 mmol). MS: m/z 622 [M+1]⁺, 624 [M+3]⁺.

To a solution of Compound 10-1 (38 mg, 0.061 mmol), INT 5 (52 mg, 0.101 mmol) and Cs₂CO₃ (62 mg, 0.19 mmol) in Toluene (8 mL) and water (2 mL) was added CataCXium A Pd G₃ (6 mg, 0.0082 mmol) and the mixture was purged with nitrogen followed by stirring at 100° C. for 16 hours. Upon completion, after concentrated under reduced pressure, the residue was diluted with EtOAc (30 mL) and water (20 mL). The collected organic layer was concentrated under reduced pressure. The residue was purified by Pre-TLC (eluting with DCM:MeOH=20:1, v/v) to give Compound 10-2 (43 mg, 0.046 mmol).MS: m/z 928 [M+1]⁺.

To a solution of Compound 10-2 (43 mg, 0.046 mmol) in CH₃CN (5 mL) was added HCl/1,4-dioxnae (2 mL, 4 M). The reaction mixture was stirred at room temperature for 1 hour. After completion, the reaction mixture was concentrated under reduced pressure, the residue was diluted with EtOAc (30 mL) and water (20 mL), the pH value of the mixture was adjusted to 8-9 with saturated NaHCO₃. The organic layer was separated and concentrated under reduced pressure to give Compound 10-3 (45 mg, crude). MS: m/z 784 [M+H]⁺.

To a mixture of Compound 10-3 (45 mg, crude) in DMF (5 mL) was added CsF (210 mg, 1.38 mmol). The mixture was stirred at room temperature for 17 hours. After completion, the mixture was diluted with EtOAc (30 mL) and water (20 mL), the mixture was adjusted to pH 8-9 with saturated NaHCO₃. The organic layer was separated and concentrated under reduced pressure. The residue was purified by Pre-HPLC (C18 column, A: 0.1% TFA in water, B: CH₃CN, Gradient: 15% B to 35% B in 35 min at a flow rate of 60 mL/min, 230 nm) to give Compound 10 (28.4 mg, 0.033 mmol, 2 TFA salt). MS: m/z 628 [M+H]⁺.

### Example 11

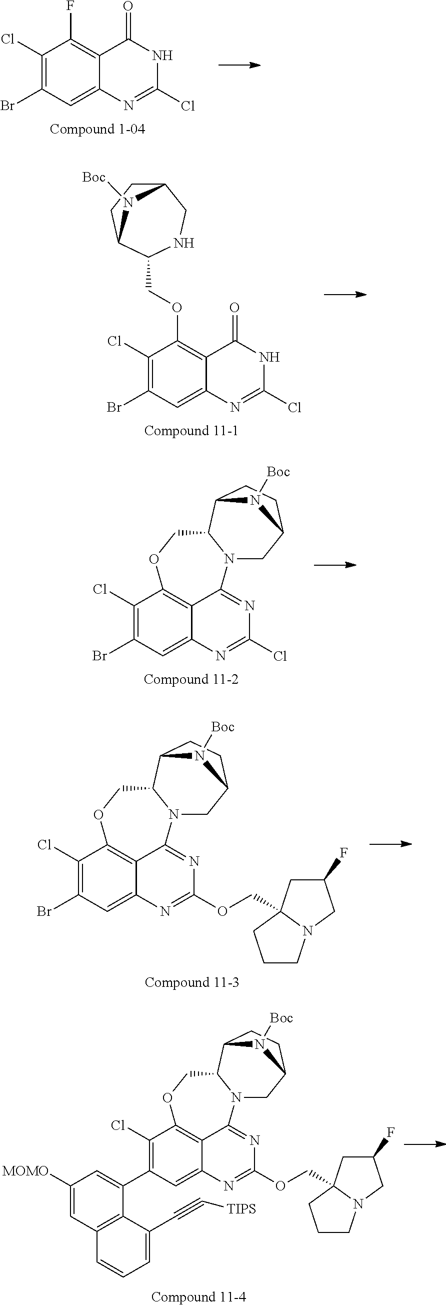

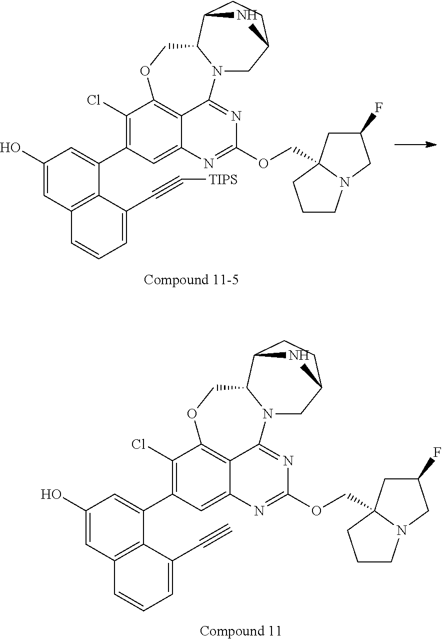

To a solution of INT 6B (258 mg, 1.06 mmol) in dry THF (10 mL) was added Sodium Hydride (60% in oil, 71 mg, 1.78 mmol) under nitrogen atmosphere at 0° C. and stirred at room temperature for 30 minutes. A solution of Compound 1-04 (387 mg, 1.24 mmol) in dry THF (5 mL) was added to the reaction mixture and stirred at room temperature for 30 minutes, and then stirred at 65° C. for 30 minutes. After completion, the reaction mixture was diluted with EtOAc (50 mL), water (40 mL), and acidified by 5% citric acid to pH=5˜6. The organic layer was separated and concentrated under reduced pressure. The residue was purified by silica gel chromatography (eluting with from Hex:EtOAc=1:1, then DCM:MeOH=15:1, v/v) to give Compound 11-1 (233 mg, 0.44 mmol). MS: m/z 533 [M+1]⁺, 535 [M+3]⁺.

To a solution of Compound 11-1 (233 mg, 0.44 mmol) and DIEA (0.1 mL) in toluene (20 mL) was added POCl₃ (0.1 mL) under nitrogen atmosphere and stirred at 80° C. for 2 hours. Upon completion, the reaction mixture was concentrated under reduced pressure and the residue was purified by silica gel chromatography (eluting with Hex:EtOAc=8:1, v/v) to give Compound 11-2 (69 mg, 0.13 mmol). MS: m/z 515 [M+1]⁺, 517 [M+3]⁺.

To a solution of Compound 11-2 (89 mg, 0.17 mmol) and ((2R,7aS)-2-fluorotetrahydro-1H-pyrrolizin-7a(5H)-yl)methanol (54 mg, 0.34 mmol) in THF (3 mL) and DMF (3 mL) were added DABCO (12 mg, 0.11 mmol) and Cs₂CO₃ (167 mg, 0.51 mmol), the mixture was purged with nitrogen followed by stirring at room temperature for 40 hours. The mixture was diluted with EtOAc (50 mL) and water (30 mL). The collected organic layer was washed with brine (20 mL), dried over anhydrous Na₂SO₄, filtered and concentrated under reduced pressure. The residue was purified by Pre-TLC (EtOAc) to give Compound 11-2 (69 mg, 0.11 mmol). MS: m/z 638 [M+1]⁺, 640 [M+3]⁺.

To a solution of Compound 11-3 (69 mg, 0.11 mmol), INT 4 (104 mg, 0.21 mmol) and Cs₂CO₃ (105 mg, 0.32 mmol) in 1,4-Dioxane (4 mL) and water (1 mL) was added PdCl₂(dppf)₂ (12 mg, 0.016 mmol) and the mixture was purged with nitrogen and stirred at 100° C. for 2 hours. Upon completion, the mixture was concentrated under reduced pressure, the residue was diluted with EtOAc (30 mL) and water (20 mL). The organic layer was collected and concentrated under reduced pressure. The residue was purified by Pre-TLC (EtOAc) to give Compound 11-4 (50 mg, 0.054 mmol).MS: m/z 926 [M+1]⁺.

To a solution of Compound 11-4 (50 mg, 0.054 mmol) in CH₃CN (5 mL) was added HCl/1,4-dioxnae (4 M, 2 mL). The reaction mixture was stirred at room temperature for 2 hours. After completion, the reaction mixture was concentrated under reduced pressure to give Compound 11-5 (212 mg, crude). MS: m/z 782 [M+H]⁺.

To a mixture of Compound 11-5 (212 mg, crude) in DMF (5 mL) was added CsF (327 mg, 2.15 mmol). The mixture was stirred at 40° C. for 18 hours. After completion, the mixture was diluted with EtOAc (30 mL) and water (20 mL), the mixture was adjusted to pH 8-9 with saturated NaHCO₃. The organic layer was separated and concentrated under reduced pressure. The residue was purified by Pre-HPLC (C18 column, A: 0.1% TFA in water, B: CH₃CN, Gradient: 10% B to 35% B in 25 min at a flow rate of 40 mL/min, 240 nm) to give Compound 11 (15.0 mg, 0.018 mmol, 2 TFA salt). MS: m/z 626 [M+H]⁺.

### Example 12

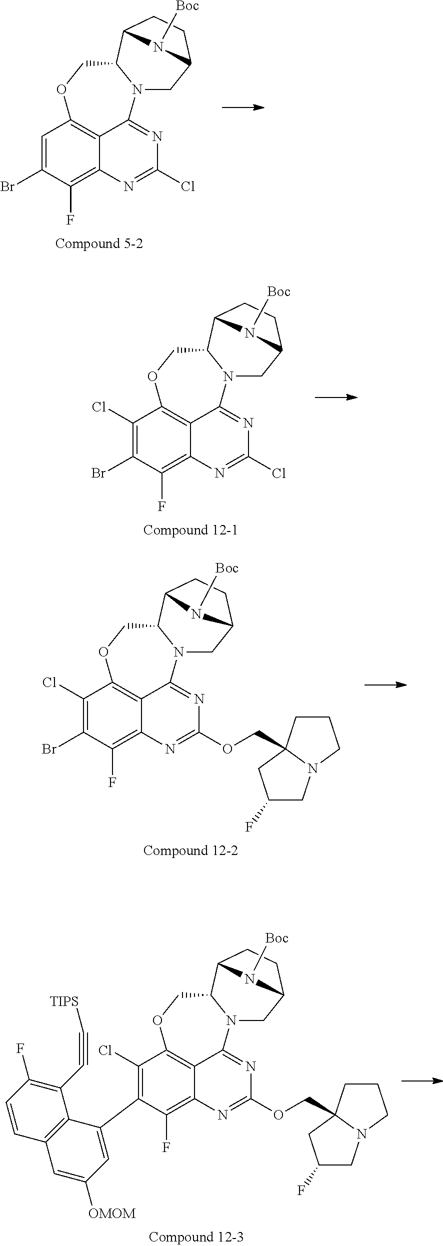

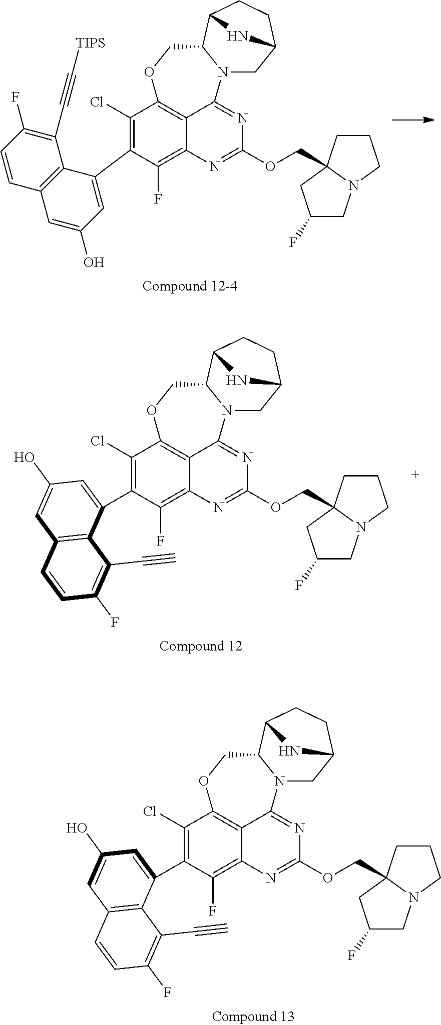

A mixture of Compound 5-2 (318.1 mg, 0.64 mmol), NCS (167 mg, 1.25 mmol) in DMF (6 mL) was stirred for 3 hours at 55° C. After cooled to room temperature, the reaction mixture was diluted with water (30 mL) and EtOAc (40 mL), separated and the organic layer was washed with brine (30 mL), dried and concentrated under reduced pressure. The residue was purified by silica gel chromatograph (eluted with EtOAc:Hex=1:5, v/v) to give Compound 12-1 (243 mg, 71.47% yield). MS: m/z 533 [M+1]⁺, 535 [M+3]⁺.

To a solution of Compound 12-1 (91 mg, 0 17mmol) and ((2R,7aS)-2-fluorotetrahydro-1H-pyrrolizin-7a(5H)-yl)methanol (41 mg, 0.26 mmol) in THF (0.3 mL) and DMF (0.3 mL) was added DABCO (3.9 mg, 0.035 mmol) and Cs₂CO₃ (166.7 mg, 0.52 mmol), then mixture was purged with nitrogen followed by stirring at room temperature for 17 hours. The mixture was diluted with EtOAc (30 mL) and water (30 mL) and the organic layer was separated. The organic layer was washed with brine (30 mL) and dried over anhydrous Na₂SO₄, filtered and concentrated under reduced pressure. The residue was purified by Pre-TLC (EtOAc) to give Compound 12-2 (99 mg, 0.15 mmol). MS: m/z 656 [M+1]⁺, 658 [M+3]⁺.

To a solution of Compound 12-2 (99 mg, 0.15 mmol), INT 5 (120 mg, 0.23 mmol) and Cs₂CO₃ (148 mg, 0.45 mmol) in toluene (12 mL) and water (3 mL) was added cataCXium A Pd G₃ (12 mg, 0.016 mmol) and the mixture was purged with nitrogen followed by stirring at 100° C. for 3 hours. Upon completion, the reaction mixture was concentrated under reduced pressure, the residue was diluted with EtOAc (30 mL) and water (30 mL), the organic layer was separated. The combined organic layer was concentrated under reduced pressure. The residue was purified by Pre-TLC (EtOAc) to give Compound 12-3 (125 mg, 0.13 mmol).MS: m/z 962 [M+1]⁺.

To a solution of Compound 12-3 (125 mg, 0.13 mmol) in acetonitrile (5 mL) was added HCl/1,4-dioxnae (4 M, 2 mL). The reaction mixture was stirred at room temperature for 1 hour. After completion, the reaction mixture was concentrated under reduced pressure, the residue was diluted with EtOAc (40 mL) and water (30 mL), the mixture was adjusted to pH 8-9 with saturated NaHCO₃. The organic layer was separated and concentrated under reduced pressure to give Compound 12-4 (102.7 mg, 0.13 mmol). MS: m/z 818 [M+H]⁺.

To a mixture of Compound 12-4 (102.7 mg, 0.13 mmol) in DMF (5 mL) was added CsF (285.1 mg, 1.88 mmol). The mixture was stirred at room temperature for 20 hours. After completion, the mixture was diluted with EtOAc (40 mL) and water (30 mL), the mixture was adjusted to pH 8-9 with saturated NaHCO₃. The organic layer was separated and concentrated under reduced pressure. The residue was purified by Pre-HPLC (C18 column, A: 0.1% TFA in water, B: CH₃CN, Gradient: 15% B to 40% B in 30 min at a flow rate of 60 mL/min, 230 nm). Then the two isomers were separated by Prep-HPLC-Gilson with the following conditions: Column, CHIRALPAK-IG column(2 cm×25 cm, 5 um); mobile phase, (Hex:DCM=3:1)(0.1%DEA)/EtOH (50:50); Flowing rate: 20 mL/min. This resulted in Compound 12 (the first eluting isomer, Ret Time 3.738 min) and Compound 13 (the second eluting isomer, Ret Time 5.245 min). MS: m/z 662 [M+H]⁺.

### Example 13

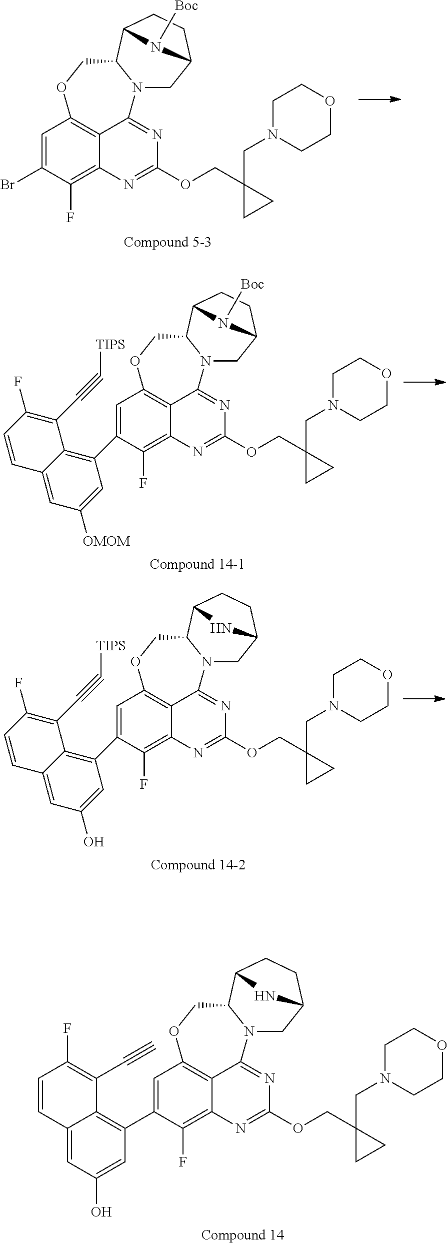

To a solution of Compound 5-3 (34 mg, 0.054 mmol), INT 5 (43.4 mg, 0.085 mmol) and Cs₂CO₃ (54.2 mg, 0.17 mmol) in toluene (10 mL) and water (2.5 mL) was added cataCXium A Pd G₃ (4.1 mg, 0.0056 mmol) and the mixture was purged with nitrogen followed by stirring at 100° C. for 18 hours. Upon completion, the mixture was concentrated under reduced pressure, the residue was diluted with EtOAc (30 mL) and water (30 mL) and the organic layer was separated. The organic layer was concentrated under reduced pressure. The residue was purified by Pre-TLC (Hex:EtOAc=1:1, v/v) to give Compound 14-1 (42 mg, 0.045 mmol).MS: m/z 940 [M+1]+.

To a solution of Compound 14-2 (42 mg, 0.045 mmol) in acetonitrile (5 mL) was added HCl/1,4-dioxnae (2 mL, 4 N). The reaction mixture was stirred at room temperature for 1 hour. After completion, the reaction mixture was concentrated under reduced pressure, the residue was diluted with EtOAc (40 mL) and water (30 mL), the mixture was adjusted to pH 8-9 with saturated NaHCO₃. The organic layer was separated and concentrated under reduced pressure to give Compound 14-3 (42 mg, crude). MS: m/z 796 [M+H]⁺.

To a mixture of Compound 14-3 (42 mg, crude) in DMF (4 mL) was added CsF (183 mg, 1.20 mmol). The mixture was stirred at room temperature for 16 hours. After completion, the mixture was diluted with EtOAc (40 mL) and water (30 mL), the mixture was adjusted to pH 8-9 with saturated NaHCO₃. The organic layer was separated and concentrated under reduced pressure. The residue was purified by Pre- HPLC (C18 column, A: 0.1% TFA in water, B: CH₃CN, Gradient: 15% B to 40% B in 30 min at a flow rate of 60 mL/min, 230 nm) to give Compound 14 (5.2 mg, TFA salt, 11.36 yield). MS: m/z 640 [M+H]⁺.

### Example 14

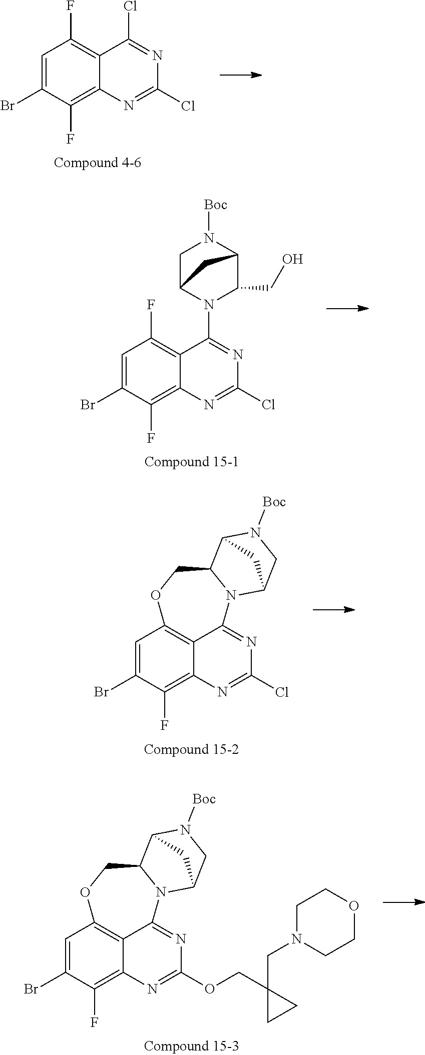 [SMILES: Fc1c(Br)cc(F)c2c(Cl)nc(Cl)nc12 ; CC(C)(C)OC(=O)N1C[C@@H]2C[C@H]1[C@H](CO)N2c1nc(Cl)nc2c(F)c(Br)cc(F)c12 ; CC(C)(C)OC(=O)N1C[C@@H]2C[C@H]1[C@@H]1COc3cc(Br)c(F)c4nc(Cl)nc(c34)N21 ; CC(C)(C)OC(=O)N1C[C@@H]2C[C@H]1[C@@H]1COc3cc(Br)c(F)c4nc(OCC5(CN6CCOCC6)CC5)nc(c34)N21]

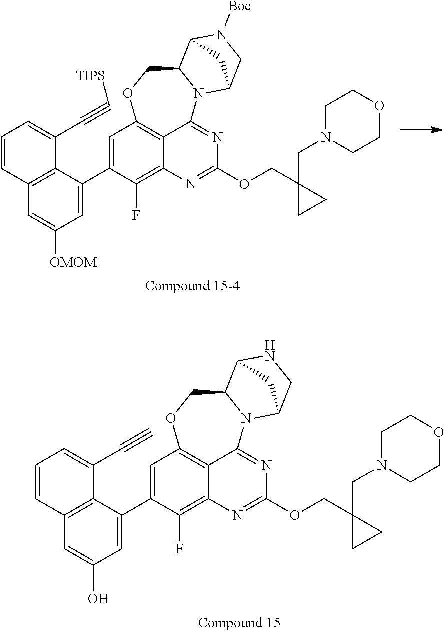

A solution of Compound 4-6 (508 mg, 1.62 mmol), DIEA (391 mg, 3.03 mmol) and INT 8 (439 mg, 1.92 mmol) in acetonitrile (10 mL) was stirred at room temperature for 1 hour. The solution was concentrated under reduced pressure, the residue was slurried with Hex:EtOAc (50 mL, v/v=10:1), filtered and the filter cake was dried under reduced pressure to afford the desired product Compound 15-1 (0.70 g, 1.38 mmol) as a yellow solid. MS m/z: 505 [M+1]⁺, 507[M+3]⁺.

A solution of Compound 15-1 (339 mg, 0.67 mmol), NaH (127 mg, 60% wt, 3.18 mmol) in THF (8 mL) was stirred at 65° C. for 1 hour under nitrogen atmosphere. The mixture was allowed to cool to room temperature and partitioned between water and EtOAc. The organic layer was dried over Na₂SO₄ and concentrated under reduced pressure. The residue was purified by Pre-TLC (MeOH:DCM=1:20, v/v) to afford the desired product Compound 15-2 (224 mg, 0.46 mmol). MS m/z: 485/487 [M+H]⁺.

A solution of Compound 15-2 (224 mg, 0.46 mmol), INT 2 (106 mg, 0.62 mmol), DABCO (34 mg, 0.30 mmol), Cs₂CO₃ (226 mg, 0.69 mmol) in DMF/THF (1 mL/1 mL) was stirred at room temperature for overnight under nitrogen atmosphere. The mixture was partitioned between water and EtOAc. The organic layer was dried over Na₂SO₄ and concentrated in reduced pressure. The residue was purified by Pre-TLC (MeOH:DCM=1:20, v/v) to afford the desired product Compound 15-3 (245 mg, 0.36 mmol). MS m/z: 620/622 [M+H]⁺.

To a solution of Compound 15-3 (145 mg, 0.23 mmol), INT 4 (140 mg, 0.28 mmol) in toluene (4 mL) and water (1 mL) was added Cs₂CO₃ (167 mg, 0.51 mmol) and cataCXium A Pd G₃ (17 mg, 0.023 mmol). The reaction mixture was stirred at 100° C. for 3 hours under nitrogen atmosphere. The reaction mixture was filtered and the filtrate was concentrated under reduced pressure. The residue was purified by Pre-TLC (Hex:EtOAc=1:1, v/v) to give the desired product Compound 15-4 (122 mg, 0.134 mmol). MS m/z: 908 [M+H]⁺.

A solution of Compound 15-4 (122 mg, 0.134 mmol), HCl (1 mL, 1 M in dioxane) in acetonitrile (3 mL) was stirred at room temperature for 1 hour. The mixture was concentrated in reduced pressure. The residue was diluted with sat. aq. NaHCO₃ (20 mL) and extracted with EtOAc (2×20 mL). The organic layers were combined, dried over Na₂SO₄ and concentrated under reduced pressure. The residue was dissolved in DMF (3 mL), and CsF (260 mg, 1.71 mmol) was added. The reaction mixture was stirred at 45° C. for 2 hours under nitrogen atmosphere. The reaction mixture was purified by Prep-HPLC (C18 column, A: 0.1% TFA in water, B: CH₃CN, Gradient: 15% B to 35% B in 35 min at a flow rate of 60 mL/min, 276 nm) to afford Compound 15 (38.9 mg, 0.465 mmol, 2TFA salt). MS m/z: 608 [M+H]⁺.

### Example 15

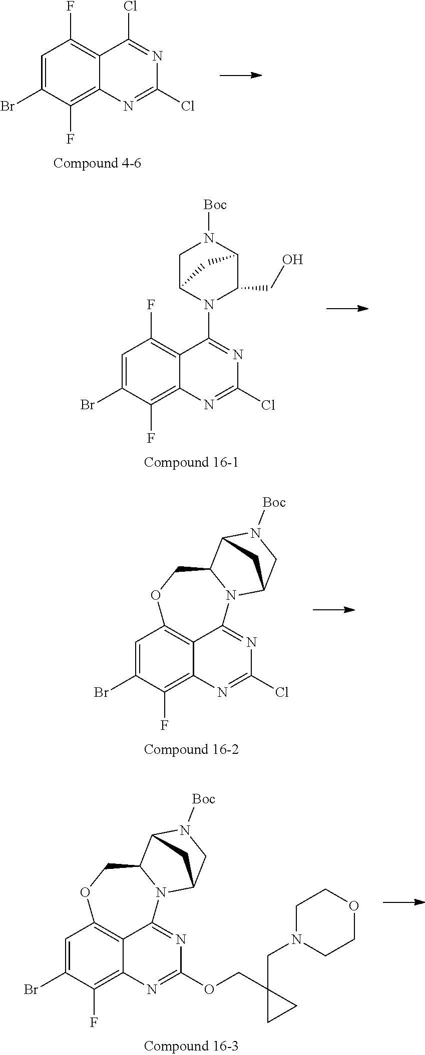 [SMILES: Fc1c(Br)cc(F)c2c(Cl)nc(Cl)nc12 ; CC(C)(C)OC(=O)N1C[C@H]2C[C@@H]1[C@H](CO)N2c1nc(Cl)nc2c(F)c(Br)cc(F)c12 ; CC(C)(C)OC(=O)N1C[C@H]2C[C@@H]1[C@@H]1COc3cc(Br)c(F)c4nc(Cl)nc(c34)N21 ; CC(C)(C)OC(=O)N1C[C@H]2C[C@@H]1[C@@H]1COc3cc(Br)c(F)c4nc(OCC5(CN6CCOCC6)CC5)nc(c34)N21]

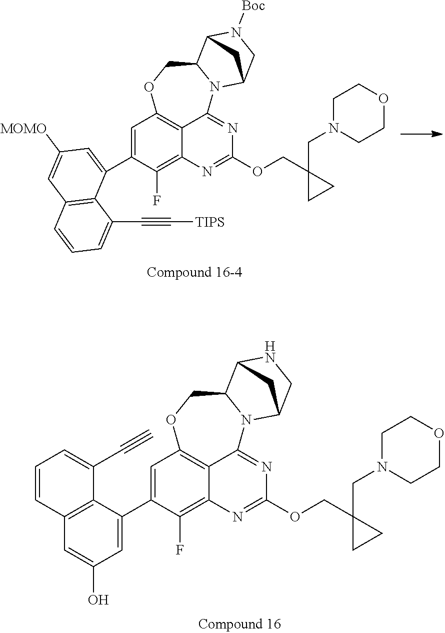

A solution of Compound 4-6 (0.91 g, 2.90 mmol), DIEA (782 mg, 6.05 mmol) and INT 9 (0.65 g, 2.85 mmol) in acetonitrile (15 mL) was stirred at room temperature for 1 hour. The solution was concentrated under reduced pressure, the residue was slurried with Hex:EtOAc (100 mL, v/v=10:1), filtered and the filter cake was dried in reduced pressure to afford the desired product Compound 16-1 (1.06 g, 2.10 mmol) as a yellow solid. MS m/z: 505/507 [M+H]⁺.

A solution of Compound 16-1 (0.50 g, 0.99 mmol), NaH (174 mg, 60% wt, 4.35 mmol) in THF (10 mL) was stirred at 65° C. for 1 hour under nitrogen atmosphere. The mixture was allowed to cool to room temperature and partitioned between water and EtOAc. The organic layer was dried over Na₂SO₄ and concentrated in reduced pressure. The residue was slurried with Hex:EtOAc (50 mL, v/v=10:1), filtered and the filter cake was dried in reduced pressure to afford the desired product Compound 16-2 (485 mg, 1.00 mmol). MS m/z: 485/487 [M+H]⁺.

A solution of Compound 16-2 (485 mg, 1.00 mmol), INT 2 (213 mg, 1.24 mmol), DABCO (66 mg, 0.59 mmol), Cs₂CO₃ (491 mg, 1.51 mmol) in DMF/THF (2 mL/2 mL) was stirred at room temperature for overnight under nitrogen atmosphere. The mixture was partitioned between water and EtOAc. The organic layer was dried over Na₂SO₄ and concentrated in reduced pressure. The residue was purified by Pre-TLC (MeOH:DCM 1:40, v/v) to afford the desired product Compound 16-3 (350 mg, 0.56 mmol). MS m/z: 620/622 [M+H]⁺.

To a solution of Compound 16-3 (201 mg, 0.32 mmol), INT 4 (194 mg, 0.39 mmol) in toluene (4 mL) and water (1 mL) was added Cs₂CO₃ (220 mg, 0.68 mmol) and cataCXium A Pd G₃ (25 mg, 0.034 mmol). The reaction mixture was stirred at 100° C. for 3 hours under nitrogen atmosphere. The mixture was filtered and the filtrate was concentrated under reduced pressure. The residue was purified by Pre-TLC (Hex:EtOAc=1:1, v/v) to give the desired product Compound 16-4 (203 mg, 0.224 mmol). MS m/z: 908 [M+H]⁺.

A solution of Compound 16-4 (149 mg, 0.164 mmol), HCl (1 mL, 1 M in dioxane) in acetonitrile (3 mL) was stirred at room temperature for 1 hour. The mixture was concentrated in reduced pressure. The residue was diluted with sat. aq. NaHCO₃ (20 mL) and extracted with EtOAc (2×20 mL). The organic layers were combined, dried over Na₂SO₄ and concentrated under reduced pressure. The residue was dissolved in DMF (3 mL) and CsF (540 mg, 3.55 mmol) was added. The reaction mixture was stirred at 45° C. for 2 hours under nitrogen atmosphere. The reaction mixture was purified by Prep-HPLC (C18 column, A: 0.1% TFA in water, B: CH₃CN, Gradient: 15% B to 32% B in 25 min at a flow rate of 60 mL/min, 240 nm) to afford Compound 16 (65.9 mg, 0.789 mmol, 2TFA salt). MS m/z: 608 [M+H]⁺.

### Pharmacological Experiments

### 1. SOS1 Catalyzed Nucleotide Exchange Assay

GDP-loaded HIS-KRAS(G12D, aa 1-169) was pre-incubated with a compound in the presence of 10 nM GDP in a 384-well plate (Greiner) for 15 min, then purified SOS1 ExD(Flag tag, aa 564-1049), BODIPY™ FL GTP (Invitrogen) and MAb(monoclonal antibody) Anti 6HIS-Tb cryptate Gold (Cisbio) were added to the assay wells (Final concentration: 1.5 nM GDP-loaded HIS-KRAS(G12D), 5 nM GDP, 0.5 μM SOS1 ExD, 80 nM BODIPY™ FL GTP, 52.5 ng/mL MAb Anti 6HIS-Tb cryptate Gold) and incubated for 4 hours at 25° C. Wells containing same percent of DMSO served as vehicle control, and wells without KRAS served as low control. TR-FRET signals were read on Tecan Spark multimode microplate reader. The parameters were F486: Excitation 340 nm, Emission 486 nm, Lag time 100 μs, Integration time 200 μs; F515: Excitation 340 nm, Emission 515 nm, Lag time 100 μs, Integration time 200 μs. TR-FRET ratios for each individual wells were calculated by equation: TR-FRET ratio=(Signal F515/Signal F486)*10000. The percent of activation of compounds treated wells were normalizedbetween vehicle control and low control. Then the data were analyzed using a 4-parameter logistic model to calculate ICso values. The results are shown in the following Table 13.

### 2. GTP-KRAS and cRAF Interaction Assay

GppNp-loaded HIS-KRAS(G12D, aa 1-169) was pre-incubated with a compound in the presence of 200 μM GTP in a 384-well plate (Greiner) for 15 min, then cRAF RBD(GST tag, aa 50-132, CreativeBioMart), MAb Anti GST-d2 (Cisbio) and MAb Anti 6HIS-Tb cryptate Gold (Cisbio) were added to the assay wells (Final concentration: 2.0 nM GppNp-loaded HIS-KRAS(G12D), 100 μM GTP, 35 nM cRAF RBD, 1 μg/mL MAb Anti GST-d2, 52.5 ng/mL MAb Anti 6HIS-Tb cryptate Gold) and incubated for 2 hours at 25° C. Wells containing same percent of DMSO served as vehicle control, and wells without KRAS served as low control. HTRF signals were read on Tecan Spark multimode microplate reader and HTRF ratios were calculated under manufacturer's instructions. The percent of activation of compounds treated wells were normalized between vehicle control and low control. Then the data were analyzed using a 4-parameter logistic model to calculate IC₅₀ values. The results are shown in the Table 13.

TABLE 13

| | | GTP-KRAS |
|---|---|---|
| | SOS1 catalyzed | and cRAF |
| | nucleotide | interaction |
| | exchange assay | assay IC₅₀ |
| Compound | IC₅₀ (nM) | (nM) |
| 1 | 3859 | 31392 |
| 2 | 880 | 23329 |
| 3 | 294 | 11638 |
| 4 | 2.50 | 378 |
| 5 | 1.16 | 20.5 |
| 6 | 0.23 | 3.9 |
| 7 | 0.659 | 9.8 |
| 8 | 32.6 | 280.2 |
| 9 | 81 | 2235.0 |
| 10 | 0.452 | 5.4 |
| 11 | 7.24 | 184 |
| 12 | 35.8 | 154.0 |
| 13 | 0.691 | 4.8 |
| 14 | 1.15 | 9.2 |
| 15 | 11.0 | 251 |
| 16 | 86.10 | 660 |

1-34. (canceled)

35. A compound of formula (I) or formula (IV), a stereoisomer thereof, an atropisomer thereof, a pharmaceutically acceptable salt thereof, a pharmaceutically acceptable salt of the stereoisomer thereof, or a pharmaceutically acceptable salt of the atropisomer thereof: 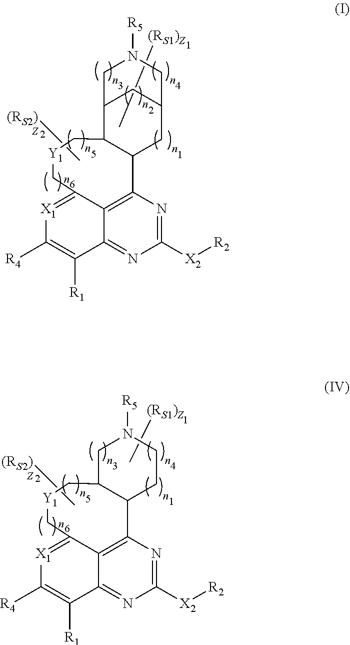
wherein,
R₁ is selected from hydrogen, halogen, —C₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, —OH, —SH, —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl; wherein, said —C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
Y₁ is selected from NH, O, S, SO, SO₂, NHCO, CONH, NHSO₂ or SO₂NH; when Y₁ is selected from NH, NHCO, CONH, NHSO₂ or SO₂NH, each of which is optionally substituted with R_{S2};
X₁ is selected from CR₃ or N;
n₁, n₂, n₃, n₄, n₅ and n₆ are each independently selected from 0, 1, 2, 3, 4, 5 or 6;
R₃ is selected from hydrogen, halogen, —C₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, —OH, —SH, —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl; wherein said —C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
X₂ is selected from CR₂₁R₂₂, NR₂₃, O, S, SO or SO₂;
R₂₁ and R₂₂ are each independently selected from hydrogen, halogen, —C₁₋₆alkyl, —OH, —OC₁₋₆alkyl, —SH, —SC₁₋₆alkyl, —NH₂, —NH(C₁₋₆alkyl) or —N(C₁₋₆alkyl)₂;
R₂₃ is selected from hydrogen or —C₁₋₆alkyl;
R₂ is selected from 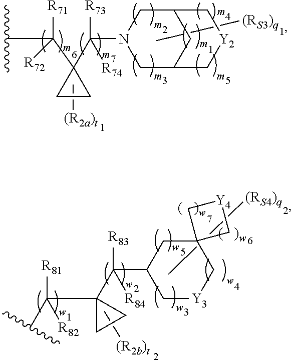 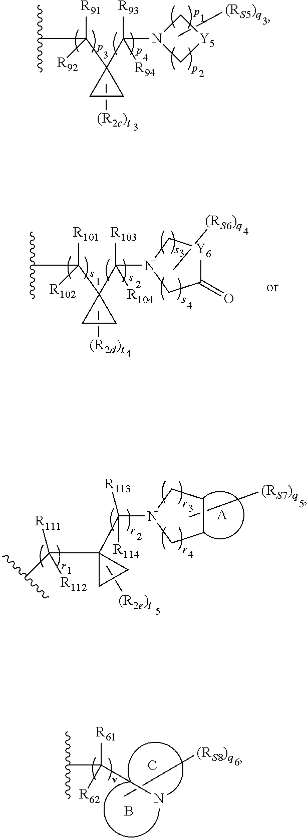
-L-(3-12 membered heterocyclyl), -L-(3-12 membered cycloalkyl), -L-(6-12 member aryl), -L-(5-12 membered heteroaryl) or -L-NR₂₄R₂₅;
Each L is independently selected from a bond or C₁₋₁₀alkylene optionally substituted with one or more R_{S9};
R₂₄ and R₂₅ are each independently selected from hydrogen or —C₁₋₁₀alkyl optionally substituted with one or more R_{S10};
Said 3-12 membered heterocyclyl in -L-(3-12 membered heterocyclyl) is optionally substituted with one or more R_{S11};
Said 3-12 membered cycloalkyl in -L-(3-12 membered cycloalkyl) is optionally substituted with one or more R_{S12};
Said 6-12 member aryl in -L-(6-12 member aryl) is optionally substituted with one or more R_{S13};
Said 5-12 membered heteroaryl in -L-(5-12 membered heteroaryl) is optionally substituted with one or more R_{S14};
Y₂ is selected from O, S, SO, SO₂, C═O, NH or CH₂, when Y₂ is selected from NH or CH₂, the NH or CH₂ is optionally substituted with 1 or 2 R_{S3};
m₁, m₂, m₃, m₄ or m₅ is independently selected from 0, 1, 2, 3, 4, 5 or 6;
m₆ or m₇ is independently selected from 0, 1, 2, 3, 4, 5 or 6;
Y₃ and Y₄ are each independently selected from O, S, SO, SO₂, C═O, NH or CH₂, when Y₃ and Y₄ is selectd from NH or CH₂, the NH or CH₂ is optionally substituted with 1 or 2 R_{S4};
w₁ and w₂ are each independently selected from 0, 1, 2, 3, 4, 5 or 6;
w₃, w₄, w₅, w₆ and w₇ are each independently selected from 0, 1, 2, 3, 4, 5 or 6; provided that w₆ and w₇ are not 0 at the same time;
Y₅ is selected from O, S, SO, SO₂, C═O, NH or CH₂, when Y₅ is selected from NH or CH₂, the NH or CH₂ is optionally substituted with 1 or 2 R_{S5};
p₁ and p₂ are each independently selected from 0, 1, 2, 3, 4, 5, or 6, provided that p₁ and p₂ are not 0 at the same time;
p₃ and p₄ are each independently selected from 0, 1, 2, 3, 4, 5 or 6;
Y₆ is selected from O, S, NH or CH₂, when Y₆ is selected from NH or CH₂, the NH or CH₂ is optionally substituted with 1 or 2 R_{S6};
s₁ and s₂ are each independently selected from 0, 1, 2, 3, 4, 5 or 6;
s₃ and s₄ are each independently selected from 0, 1, 2, 3, 4, 5, or 6, provided that s₃ and s₄ are not 0 at the same time;
r₁ and r₂ are each independently selected from 0, 1, 2, 3, 4, 5 or 6;
r₃ and r₄ are each independently selected from 0, 1, 2, 3, 4, 5, or 6;
v is selected from 0, 1, 2, 3, 4, 5 or 6;
ring A is selected from a 3-10 membered carbocyclic ring, a 3-10 membered heterocyclic ring, a 6-10 membered aryl ring or a 5-10 membered heteroaryl ring, said heterocyclic or heteroaryl ring at each occurrence is independently contains 1, 2, 3 or 4 heteroatoms selected from N, O or S;
Ring B and ring C are each independently selected from a 3-10 membered heterocyclic ring which is optionally further contains 1, 2, or 3 heteroatoms selected from N, O or S except the fused N atom;
R_{S1}, R_{S2}, R_{S3}, R_{S4}, R_{S5}, R_{S6}, R_{S7}, R_{S8}, R_{S9}, R_{S10}, R_{S11}, R_{S12}, R_{S13} and R_{S14} are each independently selected from halogen, —C₁₋₆alkyl, haloC₁₋₆alkoxy, —C₂₋₆alkenyl, —C₂₋₆alkynyl, —CN, —NO₂, —N₃, oxo, —NH₂, —NH(C₁₋₆alkyl), —N(C₁₋₆alkyl)₂, —OH, —O(C₁₋₆alkyl), —SH, —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C_{1- 6}alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein, said —C₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally substituted with one or more R_{Sa};
Each of R_{Sa} is independently selected from halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —NH₂, —NH(C₁₋₆alkyl), —N(C₁₋₆alkyl)₂, —OH, —SH, —S(C₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, phenyl or 5-6 membered heteroaryl; wherein said 3-6 membered cycloalkyl, 3-6 membered heterocyclyl, phenyl or 5-6 membered heteroaryl is independently optionally substituted with 1, 2, or 3 R_{Sb};
Each of R_{Sb} is independently selected from halogen; —C₁₋₆alkyl; haloC₁₋₆alkyl; —CN; —OH; —NH₂; —NH(C₁₋₆alkyl); —NH(C₁₋₆alkyl)₂; —OC₁₋₆alkyl; or —C₁₋₆alkyl substituted with 1, 2 or 3 substituents selected form halogen, haloC₁₋₆alkyl, —CN, —OH, —NH₂, —NH(C₁₋₆alkyl), —NH(C₁₋₆alkyl)₂ or —OC₁₋₆alkyl;
q₁, q₂, q₃, q₄, q₅ and q₆ are each independently selected from 0, 1, 2, 3, 4, 5 or 6;
R₂ₐ, R_{2b}, R_{2c}, R_{2d} and R₂ₑ are each independently selected from is selected from halogen, —C₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl; wherein, said —C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from halogen, —C₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
t₁, t₂, t₃, t₄ and is are each independently selected from 0, 1, 2, 3, 4, 5 or 6;
R₆₁, R₆₂, R₇₁, R₇₂, R₇₃, R₇₄, R₈₁, R₈₂, R₈₃, R₈₄, R₉₁, R₉₂, R₉₃, R₉₄, R₁₀₁, R₁₀₂, R₁₀₃, R₁₀₄, R₁₁₁, R₁₁₂, R₁₁₃ and R₁₁₄ are each independently selected from are independently selected from hydrogen, halogen, —C₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl; wherein, said —C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
R₄ is selected from phenyl, naphthyl, 5 membered heteroaryl, 6 membered heteroaryl, 9 membered heteroaryl or 10 membered heteroaryl, wherein said R4 is independently optionally substituted with 1, 2, 3, 4, 5, or 6 R₄ₐ;
Each of R₄ₐ is independently selected from halogen, —C₁₋₆alkyl, haloC₁₋₆alkyl, haloC₁₋₆alkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl, 5-10 membered heteroaryl or R₄₁, wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered heterocyclyl, 6-10 membered aryl or 5-10 membered heteroaryl is independently optionally substituted with 1, 2 or 3 R_{4b};
Each of R_{4b} is independently selected from halogen; —C₁₋₆alkyl; haloC₁₋₆alkyl; —CN; oxo; —OH; —NH₂; —NH(C₁₋₆alkyl); —NH(C₁₋₆alkyl)₂; 13 OC₁₋₆alkyl; or —C₁₋₆alkyl substituted with 1, 2 or 3 substituents selected form halogen, haloC₁₋₆alkyl, —CN, —OH, —NH₂, —NH(C₁₋₆alkyl), —NH(C₁₋₆alkyl)₂ or —OC₁₋₆alkyl;
R₄₁ is selected from 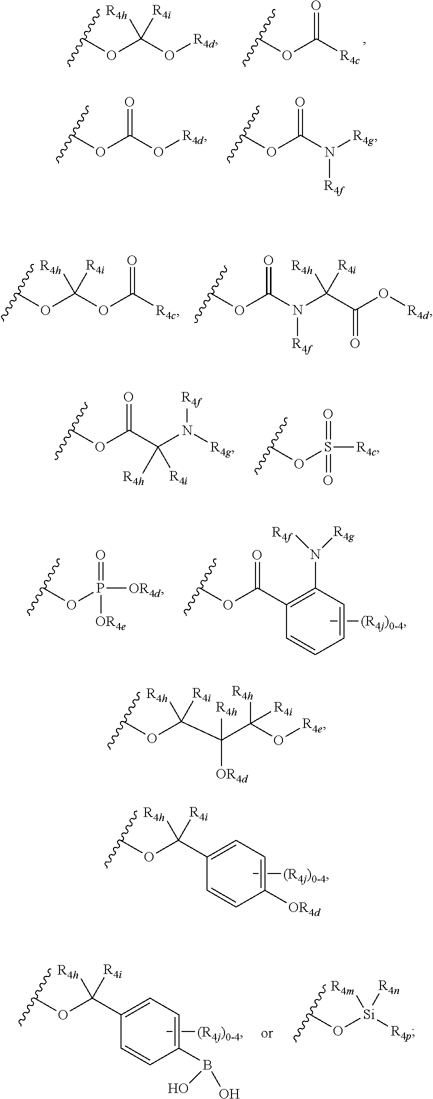
R_{4c} is selected from hydrogen, —C₁₋₃₀alkyl, —C₂₋₃₀alkenyl, —C₂₋₃₀alkynyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₄ⱼ;
R_{4d} and R₄ₑ are each selected from hydrogen, —C₁₋₃₀alkyl, —C₂₋₃₀alkenyl, —C₂₋₃₀alkynyl, —C(═O)C₁₋₆alkyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₄ⱼ;
R_{4f} and R_{4g} are each selected from hydrogen, —C₁₋₃₀alkyl, —C₂₋₃₀alkenyl, —C₂₋₃₀alkynyl, —C(═O)C₁₋₆alkyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₄ⱼ;
R₄ₕ, R₄ᵢ, R₄ₘ, R₄ₙ and R₄ₚ are each selected from hydrogen, halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl; wherein, said —C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from halogen, —C₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
Optionally, R_{4f} and R_{4g} together with the atoms to which they are respectively attached form a 4-10 membered heterocyclyl ring, said 4-10 membered heterocyclyl ring optionally further contains 1 or 2 heteratoms selected from N, O, S, S(═O) or S(═O)₂ and optionally substituted with one or more R₄ⱼ;
Optionally, R_{4f} and R₄ₕ together with the atoms to which they are respectively attached form a 4-10 membered heterocyclyl ring, said 4-10 membered heterocyclyl ring optionally further contains 1 or 2 heteratoms selected from N, O, S, S(═O) or S(═O)₂ and optionally substituted with one or more R₄ⱼ;
R₄ⱼ at each occurrence is independently selected from halogen, —C₁₋₆alkyl, haloC₁₋₆alkyl, haloC₁₋₆alkoxy, —C₂₋₆alkenyl, —C₂₋₆alkynyl, —CN, oxo, —NO₂, —NH₂, —NH(C₁₋₆alkyl), —N(C₁₋₆alkyl)₂, —OH, —O(C₁₋₆alkyl), —SH, —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl, or 5-10 membered heteroaryl, wherein said —C₁₋₆alkyl, haloC₁₋₆alkoxy, —C₂₋₆alkenyl, —C₂₋₆alkynyl, 3-10 membered cycloalkyl, 3-10 membered heterocyclyl, 6-10 membered aryl or 5-10 membered heteroaryl is independently optionally substituted with 1, 2 or 3 substituents selected from halogen; —C₁₋₆alkyl; haloC₁₋₆alkyl; —CN; oxo; —OH; —NH₂; —NH(C₁₋₆alkyl); —NH(C₁₋₆alkyl)₂; —OC₁₋₆alkyl; or —C₁₋₆alkyl substituted with 1, 2 or 3 substituents selected form halogen, haloC₁₋₆alkyl, —CN, —OH, —NH₂, —NH(C₁₋₆alkyl), —NH(C₁₋₆alkyl)₂ or —OC₁₋₆alkyl;
R₅ is selected from hydrogen or 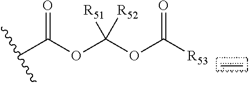
R₅₁ and R₅₂ are each independently selected from are independently selected from hydrogen, halogen, —C₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —SH, —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl; wherein, said —C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from halogen, —C₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —SH, —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
R₅₃ is selected from hydrogen, —C₁₋₃₀alkyl, —C₂₋₃₀alkenyl, —C₂₋₃₀alkynyl, —C(═O)C₁₋₆alkyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₅ₐ;
R₅ₐ at each occurrence is independently selected from halogen, —C₁₋₆alkyl, haloC₁₋₆alkyl, haloC₁₋₆alkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
z₁ and z₂ are each independently selected from 0, 1, 2, 3, 4, 5 or 6;
Each of (heterocyclyl and heteroaryl) at each occurrence is independently contain 1, 2, 3 or 4 heteroatoms selected from N, O, S, S(═O) or S(═O)₂.

36. The compound of claim 35, wherein, the compound is any one of the formulas in the Table 1 in the description of the present invention.

37. The compound of claim 35, wherein, R₁ is selected from hydrogen, —F, —Cl, —Br, —C₁₋₃alkyl, haloC₁₋₃alkoxy, —C₂₋₃alkenyl, —C₂₋₃alkynyl, —CN, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), 6-10 membered cycloalkyl, 6-10 membered heterocyclyl, 6-8 membered aryl or 5-8 membered heteroaryl.

38. The compound of claim 35, wherein, R₁ is selected from —H, —F, —Cl, —CH₃, —CH₂CH₃, —CN, —COOH, —CH₂OH, —OH, —OCH₃, —OCH₂CH₃, —CF₃, —CHF₂, —NH₂, —NHCH₃, —N(CH₃)₂, —CH₂NH₂, —CH₂CH₂NH₂, —CH₂OH, —CH₂CH₂OH, —SH, —S—CH₃, —CH₂SH, —CH₂CH₂SH, —CH=CH₂, —C≡CH, —CHCH═CH₂, —OCF₃, —OCHF₂, —C(═O)NH₂, —C(═O)OCH₃,

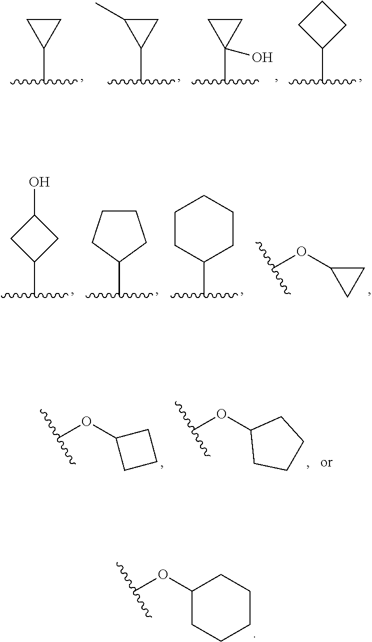

39. The compound of claim 35, wherein, R₃ is selected from hydrogen, —F, —Cl, —Br, —C₁₋₃alkyl, haloC₁₋₃alkoxy, —C₂₋₃alkenyl, —C₂₋₃alkynyl, —CN, oxo, —NH₂, —NH(C₁₋₃alkyl), —N(C₁₋₃alkyl)₂, —OH, —O(C₁₋₃alkyl), —SH, —S(C₁₋₃alkyl), —S(haloC₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), 3-10 membered cycloalkyl, 3-10 membered heterocyclyl, 6-10 membered aryl or 5-8 membered heteroaryl.

40. The compound of claim 35, wherein, the moiety of —X₂—R₂ or —O—R₂ is selected from 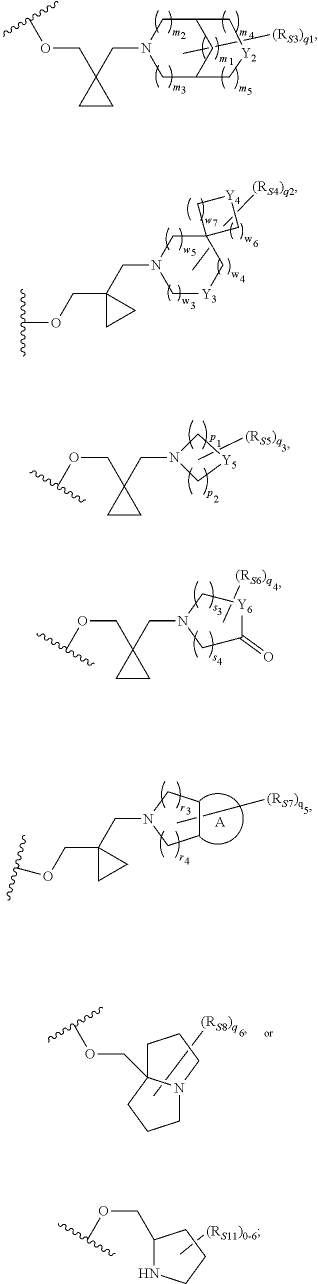
Y₂ is selected from O, S, SO, SO₂, C═O, NH or CH₂, when Y₂ is selected from NH or CH₂, the NH or CH₂ is optionally substituted with 1 or 2 R_{S3};
m₁, m₂, m₃, m₄ or m₅ is independently selected from 0, 1, 2, 3, 4, 5 or 6;
Y₃ and Y₄ are each independently selected from O, S, SO, SO₂, C═O, NH or CH₂, when Y₃ and Y₄ is selectd from NH or CH₂, the NH or CH₂ is optionally substituted with 1 or 2 R_{S4};
w₃, w₄, w₅, w₆ and w₇ are each independently selected from 0, 1, 2, 3, 4, 5 or 6; provided that w₆ and w₇ are not 0 at the same time;
Y₅ is selected from O, S, SO, SO₂, C═O, NH or CH₂, when Y₅ is selected from NH or CH₂, the NH or CH₂ is optionally substituted with 1 or 2 R_{S5};
p₁ and p₂ are each independently selected from 0, 1, 2, 3, 4, 5, or 6, provided that p₁ and p₂ are not 0 at the same time;
Y₆ is selected from O, S, NH or CH₂, when Y₆ is selected from NH or CH₂, the NH or CH₂ is optionally substituted with 1 or 2 R_{S6};
s₃ and s₄ are each independently selected from 0, 1, 2, 3, 4, 5, or 6, provided that s₃ and s₄ are not 0 at the same time;
r₃ and r₄ are each independently selected from 0, 1, 2, 3, 4, 5, or 6;
ring A at each occurrence is independently selected from a 4 membered carbocyclic ring, 5 membered carbocyclic ring, a, 6 membered carbocyclic ring, a 4 membered heterocyclic ring including 1 ring member selected from N, a 5 membered heterocyclic ring including 1 to 2 ring members selected from N, or O, a 6 membered heterocyclic ring including 1 to 2 ring members selected from N, O or S, a phenyl ring, a 5 membered heteroaryl ring including 1 to 2 ring members selected from N, O, or S, or a 6 membered heteroaryl ring including 1 ring member selected from N.

41. The compound of claim 35, wherein:
Y₂ is selected from O, C═O, NH or CH₂, when Y₂ is selected from NH or CH₂, the NH or CH₂ is optionally substituted with 1 or 2 R_{S3};
m₁ is selected from 0, 1, 2 or 3; m₂ is selected from 0, 1, 2 or 3; m₃ is selected from 0, 1, 2 or 3; m₄ is selected from 0, 1, 2 or 3; m₅ is selected from 0, 1, 2 or 3;
Y₃ and Y₄ are each independently selected from O, S, SO₂, NH or CH₂, when Y₃ and Y₄ is selectd from NH or CH₂, the NH or CH₂ is optionally substituted with 1 or 2 R_{S4};
w₃ is selected from 0, 1, 2 or 3; w₄ is selected from 0, 1, 2 or 3; w₅ is selected from 0, 1, 2 or 3; w₆ is selected from 0, 1, 2 or 3; w₇ is selected from 0, 1, 2 or 3; provided that w₆ and w₇ is not 0 at the same time;
Y₅ is selected from O, S, SO₂, NH, CH₂ or C═O, when Y₅ is selected from NH or CH₂, the NH or CH₂ is optionally substituted with 1 or 2 R_{S5};
p₁ is selected from 1, 2, 3 or 4; p₂ is selected from 1, 2, 3 or 4;
Y₆ is selected from O, S or NH, when Y₆ is selected from NH or CH₂, the NH or CH₂ is optionally substituted with R_{S6};
s₁ is selected from 1, 2, 3 or 4; s₂ is selected from 1, 2, 3 or 4;
r₃ is selected from 1, 2 or 3; r₄ is selected from 1, 2 or 3;
ring A at each occurrence is independently selected from a 4 membered carbocyclic ring, 5 membered carbocyclic ring, a, 6 membered carbocyclic ring, a 4 membered heterocyclic ring including 1 ring member selected from N, a 5 membered heterocyclic ring including 1 to 2 ring members selected from N, or O, a 6 membered heterocyclic ring including 1 to 2 ring members selected from N, O or S, a phenyl ring, a 5 membered heteroaryl ring including 1 to 2 ring members selected from N, O, or S, or a 6 membered heteroaryl ring including 1 ring member selected from N.

42. The compound of claim 35, wherein:
Each of R_{S3} is independently selected from —F, —Cl, —CH₃, —CH₂CH₃, —CH₂CH₂CH₃, —CH(CH₃)₂, —CH₂Cl, —CHCl₂, —CCl₃, —CH₂F, —CHF₂, —CF₃, —CN, oxo, —NH₂, —NH(CH₃), —N(CH₃)₂, —OH, —O—CH₃, —O—CH₂CH₃, —SH, —S—CH₃, —S—CH₂CH₃, —COOH, —COO(CH₃), —COO(CH₂CH₃), —CH₂OH, —CH₂CH₂OH, —CH(CH₃)OH, —CH₂NH₂, —CH₂CH₂NH₂, or —CH(CH₃)NH₂; preferably, each of R_{S3} is independently selected from —F, —CH₃, —CH₂CH₃, —CF₃, —CN, oxo, —NH₂, —OH, —O—CH₃, —COOH, —COO(CH₃), —CH₂OH, or —CH₂NH₂;
q₁ is selected from 0, 1, or 2;
Each of R_{S4} is independently selected from —F, —Cl, —CH₃, —CH₂CH₃, —CH₂CH₂CH₃, —CH(CH₃)₂, —CH₂Cl, —CHCl₂, —CCl₃, —CH₂F, —CHF₂, —CF₃, —CN, oxo, —NH₂, —NH(CH₃), —N(CH₃)₂, —OH, —O—CH₃, —O—CH₂CH₃, —SH, —S—CH₃, —S—CH₂CH₃, —COOH, —COO(CH₃), —COO(CH₂CH₃), —CH₂OH, —CH₂CH₂OH, —CH(CH₃)OH, —CH₂NH₂, —CH₂CH₂NH₂, or —CH(CH₃)NH₂; preferably, each of R_{S4} is independently selected from —F, —CH₃, —CH₂CH₃, —CF₃, —CN, oxo, —NH₂, —OH, —O—CH₃, —COOH, —COO(CH₃), —CH₂OH, or —CH₂NH₂;
q₂ is selected from 0, 1, or 2;
Each of R_{S5} is independently selected from —F, —Cl, —CH₃, —CH₂CH₃, —CH₂CH₂CH₃, —CH(CH₃)₂, —CH₂Cl, —CHCl₂, —CCl₃, —CH₂F, —CHF₂, —CF₃, —CN, oxo, —NH₂, —NH(CH₃), —N(CH₃)₂, —OH, —O—CH₃, —O—CH₂CH₃, —SH, —S—CH₃, —S—CH₂CH₃, —COOH, —COO(CH₃), —COO(CH₂CH₃), —CH₂OH, —CH₂CH₂OH, —CH(CH₃)OH, —CH₂NH₂, —CH₂CH₂NH₂, or —CH(CH₃)NH₂, preferably, each of R_{S5} is independently selected from —F, —CH₃, —CH₂CH₃, —CF₃, —CN, oxo, —NH₂, —OH, —O—CH₃, —OCH₂—COO(CH₂CH₃), —COOH, —COO(CH₃), —CH₂OH, or —CH₂NH₂;
q₃ is selected from 0, 1, or 2;
Each of R_{S6} is independently selected from —F, —Cl, —CH₃, —CH₂CH₃, —CH₂CH₂CH₃, —CH(CH₃)₂, —CH₂Cl, —CHCl₂, —CCl₃, —CH₂F, —CHF₂, —CF₃, —CN, oxo, —NH₂, —NH(CH₃), —N(CH₃)₂, —OH, —O—CH₃, —O—CH₂CH₃, —SH, —S—CH₂CH₃, —COOH, —COO(CH₃), —COO(CH₂CH₃), —CH₂OH, —CH₂CH₂OH, —CH(CH₃)OH, —CH₂NH₂, —CH₂CH₂NH₂, or —CH(CH₃)NH₂, preferably, each of R_{S6} is independently selected from —F, —CH₃, —CH₂CH₃, —CF₃, —CN, oxo, —NH₂, —OH, —O—CH₃, —OCH₂—COO(CH₂CH₃), —COOH, —COO(CH₃), —CH₂OH, or —CH₂NH₂;
q₄ is selected from 0, 1, or 2; Each of R_{S7} is independently selected from —F, —Cl, —CH₃, —CH₂CH₃, —CH₂CH₂CH₃, —CH(CH₃)₂, —CH₂Cl, —CHCl₂, —CCl₃, —CH₂F, —CHF₂, —CF₃, —CN, oxo, —NH₂, —NH(CH₃), —N(CH₃)₂, —OH, —O—CH₃, —O—CH₂CH₃, —SH, —S—CH₂CH₃, —COOH, —COO(CH₃), —COO(CH₂CH₃), —CH₂OH, —CH₂CH₂OH, —CH(CH₃)OH, —CH₂NH₂, —CH₂CH₂NH₂, or —CH(CH₃)NH₂, preferably, each of R_{S7} is independently selected from —F, —CH₃, —CH₂CH₃, —CF₃, —CN, oxo, —NH₂, —OH, —O—CH₃, —COOH, —COO(CH₃), —CH₂OH, or —CH₂NH₂;
q₅ is selected from 0, 1, or 2;
Each of R_{S8} at each occurrence is independently selected from halogen, —C₁₋₆alkyl, haloC₁₋₆alkyl, haloC₁₋₆alkoxy, —CN, oxo, —NR_{N1}R_{N2}, —OR_{N1}, —C(═O)R_{N1}, —C(═O)OR_{N1}, —OC(═O)R_{N1}, —C(═O)NR_{N1}R_{N2}, —NR_{N1}C(═O)R_{N2}, —OC(═O)OR_{N1}, —NR_{N1}C(═O)OR_{N2}, —OC(═O)NR_{N1}R_{N2}, —NR_{N1}C(═O)NR_{N1}R_{N2}, 3-8 membered cycloalkyl, 4-8 membered heterocyclyl containing 1, 2 or 3 heteroatoms selected from N, O or S, phenyl or 5-6 membered heteroaryl containing 1, 2 or 3 heteroatoms selected from N, O or S, wherein said —C₁₋₆alkyl, haloC₁₋₆alkyl, haloC₁₋₆alkoxy, 3-8 membered cycloalkyl, 4-8 membered heterocyclyl, phenyl or 5-6 membered heteroaryl is optionally independently substituted with 1, 2 or 3 substituents selected from halogen, —C₁₋₆alkyl, haloC₁₋₆alkyl, haloC₁₋₆alkoxy, —CN, oxo, —NR_{N1}R_{N2}, —OR_{N1}, —C(═O)R_{N1}, —C(═O)OR_{N1}, —OC(═O)R_{N1}, —C(═O)NR_{N1}R_{N2}, —NR_{N1}C(═O)R_{N2}, —OC(═O)OR_{N1}, —NR_{N1}C(═O)OR_{N2}, —OC(═O)NR_{N1}R_{N2}, —NR_{N1}C(═O)NR_{N1}R_{N2}, 3-6 membered cycloalkyl, 4-6 membered heterocyclyl, phenyl or 5-6 membered heteroaryl;
Each of (R_{N1} or R_{N2}) in R_{S8} is independently selected from hydrogen or —C₁₋₆alkyl;
Optionally, (R_{N1} and R_{N2}) in R_{S8} together with the nitrogen atom to which they are both attached form a 3-6 membered heterocyclic ring which is optionally substituted with one or more substituents selected from halogen, —C₁₋₆alkyl, —OH, —SH, —SC₁₋₆alkyl, —NH₂, —NH(C₁₋₆alkyl) or —N(C₁₋₆alkyl)₂;
q₆ is selected from 0, 1, 2 or 3;
Each of R_{S11} is selected from —C₁₋₃alkyl.

43. The compound of claim 35, wherein, the moiety of —X—R₂ or —O—R₂ is selected from any one of the structures in the Table 2 in the description of the present invention.

44. The compound of claim 35, wherein, each of R_{S8} at each occurrence is independently selected from halogen, —C₁₋₆alkyl, haloC₁₋₆alkyl, haloC₁₋₆alkoxy, —CN, oxo, —NR_{N1}R_{N2}, —C(═O)R_{N1}, —C(═O)OR_{N1}, —OC(═O)R_{N1}, —C(═O)NR_{N1}R_{N2}, —NR_{N1}C(═O)R_{N2}, —OC(═O)OR_{N1}, —NR_{N1}C(═O)OR_{N2}, —OC(═O)NR_{N1}R_{N2}, —NR_{N1}C(═O)NR_{N1}R_{N2}, 3-8 membered cycloalkyl, 4-8 membered heterocyclyl containing 1, 2 or 3 heteroatoms selected from N, O or S, phenyl or 5-6 membered heteroaryl containing 1, 2 or 3 heteroatoms selected from N, O or S, wherein said —C₁₋₆alkyl, haloC₁₋₆alkyl, haloC₁₋₆alkoxy, 3-8 membered cycloalkyl, 4-8 membered heterocyclyl, phenyl or 5-6 membered heteroaryl is optionally independently substituted with 1, 2 or 3 substituents selected from halogen, —C₁₋₆alkyl, haloC₁₋₆alkyl, haloC₁₋₆alkoxy, —CN, oxo, —NR_{N1}R_{N2}, —C(═O)R_{N1}, —C(═O)OR_{N1}, —OC(═O)R_{N1}, —C(═O)NR_{N1}R_{N2}, —NR_{N1}C(═O)R_{N2}, —OC(═O)OR_{N1}, —NR_{N1}C(═O)OR_{N2}, —OC(═O)NR_{N1}R_{N2}, —NR_{N1}C(═O)NR_{N1}R_{N2}, 3-6 membered cycloalkyl, 4-6 membered heterocyclyl, phenyl or 5-6 membered heteroaryl;
Each of (R_{N1} or R_{N2}) in R_{S8} is independently selected from hydrogen or —C₁₋₆alkyl;
Optionally, (R_{N1} and R_{N2}) in R_{S8} together with the nitrogen atom to which they are both attached form a 3-6 membered heterocyclic ring which is optionally substituted with one or more substituents selected from halogen, —C₁₋₆alkyl, —OH, —SH, —SC₁₋₆alkyl, —NH₂, —NH(C₁₋₆alkyl) or —N(C₁₋₆alkyl)₂;
q₆ is selected from 0, 1, 2 or 3.

45. The compound of claim 35, wherein, each of R_{S8} at each occurrence is independently selected from —F; methyl; —CF₃; —CN; oxo; —OH; —NH₂; —OCH₃; —NHC(═O)CH₃; —NHC(═O)OCH₃; —OC(═O)N(CH₃)₂; —NHC(═O)N(CH₃)₂; 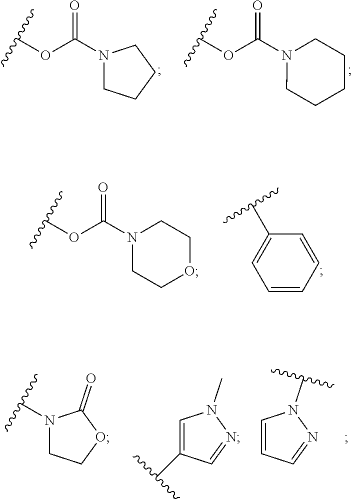
or methyl substituted with —F, —Cl, methyl, —CF₃, —CN, oxo, —OH, —NH₂, —OCH₃, —NHC(═O)CH₃, —NHC(═O)OCH₃, —OC(═O)N(CH₃)₂, —NHC(═O)N(CH₃)₂,

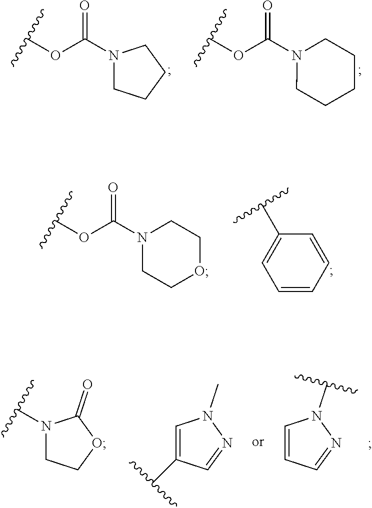

46. The compound of claim 35, wherein, each of R_{S8} at each occurrence is independently selected from —F, methyl, —CF₃, —CN, oxo, —OH, —NH₂, —OCH₃, —NHC(═O)CH₃, —NHC(═O)OCH₃, —OC(═O)N(CH₃)₂, —NHC(═O)N(CH₃)₂,

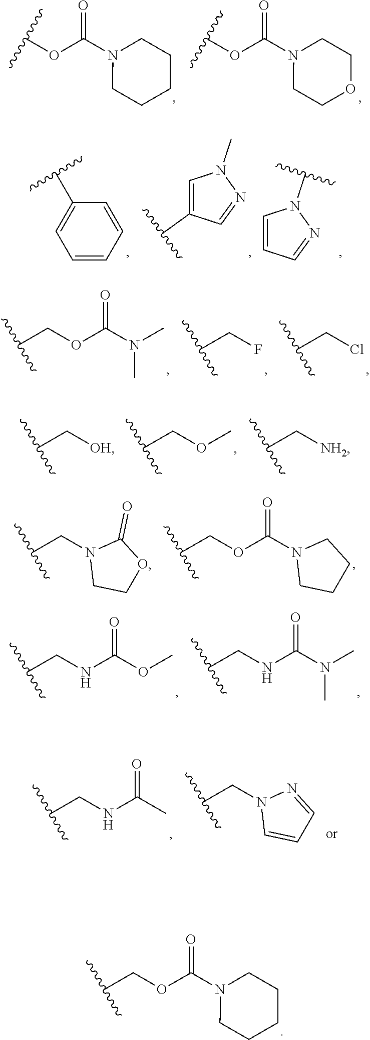

47. The compound of claim 35, wherein, the moiety of —X—R₂ or —O—R₂ is selected from

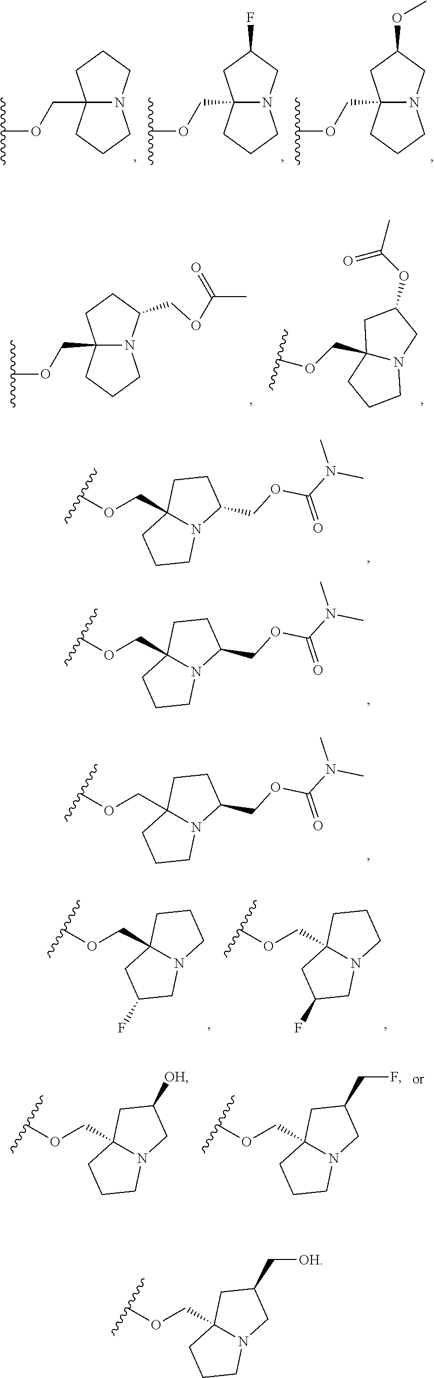

48. The compound of claim 35, wherein, R₄ is selected from 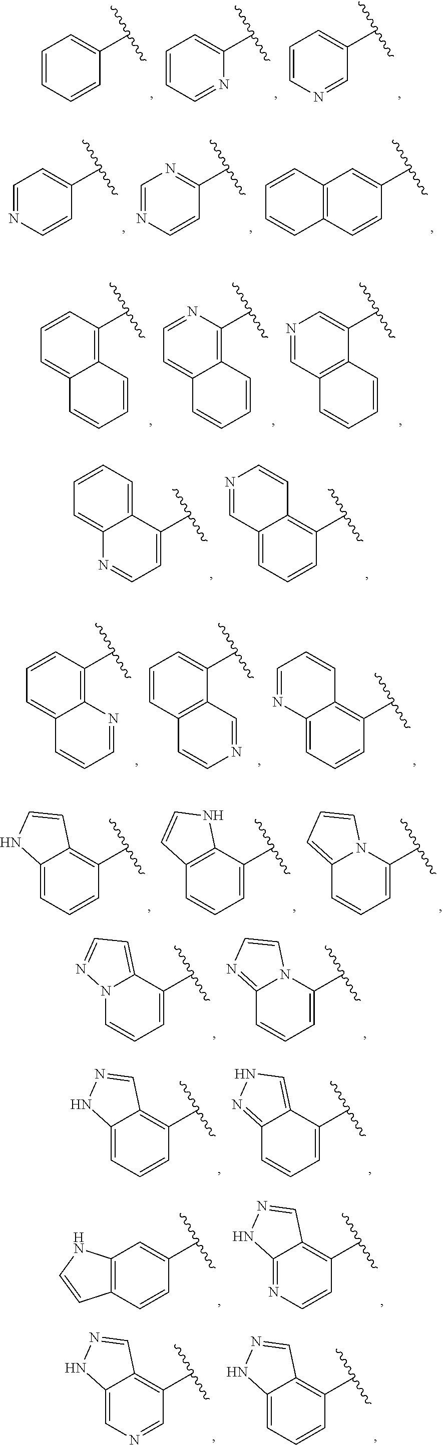 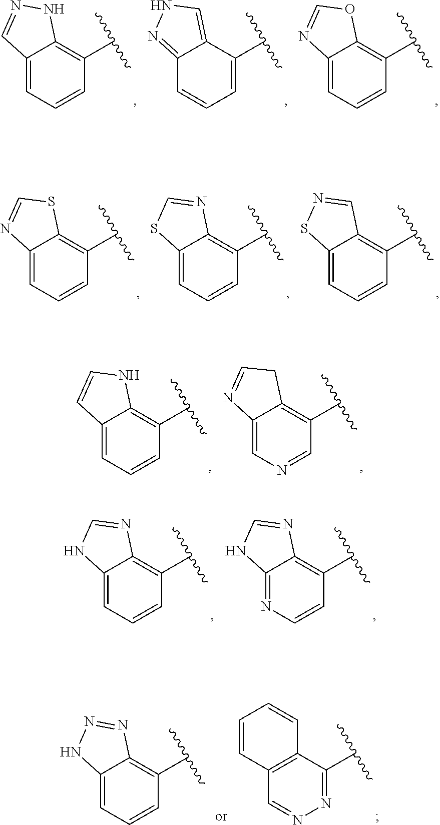
wherein said R₄ is independently optionally substituted with 1, 2, 3, 4, 5 or 6 R₄ₐ;
Each of R₄ₐ is independently selected from —F, —Cl, —C₁₋₃alkyl, haloC₁₋₃alkyl, haloC₁₋₃alkoxy, —C₂₋₃alkenyl, —C₂₋₃alkynyl, —CN, —NH₂, —NH(C₁₋₃alkyl), —N(C₁₋₃alkyl)₂, —OH, —O(C₁₋₃alkyl), —SH, —S(C₁₋₃alkyl), 3-6 membered cycloalkyl or 3-6 membered heterocyclyl, wherein said —C₁₋₃alkyl, haloC₁₋₃alkyl, haloC₁₋₃alkoxy, —C₂₋₃alkenyl, —C₂₋₆alkynyl, 3-6 membered cycloalkyl or 3-6 membered heterocyclyl is independently optionally substituted with 1, 2 or 3 R_{4b};
Each of R_{4b} is independently selected from —F; —C₁₋₃alkyl; haloC₁₋₃alkyl; —CN; —OH; —NH₂; —NH(C₁₋₃alkyl); —NH(C₁₋₃alkyl)₂; —OC₁₋₃alkyl; or —C₁₋₃alkyl substituted with 1, 2 or 3 substituents selected form —F, haloC₁₋₃alkyl, —CN, —OH, —NH₂, —NH(C₁₋₃alkyl), —NH(C₁₋₃alkyl)₂ or —OC₁₋₃alkyl.

49. The compound of claim 35, wherein, R₄ is selected from 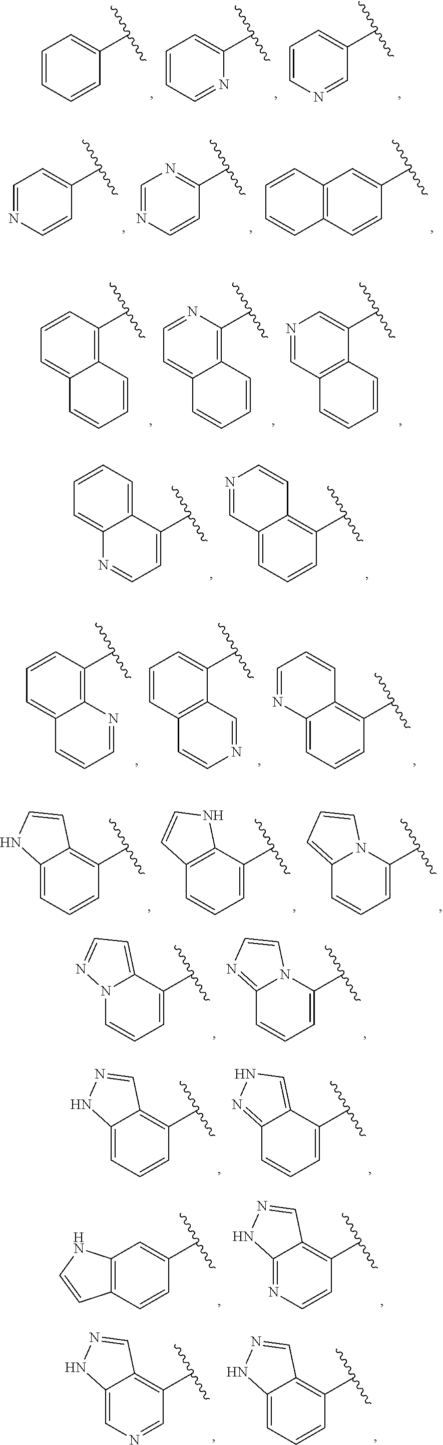 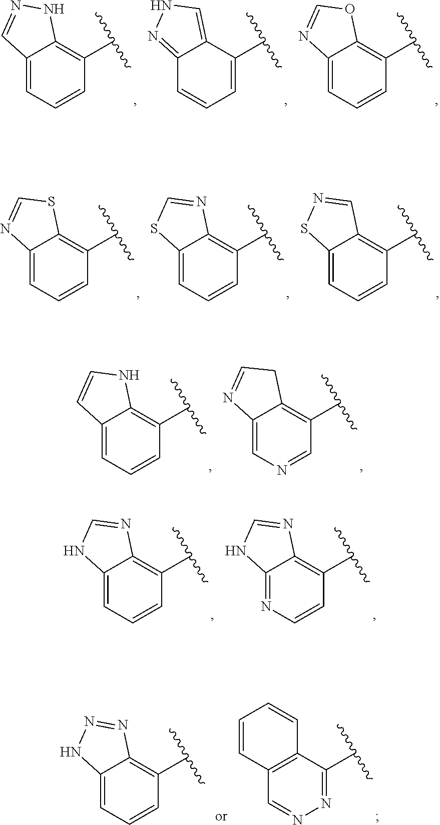
wherein said R₄ is independently optionally substituted with 1, 2 or 3 R₄ₐ;
Each of R₄ₐ is independently selected from —F, —Cl, methyl, ethyl, isopropyl, —CH═CH₂, —C≡CH, —C≡CCH₃, —C≡CD, —CH₂C≡CH, —CHF₂, —CF₃, —CH₂CF₃, —CH₂CHF₂, —CH₂CH₂F, —CH₂CH₂CH₂F, —OCF₃, —CN, —CH₂CH₂CN, —NH₂, —N(CH₃)₂, —NHCH₂CH₃, —CH₂—N(CH₃)₂, —OH, —CH₂OH, —CH₂CH₂OH, —OCH₃, —OC(CH₃)₂, —CH₂CH(CH₃)₂, —CH(CH₃)CH₂CH₃, —CH₂OCH₃, —SH, —SCH₃, —SCF₃, —OCHF₂, —CH(CF₃)OCH₃, —C(CH₃)₂OH, —CF(CH₃)₂, —OCH(CH₃)₂, cyclopropyl,

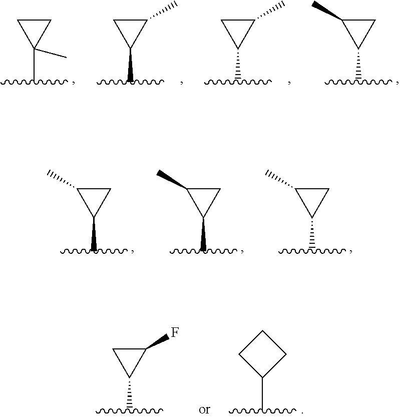

50. A pharmaceutical composition comprising a compound, a stereoisomer thereof, an atropisomer thereof, a pharmaceutically acceptable salt thereof, a pharmaceutically acceptable salt of the stereoisomer thereof or a pharmaceutically acceptable salt of the atropisomer thereof of claim 35, and at least one pharmaceutically acceptable excipient.

51. A method of treating a subject having a cancer related to KRAS G12D mutant protein, said method comprising administering to the subject a therapeutically effective amount of a compound, a stereoisomer thereof, an atropisomer thereof, a pharmaceutically acceptable salt thereof, a pharmaceutically acceptable salt of the stereoisomer thereof or a pharmaceutically acceptable salt of the atropisomer thereof of claim 35.

52. The method for treating cancer of claim 51, wherein the cancer is selected from pancreatic cancer, colorectal cancer, endometrial cancer or lung cancer.

53. The method for treating cancer of claim 52, the lung cancer is selected from non-small cell lung cancer or small cell lung cancer.
